(19) United States
(12) Patent Application Publication
(10) Pub. No.: US 2026/0188423 A1
(43) Pub. Date: Jul. 2, 2026

(54) IDENTIFICATION OF CELL SURFACE ANTIGENS WHICH INDUCE T-CELL RESPONSES AND THEIR USES

(71) Applicant: VERDI SOLUTIONS GMBH, Wien (AT)

(72) Inventors: Julianna Lisziewicz, Wien (AT); Robert Gergely Lisziewicz, Wien (AT)

(73) Assignee: VERDI SOLUTIONS GMBH, Wien (AT)

(21) Appl. No.: 19/127,401

(22) PCT Filed: Nov. 6, 2023
(86) PCT No.: PCT/EP2023/080888

§ 371 Date: May 5, 2025

(30) Foreign Application Priority Data
Nov. 4, 2022 (GB) 22164537

(51) Int. Cl. G16B 20/00; A61K 39/00; A61K 39/12; A61P 37/04

(52) U.S. Cl. CPC G16B 20/00; A61K 39/0011; A61K 39/12; A61P 37/04

(57) ABSTRACT

Disclosed is a computer-implemented method for identifying, for a subject, at least one antigen which is expected to induce a T-cell response to attack unhealthy cells in the subject. The invention further relates to using said antigens for designing personalized vaccines and to the use of such personalized vaccine and personalized vaccine compositions in kits and methods for the treatment of disease.

### CROSS REFERENCE TO RELATED APPLICATIONS

The present application claims the priority benefits of International Patent Application No. PCT/EP2023/080888, filed on Nov. 6, 2023, and claims benefit of GB 2216453.7, filed on Nov. 4, 2022, which are hereby incorporated herein by reference in their entireties.

### FIELD OF THE INVENTION

The present invention relates to a system and a computer-implemented method for predicting the potency of T-cell responses induced by HLA-presented surface antigens for different individuals, in particular but not limited to infectious diseases, cancer, and autoimmune disorders. The prediction may be used to develop a range of personalized medicinal products including vaccines, T-cell therapies, and diagnostic tests. Furthermore, the disclosure details a method of creating personalized vaccines from peptide antigens that encompass a set of highly immunogenic epitopes capable of eliciting robust CD8 and CD4 T-cell responses. Additionally, the techniques provide insights into predicting individual responses to vaccines and T-cell therapies, enhancing the precision and efficacy of personalized medical interventions.

### BACKGROUND OF THE INVENTION

An antigen is defined as a peptide which induces an immune response. It is known that immune responses to illnesses such as cancer and viral infections are regulated by the most polymorphic group of related proteins encoded by the human leukocyte antigen (HLA) which is also known interchangeably as MHC (Major Histocompatibility Complex). After infection, viral proteins are processed to epitopes, which are peptides that bind to an HLA molecule. A sub-set of these epitopes are transported to the cell surface to trigger T-cell receptors (TCR). This mechanism is schematically illustrated in FIG. 1 which shows a TCR in a T-cell being triggered by an epitope 150 which is strongly bound to an HLA-allele of an individual. An antigen may thus be defined by its specificity (i.e. its amino acid sequence) and its potency (i.e. its intensity to trigger a TCR leading to a T-cell response). Once triggered, the responding T-cells proliferate, and after their TCRs recognize infected cells presenting the same epitopes, kill these cells. After the clearance of infected cells, a subset of epitope-specific T-cells forms the memory population that can quickly respond to a new illness which contains the same epitopes.

The HLA-genotype in humans is extremely diverse. An individual inherits from each of their parents an HLA-A, an HLA-B, and an HLA-C allele and thus an individual has six different HLA class I molecules. These HLA class I molecules present epitopes which typically have a length of between 8 to 14 amino acids. These epitopes present to CD8⁺ cytotoxic T-cells. An individual also has HLA class II molecules which present epitopes to CD4⁺ helper T-cells. The epitopes presented by HLA class II molecules are longer than those presented by HLA class I molecules, generally between 11 and 20 amino acids long. HLA class II molecules are encoded by three different loci, HLA-DR, HLA-DQ, and HLA-DP and have two homogenous peptides, an a and B chain (e.g., DQA and DQB). HLA-DR is the most polymorphic with HLA-DRB having more than 700 known alleles and HLA-DRA having only three variants. By contrast, both chains of HLA-DQ and HLA-DP are polymorphic. However, for HLA-DP, only a few alleles are prevalent, most notably the heterodimer DPA1*0103/DPB1*0401 (DP401).

The potency of T-cell responses is one of the most complex traits of human immunology associated with a multitude of diseases, including but not limited to COVID-19 which is caused by the SARS-COV-2 infection. For example, over 1400 epitopes from SARS-COV-2 have been identified that induced T-cell responses in at least one of 1187 individuals (see reference 5 (Bukhari et al)). Experimental methods can only test a small subset of putative epitopes due to specimen limitations, and antigen-specific T-cell responses are not reproducible in different individuals due to HLA heterogeneity and disease variability. Generally, the prior art is thus limited to measuring the potency of few epitopes predicted to bind to one or two HLA molecules. For example, EP3370065 describes a method of identifying a fragment of a polypeptide as immunogenic for a specific human subject when the fragment is capable of binding to at least two HLA molecules of the subject.

Systems to predict epitope-HLA binding have also been developed. For example, reference 5 (Bukhari et al) describes several machine learning (ML) models which are available to predict epitope-HLA binding. One of these models, known as the NetMHCpan-4.1 model and described in more detail in “NetMHCpan-4.1 and NetMHCIIpan-4.0: improved predictions of MHC antigen presentation by concurrent motif deconvolution and integration of MS MHC eluted ligand data” by Reynisson et al published in Nucleic Acids Research published in 2020 has been trained on 850,000 quantitative binding affinity (BA) and mass-spectrometry eluted ligands (EL) to accurately predict the strength of the epitope-HLA interaction. The NetMHCpan-4.1 model computes a score for any paired epitope and HLA allele which is termed an EL-score and which represents the likelihood of the epitope being presented by the HLA allele on the cell surface. However, as noted in reference 7 (Saini et al), such predicted epitopes rarely induce T-cell responses in HLA-allele matched individual.

The present applicant has recognised the need for a new method for determining the T-cell response for an individual.

### SUMMARY OF THE INVENTION

### Summary of Invention

In a first aspect of the invention, there is provided a computer-implemented method for identifying, for a subject, at least one antigen which is expected to induce a T-cell response to attack unhealthy cells in the subject, the method comprising:
receiving subject data identifying multiple major histocompatibility complex, MHC, molecules for the subject;
receiving sequence data for a plurality of epitopes within a protein which is expressed in the unhealthy cells, wherein each epitope is an amino acid sequence within the protein;
obtaining for each epitope in the plurality of epitopes, a potency score which is indicative of the likelihood that each of the identified multiple MHC molecules displays the epitope on their surface;
generating a ranked list of epitopes based on the determined potency scores;
identifying at least one antigen by selecting at least one epitope which is a highly ranked in the ranked list; and
outputting at least one of the ranked list and sequence data for the identified at least one antigen.

In one embodiment, further comprising
selecting multiple epitopes which are highly ranked in the ranked list,
identifying the subitopes of each of the selected epitopes using a directed graph network, and
identifying each subitope which is common to more than one of the selected epitopes.

In one embodiment the first aspect of the invention further comprising identifying the at least one antigen by selecting a common subitope which is highly ranked in the ranked list.

In one embodiment, further comprising identifying the at least one antigen by selecting a longest common subitope.

In one embodiment, further comprising identifying a shortest common subitope as a target sequence.

In one embodiment, obtaining a potency score comprises calculating an epitope weight score for each epitope in the plurality of epitopes by
selecting an epitope from the plurality of epitopes,
obtaining, for each of the identified MHC molecules, a probability score which is indicative of the likelihood that each MHC molecule transports the selected epitope; and
calculating the epitope weight score for the selected epitope by aggregating at least some of the probability scores.

In one embodiment, the method of aspect 4, wherein the epitope weight score is calculated from:

$$E W {S}_{( x )} = {\sum }_{i = 1}^{n} {w}_{i} * E L {S}_{( x , i )} for all {w}_{i} * E L {S}_{( x , i )} > E L T$$

Where x is a peptide, i is between 1 and n with n being the number of identified MHC molecules, ELS_{(x,i)} is the probability score, i.e. eluted ligand score for each MHC molecule and epitope pair, ELT is an eluted ligand threshold and wᵢ is a weighting parameter.

In one embodiment, further comprising calculating at least one additional score for each epitope which is indicative of whether the subitopes of each epitopes are capable of triggering the same T-cell response.

In one embodiment, further comprising calculating at least one additional score for each epitope which is indicative of whether the subitopes of each epitopes are capable of triggering the same T-cell response.

In a second aspect of the invention, there is provided a method of designing a personalized vaccine to induce a T-cell response to attack unhealthy cells in a subject, the method comprising
receiving subject data identifying multiple major histocompatibility complex, MHC, molecules for the subject, wherein the subject data identifies at least one of a set of HLA class I molecules and a set of HLA class II molecules;
receiving sequence data for a plurality of epitopes within a protein expressed in the unhealthy cells wherein each epitope is an amino acid sequence within the protein;
obtaining for each epitope in the plurality of epitopes, a first potency score which is indicative of the likelihood that each of the identified set of HLA class I molecules displays the epitope on their surface;
generating a first ranked list of epitopes based on the determined first potency scores; selecting multiple epitopes which are highly ranked in the first ranked list; identifying the subitopes of each of the selected epitopes using a directed graph network;
identifying each subitope which is common to more than one of the selected epitopes; identifying at least one first antigen by selecting at least one subitope which is itself a highly ranked epitope in the first ranked list; and
outputting the identified at least one first antigen for the personalized vaccine.

In one embodiment further comprising
obtaining for each epitope in the plurality of epitopes, a second potency score which is indicative of the likelihood that each of the identified set of HLA class II molecules displays the epitope on their surface;
generating a second ranked list of epitopes based on the determined second potency scores;
selecting multiple epitopes which are highly ranked in the second ranked list;
identifying the subitopes of each of the selected epitopes using a directed graph network;
identifying each subitope which is common to more than one of the selected epitopes;
identifying at least one second antigen by selecting at least one subitope which is itself a highly ranked epitope in the second ranked list; and
outputting the identified at least one second antigen for the personalized vaccine.

In one embodiment, further comprising
determining whether the identified first antigen is a highly ranked epitope in the second ranked list, and
when the first antigen is a highly ranked epitope in the second ranked list, designing the personalized vaccine based on the first antigen.

In one embodiment, the antigen comprises multiple epitopes which are highly ranked in the ranked lists, capable of being displayed by the subject's MHC class I and/or MHC class II molecules on the cell surface, induce CD4 and/or CD8 T cell responses, and share at least one common sequence capable of triggering the same T-cell response and optionally, wherein the multiple epitopes are overlapping.

In a third aspect of the invention, there is provided a method for determining the potency of an immune response as a control measure for safety and efficacy in a recipient of the personalized vaccine composition comprising the at least one antigen identified using the method of an aspect of the invention, the method comprises:
generating all potential epitopes derived from the sequence of the selected antigen creating a ranked list based on the potency scores of these epitopes,
verifying those epitopes derived from proteins expressed in the unhealthy cells of the recipient, induce potent immune responses, as indicated by their high potency scores, thereby ensuring efficacy,
ensuring that epitopes, derived from the cell penetrating peptide and/or excipient, are immunologically inert as evidenced by their low potency scores, thereby ensuring safety,
confirming that epitopes with high potency scores are not components of proteins expressed in healthy cells, further contributing to the safety of the personalized vaccine.

In a fourth aspect of the invention, there is provided a personalised vaccine composition comprising a peptide antigen derived from a protein expressed in the unhealthy cell of a recipient comprising multiple highly ranked epitopes selected according to the method to an aspect of the invention and, optionally, a cell penetrating peptide and/or excipient.

In one embodiment, further comprising at least two peptide antigens derived from proteins expressed in the unhealthy cells of the recipient both comprising multiple highly ranked epitopes selected according to the method of an aspect of the invention.

In one embodiment, the cell penetrating peptide is positioned between the two antigens.

According to a fifth aspect of the invention, there is provided a method of treating or prevention a disease of a subject comprising administering at least one personalised vaccine according to an aspect of the invention, wherein the at least one personalised vaccine composition is administered alone or in conjunction with an additional therapeutic agent and wherein said administration is concurrently or sequentially, optionally, wherein at least two personalised vaccine compositions are administered.

In one embodiment, the disease is cancer or autoimmune disease, or a viral infection.

According to a sixth aspect of the invention, there is provided a method for inducing antigen-specific immune responses in a subject comprising administering the personalised vaccine composition according to an aspect of the invention to the subject.

According to a seventh aspect of the invention, there is provided a kit comprising the several items required to prepare the personalised vaccine composition for the individual according to an aspect of the invention comprising:
two synthetic peptides wherein each synthetic peptide comprising at least one antigen selected according to an aspect of the invention and further comprising at least a portion of a cell-penetrating peptide; and
means to perform covalent linkage of the two synthetic peptides during the preparation of the personalized vaccine results in reconstitution of the function of the cell-penetrating peptide.

According to an eighth aspect of the invention, there is provided a method of preparing a personalised peptide vaccine or treatment composition comprising preparing a first amino acid sequence and preparing a second amino acid sequence, wherein both amino acid sequences comprises antigens comprising a set of high-ranked epitopes derived from a protein expressed in the unhealthy cells of the individual and at least a portion of a cell penetrating peptide, and covalently linking the first and second amino acid sequences to form a personalised vaccine to reconstitute the function of cell penetrating peptide positioned between the first antigen and the second antigen.

According to the present invention there is provided an apparatus and method as set forth in the appended claims. Other features of the invention will be apparent from the dependent claims, and the description which follows.

We describe a computer-implemented method for identifying, for a subject, at least one antigen which is expected to induce a T-cell response to attack unhealthy (also termed sick or infected) cells in the subject. Each of the unhealthy cells expresses a set of illness associated proteins and thus, the health of a patient can be restored by T cells that destroy specifically the unhealthy cells and leaves the healthy cells intact. The method comprises: receiving subject data identifying multiple major histocompatibility complex, MHC, molecules for the subject; receiving sequence data for a plurality of epitopes within the protein, wherein each epitope is an amino acid sequence within the protein; generating for each epitope in the plurality of epitopes, a potency score which is indicative of the likelihood that each of the identified MHC molecules display the epitope on the cell surface; generating a ranked list of epitopes which is a subset of the plurality of epitopes which are ranked based on the determined potency scores; identifying at least one antigen by selecting at least one highly ranked epitope; and outputting at least one of the ranked list and sequence data for the identified at least one antigen.

The illness may be a viral infectious disease such as SARS-COV-2 or cancer. The unhealthy cells may be virus-infected cells or cancer cells. An antigen may be defined as a peptide which induces an immune response. An antigen may be defined by its specificity (i.e. its amino acid sequence) and its potency (i.e. its intensity to trigger a T-cell receptor (TCR) leading to a T-cell response).

The method may further comprise selecting multiple epitopes which are highly ranked in the ranked list, identifying the subitopes of each of the selected epitopes using a directed graph network, and identifying each subitope which is common to more than one of the selected epitopes. Each subitope which is common to more than one epitope may be termed a core amino acid sequence.

The at least one antigen may be identified by selecting a common subitope (also termed sub-epitopes) which is highly ranked in the ranked list (i.e. by selecting a highly ranked core). It will be appreciated that the most common cores are the typically the shortest sequences that recognized by the TCR. A longer core will thus contain multiple smaller cores and may thus be more likely to trigger a strong T-cell response. The method may thus comprise identifying the at least one antigen by selecting a longest epitope containing a set of highly ranked epitopes having the common subitope. Typically, a cell surface antigen is longer than an highly ranked epitope with the subitope. The potency score of such an antigen may be computed by aggregating potency score of the overlapping epitopes with the same score.

The method may also comprise identifying a shortest common subitope as a target sequence of a TCR, for example for T-cell therapy or other treatments as discussed below.

Obtaining a potency score may comprise calculating an epitope weight score for each epitope in the plurality of epitopes. This may comprise selecting an epitope from the plurality of epitopes, obtaining, for each of the identified MHC molecules, a probability score which is indicative of the likelihood that each MHC molecule transports the selected epitope; and calculating the epitope weight score for the selected epitope by aggregating at least some of the probability scores. In other words, each epitope is paired with each autologous MHC molecule to calculate the epitope weight score for an epitope.

Each probability score may be obtained from a database containing probability scores for a plurality of pairs of MHC molecules and epitopes. The probability score used in the calculation of the epitope weight score may be an eluted ligand score (ELS). The eluted ligand score may be calculated using the scoring mechanism (algorithms) taught in “NetMHCpan-4.1 and NetMHCIIpan-4.0: improved predictions of MHC antigen presentation by concurrent motif deconvolution and integration of MS MHC eluted ligand data” by Reynisson et al published in Nucleic Acids Research published in 2020. The algorithms are trained on experimental data including binding affinity between epitopes (ligands), MHC molecules and ligand elution data. Eluted ligands pass through the natural antigen processing and presentation pathway and thus ligand elution data inherently contains information that is not available when only epitope-HLA binding is considered. Furthermore, high-throughput ligand elution assays allow the identification of thousands of natural ligands with a single experiment and thus large training datasets are available. The NetMHC methods described in the paper above, provide an eluted ligand score to estimate the likelihood that the epitope can be eluted from a given MHC molecule and have been shown to perform better in predicting epitopes on cell surface than methods based on binding affinity data. As explained in more detail below, the database may be built by exploiting the large amount of experimental data for the prediction of the density of epitopes presented on the cell surface by the MHC molecules. The database may store an eluted ligand score for each of the paired MHC molecules and epitopes.

As set out above, the epitope weight score EWS may be calculated by aggregating at least some of the probability scores. The EWS may thus be a weighted sum of at least some of the probability scores. The epitope weight score may be expressed as:

$$E W {S}_{( x )} = {\sum }_{i = 1}^{n} {w}_{i} * E L {S}_{( x , i )} for all {w}_{i} * E L {S}_{( x , i )} > E L T$$

where x is a peptide, i is between 1 and n with n being the number of identified MHC molecules, ELS (1) is the probability score, i.e. eluted ligand score for each MHC molecule and epitope pair, ELT is an eluted ligand threshold and wᵢ is a weighting parameter. By using the eluted ligand threshold and weight parameters, only contributing pairs of MHC molecules and epitopes may be included in the epitope weight score. The eluted ligand threshold may be relatively low, e.g. 0.2. In other words, when a pair of MHC molecule and epitope has a weighted eluted ligand score below this threshold, it will be excluded from the epitope weight score because the MHC molecule is unlikely to transfer the epitope to the cell surface.

The method may further comprise calculating at least one additional score for each epitope which takes into account overlapping sub-epitopes (also termed subitopes) which are capable of triggering the same T-cell receptor. A subitope is a fragment of the epitope being considered. The at least one additional score may be selected from a left (or first) subitope score and a right (or second) subitope score which are based on the epitope weight scores for the subitopes in the left and right subgraphs respectively. The left subgraph contains all the subitopes which have one amino acid removed from the right hand-side of the epitope in the layer above and which have a length greater than a sequence threshold. Similarly, the right subgraph contains all the subitopes which have one amino acid removed from the left hand-side of the epitope in the layer above and which have a length greater than a sequence threshold. It will be appreciated that there is overlap between the right and left subgraphs. Thus, the right subitope score (SWS2) may exclude the sub-epitopes which have already been included in the left subitope score (SWS1).

The left subitope score (SWS1) and the right subitope score (SWS2) may be calculated from:

$$SWS {1}_{( {x}_{i} )} = {EWS}_{( {lx}_{i - 1} )} + SWS {1}_{( {lx}_{i - 1} )} + SWS {2}_{( {lx}_{i - 1} )} \\ SWS {2}_{( {x}_{i} )} = {EWS}_{( {rx}_{i - 1} )} + SWS {2}_{( {rx}_{i - 1} )}$$

where xᵢ is an epitope with the length of i, where i has a range of between a to b amino acids, lxᵢ₋₁ is the left sub-epitope of xᵢ and rxᵢ₋₁ is the right sub-epitope of xᵢ and the initial values are SWS1(xₐ)=0, and SWS2(xₐ)=0.

The method may further comprise calculating, for each epitope in the plurality of epitopes, an overall weight score OWS. For example, the overall weight score OWS may be a combination of the epitope weight score with the at least one additional score. The overall weight score OWS may be calculated from:

$$OWS ( x ) = EWS ( x ) + {w}_{i} * SWS 1 ( x ) + {v}_{i} * SWS 2 ( x ) when EWS exceeds an EWS threshold \\ otherwise OWS ( x ) = SWS 1 ( x ) + SWS 2 ( x )$$

where EWS is the epitope weight score, SWS1 is the left subitope score, SWS2 is the right subitope score and \hat{ }\hat{ } and \hat{ }\hat{ } is a weight. The weights determine the contributions of the subitopes. The weights can depend on various aspects, including which sub-epitopes are cut out during the processing stages and the amount in which they are present. Setting the epitope weight score (EWS) threshold may comprise ranking each of the epitopes based on their epitope weight score and selecting the value of the epitope weight score for a particular ranking as the EWS threshold. For example, the particular ranking may be the hundredth epitope when 10,000 epitopes are being considered and lower when fewer epitopes are being considered.

The subject may be an animal or a human. When the subject is a human, the subject data may identify at least part of the human leukocyte antigen (HLA) genotype for the subject. The subject data may identify at least one of a set of HLA class I molecules and a set of HLA class II molecules. The set of HLA class I molecules may comprise six molecules (encoded by three different loci: 2 HLA-A, 2 HLA-B, 2 HLA-C, respectively). The set of HLA class II molecules may comprise twelve molecules (encoded by three different loci HLA-DR, HLA-DQ, and HLA-DP, respectively). The method thus uses multiple HLA molecules (or alleles) rather than a single HLA allele (or a small subset of the HLA alleles). Considering all autologous HLAs reflects the fact that immune responses are based on T-cells responding to epitopes presented by several HLAs.

The data identifying the HLA genotype for the subject may be a plurality of digits (e.g. between a minimum 4 digits and maximum 8 digits for the HLA class I and II molecules). The plurality of digits represent the amino acid sequences of the epitope binding pockets of the HLA molecules. The data identifying the HLA genotype may be received, for example, by a clinician, a nurse or even the subject entering the data into the system. In the era of modern molecular pathology, the entire HLA genome may be sequenced from a single swipe of buccal mucosa or from a small blood sample. The HLA-genotype can be accurately determined using standard techniques. The data identifying the HLA genotype may be the complete sequence of the HLA genes, when it is available.

When the subject data identifies the set of HLA class I molecules, each of the plurality of epitopes may be an amino acid sequence of between 8 to 14 amino acids. When calculating the left and right subitope scores as described above, i has a range of between a to b amino acids and thus a is 8 and bis 14. When the subject data identifies the set of HLA class II molecules, each of the plurality of epitopes is an amino acid sequence of at least 9 amino acids. The maximum length may be 20 amino acids but there may be more than 20 amino acids. In this example, a is 9 and b is 20. Merely as an example, for the SARS-COV-2 protein, there may be 70,000 and 100,000 smaller sequences of subitopes and epitopes to be paired with HLA class I and HLA class II molecules respectively.

The method may further comprise obtaining for each epitope in the plurality of epitopes, a first potency score which is indicative of the likelihood that each of the identified molecules in the set of HLA class I molecules displays the epitope on the cell surface. The method may also comprise obtaining for each epitope in the plurality of epitopes, a second potency score which is indicative of the likelihood that each of the identified molecules in the set of HLA class II molecules displays the epitope on the cell surface. The first and second potency scores may be used to generate first and second ranked lists: one for the epitopes which are highly ranked for the HLA class I molecules and one for the epitopes which are highly ranked for the HLA class II molecules. It will be appreciated that there may be some overlap between the first and second ranked list and a highly ranked epitope which appears in both lists may trigger both CD4+ T-cell responses and CD8+ T-cell responses.

The protein may be any suitable protein expressed in the cells. For example, for T cell based medicinal product development the protein is one that is specifically expressed in diseased cells in order to avoid T-cell mediated killing of healthy cells. The proteins may be expressed in infected cells, like one or more SARS-COV-2 proteins and the illness may be COVID-19. Alternatively, the proteins may be expressed in tumor cells, e.g. AKAP-4, and the illness may be cancer, e.g. metastatic breast cancer.

### Potency of T-Cell Responses

The potency of T-cell responses is one of the most complex traits of human immunology associated with a multitude of diseases, not only cancer and COVID-19. Experimental methods could measure only a small subset of immunogenic epitopes in a subject due to specimen limitation. So far, no methods to rank epitopes of proteins expressed in tumors and infected cells in a subject based on potency to trigger T-cells in an individual have been developed. Similarly, there are no methods to determine the potency of the T-cell responses of individuals to tumors or viruses like SARS-COV-2, and to identify T-cell antigens of individuals that are the top-ranked epitopes most likely induce T-cell responses. Investigation of the T-cell response phenotype as described here required the development of a novel methodology. The breadth and magnitude of T-cell responses against tumor and viral proteins are determined by the highest density (top-ranked) of epitopes capable to activate T-cells.

The extreme diversity of HLA-genotype in humans, through the unique epitope-specific T-cell responses, is essential to maximize the probability that at least some individuals within the general population can mount successful immune responses against an emerging infection, like SARS-COV-2, and survive. The polymorphic HLA-genotype is likely the reason behind the potent CD8+ and CD4+ T-cells responses in asymptomatic individuals infected with SARS-COV-2 or other viruses. It seems likely that subjects with weak T-cell responses experience symptomatic illness (e.g. COVID-19).

The method may thus comprise determining an aggregated score using at least some of the potency weight scores for the plurality of epitopes; and predicting the subject's response to an illness by comparing the aggregated score to an overall threshold. Thus, according to another aspect, we describe a method of predicting a subject's response to an illness which results in unhealthy cells in the subject, the method comprising receiving subject data identifying multiple major histocompatibility complex, MHC, molecules for the subject; receiving sequence data for a plurality of epitopes within a protein which is expressed in the unhealthy cells, wherein each epitope is an amino acid sequence within the protein; obtaining for each epitope in the plurality of epitopes, a potency score which is indicative of the likelihood that each of the identified MHC molecules displays the epitope on their surface; generating a ranked list of epitopes based on the determined potency scores; determining an aggregated score using at least some of the potency weight scores for the plurality of epitopes and outputting at least one of the ranked list and the aggregated score.

Determining the aggregated score may comprise summing the potency scores which are higher than a potency threshold. In other words, the sum may be considered to be a weighted sum with the weight set to 0 for scores which are below the threshold and the weight set to 1 for scores above the threshold. Setting the potency threshold may comprise ranking each of the epitopes based on their potency score and selecting the value of the potency score for a particular ranking as the potency threshold. Alternatively, the aggregated score may the average potency score for a set number (e.g. 100) of the highest ranked epitopes (when ranked by their score).

As another alternative, the aggregated score may be determined by a machine learning model. The aggregated score may be a probability that the epitope is an immunogenic epitope for that individual. The machine learning model may determine the aggregated score based on a feature vector which includes some or all of the epitope weight score, the left subitope score, the right subitope score and the overall weight score. Other features may be input to the feature vector, including “log-fold change” which quantifies the potency of the epitope compared to no epitope or a baseline indicating the expansion of antigen-specific T-cells after an infection in the body. Other features may include for example the dominant core for each of the HLA-A, HLA B and HLA-C class I alleles of the subject. The core is the pattern or sequence of amino acids in the epitope which is recognized by the TCR.

### Stratifying Patients

A subject may be classified as having a weak T-cell response when the aggregated score is below the potency threshold. A weak T-cell response is indicative of a poor outcome

to the illness. In other words, the subject is at high risk from the illness and thus requires treatment and/or vaccination. A subject may be classified as having a good T-cell response when the aggregated score is above or equal to the potency threshold. A good T-cell response is indicative of a good outcome to the illness and may thus be able to avoid treatment and/or vaccination.

For some illnesses, such as COVID-19 and cancer, the challenge is how to vaccinate individuals with high risk of developing the disease. One strategy may be to induce T-cell responses in individuals having an HLA genotype that only support weak T-cell responses to kill infected cells and tumor cells. These individuals likely experience symptomatic disease. COVID-19 patients with symptomatic disease are also more likely to transmit the virus to more people than someone without symptoms. According to another aspect, there is thus a method of stratifying a group of subjects for vaccination by predicting each subject's response to the tumor or illness as described above and classifying subjects having aggregated scores below the potency threshold as a priority for vaccination.

The method may similarly be used to stratify patient groups to determine treatment. According to another aspect, there is thus a method of stratifying a group of subjects for treatment by predicting each subject's response to the tumor or illness as described above and classifying subjects having aggregated scores below the potency threshold as a priority for treatment. Similarly, according to another aspect, there is a method of treating a subject, the method comprising: predicting the subject's response to the illness as described above. A first type of treatment may be recommended when the potency score is below the overall threshold and a second type of treatment which is less aggressive than the first type of treatment may be recommended when the potency is above or equal to the overall threshold. The aggressive treatment may be a T-cell therapy or a vaccine in combination with other treatment approaches, e.g. drug combinations. The less aggressive treatment may be a vaccine alone, or no treatment, because the immune system likely eliminates the illness.

### Vaccination

According to another aspect, there is a method of designing a personalized vaccine to induce a T-cell response against a protein associated with an illness, the method comprising selecting one or more of the at least one output antigens generated by the method described above to incorporate in the vaccine. As explained above, the subject data may identify at least one of a set of HLA class I molecules and a set of HLA class II molecules. Two or more of T-cell antigens are included into the personal vaccines to induce both CD8+ and CD4+ T-cell responses against proteins expressed in the unhealthy cells, such as tumor or infected cells. Such a personal vaccine may be designed by covalently joining two T-cell antigens derived from the same protein or different proteins simultaneously expressed in the sick cells.

Alternatively, the personal vaccine may be designed by identifying T-cell antigens with a set of epitopes which can be simultaneously presented by both HLA class I and class II molecules of the subject. Such antigens presented by the autologous HLA molecules contain the “core” that stimulate the same TCR.

According to another aspect, there is method of designing a personalized vaccine to induce a T-cell response to attack unhealthy cells in a subject, the method comprising receiving subject data identifying multiple major histocompatibility complex, MHC, molecules for the subject, wherein the subject data identifies at least one of a set of HLA class I molecules and a set of HLA class II molecules; receiving sequence data for a plurality of epitopes within a protein expressed in the unhealthy cells wherein each epitope is an amino acid sequence within the protein; obtaining for each epitope in the plurality of epitopes, a first potency score which is indicative of the likelihood that each of the identified set of HLA class I molecules displays the epitope on the cell surface; generating a first ranked list of epitopes based on the determined first potency scores; selecting multiple epitopes which are highly ranked in the first ranked list, identifying the subitopes of each of the selected epitopes using a directed graph network, identifying each subitope which is common to more than one of the selected epitopes; identifying at least one first antigen by selecting at least one subitope which is itself a highly ranked epitope in the first ranked list; and outputting the identified at least one first antigen for the personalized vaccine for preventing or treating the disease caused by the unhealthy cells of the subject.

The method may then comprise determining obtaining for each epitope in the plurality of epitopes, a second potency score which is indicative of the likelihood that each of the identified set of HLA class II molecules displays the epitope on their surface; generating a second ranked list of epitopes based on the determined second potency scores; selecting multiple epitopes which are highly ranked in the second ranked list; identifying the subitopes of each of the selected epitopes using a directed graph network or other methods; identifying each subitope which is common to more than one of the selected epitopes; identifying at least one second antigen by selecting at least one subitope which is itself a highly ranked epitope in the second ranked list; and outputting the identified at least one second antigen for the personalized vaccine. As an alternative, there are occasions when the first highly ranked epitope set overlaps with the second highly ranked epitope set. In this example, the antigen to be included in the personalized vaccine may be comprises a set of epitopes presented by both class I and class II HLA molecules on the cell surface. Such singe antigen can stimulate both CD8 and CD4 T cell responses in the subject.

Another aspect of the invention is a computer-implemented method for developing an industrial vaccine (or general-purpose vaccine) which is suitable to protect a sub-group of patients. The industrial vaccine may be designed by selecting a plurality of personal vaccines, selecting at least one antigen which is more frequently used in the personalized vaccines and including the at least one selected antigen in the general-purpose vaccine.

Although each personal vaccine is designed to induce potent T-cell responses against tumor-specific antigens in the associated individual (e.g. HLA-genotyped matched subjects), the general vaccine may not be appropriate for each individual. Accordingly, before administering the vaccine, the potency of the general vaccine may be assessed for the new subject. The subgroup of patients who likely respond to the vaccine can be identified by the computer-implemented method invented here. The potential responder must have HLA genotype that support potent T-cell responses against at least one epitope in the industrial vaccine. Thus, the method above can be used to predict the subject's response to the industrial vaccine using the HLA genotype of the subject. For example, the method of predicting may comprise receiving subject data identifying multiple major histocompatibility complex, MHC, molecules for the subject; receiving sequence data for each epitope within the vaccine, wherein each epitope is an amino acid sequence; obtaining for each epitope in the vaccine, a potency score which is indicative of the likelihood that each of the identified MHC molecules displays the epitope on the cell surface; and predicting the subject's response to the vaccine based on the potency score. The prediction based on the potency score may be done as described above, e.g. by a machine learning model and/or by comparison with a threshold.

### T-Cell Therapy

According to another aspect, there is a method of identifying targets for T-cell therapy derived from a protein expressed in unhealthy cells, the method comprising receiving subject data identifying multiple major histocompatibility complex, MHC, molecules for the subject; receiving sequence data for a plurality of epitopes within the protein, wherein each epitope is an amino acid sequence within the protein; obtaining for each epitope in the plurality of epitopes, a potency score which is indicative of the likelihood that each of the identified multiple MHC molecules displays the epitope on their surface; selecting multiple epitopes which are highly ranked in the ranked list, identifying the subitopes of each of the selected epitopes using a directed graph network, identifying each subitope which is common to more than one of the selected epitopes; determining a length and the sequence each identified subitope and outputting a subitope with the shortest length (also called a core) as the target of TCR.

As explained above, the subject data may identify at least one of a set of HLA class I molecules and a set of HLA class II molecules. One or more cores are selected as target for the personal T-cell therapy. Such a personal T-cell therapy may be designed against two or more cores of T-cell antigens derived from the same protein or different proteins

simultaneously expressed in the unhealthy cells. Alternatively, the personal T-cell therapy may be designed by targeting the core of T-cell antigens which are simultaneously presented by both HLA class I and class II molecules. Such antigens presented by the autologous HLA molecules contain the “core” that stimulate the TCR of the therapeutic T-cells.

Another aspect of the invention is a computer-implemented method for developing an industrial T-cell therapy (or general-purpose T-cell therapy) which is suitable in a sub-group of patients. The industrial T-cell therapy may be designed by selecting a plurality of targets for personal T-cell therapies, selecting at least one target which is more frequently used in the personalized T-cell therapies and including the at least one selected target in the general-purpose T-cell therapy.

Although each personal T-cell therapy is designed to kill cells expressing tumor-specific antigens in the associated individual, the general T-cell therapy may not be appropriate for each individual. Accordingly, before administering the T-cell therapy, the potency of the T-cell therapy may be assessed for the subject. The subgroup of patients who likely respond to the T-cell therapy can be identified by the computer-implemented method invented here. The potential responder must have HLA genotype that support at least one highly ranked epitope targeted by the T-cell therapy. Thus, the method above can be used to predict the subject's response to the industrial T-cell therapy using the HLA genotype of the subject. For example, the method of predicting the subject response to a T-cell therapy may comprise receiving subject data identifying multiple major histocompatibility complex, MHC, molecules for the subject; receiving sequence data for each epitope targeted by the T-cell therapy, wherein each epitope is an amino acid sequence; obtaining for each epitope targeted by the T-cell therapy a potency score which is indicative of the likelihood that each of the identified MHC molecules displays the epitope on the cell surface; and predicting the subject's response to the T-cell therapy based on the potency score. The prediction based on the potency score may be done as described above, e.g. by a machine learning model and/or by comparison with a threshold.

### Personalised Vaccines and Treatment Compositions and Method of Testing the Safety and Efficacy in the Recipient

According to one aspect of the invention, there is provided a personalised vaccine or treatment composition prepared according to the method of an aspect of the invention described above and, optionally, a cell penetrating peptide.

According to another aspect, there is provided a personalised vaccine or treatment composition prepared according to the methods of any other aspect of the invention. In one embodiment, the personalised vaccine or treatment composition comprises at least one

peptide antigen derived from a protein expressed in an unhealthy cell of the subject wherein the peptide antigen comprises multiple highly ranked epitopes that is capable of being displayed by the subject's HLA class I and/or HLA class II molecules on the cell surface, induces a CD4 and/or CD8 T cell response and shares at least one common sequence and optionally a cell penetrating peptide.

In a further embodiment, the cell penetrating peptide is an immunologically inert cell penetrating peptide. Immunologically inert cell penetrating peptide comprises multiple putative epitopes, wherein none of these putative epitopes are in highly ranked in the first or second list according to the computer implemented method invented here, therefore unlikely induce immune responses in the subject. One example of an immunologically inert cell penetrating peptide is a peptide consist of at least 8-mer polyarginine.

In one embodiment, the personalised vaccine or treatment composition comprises at least two peptide antigens, wherein both antigens consist multiple highly ranked epitopes, at least one antigen which induces a CD4 T-cell response and at least one antigen which induces a CD8 T-cell response. In a further embodiment, the personalised vaccine or treatment composition comprises at least two antigens, wherein at least one antigen induce both a CD4 T-cell response and a CD8 T-cell response. In one embodiment, the cell penetrating peptide is positioned between the at least two antigens.

According to another aspect of the invention, there is a method of determining the potency of immune responses in the recipient of the personalized vaccine composition, serving as a control measure for safety and efficacy. The method comprises generating all potential epitopes derived from the sequence of the selected antigen, creating a ranked list based on the potency scores of these epitopes, verifying those epitopes derived from proteins expressed in the unhealthy cells of the recipient, induce potent immune responses, as indicated by their high potency scores, thereby ensuring efficacy, ensuring that epitopes, derived from the cell penetrating peptide and/or excipient, are immunologically inert as evidenced by their low potency scores, thereby ensuring safety, confirming that epitopes with high potency scores are not components of proteins expressed in healthy cells, further contributing to the safety of the personalized vaccine.

### Methods of Treatment

According to another aspect, there may be a method of treating a subject with a vaccine or T cell therapy described above. The method may comprise predicting the subject's response to the vaccine or T cell therapy and selecting the subject for treatment when the subject is predicted to respond. According to another aspect, there may be a method of treating a subject with a vaccine which may be a personalised vaccine, or a group vaccine as described above. The method may comprise predicting the subject's response to the vaccine and selecting the subject for treatment when the subject is predicted to respond well to the vaccine.

In a further aspect of the invention, there is provided a method of treating or preventing a disease of the subject, comprising administering a personalised vaccine or treatment composition according to any of the other aspects of the invention to the subject.

In one embodiment, the personalised vaccine or treatment composition is administered in conjunction with an additional therapeutic agent and wherein said administration is concurrently or sequentially. In a further embodiment, the at least two personalised vaccine or treatment compositions comprising different peptide antigens comprising a set of highly ranked epitopes are administered to the subject concurrently or sequentially. In a yet further embodiment, the personalised vaccine or treatment composition is administered with an adjuvant and wherein administration is concurrently or sequentially.

In one embodiment, the disease is cancer, autoimmune disease, or viral infection.

In another aspect of the invention, there is provided a method for inducing antigen-specific immunity in a subject comprising administering the personalised vaccine or treatment composition according to any of the other aspect of the invention to the subject.

In another aspect of the invention, there is provided a personalised vaccine or treatment composition according to any of the other aspects of the invention for use in the prevention or treatment of a disease.

### Vaccine Preparation and Manufacture

In another aspect of the invention, there is provided a method of preparing a personalised vaccine or treatment composition comprising preparing a first amino acid sequence and preparing a second amino acid sequence, wherein the first amino acid sequences comprises a first peptide antigen which induces a CD4 and/or CD8 T-cell response and at least a portion of a cell penetrating peptide, wherein the second amino acid sequences comprises a second peptide antigen which induces a CD4 and/or CD8 T-cell response and at least a portion of a cell penetrating peptide, and covalently linking the first and second amino acid sequences to form a personalised vaccine comprising a cell penetrating peptide positioned between the first antigen and the second antigen.

In another aspect of the invention, there is provided a kit for the preparation of the personalised vaccine or treatment composition according to any of the other aspects of the invention, optionally, wherein the kit further comprising a pharmaceutically acceptable excipient, and, further optionally, including instructions for use.

In another aspect of the invention, the kit comprising the several items required to prepare the personalised vaccine composition for the individual comprising two synthetic peptides wherein each synthetic peptide comprising at least one selected antigen and further comprising at least a portion of a cell-penetrating peptide; and covalent linkage of the two synthetic peptides during the preparation of the personalized vaccine in order to result in reconstitution of the function of the cell-penetrating peptide.

### BRIEF DESCRIPTION OF THE DRAWINGS

For a better understanding of the invention, and to show how embodiments of the same may be carried into effect, reference will now be made, by way of example only, to the accompanying diagrammatic drawings in which:

FIG. 1 is a schematic illustration the state-of-the-art showing an HLA allele transport an epitope to a cell surface and a T cell receptor (TCR) triggered by the epitope;

FIG. 2a is a flowchart illustrating the key steps for investigating the potency of T-cell responses;

FIG. 2b is a schematic illustration of the algorithm of the computer-implemented method for identification of cell surface antigen comprising a set of highly ranked overlapping epitopes presented by the HLA molecules of the subject and the T cell receptor (TCR) triggered by the “core” of overlapping epitopes;

FIG. 3 is a multi-levelled directed graph network useful to identify and manage epitopes and subepitope relations;

FIG. 4 is a flowchart of the scores which may be calculated in scoring module of the system of FIG. 2a;

FIG. 5a is a flowchart of an example process for selecting a core, e.g. for a vaccine;

FIG. 5b is a flowchart showing how the selected cores may be used in a vaccine;

FIG. 5c is a flowchart of an example process for selecting a core, e.g. for a therapy;

FIG. 6 is a block diagram of a system for implementing the methods described;

FIGS. 7a and 7b illustrate the data sets which may be used for class-I HLAS;

FIG. 7c illustrates the data sets which may be used for class-II HLAS;

FIG. 8a is a schematic illustration of a system incorporating a machine learning model which is used to verify the methods described;

FIG. 8b is a flowchart showing how the machine learning model of FIG. 8a is trained and used;

FIGS. 9a and 9b plot the ROC and PR curves respectively for the current method and two comparison methods;

FIG. 9c is a bar chart comparing the mean ranked accuracy by the current method and the EL-Max model for the top-10, top-20, top-50 and top-100 epitopes for each individual averaged across all individuals;

FIGS. 9d to 9f compare the accuracy, precision and recall metrics for the top-20 ranked epitopes across individuals;

FIG. 10a plots the mean ranked accuracy for all individuals for the top-1, top-2, top-3, top-5 and top-10 ranked epitopes using the proposed method (VERDI) and the EL Max model for comparison;

FIG. 10b plots the mean PU metric for all individuals for the top-1, top-2, top-3, top-5 and top-10 ranked epitopes using the proposed method (VERDI) and the EL Max model for comparison;

FIG. 10c plots the number of individuals against the ranked accuracy for the top-3 rankings using the proposed method (VERDI) and the EL Max model for comparison;

FIG. 10d plots the number of individuals against the PU metric for the top-3 rankings using the proposed method (VERDI) and the EL Max model for comparison;

FIGS. 11a and 11b plot the potency against specificity for the top-50 ranked epitopes as 9-mer cores for two anonymised individuals from the Adaptive dataset; and

FIGS. 11c and 11d plot the number of individuals (as a percentage) in a population against average potency across the top-50 ranked epitopes.

FIG. 12a. VERDI Vaccine composition: two cell surface antigen comprising a set of highly ranked overlapping epitopes of the subject selected using the method invented here and optionally an immunologically inert cell penetrating peptide; the mechanism of induction of CD8 cytotoxic and CD4 helper T-cell responses.

FIG. 12b. illustration of cellular uptake of the VERDI Vaccine composition designed with model antigens compared to control vaccines. The figure illustrates the cellular uptake of the VERDI Vaccine and control vaccines in HeLa cells after 30 minutes and 120 minutes of incubation. Cellular uptake was visualized using fluorescence microscopy. The results clearly depict a substantial increase in the uptake of the VERDI Vaccine compared to the control vaccines. The enhanced cellular delivery highlights the potential efficacy of the VERDI Vaccine in immunization.

FIG. 13a. Structure of E7 (HPV16)-specific VERDI Vaccine personalized for the HLA genotype of one of the inventor.

FIG. 13b. Quantification of cellular uptake of personalized HPV-specific VERDI Vaccine (blue) compared to control (red) after 30 minutes incubation with human cells. The cellular uptake was assessed by flow cytometry, and the intensity of fluorescence was measured as an indicator of vaccine uptake by the cells. The results demonstrate a significantly higher uptake of the VERDI Vaccine (blue) compared to the control (red), indicating its enhanced cellular delivery and potential efficacy in HPV-specific immunization.

FIG. 14a. Structure of AKAP-specific VERDI Vaccine personalized for the HLA genotype of one of the inventor.

FIG. 14b. Quantification of cellular uptake of personalized AKAP-4-specific VERDI Vaccine (blue) compared to control (red). The cellular uptake was assessed by flow cytometry, and the intensity of fluorescence was measured as an indicator of vaccine uptake by the cells. The results demonstrate a significantly higher uptake of the VERDI Vaccine (blue) compared to the control (red), indicating its enhanced cellular delivery and potential efficacy in AKAP-specific immunization.

FIG. 15a. Illustration of dendritic cell uptake of VERDI Vaccine designed with model antigens compared to control vaccines. The figure illustrates the cellular uptake of the VERDI Vaccine and control vaccines in dendritic cells after 120 minutes of incubation. Cellular uptake was visualized using fluorescence microscopy. The results clearly depict a substantial increase in the uptake of the VERDI Vaccine compared to the control vaccines. This enhanced dendritic cell delivery of VERDI Vaccines similarly to HeLa cells and suggest the potential efficacy of the VERDI Vaccine in immunization.

FIG. 15b. Illustration of the T cell response diagnostic test of a cancer patient. Several proteins expressed specifically in the tumor (CEP55, BIRC5, ATAD2, WT1) were identified by transcriptome analysis from the tumor biopsy of the patient. The diagram shows the potency (y axis) of the top-ranked epitopes (epitope location on the protein sequence is indicated on the x axis) identified with the method invented here. Top-ranked epitopes presented on the cell surface by HLA class I molecules (blue) of the patient are involved in the CD8 responses. Top-ranked epitopes presented on the cell surface by HLA class II molecules (orange) of the patient are involved in the CD4 responses.

FIG. 16a. Illustration of the cell surface antigen selection for a cancer patient using the method and the T cell response diagnostic test invented here. The selected potent antigens are indicated with green are derived from CEP55, BIRC5, ATAD2, WT1 proteins (FIG. 22b). These antigens comprise a set of highly ranked epitopes involved in the CD8 and CD4 T cell responses of the patient. The selection of antigens is based on the number and the potency of the epitopes predicted by the computer implemented method invented here.

FIG. 16b. Illustration of the cell surface antigen selection for the Murcia patient suffering in metastatic Signet Ring Cell adenocarcinoma using the method and the T cell response diagnostic test invented here. The selected potent antigens useful for personalized VERDI Vaccine design are indicated with green, derived from KKLC1 and SSX4 tumor-specific proteins. These antigens of this patient comprise a set of highly ranked epitopes involved in the CD8 and CD4 T cell responses as indicated with blue and orange (location on the protein and potency of the epitopes). The selection of the antigens is based on the number and the potency of the epitopes predicted by the computer implemented method invented here.

FIG. 17. T Cell Responses in the Murcia Patient Pre and Post Personalized VERDI Vaccination. 10 different VERDI Vaccines were administered to the Murcia patient a single time. The QuantiFERON test quantifies the cumulative CD4 and CD8 responses elicited by ten distinct vaccines. Notably, the two KKLC1-specific VERDI vaccines (C1, C2, serving as positive controls) consistently induced robust T cell responses following a single administration. In stark contrast, the SSX4-specific VERDI vaccines (C4, C5, utilized as negative controls) failed to generate detectable responses. This evidence supports a compelling hypothesis: The KKLC1 antigen identified by the computer-implemented method invented here induced the immune responses against KKLC1-specific protein expressed in the patient tumor cells, while the lack of detectable responses to SSX4-specific vaccines suggests that the patient's tumor does not express this specific target. The transcriptome analysis results of tumor biopsy taken after vaccinations confirmed the absence of tumor cells expressing any of the proteins targeted by the personalised VERDI vaccines.

FIG. 18. Blood biomarker responses in the Murcia Patient Pre and Post Personalized VERDI Vaccination. A high level of alkaline phosphatase is common among bone metastasis patients. High level of carbohydrate antigen 19-9 (CA 19-9) also predicts clinicopathological status, recurrence, and prognosis of gastric cancer.

FIG. 19a. Patient journey during personalized VERDI Vaccination

FIG. 19b. Illustration of the predictive diagnosis and vaccine design software

FIG. 20a. Illustration of tumor transcriptome analysis software as feeding the diagnostic software

FIG. 20b. Secure web-based application to download the cancer patient's predictive diagnostic and vaccine design results and upload clinical data.

### DESCRIPTION OF THE PREFERRED EMBODIMENTS

As described below, we investigate the potency of T-cell responses to proteins expressed in the cells after ranking all the putative epitopes by predicted density on the cell surface (e.g. tumor cells or infected cells, including for example the SARS-COV-2 infection). A computer implemented method is used to determine the potency of all viral epitopes presented by a plurality of HLA alleles, encoding the 6 HLA class I alleles and/or 12 class II molecules. As explained below, the method exploits a large amount of experimental data to evaluate epitope densities on the cell surface of an individual and rank the epitopes which induce a T-cell response. The analysis method described below may be termed VERDI (Viral Epitopes Ranked by Digital Intelligence). As demonstrated, the method is rapid and accurate and provides a complete profiling of T-cell responses of an individual against one or more proteins. The method may thus be applied to develop a range of novel medicinal products, including vaccines that induce potent T-cell response in the 12 HLA-allele-matched recipients, precision vaccines that induce T-cell responses in a selected sub-groups, companion diagnostic test for the vaccines to predict the potency of the vaccine induced T-cell responses in the recipient, T-cell therapies targeting high-density epitopes with the same “core”, and diagnostic tests for the HLA-genotype predisposition of an individual to illnesses/diseases, including cancers, infectious, and autoimmune diseases like COVID-19.

Predictive Diagnosis of Antigen-Specific T-Cell Responses with VERDI

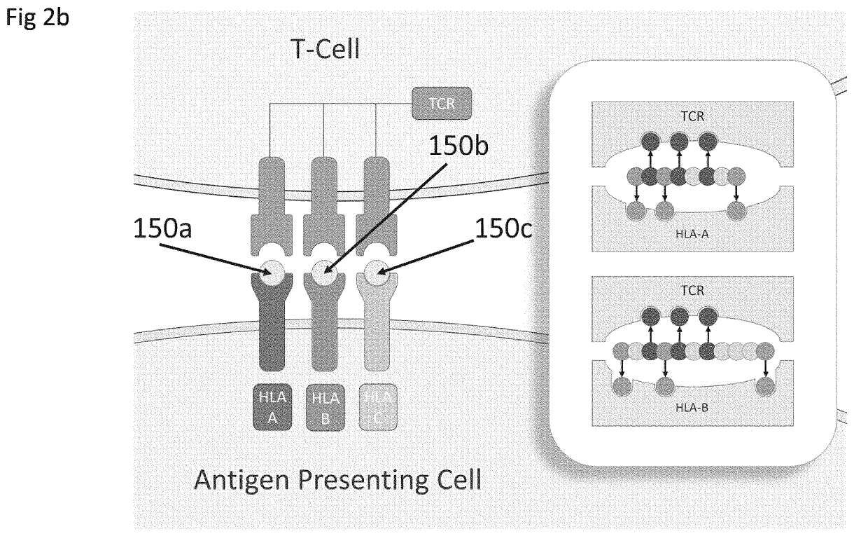

FIG. 2a is a flowchart representing a schematic overview of the method used in the predictive diagnosis of antigen-specific T-cell responses. As shown, there are two separate inputs: the HLA genotype data for a test subject which is obtained in step S210 and an input epitope which is obtained in step S212. As shown in the Figure, the data may be obtained simultaneously but it will be appreciated that it may be obtained sequentially in any order. The HLA genotype data may be obtained using any suitable technique, including the common HLA genotype diagnostic test used for transplantation. The HLA genotype data may include the 4-digit HLA class I genotype data representing the amino acid sequence of the peptide-binding pockets of HLA molecules and/or the 4-digit HLA class II genotype data representing the amino acid sequence of the peptide-binding pockets of HLA molecules. The full set of HLA genotype data is listed in Table 1 below and may be collected together with the ID of the person from whom it is collected and optionally the region from which they come:

Table 1a - Database of HLA genotype data for individuals
in any population (e.g. training or validation)

| | |
|---|---|
| Name | Description |

HLA class I alleles

| | |
|---|---|
| HLA_A_1 | First HLA-A of the person |
| HLA_A_2 | Second HLA-A of the person |
| HLA_B_1 | First HLA-B of the person |
| HLA_B_2 | Second HLA-B of the person |
| HLA_C_1 | First HLA-C of the person |
| HLA_C_2 | Second HLA-C of the person |

HLA class II alleles
(HLA-DR can pair only with the same alpha chain)

| | |
|---|---|
| HLA_DPA1_1 | First alpha chain of HLA-DP of the person |
| HLA_DPA1_2 | Second alpha chain of HLA-DP of the person |
| HLA_DPB1_1 | First beta chain of HLA-DP of the person |
| HLA_DPB1_2 | Second beta chain of HLA-DP of the person |
| HLA_DQA1_1 | First alpha chain of HLA-DQ of the person |
| HLA_DQA1_2 | Second alpha chain HLA-DQ of the person |
| HLA_DQB1_1 | First beta chain of HLA-DQ of the person |
| HLA_DQB1_2 | Second beta chain HLA-DQ of the person |
| HLA_DRB1_1 | First beta chain of HLA-DR of the person |
| HLA_DRB1_2 | Second beta chain HLA-DR of the person |
| HLA_DRBX_1 | Third beta chain of HLA-DR of the person |
| HLA_DRBX_2 | Fourth beta chain HLA-DR of the person |

Table 1b - HLA genotype data for two individuals in the database

| Sample | Person 1 | | Person 2 | |
|---|---|---|---|---|
| Allele | Allele 1 | Allele 2 | Allele 1 | Allele 2 |
| HLA-A | HLA-A*03.01 | HLA-A*68.01 | HLA-A*11.01 | HLA-A*33.01 |
| HLA-B | HLA-B*35.03 | HLA-B*38.01 | HLA-B*14.02 | HLA-B*49.01 |
| HLA-C | HLA-C*04.01 | HLA-C*12.03 | HLA-C*07.01 | HLA-C*08.02 |
| HLA- | HLA- | HLA- | HLA- | HLA- |
| DPA1 | DPA1*01.03 | DPA1*01.03 | DPA1*01.03 | DPA1*02.02 |
| HLA- | HLA- | HLA- | HLA- | HLA- |
| DPB1 | DPB1*03.01 | DPB1*104.01 | DPB1*02.01 | DPB1*05.01 |
| HLA- | HLA- | HLA- | HLA- | HLA- |
| DQA1 | DQA1*01.02 | DQA1*05.05 | DQA1*01.01 | DQA1*02.01 |
| HLA- | HLA- | HLA- | HLA- | HLA- |
| DQB1 | DQB1*03.01 | DQB1*06.04 | DQB1*02.02 | DQB1*07.01 |
| HLA- | HLA- | HLA- | | |
| DRB3 | DQB3*02.02 | DQB3*03.01 | | |
| HLA- | | | HLA- | HLA- |
| DRB4 | | | DQB4*01.01 | DQB4*01.01 |
| HLA- | | | | |
| DRB5 | | | | |

Since HLA allele concordance between transplant recipient and donors is essential for clinical outcome, the 4-digit HLA class I data is routinely performed from blood or buccal swab samples in clinics, diagnostic laboratories, and by stem cell donor registries. The currently available methods are reviewed in “Bioinformatics Strategies, Challenges and Opportunities for Next Generation Sequencing-Based HLA Genotyping” by Klasberg et al published in Transfusion Medicine and Hemotherapy in 2019. The data may be obtained for any individual, including an individual whose T-cell response is to be classified for the purposes of making vaccines for the individual, matching existing vaccines to a group of individuals by predicting the potency of T-cell responses induced by a vaccine, prioritize vaccinations, matching TCR-T-cell therapy to recipients, and determine predisposition to certain diseases.

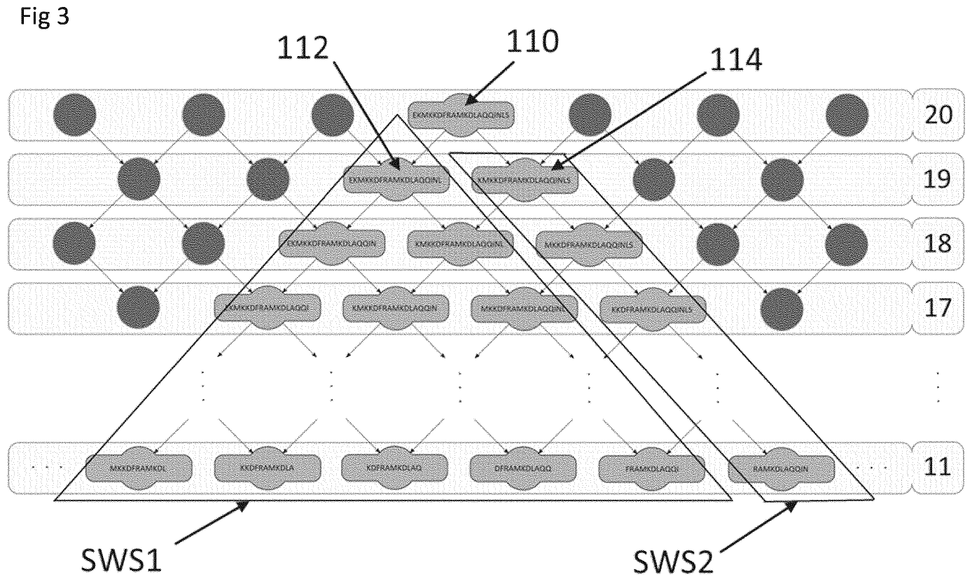

The epitope is determined by the illness being considered. For example, when considering a T-cell response to SARS-COV-2, the epitope may be one of the 70,000 epitopes which are derived from the viral proteins expressed in the infected cells. Each epitope may be input at step S212 with its sub-epitopes and these are defined in more detail in FIG. 3. For example, when considering HLA class I data, the input epitope may be a sequence of 8 to 14 amino acids and for an epitope having 14 amino acids, its sub-epitopes may be sequences of 8 to 13 amino acids.

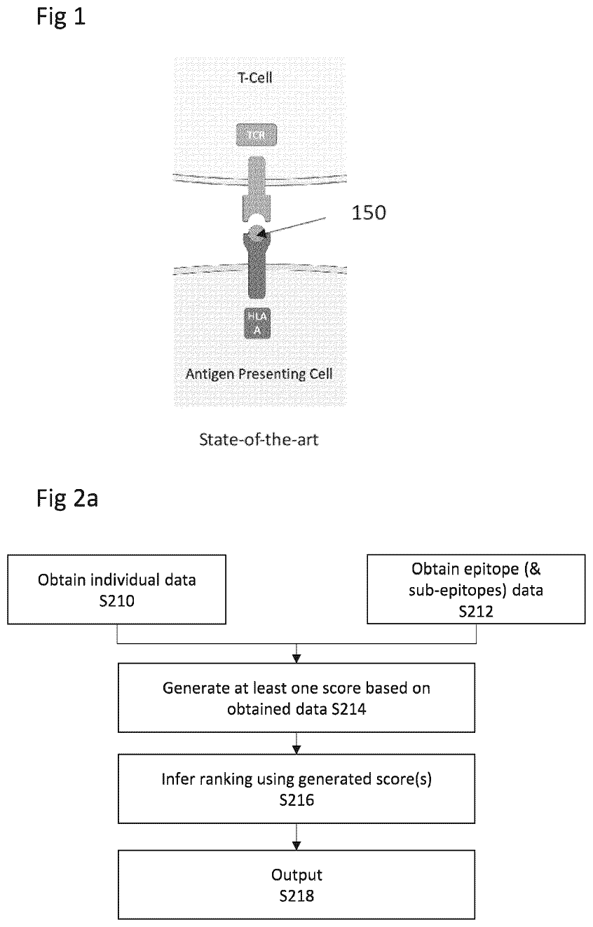

The separate inputs are input as a pair of epitope and HLA data to generate at least one score at step S214 for each pair. For example, when considering HLA class I molecules, six scores may be calculated as described below. Each score may be derived from an EL score generated by the NetMCHPan4.1 or any other suitable method. As described in the background section and illustrated in FIG. 1, HLAs transport epitopes derived from a protein to the surface of the cells to induce T-cell responses against the cell expressing that protein. In the background method, each HLA is typically considered in isolation. FIG. 2b illustrates the biological mechanism which is represented in the computer implemented method of FIG. 2a.

FIG. 2b shows that as in FIG. 1, there is an antigen presenting cell and a T-cell. However, instead of considering a single HLA molecule, FIG. 2b illustrates that multiple HLA molecules are considered. For the case of reference, one of each of the HLA-A, HLA B and HLA-C class I alleles is shown and each one presents an epitope 150a, 150b, 150c. These are overlapping epitopes with a common core that stimulates the same TCR. The TCR core is illustrated in the insert which shows the detail of the binding between the TCR core (red) and the 6 HLA class I molecules of the individual. T-cells of the individual respond sequentially as the core repeatedly triggers the TCR. The core-TCR binding is much more important for triggering a TCR than the HLA-TCR binding, and thus a TCR may be triggered by a core presented by any HLA molecules (class I and class II). As explained in more detail below, the current method and system discovers different lengths of epitopes which are transported to the cell surface by autologous HLAs. Overlapping epitopes with the same core are identified and from these overlapping epitopes the T-cell antigens to be included in the vaccine are identified as well as the T-cell antigens to be targeted by T-cell therapy. Although FIG. 2b illustrates only HLA class I molecules, it will be appreciated that the method is applicable for identifying both CD8 and CD4 T-cell antigens and cores of them.

Returning to FIG. 2a, steps S212 and S214 are repeated for multiple input epitopes. The at least one score generated in step S214 for each paired epitope and individual, is used in step S216 to rank each of the multiple input epitopes to generate a ranked list. The ranked list may comprise only the top-ranked epitopes, e.g. the top 50 or top 100 epitopes. The ranked list defines the T-cell antigen repertoire for an individual and can be used to predict the strength of the T-cell responses of an individual to a specific illness such as SARS-COV-2 or cancer. At step S218, there is an output of the ranking which may include the specificity of each epitope (namely the sequence of amino acids in the epitope) and the potency of each epitope (e.g. based on the at least one score which has been generated).

FIG. 3 shows a multi-levelled directed graph network which can be used to identify and manage epitope and sub-epitope relations. Every node (e.g. 110, 112, 114) identifies a peptide and each peptide is distributed into a different level based on its amino acid length. Every node that is not on the lowest level is considered to be a parent node and every parent node have a left-child and a right-child, each of which are one amino acid shorter than the parent node. The left child omits the prefix (i.e. first amino acid) of the parent peptide.

The right child omits the suffix (i.e. last amino acid) of the parent peptide. Parent-child relations are defined by edges with the source being the parent and the sink being the child.

Merely as an example, the parent node 110 could be the input epitope EKMKKDFRAMKDLAQQINLS (SEQ ID NO: 1111) which is a CD4 epitope. The left child 112 for this parent node is EKMKKDFRAMKDLAQQINL (SEQ ID NO: 1112) and the right child 114 is KMKKDFRAMKDLAQQINLS (SEQ ID NO: 113). Each child is also a node and has a left and right child. The epitope EKMKKDFRAMKDLAQQINLS (SEQ ID NO: 1114) is a 20mer peptide and the shortest length for CD4 in the method implemented in FIG. 2a is 11 at present which means that the graph of all sub-epitopes for this input epitope will have ten levels including the parent node. The uppermost level has the longest amino acid length which is 20 and the lowest level has the shortest amino acid length which is 11. The lowest level of the graph will be different for CD8 and CD4 epitopes. The shortest length for each CD4 sub-epitope is 11; and the shortest length for a CD8 sub-epitope is currently 8.

### Score Calculations

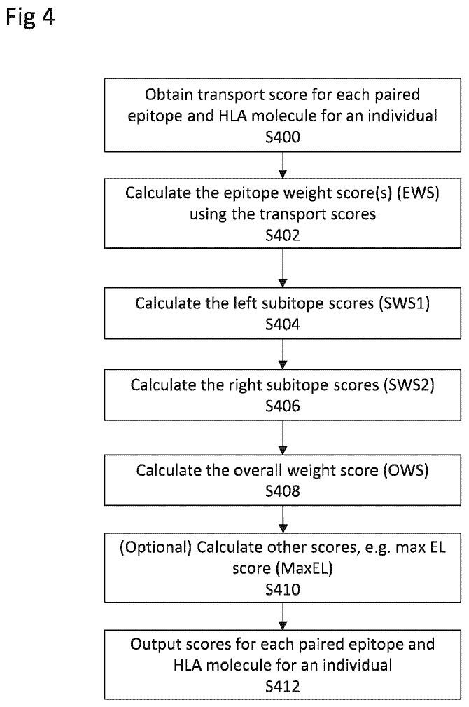

FIG. 4 is a flowchart illustrating the scores which may be calculated in the method of FIG. 2a. Each input epitope is paired with each HLA allele (class I or class II separately). For each pair, a transport score is obtained (step S400). The aim of the transport score is to identify either the likelihood that an epitope is presented by an HLA allele at the surface of the cell (i.e. transported by the HLA) or the amounts of epitopes that the HLA alleles are most likely to transport to the cell surface. Both types of HLA alleles transport and present peptides from degraded proteins on the cell surface but the proteins are degraded differently in the cells. In other words, the transport score may be defined as the contribution of the HLA-allele to present the epitope on the cell surface.

The transport score may be the Eluted Ligand Score (EL-score) which is predicted by the scoring mechanism taught in “NetMHCpan-4.1 and NetMHCIIpan-4.0: improved predictions of MHC antigen presentation by concurrent motif deconvolution and integration of MS MHC eluted ligand data” by Reynisson et al published in Nucleic Acids Research published in 2020. This system exploits the largest collection of experimental data, including data from eluted ligands (EL), that identifies the epitopes on the cell surface capable to transmit signals from HLAs to T-cell receptors (TCRs). The EL-score may be considered to be a measure of the efficiency of the transport that is proportional to the epitope density on the cell surface. The EL-score may also be considered to be the probability or likelihood of epitope transport. The EL-score may have a value between 0 and 1. Optionally, the ID of each epitope and HLA allele may be stored with the associated EL-score and such a data set may be termed a ligand data set.

When using all the HLA class I alleles, there will be six transport scores (or EL scores—the terms can be used interchangeably) calculated for each epitope in step S400. Similarly, when using all the HLA class II alleles, there will be twelve transport scores per epitope calculated in step S400. Accordingly, at step S402, the number of scores which are input to the feature vector may be reduced by aggregating at least some of the transport scores. The aggregated transport scores may be termed epitope weight scores (EWS). Aggregation may be done for each locus of the HLA genotype data. In other words, for the HLA class I data, there may be a first epitope weight score for the HLA-A locus, a second epitope weight score for the HLA-B locus, and a third epitope weight score for the HLA-C locus. Similarly, for the HLA class II data, there may be a first epitope weight score for the HLA-DP locus, a second epitope weight score for the HLA-DQ locus, and a third epitope weight score for the HLA-DR locus.

When calculating each EWS, only contributing pairs may be selected to reduce the complexity of the calculation. Such a selection may also exclude HLA alleles that do not bind to the epitope. A contributing HLA-epitope pair may be defined as one in which the weighted EL score is above an EL threshold (ELT) and thus only the weighted EL scores above an EL threshold are summed. Merely as an example, the EL threshold may be set at 0.2 which will typically filter out low scoring HLA-epitope pairs which are unlikely to transfer to the cell surface. Each EWS may thus be expressed as:

$${EWS}_{( x )} = {\sum }_{i = 1}^{n} {w}_{i} * {ELS}_{( x , i )} for all {w}_{i} * {ELS}_{( x , i )} > ELT$$

where x is a peptide, i is between 1 and n with n being the number of autologous HLA alleles which are being grouped together (normally 2 for each HLA class I molecule or 4 for each HLA class II molecule), ELT is the threshold and wᵢ is a parameter weighting the HLAs. The EL-score may be a probability score and thus may be between 0 and 1. Thus, for the HLA class I alleles, the value of each EWS (EWS_A, EWS_B, EWS_C) is between 0 and 2 and similarly for the 12 HLA class II alleles, the value of each EWS (EWS_DP, EWS_DQ, EWS_DR) is between 0 and 4.

It will be appreciated that other aggregate scores could be generated. For example, a first EWS could aggregate all the EL scores for the HLA class I molecules and may thus range in value from 0 to 6. Such an EWS may be considered to be a measure of the likelihood that the epitope is processed, transported and presented by each of the 6 HLA class I molecules to T-cells. Similarly, a second EWS could aggregate all the EL scores for the HLA class II molecules and may thus range in value from 0 to 12. Such a second EWS may be considered to be a measure of the likelihood that the epitope is processed, transported and presented by each of the 12 HLA class II molecules to T-cells. Any or all of the EWS values may optionally be used to rank the epitopes with the higher-ranking epitopes being more likely to be eliciting T-cell responses.

Merely as an example, the EWS scores for some of the epitopes derived from proteins expressed in tumor cells which are presented by HLA Class I and II molecules of a patient with metastatic breast cancer are shown in the tables below. The epitopes are ranked by their EWS score.

Top-ranked HLA class I epitopes from proteins expressed specifically
in breast cancer cells of a woman with metastatic breast cancer

| | Tumor Protein Name | | |
|---|---|---|---|
| UNIPROT | (expressed only in | | |
| ID | tumo rcell) | Epitope | EWS Class I |
| P16422 | EPCAM | YVDEKAPEF (SEQ ID | 2.9 |
| | | NO: 1) | |
| P43362 | MAGE9 | SVDPAQLEF (SEQ ID | 2.3 |
| | | NO: 2) | |
| Q9BQY4 | RHXF2 | MPPFPPPSL (SEQ ID | 2.3 |
| | | NO: 3) | |
| Q5JQC9 | AKAP4 | APSDPVSVL (SEQ ID | 2.3 |
| | | NO: 4) | |
| Q5JQC9 | AKAP4 | KPIPASVVL (SEQ ID | 2.1 |
| | | NO: 5) | |
| Q8N4B4 | FBX39 | SLDEELHLL (SEQ ID | 2.0 |
| | | NO: 6) | |
| P78395 | PRAME | SPEPLQALL (SEQ ID | 2.0 |
| | | NO: 7) | |
| Q8N4B4 | FBX39 | FFFERIMKY (SEQ ID | 2.0 |
| | | NO: 8 | |
| Q9BQY4 | RHXF2 | FPMPPFPPPSL (SEQ | 2.0 |
| | | ID NO: 9) | |
| P43363 | MAGAA | EVDPTGHSF (SEQ ID | 2.0 |
| | | NO: 10) | |
| Q8TC59 | PIWL2 | TPSRGPPQL (SEQ ID | 2.0 |
| | | NO: 11) | |

Top-ranked HLA class II epitopes from proteins expressed
in tumor cells of a woman with metastatic breast cancer

| | Tumor Protein | | |
|---|---|---|---|
| UNIPROT | Name (expressed | | EWS |
| ID | only in tumor cell) | Epitope | Class II |
| Q8TC59 | PIWL2 | KKDFRAMKDLAQQIN | 3.7 |
| | | (SEQ ID NO: 12) | |
| P43364 | MAGAB | MKVLEYIANANGRDP | 3.0 |
| | | (SEQ ID NO: 13) | |
| Q8NA03 | FSIP1 | VKGYELAVTQHQQLA | 2.4 |
| | | (SEQ ID NO: 14) | |
| Q9BXX3 | AN30A | VEFLLIKNANANAVNK | 2.3 |
| | | (SEQ ID NO: 15) | |
| O60271 | JP4 | SLPQKYKQVTNGQGENK | 2.2 |
| | | (SEQ ID NO: 16) | |
| P43363 | MAGAA | SPSVVASLPLDQSDE | 2.2 |
| | | (SEQ ID NO: 17) | |
| Q8NA03 | FSIP1 | VPVKGYELAVTQHQQ | 2.1 |
| | | (SEQ ID NO: 18) | |
| Q9BXX3 | AN30A | KPAIEMQNSVPNKAF | 2.1 |
| | | (SEQ ID NO: 19) | |
| Q8NA03 | FSIP1 | DDPLYGISVSLSSED | 2.0 |
| | | (SEQ ID NO: 20) | |
| P16422 | EPCAM | DVAYYFEKDVKGESL | 2.0 |
| | | (SEQ ID NO: 21) | |

The high-ranked epitopes presented by HLA class I molecules are likely to induce cytotoxic CD8 T-cell responses. The high-ranked epitopes presented by HLA class II molecules are likely to induce CD4 T-cell responses.

For the peptides have a length greater than the shortest number, e.g. at least 9 when considering HLA class I alleles and at least 12 when considering HLA class II alleles, additional scores may also be calculated. These scores may be termed left and right subitope scores and are calculated at steps S406 and S408 respectively. The left subitope score, Subitope WeightScore_1 (SWS1), is the sum of the Epitope WeightScores of the epitopes in the left subgraph (LEWS). The right subitope score, Subitope WeightScore_2 (SWS2) is the sum of the EpitopeWeightScores of the epitopes in the right subgraph (REWS) excluding the epitopes which are also part of the left subgraph (LEWS).

Returning to FIG. 3, it can be seen that the left subgraph contains all the sub-epitopes which have one amino acid removed from the right hand-side of the peptide in the layer above. Similarly, the right subgraph contains all the sub-epitopes which have one amino acid removed from the left hand-side of the peptide in the layer above. It will be appreciated that there is overlap between the right and left sub-graphs. Thus, the right subitope score, (SWS2) excludes the sub-epitopes which have already been included in the left subitope score (SWS1). The sub-epitopes which are included in each score are shown in FIG. 3.

Thus, returning to FIG. 4, for each individual, at step S404 the Subitope WeightScore_1 (SWS1) is calculated and at step S406, the Subitope WeightScore_2 (SWS2) is calculated. The calculations may be defined as:

$$SWS {1}_{( {x}_{i} )} = {EWS}_{( l {x}_{i - 1} )} + SWS {1}_{( l {x}_{i - 1} )} + SWS {2}_{( l {x}_{i - 1} )} \\ SWS {2}_{( {x}_{i} )} = {EWS}_{( {rx}_{i - 1} )} + SWS {2}_{( {rx}_{i - 1} )}$$

where xᵢ is an epitope with the length of i, where i is between 9 and 14 for Class I and between 12 and 20 for Class 2, lxᵢ₋₁ is the left sub-epitope of xᵢ and rxᵢ₋₁ is the right sub-epitope of xᵢ. The initial values for Class I are SWS1(x₈)=0, SWS2(x₈)=0 and for class II are SWS1(x₁₁)=0, SWS2(x₁₁)=0.

At step S408, there is also the option to calculate a score entitled “OveallWeightScore” (OWS). For the shortest possible peptides, the overall weight score may be calculated as:

$$OWS = {\sum }_{j = 1}^{m} {EWS}_{j} for all {EWS}_{j} > EWT$$

where m is the number of previously calculated EWS values, e.g. 3 for each of the HLA class I or class II molecules and EWT is an EWS threshold. If there are no EWS values above the threshold, the OWS is set to zero.

For each peptide having a length greater than the minimum (e.g. at least 9 when considering HLA class I alleles and at least 12 when considering HLA class II alleles,) the OverallWeightScore (OWS) is the sum of each EpitopeWeightScore, the SubitopeWeightScore_1 and the SubitopeWeightScore_2. In other words:

$$OWS = {\sum }_{j = 1}^{m} ( {EWS}_{j} + {w}_{j} * SWS {1}_{j} + {v}_{j} * SWS {2}_{j} )$$

where wⱼ and vₖ are weights which determines the contributions of the sub-epitopes. The weights can depend on various aspects, including which sub-epitopes are cut out during the processing stages and the amount in which they are present.

Optionally, when generating this overall score, only the EWS scores above an EWS threshold may be included. Where a threshold is used, the OWS may be calculated as follows:

$$OWS = {\sum }_{j = 1}^{m} ( {EWS}_{j} + {w}_{j} * SWS {1}_{j} + {v}_{j} * SWS {2}_{j} ) of all {EWS}_{j} > EWT$$

For example, the EWS threshold may be set by ranking the epitopes for a population based on their EWS and then selecting as the EWS threshold, the EWS of the particular epitope, e.g. the 100th epitope when say 10,000 amino acids are in the set of proteins being analysed like with SARS-COV-2. For shorter length set of proteins e.g. Hepatitis B which is approximately 4,000 amino acids in length, a lower number may be selected. Such EWS thresholds may be a suitable definition for a population. Individuals may have different thresholds, depending on the EWS of their epitopes. The EWS of an epitope is approximating the density of a given epitope on the surface of the cells. The EWS threshold is suitable to compare the epitope density against a set of proteins (e.g. all proteins expressed by a virus including from alternative reading frames) among individuals and among populations. Epitopes above the EWS threshold may be considered for the selection of frequent and high-density epitopes in a population. Merely as an example, the EWS threshold for an individual may be calculated by ranking the epitopes based on their EWS for the individual and using the average value of the EWS for a plurality (e.g. 100) of the highest ranked epitopes. The EWS threshold for a population could then be the average of the all the individual average values for the EWS threshold. In other words, the EWS threshold can be adapted for individuals and each virus being considered. The example scores for epitopes which are presented by HLA class II alleles of an individual is shown in the table below:

Tumour protein (PIWL2) HLA class II epitope
analysis in a patient with metastatic breast cancer

| | | | | | Number | Number |
|---|---|---|---|---|---|---|
| | | | | | of SWS1 | of SWS2 |
| ID | Epitope | EWS | SWS1 | SWS2 | Subitopes | Subitopes |
| 1 | KKDFRAMKDLAQQIN | 3.7 | 17.1 | 4.6 | 8 | 2 |
| | (SEQ ID NO: 22) | | | | | |
| 2 | KKDFRAMKDLAQQ | 3.5 | 3.1 | 3.1 | 2 | 2 |
| | (SEQ ID NO: 23) | | | | | |
| 3 | KKDFRAMKDLAQQI | 3.5 | 9.7 | 3.9 | 5 | 2 |
| | (SEQ ID NO: 24) | | | | | |
| 4 | MKKDFRAMKDLAQQIN | 3.5 | 26.2 | 8.2 | 11 | 3 |
| | (SEQ ID NO: 25) | | | | | |
| 5 | MKKDFRAMKDLAQQ | 3.4 | 5.5 | 6.6 | 3 | 3 |
| | (SEQ ID NO: 26) | | | | | |
| 6 | MKKDFRAMKDLAQQI | 3.3 | 15.5 | 7.4 | 7 | 3 |
| | (SEQ ID NO: 27) | | | | | |
| 7 | KKDFRAMKDLAQQINL | 3.2 | 25.3 | 3.9 | 11 | 2 |
| | (SEQ ID NO: 28) | | | | | |
| 8 | KMKKDFRAMKDLAQQIN | 3.1 | 34.5 | 11.7 | 14 | 4 |
| | (SEQ ID NO: 29) | | | | | |
| 9 | KMKKDFRAMKDLAQQ | 3.1 | 7.6 | 10.0 | 4 | 4 |
| | (SEQ ID NO: 30) | | | | | |
| 10 | KMKKDFRAMKDLAQQI | 3.0 | 20.7 | 10.7 | 9 | 4 |
| | (SEQ ID NO: 31) | | | | | |
| 11 | MKKDFRAMKDLAQQINL | 3.0 | 37.9 | 7.1 | 15 | 3 |
| | (SEQ ID NO: 32) | | | | | |
| 12 | KDFRAMKDLAQQIN | 2.9 | 8.0 | 1.7 | 5 | 1 |
| | (SEQ ID NO: 33) | | | | | |
| 14 | EKMKKDFRAMKDLAQQ | 2.8 | 9.5 | 13.1 | 5 | 5 |
| | (SEQ ID NO: 34) | | | | | |
| 15 | EKMKKDFRAMKDLAQQI | 2.8 | 41.8 | 14.8 | 17 | 5 |
| | N (SEQ ID NO: 35) | | | | | |
| 16 | KKDFRAMKDLAQQINLS | 2.8 | 32.4 | 3.2 | 14 | 2 |
| | (SEQ ID NO: 36) | | | | | |
| 17 | KDFRAMKDLAQQI | 2.7 | 4.2 | 1.1 | 3 | 1 |
| | (SEQ ID NO: 37) | | | | | |
| 18 | KMKKDFRAMKDLAQQIN | 2.7 | 49.3 | 10.1 | 19 | 4 |
| | L (SEQ ID NO: 38 | | | | | |
| 19 | EKMKKDFRAMKDLAQQI | 2.7 | 25.4 | 13.8 | 11 | 5 |
| | (SEQ ID NO: 39) | | | | | |

Among the overlapping potent epitopes of this cancer patient the core is the 12 amino acids long KDFRAMKDLAQQ (SEQ ID NO: 40). This core contains the pattern that triggers a TCR as described above in relation to FIG. 2b.

Optionally, other scores may be calculated at step S410. For example, any or all of the maximum EL score, the minimum EL score and the average EL score may be determined. The scores calculated at any of steps S402 to S408 may then be output at step S412 to characterise the potency of each epitope to trigger a T-cell response, and also the potency of triggering T-cell responses by the top ranked set of epitopes derived from a viral proteome, for the individual in question. As explained above in relation to FIG. 2a, some or all of these scores may be output as features in a feature vector which is then processed by a machine learning module to determine a probability that the epitope is an immunogenic epitope for that individual.

### Core Selection

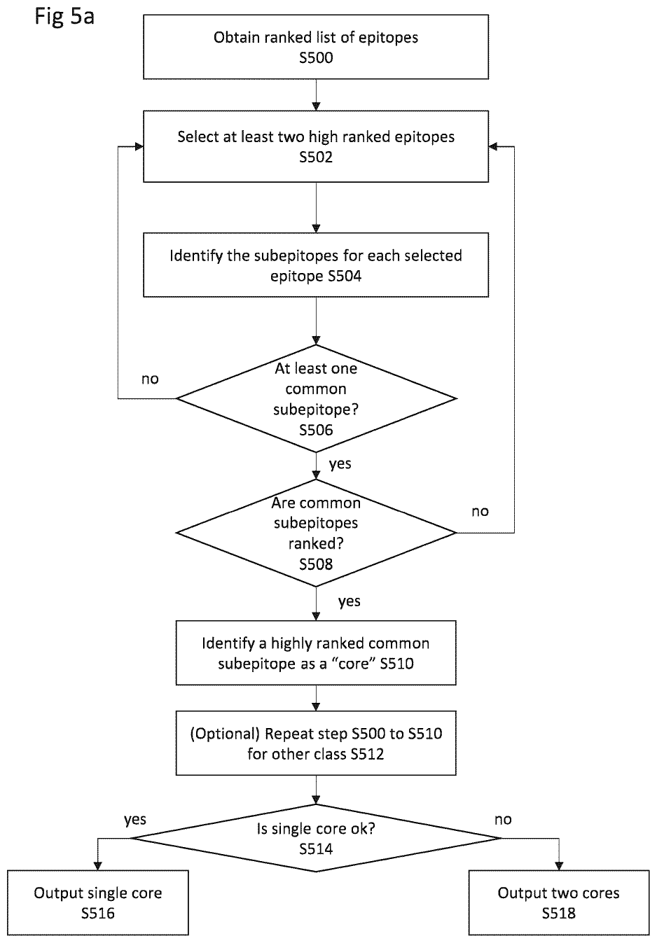

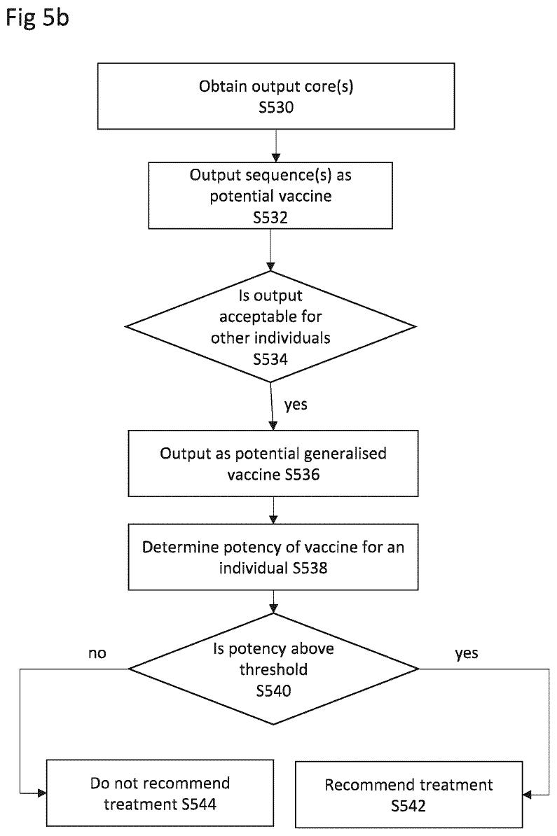

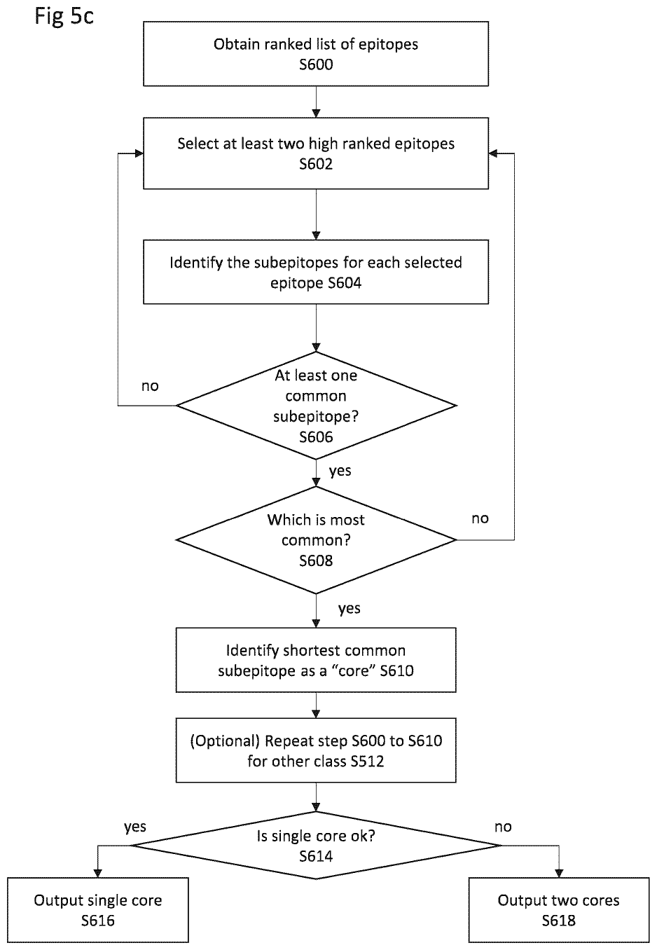

FIG. 5a illustrates one method for selecting a core epitope using the scores calculated above. In a first step S500, a ranked list of epitopes is obtained, for example using the method of FIG. 2a. Merely, as an example, the table below shows an example of such a ranked list for an individual in relation to the SP17 protein which is a known antigen for patients with metastatic breast cancer. The 16 examples shown are all CD4 epitopes and thus the EWS for the HLA class II alleles is shown together with the SWS1 score for both class I and class II alleles and the SWS2 score for both class I and class II alleles. Several epitopes of this small SP17 protein have a high EWS score for the HLA class II alleles and thus are likely to induce potent CD4+ T-cell responses in the individual with metastatic breast cancer. By including the SWS1 and SWS2 scores for the class I alleles, we are checking whether these epitopes have good CD8 epitopes included in their sequence (sub-epitopes).

Tumor antigen (SP17) HLA class I and II epitope analysis
in a patient with metastatic breast cancer

| | | EWS | SWS1 | SWS2 | Number of | Number of |
|---|---|---|---|---|---|---|
| | | Class | Class | Class | SWS1 | SWS2 |
| ID | Epitope | II | II | II | Subitopes | Subitopes |
| 1 | IPAFAAAYFESLLEK | 3.3 | 11.6 | 4.5 | 7 | 2 |
| | (SEQ ID NO: 41) | | | | | |
| 2 | EDRFYNNHAFEEQEP | 3.2 | 8.5 | 3.9 | 6 | 2 |
| | (SEQ ID NO: 42) | | | | | |
| 3 | NIPAFAAAYFESLLEK | 3.1 | 18.6 | 7.8 | 10 | 3 |
| | (SEQ ID NO: 43) | | | | | |
| 4 | KEEVAAVKIQAAFRG | 3.1 | 9.2 | 3.2 | 6 | 2 |
| | (SEQ ID NO: 44) | | | | | |
| 5 | PAFAAAYFESLLEK | 3.0 | 5.2 | 1.5 | 4 | 1 |
| | (SEQ ID NO: 45) | | | | | |
| 6 | EDRFYNNHAFEEQEPP | 3.0 | 15.6 | 4.0 | 9 | 2 |
| | (SEQ ID NO: 46) | | | | | |
| 7 | DNIPAFAAAYFESLLEK | 2.9 | 26.4 | 11.0 | 14 | 4 |
| | (SEQ ID NO: 47) | | | | | |
| 8 | IPAFAAAYFESLLEKR | 2.9 | 19.4 | 3.9 | 10 | 2 |
| | (SEQ ID NO: 48) | | | | | |
| 9 | KEEVAAVKIQAAFR | 2.9 | 4.0 | 2.3 | 3 | 2 |
| | (SEQ ID NO: 49) | | | | | |
| 10 | VEDRFYNNHAFEEQEP | 2.9 | 13.4 | 7.1 | 9 | 3 |
| | (SEQ ID NO: 50) | | | | | |
| 11 | NIPAFAAAYFESLLE | 2.8 | 10.0 | 5.8 | 6 | 3 |
| | (SEQ ID NO: 51) | | | | | |
| 12 | EKEEVAAVKIQAAFR | 2.8 | 7.5 | 5.2 | 5 | 3 |
| | (SEQ ID NO: 52) | | | | | |
| 13 | EKEEVAAVKIQAAFRG | 2.8 | 15.4 | 6.3 | 9 | 3 |
| | (SEQ ID NO: 53) | | | | | |
| 14 | PAFAAAYFESLLEKR | 2.7 | 9.7 | 1.2 | 6 | 1 |
| | (SEQ ID NO: 54) | | | | | |
| 15 | NIPAFAAAYFESLLEKR | 2.7 | 29.6 | 6.8 | 14 | 3 |
| | (SEQ ID NO: 55) | | | | | |
| 16 | IPAFAAAYFESLLE | 2.7 | 5.8 | 3.1 | 4 | 2 |
| | (SEQ ID NO: 56) | | | | | |

In a next step at step S502, at least two high ranked epitopes are selected, e.g. each of epitopes 1 to 16 listed above. At step S504, each of the subepitopes for the selected epitopes are identified, for example using the directed graph shown in FIG. 3. A step S506, the subepitopes are compared to determine whether there is at least one common subepitope, e.g. by performing an intersection on the identified subepitopes. If there are no common subepitopes, the method may loop back to step S502 to select more highly ranked epitopes.

For example, using the Table shown above, the first listed epitope is selected together with the epitopes with the ID numbers 3, 5, 7, 8, 11, 14, 15 and 16. Each of these epitopes have a common subepitope (PAFAAAYFESLLE (SEQ ID NO: 57)) which is 13 amino acids in length. The next step S508 is to determine whether any of the common subepitopes are ranked in the list. For example, the common subepitope (PAFAAAYFESLLE (SEQ ID NO: 57)) is not separately listed in the highest-ranked epitopes which are presented by the autologous HLA molecules and is thus less likely to induce a T-cell response than any of the higher ranked epitopes. If none are ranked, the method may loop back to step S502 to select more highly ranked epitopes to begin the process again.

It will be appreciated that a negative outcome at step S506 is likely to be avoided if a large number of epitopes are selected in step S502. For example, the top 30 epitopes may be selected at step S502, it is statistically unlikely that there are no common subepitopes. Similarly, a negative outcome at step S508 is likely to be avoided if the highly ranked list contains sufficient numbers of epitopes, e.g. there are 200 epitopes in the ranked list. In other words, we could identify the top 30 highly ranked epitopes at step S502 and look for common subepitopes among the top 200 highly ranked epitopes at step S508.

At step S510, at least one core is identified from the common subepitopes. Any suitable mechanism for this decision can be used. For example, the ranking of all common subepitopes may be considered and the highest ranked common subepitope may be identified as the core. For example, NIPAFAAAYFESLLE (SEQ ID NO: 58) is a common subepitope for each of the epitopes with the ID numbers 3, 7 and 15 and is separately listed at number 11. Thus, this common subepitope is the highest ranked and could be output as the core. Alternatively, the potency score of each common subepitope may be compared to a threshold, and each common subepitope with a potency score above the threshold may be identified as a core. Alternatively, the EWS and the SWS2 scores may be separately compared to thresholds and when one or both scores is above a threshold, the common subepitope may be output as a core. It is noted that the core is longer than the smallest core which is common to many high ranked epitopes and comprises the sequences of several overlapping portions. Using the method of FIG. 5a, the core which is selected is usually longer that the shortest sequence which is common to many examples.

FIG. 5a shows a process of selecting a core based on a ranked list of epitopes and the table used in the example shows the scores relating to the HLA class II alleles. Once a core which is suitable has been selected for a particular type of HLA data, the process can be optionally repeated for the other type of HLA data to identify at least one core which is acceptable for the other HLA data at step S512.

Sometimes, there may be a core which has been identified at step S510 which is also identified at step S512. In other words, there is a core which is likely to induce both potent CD8+ and potent CD4+ T-cell responses. An example is shown in the data below.

Table (cont'd)
Tumor antigen (SP17) HLA class I and II epitope
analysis in a patient with metastatic breast cancer

| | | | | | Number | Number |
|---|---|---|---|---|---|---|
| | | EWS | SWS1 | SWS2 | of SWS1 | of SWS2 |
| ID | Epitope | Class II | Class I | Class I | Subitopes | Subitopes |
| 1 | IPAFAAAYFESLLEK | 3.3 | 1.7 | 0 | 2 | 0 |
| | (SEQ ID NO: 41) | | | | | |
| 2 | EDRFYNNHAFEEQEP | 3.2 | 0.0 | 0 | 0 | 0 |
| | (SEQ ID NO: 42) | | | | | |
| 3 | NIPAFAAAYFESLLEK | 3.1 | 2.1 | 0 | 3 | 0 |
| | (SEQ ID NO: 43) | | | | | |
| 4 | KEEVAAVKIQAAFRG | 3.1 | 0.0 | 0 | 0 | 0 |
| | (SEQ ID NO: 44) | | | | | |
| 5 | PAFAAAYFESLLEK | 3.0 | 0.7 | 0 | 1 | 0 |
| | (SEQ ID NO: 45) | | | | | |
| 6 | EDRFYNNHAFEEQEPP | 3.0 | 0.0 | 0 | 0 | 0 |
| | (SEQ ID NO: 46) | | | | | |
| 7 | DNIPAFAAAYFESLLEK | 2.9 | 2.1 | 0 | 3 | 0 |
| | (SEQ ID NO: 47) | | | | | |
| 8 | IPAFAAAYFESLLEKR | 2.9 | 1.7 | 0 | 2 | 0 |
| | (SEQ ID NO: 48) | | | | | |
| 9 | KEEVAAVKIQAAFR | 2.9 | 0.0 | 0 | 0 | 0 |
| | (SEQ ID NO: 49) | | | | | |
| 10 | VEDRFYNNHAFEEQEP | 2.9 | 0.0 | 0 | 0 | 0 |
| | (SEQ ID NO: 50) | | | | | |
| 11 | NIPAFAAAYFESLLE | 2.8 | 2.1 | 0 | 3 | 0 |
| | (SEQ ID NO: 51) | | | | | |
| 12 | EKEEVAAVKIQAAFR | 2.8 | 0.0 | 0 | 0 | 0 |
| | (SEQ ID NO: 52) | | | | | |
| 13 | EKEEVAAVKIQAAFRG | 2.8 | 0.0 | 0 | 0 | 0 |
| | (SEQ ID NO: 53) | | | | | |
| 14 | PAFAAAYFESLLEKR | 2.7 | 0.7 | 0 | 1 | 0 |
| | (SEQ ID NO: 54) | | | | | |
| 15 | NIPAFAAAYFESLLEKR | 2.7 | 2.1 | 0 | 3 | 0 |
| | (SEQ ID NO: 55) | | | | | |
| 16 | IPAFAAAYFESLLE | 2.7 | 1.7 | 0 | 2 | 0 |
| | (SEQ ID NO: 56) | | | | | |

Each of the epitopes having the shortest common subepitope PAFAAAYFESLLE (SEQ ID NO: 57) also has a high SWS1 score ranging between 1.7 and 2.1. More particularly, the core NIPAFAAAYFESLLE (SEQ ID NO: 58) is also highly ranked in this table above. This suggests that the antigen is likely to induce potent CD8+ T-cell responses. This core may be output at step S516 as being suitable for triggering both CD4+ and CD8+ T-cell responses. It will be appreciated that it is more likely that two different cores may be output: one for the HLA class I molecules and one for the HLA class II molecules as shown at step S518.

### Vaccine Development

An important use of the top-ranked epitopes is to select the T-cell antigens to be included in vaccines that induce T-cell responses in an individual. A personalized vaccine typically contains several T cell antigens derived from one or more proteins expressed in the infected cells (e.g. T cell antigens derived from the SARS-COV-2 proteome). Including more than one T cell antigens should increase the likelihood of killing of the sick cells (whether tumour cells or virus-infected cells). Preferably, a vaccine contains at least two T cell antigens to induce both CD8+ and CD4+ T cell responses against a protein expressed in the sick cell. FIG. 5b illustrates some steps that can be carried out when developing a vaccine.

As shown at step S530, an output core or cores are obtained, for example using the method of FIG. 5a. NIPAFAAAYFESLLE (SEQ ID NO: 58) is a rare T cell antigen which appears capable of inducing highly potent CD4+ and CD8+ T-cell responses for this individual by triggering the same TCR but it does not exclude that the epitopes trigger different TCRs. Thus, the core has been used to identify an antigen which is suitable as a personalised vaccine for this individual. Thus at step S532, the sequence for this core could be output as a potential vaccine for this person.

As an example of using two different antigens, for the metastatic breast cancer patient the vaccine targeting the EPCAM protein may be formed by joining the two output cores: YVDEKAPEF (SEQ ID NO: 59) (Class I EWS=2.9) and DVAYYFEKDVKGESL (SEQ ID NO: 60) (class II EWS=2.0) to have a potent vaccine with an aggregated score (i.e. first and second EWS summed) of 4.9. Such a potent T cell vaccine is likely to kill cells expressing the EPCAM protein of the patient. Such a vaccine is personalized to the patient. The joined sequence may thus be output at step S532. It will be appreciated that occasionally the two separate output cores may partially overlap or are so close to each other so that they fit into one longer peptide and the sequence of this longer peptide can be output as a potential vaccine.

As another example, the ranked epitopes below may be used in for the development of a vaccine for Hepatitis B for the three individuals listed below.

Top-ranked epitopes by EWS from Hepatitis B of three individuals

| Subject: 3819 | | | Subject: 3821 | | | Subject: 10881 | | |
|---|---|---|---|---|---|---|---|---|
| Potency: 0.81 [0.58, 1.65] EWS | | | Potency: 0.92 [0.58, 2.68] EWS | | | Potency: 1.32 [0.81, 3.74] | | |
| Peptide | EWS | Length | Peptide | EWS | Length | Peptide | EWS | Length |
| HLYSHPIIL (SEQ | 1.65 | 9 | HLYSHPIIL | 2.68 | 9 | AAYPAVSTF | 3.74 | 9 |
| ID NO: 61) | | | (SEQ ID NO: | | | (SEQ ID NO: 261) | | |
| | | | 161) | | | | | |
| RFYPKVTKY | 1.50 | 9 | FAVPNLQSL | 1.92 | 9 | RAFPHCLAF | 3.13 | 9 |
| (SEQ ID NO: 62) | | | (SEQ ID NO: | | | (SEQ ID NO: 262) | | |
| | | | 162) | | | | | |
| SRPPVGSSL (SEQ | 1.35 | 9 | SLYADSPSV | 1.82 | 9 | YSHPIILGF (SEQ | 3.03 | 9 |
| ID NO: 63) | | | (SEQ ID NO: | | | ID NO: 263) | | |
| | | | 163) | | | | | |
| FRPTTGRTSL | 1.19 | 10 | LLDTASAL | 1.63 | 8 | RFYPKVTKY | 2.73 | 9 |
| (SEQ ID NO: 64) | | | (SEQ ID NO: | | | (SEQ ID NO: 264) | | |
| | | | 164) | | | | | |
| RPPNAPIL (SEQ ID | 1.12 | 8 | VSIPWTHKV | 1.63 | 9 | SSSGHAVEF | 2.36 | 9 |
| NO: 65) | | | (SEQ ID NO: | | | (SEQ ID NO: 265) | | |
| | | | 165) | | | | | |
| HYFQTRHYL | 1.12 | 9 | SRPPVGSSL | 1.62 | 9 | LYSSTVPVF | 2.26 | 9 |
| (SEQ ID NO: 66) | | | (SEQ ID NO: | | | (SEQ ID NO: 266) | | |
| | | | 166) | | | | | |
| FYPKVTKYL | 1.12 | 9 | YQHFRRLLL | 1.59 | 9 | FAVPNLQSL | 2.24 | 9 |
| (SEQ ID NO: 67) | | | (SEQ ID NO: | | | (SEQ ID NO: 267) | | |
| | | | 167) | | | | | |
| FAVPNLQSL | 1.09 | 9 | EQFVGPLTV | 1.45 | 9 | FASPLHVAW | 2.23 | 9 |
| (SEQ ID NO: 68) | | | (SEQ ID NO: | | | (SEQ ID NO: 268) | | |
| | | | 168) | | | | | |
| SLYADSPSV (SEQ | 1.07 | 9 | SRNLYVSLL | 1.44 | 9 | TFSPTYKAF | 2.16 | 9 |
| ID NO: 69) | | | (SEQ ID NO: | | | (SEQ ID NO: 269) | | |
| | | | 169) | | | | | |
| HPAAMPHLL | 1.06 | 9 | RQQGRSWSI | 1.43 | 9 | HLYSHPIIL (SEQ | 2.15 | 9 |
| (SEQ ID NO: 70) | | | (SEQ ID NO: | | | ID NO: 270) | | |
| | | | 170) | | | | | |
| RPTTGRTSL (SEQ | 1.05 | 9 | LRNTHPQAM | 1.43 | 9 | IQSKQAFTF (SEQ | 2.11 | 9 |
| ID NO: 71) | | | (SEQ ID NO: | | | ID NO: 271) | | |
| | | | 171) | | | | | |
| NPHWKTPSF | 1.02 | 9 | GLYSSTVPV | 1.32 | 9 | HYFQTRHYL | 2.11 | 9 |
| (SEQ ID NO: 72) | | | (SEQ ID NO: | | | (SEQ ID NO: 272) | | |
| | | | 172) | | | | | |
| RPPVGSSL (SEQ | 1.00 | 8 | AAYPAVSTF | 1.32 | 9 | KAAYPAVSTF | 2.09 | 10 |
| ID NO: 73) | | | (SEQ ID NO: | | | (SEQ ID NO: 273) | | |
| | | | 173) | | | | | |
| APLPIHTAEL | 1.00 | 10 | ARFSWLSL | 1.30 | 8 | RKAAYPAVSTF | 2.03 | 11 |
| (SEQ ID NO: 74) | | | (SEQ | | | (SEQ ID NO: 274) | | |
| | | | ID NO: 174) | | | | | |
| QILPKVLHKSEQ | 0.99 | 9 | FLLSLGIHL | 1.27 | 9 | MQWNSTTF | 1.92 | 8 |
| ID NO: 75) | | | (SEQ ID NO: | | | (SEQ ID NO: 275) | | |
| | | | 175) | | | | | |
| SERPVFPCW | 0.98 | 9 | IGDPALNM | 1.25 | 8 | QAFTFSPTY | 1.91 | 9 |
| (SEQ ID NO: 76) | | | (SEQ ID NO: | | | (SEQ ID NO: 276) | | |
| | | | 176) | | | | | |
| VPTDHGAHL | 0.97 | 9 | RVTGGVFLV | 1.18 | 9 | FYPKVTKYL | 1.89 | 9 |
| (SEQ ID NO: 77) | | | (SEQ ID NO: | | | (SEQ ID NO: 277) | | |
| | | | 177) | | | | | |
| TPSFPNIHL (SEQ | 0.97 | 9 | QLDPARDVL | 1.17 | 9 | AYPAVSTF (SEQ | 1.82 | 8 |
| ID NO: 78) | | | (SEQ ID NO: | | | ID NO: 278) | | |
| | | | 178) | | | | | |
| RPFSGSLGTL | 0.96 | 10 | KQYLNLYPV | 1.17 | 9 | VSIPWTHKV | 1.77 | 9 |
| (SEQ ID NO: 79) | | | (SEQ ID NO: | | | (SEQ ID NO: 279) | | |
| | | | 179) | | | | | |
| FLLSLGIHL (SEQ | 0.96 | 9 | RFYPKVTKY | 1.17 | 9 | FTFSPTYKAF | 1.74 | 10 |
| ID NO: 80) | | | (SEQ ID NO: | | | (SEQ ID NO: 280) | | |
| | | | 180) | | | | | |
| FLGPLLVL (SEQ | 0.95 | 8 | YMDDVVLGA | 1.16 | 9 | YYPEHLVNHYF | 1.72 | 11 |
| ID NO: 81) | | | (SEQ ID NO: | | | (SEQ ID NO: 281) | | |
| | | | 181) | | | | | |
| RPTTGRTSLY | 0.95 | 10 | QHFRRLLLL | 1.14 | 9 | LSLDVSAAF | 1.61 | 9 |
| (SEQ ID NO: 82) | | | (SEQ ID NO: | | | (SEQ ID NO: 282) | | |
| | | | 182) | | | | | |
| QPTPLSPPL (SEQ | 0.94 | 9 | RQRPGLCQV | 1.11 | 9 | RSQSERPVF | 1.54 | 9 |
| ID NO: 83) | | | (SEQ ID NO: | | | (SEQ ID NO: 283) | | |
| | | | 183) | | | | | |
| VPTDHGAHLSL | 0.92 | 11 | HYFQTRHYL | 1.11 | 9 | KYTSFPWLL | 1.50 | 9 |
| (SEQ ID NO: 84) | | | (SEQ ID NO: | | | (SEQ ID NO: 284) | | |
| | | | 184) | | | | | |
| AESRLVVDF | 0.91 | 9 | FLGPLLVL (SEQ | 1.11 | 8 | SLFTAVTNF | 1.48 | 9 |
| (SEQ ID NO: 85) | | | ID NO: 185) | | | (SEQ ID NO: 285) | | |
| RLPFRPTTGRTSL | 0.90 | 13 | YADSPSVPSHL | 1.02 | 11 | FFLLTRIL (SEQ ID | 1.47 | 8 |
| (SEQ ID NO: 86 | | | (SEQ ID NO: | | | NO: 286) | | |
| | | | 186) | | | | | |
| APLPIHTAELL | 0.89 | 11 | GLLGWSPQA | 1.02 | 9 | FFPSVRDL (SEQ | 1.45 | 8 |
| (SEQ ID NO: 87) | | | (SEQ ID NO: | | | ID NO: 287) | | |
| | | | 187) | | | | | |
| TLWKAGILYK | 0.89 | 10 | VRRAFPHCL | 1.01 | 9 | LYSHPIILGF | 1.42 | 10 |
| (SEQ ID NO: 88) | | | (SEQ ID NO: | | | (SEQ ID NO: 288) | | |
| | | | 188) | | | | | |
| GLYSSTVPV | 0.88 | 9 | QILPKVLHK | 0.99 | 9 | WYWGPSLY | 1.41 | 8 |
| (SEQ ID NO: 89) | | | (SEQ ID NO: | | | (SEQ ID NO: 289) | | |
| | | | 189) | | | | | |
| LRNTHPQAM | 0.88 | 9 | RQLLWFHI | 0.98 | 8 | LYQTFGRKL | 1.41 | 9 |
| (SEQ ID NO: 90) | | | (SEQ ID NO: | | | (SEQ ID NO: 290) | | |
| | | | 190) | | | | | |
| AAYPAVSTF | 0.88 | 9 | SQFSRGNYRV | 0.98 | 10 | VFADATPTGW | 1.39 | 10 |
| (SEQ ID NO: 91) | | | (SEQ ID NO: | | | (SEQ ID NO: 291) | | |
| | | | 191) | | | | | |
| SLPQEHIIQK | 0.88 | 10 | HLPDRVHFA | 0.96 | 9 | LYSILSPF (SEQ ID | 1.38 | 8 |
| (SEQ ID NO: 92) | | | (SEQ ID NO: | | | NO: 292) | | |
| | | | 192) | | | | | |
| YMDDVVLGA | 0.88 | 9 | LLDYQGML | 0.94 | 8 | HFASPLHVAW | 1.35 | 10 |
| (SEQ ID NO: 93) | | | (SEQ I DNO: | | | (SEQ ID NO: 293) | | |
| | | | 193) | | | | | |
| RQPTPLSPPL | 0.87 | 10 | LRFTSARRM | 0.94 | 9 | SYQHFRRLL | 1.34 | 9 |
| (SEQ ID NO: 94) | | | (SEQ ID NO: | | | (SEQ ID NO: 294) | | |
| | | | 194) | | | | | |
| RLPFRPTTGR | 0.87 | 10 | KCEQFVGPLTV | 0.93 | 11 | YYPEHLVNHY | 1.33 | 10 |
| (SEQ ID NO: 95) | | | (SEQ ID NO: | | | (SEQ ID NO: 295) | | |
| | | | 195) | | | | | |
| HQILPKVLHK | 0.86 | 10 | GLSRPLLRL | 0.92 | 9 | AMQWNSTTF | 1.31 | 9 |
| (SEQ ID NO: 96) | | | (SEQ ID NO: | | | (SEQ ID NO: 296) | | |
| | | | 196) | | | | | |
| HLPDRVHFA | 0.86 | 9 | RTSLYADSPSV | 0.91 | 11 | STTDLEAYF | 1.30 | 9 |
| (SEQ ID NO: 97) | | | (SEQ ID NO: | | | (SEQ ID NO: 297) | | |
| | | | 197) | | | | | |
| SPQAQGILQTL | 0.85 | 11 | TRHYLHTL | 0.90 | 8 | SHLPDRVHF | 1.27 | 9 |
| (SEQ ID NO: 98) | | | (SEQ | | | (SEQ ID NO: 298) | | |
| | | | ID NO: 198) | | | | | |
| LPFRPTTGRTSL | 0.85 | 12 | RAFPHCLAF | 0.90 | 9 | MWYWGPSL | 1.26 | 8 |
| (SEQ ID NO: 99) | | | (SEQ ID NO: | | | (SEQ ID NO: 299) | | |
| | | | 199) | | | | | |
| SPSVPSHL (SEQ | 0.84 | 8 | TLWKAGILYK | 0.89 | 10 | FYPKVTKY (SEQ | 1.25 | 00 8 |
| ID NO: 100) | | | (SEQ ID NO: | | | ID NO: 300) | | |
| | | | 200) | | | | | |
| SCLHQSPVRK | 0.84 | 10 | HQILPKVLHK | 0.88 | 10 | FADATPTGW | 1.23 | 9 |
| (SEQ ID NO: 101) | | | (SEQ ID NO: | | | (SEQ ID NO: 301) | | |
| | | | 201) | | | | | |
| GLLGWSPQA | 0.83 | 9 | HQLDPAFRA | 0.87 | 9 | KQAFTFSPTY | 1.22 | 10 |
| (SEQ ID NO: 102 | | | (SEQ ID NO: | | | (SEQ ID NO: 302) | | |
| | | | 202) | | | | | |
| GPSLYSIL (SEQ ID | 0.82 | 8 | SLPQEHIIQK | 0.87 | 10 | RQRPGLCQVF | 1.18 | 10 |
| NO: 103) | | | (SEQ ID NO: | | | (SEQ ID NO: 303) | | |
| | | | 203) | | | | | |
| HLHQDIIKK (SEQ | 0.81 | 9 | ILSPFLPL (SEQ | 0.87 | 8 | STSNPLGFF (SEQ | 1.16 | 9 |
| ID NO: 104) | | | ID NO: 204) | | | ID NO: 304) | | |
| AHQILPKVLHK | 0.81 | 11 | VHFASPLHV | 0.86 | 9 | RSWSIRAGF | 1.15 | 9 |
| (SEQ ID NO: 105) | | | (SEQ ID NO: | | | (SEQ ID NO: 305) | | |
| | | | 205) | | | | | |
| RETTHSASF (SEQ | 0.81 | 9 | RLPFRPTTGR | 0.85 | 10 | SLYADSPSV | 1.15 | 9 |
| ID NO: 106) | | | (SEQ ID NO: | | | (SEQ ID NO: 306) | | |
| | | | 206) | | | | | |
| AAYPAVSTFEK | 0.79 | 11 | SCLHQSPVRK | 0.84 | 10 | NYRVSWPKF | 1.15 | 9 |
| (SEQ ID NO: 107) | | | (SEQ ID NO: | | | (SEQ ID NO: 307) | | |
| | | | 207) | | | | | |
| LLYQTFGRK | 0.79 | 9 | AQFTSAICSV | 0.83 | 10 | NTHPQAMQW | 1.12 | 9 |
| (SEQ ID NO: 108) | | | (SEQ ID NO: | | | (SEQ ID NO: 308) | | |
| | | | 208) | | | | | |
| LPRLADEGL | 0.78 | 9 | QTFGRKLHL | 0.83 | 9 | YSSTVPVF (SEQ | 1.12 | 8 |
| (SEQ ID NO: 109) | | | (SEQ ID NO: | | | I DNO: 309) | | |
| | | | 209) | | | | | |
| ADDPSRGRLGL | 0.78 | 11 | FLLAQFTSA | 0.82 | 9 | NWILRGTSF | 1.10 | 9 |
| (SEQ ID NO: 110) | | | (SEQ ID NO: | | | (SEQ ID NO: 310) | | |
| | | | 210) | | | | | |
| CLHQSPVRK | 0.77 | 9 | HLHQDIIKK | 0.81 | 9 | SFVYVPSAL | 1.07 | 9 |
| (SEQ ID NO: 111) | | | (SEQ ID NO: 211) | | | (SEQ I DNO: 311) | | |
| FPSVRDLL (SEQ | 0.77 | 8 | AHQILPKVLHK | 0.81 | 11 | LSTSNPLGF (SEQ | 1.05 | 9 |
| ID NO: 112) | | | (SEQ ID NO: 212) | | | ID NO: 312) | | |
| FLLAQFTSA (SEQ | 0.76 | 9 | RLADEGLNRRV | 0.81 | 11 | FLGPLLVL (SEQ ID | 1.05 | 8 |
| ID NO: 113) | | | (SEQ ID NO: 213) | | | NO: 313 | | |
| TPTGWGLVM | 0.75 | 9 | SRVTGGVFL | 0.80 | 9 | KSVQHLESL | 1.04 | 9 |
| (SEQ ID NO: 114) | | | (SEQ ID NO: 214) | | | (SEQ ID NO: 314) | | |
| KTPSFPNIHL | 0.75 | 10 | AAYPAVSTFEK | 0.80 | 11 | ARFYPKVTKY | 1.04 | 10 |
| (SEQ ID NO: 115) | | | (SEQ ID NO: 215) | | | (SEQ ID NO: 315) | | |
| WPKFAVPNL | 0.75 | 9 | ALMPLYACI | 0.79 | 9 | FLLSLGIHL (SEQ ID | 1.03 | 9 |
| (SEQ ID NO: 116) | | | (SEQ ID NO: 216) | | | NO: 316) | | |
| IPMGVGLSPFL | 0.74 | 11 | LLYQTFGRK | 0.79 | 9 | RAFPHCLAFSY | 1.03 | 11 |
| (SEQ ID NO: 117) | | | (SEQ ID NO: 217) | | | (SEQ ID NO: 317) | | |
| TPSRVTGGVF | 0.73 | 10 | GLSPTVWL | 0.78 | 8 | VLSRKYTSF | 1.03 | 9 |
| (SEQ ID NO: 118) | | | (SEQ | | | (SEQ ID NO: 318) | | |
| | | | ID NO: 218) | | | | | |
| ILSPFLPL (SEQ ID | 0.73 | 8 | VLHKRTLGL | 0.77 | 9 | SWWTSLNF (SEQ | 1.03 | 8 |
| NO: 119) | | | (SEQ ID NO: 219) | | | ID NO: 319) | | |
| ASCLHQSPVRK | 0.72 | 11 | CLHQSPVRK | 0.77 | 9 | VFNPHWKTPSF | 1.03 | 11 |
| (SEQ ID NO: 120) | | | (SEQ ID NO: 220) | | | (SEQ DI NO: 320) | | |
| IPRTPSRVT (SEQ ID | 0.72 | 9 | SLQAVPWVA | 0.75 | 9 | RRAFPHCLAF | 1.03 | 10 |
| NO: 121) | | | (SEQ ID NO: 221) | | | (SEQ ID NO: 321) | | |
| YPVARQRPGL | 0.72 | 10 | FYPKVTKYL | 0.74 | 9 | ASFCGSPYSW | 1.02 | 10 |
| (SEQ ID NO: 122) | | | (SEQ ID NO: 222) | | | (SEQ ID NO: 322) | | |
| TVNAHQILPK | 0.71 | 10 | AVTNFLLSL | 0.74 | 9 | TVPVFNPHW | 1.00 | 9 |
| (SEQ ID NO: 123) | | | (SEQ ID NO: 223) | | | (SEQ ID NO: 323) | | |
| IPMGVGLSPF | 0.71 | 10 | ASCLHQSPVRK | 0.72 | 11 | VFPCWWLQF | 0.99 | 9 |
| (SEQ ID NO: 124) | | | SEQ ID NO: 224) | | | (SEQ ID NO: 324) | | |
| YYPEHLVNHYF | 0.71 | 11 | GLSRYVARL | 0.72 | 9 | FFPDHQLDPAF | 0.99 | 11 |
| (SEQ ID NO: 125) | | | (SEQ ID NO: 225) | | | (SEQ ID NO: 325) | | |
| AVPTDHGAHL | 0.71 | 10 | LLDDEAGPL | 0.72 | 9 | STVPVFNPHW | 0.98 | 10 |
| (SEQ ID NO: 126) | | | (SEQ ID NO: 226) | | | (SEQ I DNO: 326) | | |
| RPPVGSSLQSK | 0.70 | 11 | TVNAHQILPK | 0.71 | 10 | RLVVDFSQF | 0.98 | 9 |
| (SEQ ID NO: 127) | | | (SEQ ID NO: 227) | | | (SEQ I DNO: 327) | | |
| GEEIRLKVF (SEQ | 0.70 | 9 | SRNLYVSL (SEQ | 0.70 | 8 | YFQTRHYL (SEQ | 0.97 | 8 |
| ID NO: 128) | | | ID NO: 228) | | | ID NO: 328) | | |
| VPNLQSLTNL | 0.69 | 10 | LSYQHFRRL | 0.69 | 9 | SFCGSPYSW | 0.96 | 9 |
| (SEQ ID NO: 129) | | | (SEQ ID NO: 229) | | | (SEQ ID NO: 329) | | |
| HTLWKAGILYK | 0.69 | 11 | HTLWKAGILYK | 0.69 | 11 | HQSPVRKAAY | 0.95 | 10 |
| (SEQ ID NO: 130) | | | (SEQ ID NO: 230) | | | SEQ ID NO: 330 | | |
| LSRPPVGSSL | 0.69 | 10 | GTDNSVVL | 0.69 | 8 | SAGPCALRF | 0.95 | 9 |
| (SEQ ID NO: 131) | | | (SEQ | | | (SEQ ID NO: 331) | | |
| | | | ID NO: 231) | | | | | |
| RLGLSRPLLR | 0.68 | 10 | ILTIPQSL (SEQ | 0.69 | 8 | SASFCGSPYSW | 0.93 | 11 |
| (SEQ ID NO: 132) | | | ID NO: 232) | | | (SEQ ID NO: 332) | | |
| HLYSHPIILGFRK | 0.68 | 13 | RPPVGSSLQSK | 0.69 | 11 | SFLPSDFF (SEQ ID | 0.93 | 8 |
| (SEQ ID NO: 133) | | | (SEQ ID NO: 233) | | | NO: 333) | | |
| RLADEGLNR | 0.68 | 9 | RLGLSRPLLR | 0.68 | 10 | RFYPKVTKYL | 0.93 | 10 |
| (SEQ ID NO: 134) | | | (SEQ ID NO: 234) | | | (SEQ ID NO: 334) | | |
| HPAAMPHL (SEQ | 0.67 | 8 | LLAQFTSAI | 0.68 | 9 | GYPALMPLY | 0.92 | 9 |
| ID NO: 135) | | | (SEQ | | | (SEQ ID NO: 335) | | |
| | | | ID NO: 235) | | | | | |
| SPYSWEQDL | 0.67 | 9 | HLYSHPIILGFRK | 0.68 | 13 | RETTHSASF | 0.92 | 9 |
| (SEQ I DNO: 136) | | | (SEQ ID NO: 236) | | | (SEQ ID NO: 336) | | |
| YPKVTKYL (SEQ | 0.67 | 8 | RLADEGLNR | 0.67 | 9 | QAQGILQTL | 0.91 | 9 |
| ID NO: 137) | | | (SEQ ID NO: 237) | | | (SEQ ID NO: 337) | | |
| GSLPQEHIIQK | 0.66 | 11 | RVHFASPLHV | 0.67 | 10 | HYFQTRHY (SEQ | 0.91 | 8 |
| (SEQ I DNO: 138) | | | (SEQ ID NO: 238) | | | ID NO: 338) | | |
| YPKVTKYLPL | 0.66 | 10 | LQAVPWVAL | 0.67 | 9 | SQFSRGNY (SEQ | 0.90 | 8 |
| (SEQ I DNO: 139) | | | (SEQ ID NO: 239) | | | ID NO: 339) | | |
| RPLLRLPF (SEQ | 0.64 | 8 | GSLPQEHIIQK | 0.66 | 11 | GLYSSTVPV SEQ | 0.89 | 9 |
| ID NO: 140) | | | (SEQ ID NO: 240) | | | ID NO: 340) | | |
| KYTSFPWLL | 0.64 | 9 | FADATPTGWGL | 0.66 | 11 | RNTHPQAMQW | 0.89 | 10 |
| (SEQ ID NO: 141) | | | (SEQ ID NO: 241) | | | (SEQ ID NO: 431) | | |
| SERPVFPCWW | 0.63 | 10 | YSHPIILGF (SEQ | 0.65 | 9 | FTPPHGGLL | 0.89 | 9 |
| (SEQ ID NO: 142) | | | ID NO: 242) | | | (SEQ ID NO: 342) | | |
| LPKVLHKRTL | 0.63 | 10 | FADATPTGW | 0.65 | 9 | KSVQHLESLF | 0.89 | 10 |
| (SEQ ID NO: 143) | | | (SEQ ID NO: 243) | | | (SEQ ID NO: 343) | | |
| LPIHTAEL (SEQ | 0.63 | 8 | QHFRRLLL (SEQ | 0.65 | 8 | KHSSSGHAVEF | 0.88 | 11 |
| ID NO: 144) | | | ID NO: 244) | | | (SEQ ID NO: 344) | | |
| VSIPWTHKV | 0.62 | 9 | NLYVSLLLL | 0.64 | 9 | NHYFQTRHY | 0.88 | 9 |
| (SEQ ID NO: 145) | | | (SEQ ID NO: 245) | | | (SEQ ID NO: 345) | | |
| RTSLYADSPSV | 0.62 | 11 | TSLYADSPSV | 0.64 | 10 | SLLLLYQTF | 0.87 | 9 |
| (SEQ ID NO: 146) | | | (SEQ ID NO: 246) | | | (SEQ ID NO: 346) | | |
| RAFPHCLAF | 0.62 | 9 | KLHLYSHPI | 0.64 | 9 | SQSERPVF (SEQ | 0.87 | 8 |
| (SEQ ID NO: 147) | | | (SEQ | | | ID NO: 347) | | |
| | | | ID NO: 247) | | | | | |
| KPYYPEHL (SEQ | 0.62 | 8 | FTPPHGGLL | 0.64 | 9 | SRDLVVSY (SEQ | 0.87 | 8 |
| ID NO: 148) | | | (SEQ ID NO: 248) | | | ID NO: 348) | | |
| MENITSGF (SEQ | 0.61 | 8 | KRWGYSLNF | 0.62 | 9 | PYYPEHLVNHYF | 0.86 | 12 |
| ID NO: 149) | | | (SEQ ID NO: 249) | | | (SEQ ID NO: 349) | | |
| SRDLVVSY (SEQ | 0.61 | 8 | KKCEQFVGPLT | 0.61 | 12 | ILRGTSFVY (SEQ | 0.85 | 9 |
| ID NO 150) | | | V | | | ID NO: 350) | | |
| | | | (SEQ ID NO: 250) | | | | | |
| KPYYPEHLV | 0.61 | 9 | YQTFGRKLHL | 0.61 | 10 | YMDDVVLGA | 0.85 | 9 |
| (SEQ ID NO 151) | | | (SEQ ID NO: 251) | | | (SEQ ID NO: 351) | | |
| SPTYKAFLCK | 0.60 | 10 | RVSWPKFAV | 0.61 | 9 | RANTANPDW | 0.84 | 9 |
| (SEQ ID NO: 152) | | | (SEQ ID NO: 252) | | | (SEQ ID NO: 352) | | |
| RVTGGVFLVDK | 0.59 | 11 | SLDVSAAF (SEQ | 0.60 | 8 | KRWGYSLNF | 0.84 | 9 |
| (SEQ ID NO: 153) | | | ID NO: 253) | | | (SEQ ID NO: 353) | | |
| DPSRGRLGL | 0.59 | 9 | RVTGGVFLVDK | 0.60 | 11 | FAAPFTQCGY | 0.83 | 10 |
| (SEQ ID NO: 154) | | | (SEQ ID NO: 254) | | | (SEQ ID NO: 354) | | |
| KVFVLGGCRHK | 0.59 | 11 | SPTYKAFLCK | 0.59 | 10 | HSSSGHAVEF | 0.83 | 10 |
| (SEQ ID NO: 155) | | | (SEQ ID NO: 255) | | | (SEQ ID NO: 355) | | |
| FNPHWKTPSF | 0.59 | 10 | IQSKQAFTF | 0.59 | 9 | LQLIMPARF | 0.83 | 9 |
| (SEQ ID NO: 156) | | | (SEQ | | | (SEQ ID NO: 356) | | |
| | | | ID NO: 256) | | | | | |
| YYPEHLVNHY | 0.59 | 10 | KVFVLGGCRHK | 0.59 | 11 | YQHFRRLLL | 0.83 | 9 |
| (SEQ ID NO: 157) | | | (SEQ ID NO: 257) | | | (SEQ ID NO: 357) | | |
| LYSSTVPVF (SEQ | 0.59 | 9 | LQAGFFLL (SEQ | 0.59 | 8 | GLLGWSPQA | 0.83 | 9 |
| ID NO: 158) | | | ID NO: 258) | | | (SEQ I DNO: 358) | | |
| MPHLLVGSSGL | 0.58 | 11 | DRVHFASPL | 0.58 | 9 | VHFASPLHVAW | 0.82 | 11 |
| (SEQ ID NO: 159) | | | (SEQ ID NO: 259) | | | (SEQ ID NO: 359) | | |
| SPPLRNTHPQAM | 0.58 | 12 | ARFYPKVTKY | 0.58 | 10 | LTNLLSSNLSW | 0.81 | 11 |
| (SEQ ID NO: 160) | | | (SEQ ID NO: 260) | | | (SEQ ID NO: 360) | | |

Examples of overlapping epitopes in the above table entitled “Top-ranked epitopes by EWS from Hepatitis B of three individuals” for the subject 10881 include: AAYPAVSTF (SEQ ID NO: 261) (EWS=3.74), KAAYPAVSTF (SEQ ID NO: 273) (EWS=2.09), RKAAYPAVSTF (SEQ ID NO: 274) (EWS=2.03), AYPAVSTF (SEQ ID NO: 278) (EWS=1.82) each of which have the common subepitope: AYPAVSTF (SEQ ID NO: 278). From the four overlapping epitopes RKAAYPAVSTF (SEQ ID NO: 274) could be selected as the T-cell antigen to trigger CD8+ T cell responses because this epitope comprises the sequences of all 4 epitopes. After delivering RKAAYPAVSTF (SEQ ID NO: 274) antigen in the cell, it is processed to smaller peptides to be presented on the surface of the cells of 10881. The potency of T cell response induced by the RKAAYPAVSTF (SEQ ID NO: 274) antigen may be estimated by the aggregation of the EWS of the four epitopes. The “core” of the four epitopes is AYPAVSTF (SEQ ID NO: 278) that would trigger the same TCR and induces potent T cell responses.

For the subject 3821 only two epitopes AAYPAVSTFEK (SEQ ID NO: 215) (EWS=0.80), AAYPAVSTF (SEQ ID NO. 173) (EWS=1.32) have a common subepitope:

AYPAVSTF (SEQ ID NO.278). From these overlapping epitopes the selected T cell antigen for vaccine development could be AAYPAVSTFEK (SEQ ID NO.215). The T cell antigen of 3821 is not only different from the T cell antigen of 10881 but aggregation of their EWS suggest that it induces a much weaker T cell response in the recipient. Subject 3819 has two top-ranked epitopes with a common subepitope (AYPAVSTF): AAYPAVSTFEK (SEQ ID NO: 107) (EWS=0.79) AAYPAVSTF (SEQ ID NO: 91) (EWS=0.88). The selected T cell antigen is AAYPAVSTFEK (SEQ ID NO: 107), the same as for 3821. However, the aggregated EWS suggest a lower “core” density on the cell surface compared to subject 10881. Subject 3819 has very low epitope density from HBV, suggesting that HBV induce a weak T cell responses according to EWS, the top epitope is HLYSHPIIL (SEQ ID NO: 61) (EWS=1.65) which does not have any overlapping epitope among the top 100 epitopes. T cell antigens for subject 3821 could be AAYPAVSTFEK (SEQ ID NO: 107) and HLYSHPIIL (SEQ ID NO: 61) that likely have similarly weak potency to stimulate T cells. These two T-cell antigens would be candidates for the development of an HBV vaccine for 3821.

In the example above, for each of the patients, AAYPAVSTFEK (SEQ ID NO: 107) is an antigen which could be included in the personalised vaccine. It will be appreciated that this step could be repeated for multiple individuals and simple statistical methods can be used to identify the T cell antigen presented by the diseased cells of most individuals suffering in the indicated disease (e.g. COVID, Hepatitis B or cancer). Such repeatedly reported antigens could be included in a general vaccine which is suitable for the general population. For example, as shown in FIG. 5b, the output (either a core or a longer sequence including at least one core) could be analyzed to determine if it is suitable for multiple individuals at step S534. If the output is acceptable, the output may be proposed as a component for a generalized vaccine as shown at step S536. If the output is not acceptable, there is no proposed generalized vaccine.

### Predicting a Patient Response to the Vaccine

The potency of the vaccines for a subject may be determined by the method invented here for example as shown in step S538 and reported in a diagnostic test. Merely as an example, the potency may be determined as the average EWS of the top-ranked epitopes of the vaccine. The responder selection includes obtaining the 4-8 digits HLA genotype of the patient and verifying that the proteins targeted by the vaccine are expressed on the unhealthy cells of the patient. For example, the target protein may be the Spike of the circulating virus variant or AKAP4 which is expressed in breast tumors but not expressed in healthy cells.

The vaccines which induce potent T-cell responses that quickly kill the unhealthy cells (e.g., SARS-COV-2 infected cells or tumor cells) are useful for the prevention and treatment of the disease (e.g., COVID-19 or cancer). Thus, the VERDI method can also be used for the selection of patient(s) who will have potent T cell responses to the vaccine, for example by comparing the potency to a threshold as shown at step S540. In other words, the VERDI method is being used to determine whether any of the putative epitopes in the vaccine are presented at high-density on the unhealthy cells of the individual. When there are highly ranked epitopes presented on the unhealthy cells of the individual which are in common with those in the vaccine, it is more likely the individual will respond well to the vaccine. In other words, there will be more success when there are more highly ranked epitopes which are found in both the circulating virus and the vaccine. Thus, as shown at step S542, an individual is recommended to receive the vaccine when the potency is above a threshold and may be recommended at step S544 not to receive the vaccine when the potency is below or equal to the threshold.

In the prior art, HLA-binding based epitope selection methods have been used for vaccine development. The epitopes in the personal vaccines made for the breast cancer patient were selected to bind at least 3 HLAs of the subject as described in the published US patent application 2018/264094. As example for the prediction of the potency of T-cell responses induced by vaccines, the table below illustrates the potency of eight long peptide vaccines used for the immunization of a metastatic breast cancer patient. The potency of T-cell responses against the tumor antigens were predicted by the system described above (using HLA class I EWS, HLA class II EWS and overall EWS). The potency of T-cell responses to the vaccine peptides after the 1^{st} immunization were measured with ELISPOT assay after 5 days culture of the peripheral mononuclear cells isolated from a blood sample of the patient (Spots/10⁶ PBMC).

Analysis of personal vaccines used for the treatment of a woman with metastatic breast cancer

| | | Tumor | Expression rate in | Class I | Class II | Overall | |
|---|---|---|---|---|---|---|---|
| Long-peptide vaccine | ID | antigen | breast tumors | EWS | EWS | (EWS) | ELISpot |
| NEQDLVREEAQKMS | O6027 1 | SPAG9 | 88% | 0.8 | 0.7 | 1.5 | 0 |
| SLLPTM (SEQ ID NO: | | | | | | | |
| 361) | | | | | | | |
| SVYADQVNIDYLMNR | Q5JQC 9 | AKAP4 | 85% | 1.8 | 1.9 | 3.7 | 366 |
| PQNLR (SEQ ID NO: 362) | | | | | | | |
| FTSSRMSSFNRHMKT | Q8NI5 1 | CTCFL | 71% | 0.3 | 0.2 | 0.5 | 48 |
| HTSEK (SEQ ID NO: 363) | | | | | | | |
| TLPPAWQPFLKDHRI | O1539 2 | BIRC5 | 71% | 0.7 | 0.7 | 1.4 | 71 |
| STFKN (SEQ ID NO: 364) | | | | | | | |
| QEESFSPTAMDAIFG | P4336 3 | MAGEA | 59% | 0.7 | 0.2 | 0.9 | 0 |
| SLSDE (SEQ ID NO: 365) | | A | | | | | |
| RLDQLLRHVMNPLET | P7839 5 | PRAME | 55% | 1.1 | 0.6 | 1.7 | 66 |
| LSITN (SEQ ID NO: 366) | | | | | | | |
| SGSSYFVLANGHILPN | Q8N0 W7 | FMRIN | 49% | 0.5 | 1.4 | 1.9 | 130 |
| SENA (SEQ ID NO: 367) | | B | | | | | |
| SDTKDYFMSKTLGIG | Q8NA0 3 | FSIP1 | 49% | 0.3 | 2.3 | 2.6 | 257 |
| RLKRP (SEQ ID NO: 368) | | | | | | | |

The results show that only one peptide (SVYADQVNIDYLMNRPQNLR (SEQ ID NO: 362)) has acceptable potency as shown by HLA class I and class II EWS, 1.8 and 1.9, respectively. As predicted, this peptide induced potent T cell responses that can attack the tumor cells (366 spots/million cells). Most of the other peptides induced weak T cell responses (less than 100 spots/million cells). Only two peptides might induce relevant T cell responses (between 100-300 spots/million cells), probably because their acceptable HLA class II EWS (2.3, 1.4). However, these peptides have low HLA class I EWS and might not induce CD8 cytotoxic T cell responses that can kill the tumor cells. Pls note that HLA class I and class II EWS aggregation might not be linear as demonstrated in the above example, but CD4+ T cell responses is required for potent CD8+ T cell responses to kill the tumor cells. Results show that the predicted potencies of the peptides included in the personal vaccine for the breast cancer patient were lower than the potencies of the top-ranked epitopes identified in the section above. The results also illustrate the consequence of the poor T cell antigen selection for vaccine development resulting in weak T cell responses.

### Development of T-Cell Therapies

FIG. 5c shows a method of selecting a core which is suitable as a target for T-cell therapy. There is some overlap with the method of FIG. 5a and thus in a first step S600, a ranked list of epitopes is obtained, for example using the method of FIG. 2a. At step S602, at least two high ranked epitopes are selected and at step S604, each of the subepitopes for the selected epitopes are identified, for example using the directed graph shown in FIG. 3. A step S606, the subepitopes are compared to determine whether there is at least one common subepitope, e.g. by performing an intersection on the identified subepitopes. If there are no common subepitopes, the method may loop back to step S602 to select more highly ranked epitopes.

At step S610, at least one core is identified from the common subepitopes. When developing a T-cell therapy, the core is the target for the therapy and thus one method of selecting the core is to identify the shortest overlapping sequence which is common to multiple epitopes. The core which is selected may be the most common core amongst the selected highly ranked epitopes. Once a core which is suitable has been selected for a particular type of HLA data, the process can be optionally repeated for the other type of HLA data to identify at least one core which is acceptable for the other HLA data at step S612. If the identifies cores are the same (which is unlikely) as determined at step S614, then a single core is output at step S616. Otherwise, two cores are output as the targets at step S616.

Returning to the Hepatitis B example, for all the three subjects AYPAVSTF (SEQ ID NO: 278) is the high density “core” on the HBV infected cells that triggers one TCR on the T cells. This highly specific and abundant “core” among subjects exemplifies the identification of one target for TCR-based T cell therapies. This high-density target would be suitable to kill the HBV-infected cells in the three individuals exemplified here.

During the past 10 years, T cell therapies have become important new therapeutic agents to treat tumors and infectious diseases. TCR-based T cell therapies utilize engineered T cells that recognize 9-11 amino acids long epitopes presented by HLA molecules. Since the epitopes are derived from proteins expressed inside the cell, TCR-based T cell therapies are different from conventional antibody-based or chimeric antigen receptor (CAR) therapeutics, which can only bind to proteins expressed on the surface of cells. So far, developers focused on targeting HLA-restricted epitopes that offered modest therapeutic success accompanied with undesired toxicities due to off-target recognition, which is the main limitation for TCR-based T cell therapies. Our computer implemented method may revolutionize the target identification of TCR-based T cell therapies by identification of the high-density “cores” on the cell surface that are recognized by TCRs. The “cores” are HLA unrestricted targets suitable for “core”-specific TCR-based T cell therapies. Likely responders to such therapies can be identified using the invented computer implemented method by the calculation of the targeted “core” density on the surface of the sick cell of the patient.

### Determining Treatment Regimens

As described above, the HBV-specific T cell antigen repertoire of subjects 3819, 3821 and 10881 not only have different specificity (epitope sequence) but aggregation of their EWS suggest different strengths of T-cell responses. The ranked data for the same individuals is also shown for the Human Papillomavirus and for SARS-COV-2. The ranked data may be used to select antigens for the development of a vaccine or T-cell therapy in a similar manner to that described above.

The results illustrated below show the HPV and SARS-COV-2 specific top-ranked epitopes presented by HLA class I alleles of the three subjects.

Top-ranked epitopes from Human Papillomavirus Type 16 by EWS of three individuals

| Subject: 3819 | | | Subject: 3821 | | | Subject: 10881 | | |
|---|---|---|---|---|---|---|---|---|
| Potency: 0.88 [0.70, 1.43] EWS | | | Potency: 1.05 [0.75, 2.05] EWS | | | Potency: 1.28 [0.91, 2.71] EWS | | |
| Peptide | EWS | Length | Peptide | EWS | Length | Peptide | EWS | Length |
| FYNPDTQRL | 1.43 | 9 | SMDYKQTQL | 2.05 | 9 | YVHEGIRTY | 2.71 | 9 |
| (SEQ ID NO: 369) | | | (SEQ ID NO: | | | (SEQ ID NO: 569) | | |
| | | | 469) | | | | | |
| YLHPSYYML | 1.37 | 9 | YRFKKHCTL | 1.94 | 9 | VVFTFPNEF | 2.70 | 9 |
| (SEQ ID NO: 370) | | | (SEQ ID NO: | | | (SEQ ID NO: 570) | | |
| | | | 470) | | | | | |
| SMDYKQTQL | 1.16 | 9 | ALDGNLVSM | 1.92 | 9 | RAHYNIVTF | 2.64 | 9 |
| (SEQ ID NO: 371) | | | (SEQ ID NO: | | | (SEQ ID NO: 571) | | |
| | | | 471) | | | | | |
| YRDGNPYAV | 1.16 | 9 | YLHPSYYML | 1.81 | 9 | FYNPDTQRL | 2.15 | 9 |
| (SEQ ID NO: 372) | | | (SEQ ID NO: | | | (SEQ ID NO: 572) | | |
| | | | 472) | | | | | |
| RPIPKPSPW | 1.10 | 9 | TLQDVSLEV | 1.80 | 9 | YYVHEGIRTY | 2.10 | 10 |
| (SEQ ID NO: 373) | | | (SEQ ID NO: | | | (SEQ ID NO: 573) | | |
| | | | 473) | | | | | |
| VPSVPSTSL | 1.09 | 9 | NLDTASTTL | 1.69 | 9 | KFYSKISEY | 2.07 | 9 |
| (SEQ ID NO: 374) | | | (SEQ ID NO: | | | (SEQ ID NO: 574) | | |
| | | | 474) | | | | | |
| YLPPVPVSKV | 1.08 | 10 | VKIPKTITV | 1.63 | 9 | FTFPNEFPF | 2.00 | 9 |
| (SEQ ID NO: 375) | | | SEQ ID NO: | | | (SEQ ID NO: 575) | | |
| | | | 475) | | | | | |
| ALDGNLVSM | 1.08 | 9 | SRIGNKQTL | 1.61 | 9 | YLHNRLVVF | 1.99 | 9 |
| (SEQ ID NO: 376) | | | (SEQ ID NO: | | | (SEQ ID NO: 576) | | |
| | | | 476) | | | | | |
| TLQDVSLEV | 1.08 | 9 | AILDINNTV | 1.52 | 9 | KSAIVTLTY | 1.95 | 9 |
| (SEQ ID NO: 377) | | | (SEQ ID NO: | | | (SEQ ID NO: 577) | | |
| | | | 477) | | | | | |
| TLAPVRPPL | 1.07 | 9 | KLLSKLLCV | 1.50 | 9 | KSIGAKVHY | 1.89 | 9 |
| (SEQ ID NO: 378) | | | (SEQ ID NO: | | | (SEQ ID NO: 578) | | |
| | | | 478) | | | | | |
| YLPPVPVSK | 1.06 | 9 | YRFVTSQAI | 1.44 | 9 | YLHPSYYML | 1.87 | 9 |
| (SEQ ID NO: 379) | | | (SEQ ID NO: | | | (SEQ ID N: 579) | | |
| | | | 479) | | | | | |
| SRIGNKQTL | 1.05 | 9 | ALQAIELQL | 1.42 | 9 | FYLRREQMF | 1.86 | 9 |
| (SEQ ID NO: 380) | | | (SEQ | | | (SEQ ID NO: 580) | | |
| | | | ID NO: 480) | | | | | |
| AETETAHAL | 1.02 | 9 | SIADSIKTL | 1.39 | 9 | RQTVLQHSF | 1.72 | 9 |
| (SEQ ID NO: 381) | | | (SEQ ID NO: | | | (SEQ ID NO: 581) | | |
| | | | 481) | | | | | |
| YRFKKHCTL | 1.01 | 9 | IADSIKTLL | 1.37 | 9 | FYLHPSYYM | 1.70 | 9 |
| (SEQ ID NO: 382) | | | (SEQ ID NO: | | | (SEQ ID NO: 582) | | |
| | | | 482) | | | | | |
| RPRKLPQL (SEQ | 0.99 | 8 | KVSGLQYRV | 1.35 | 9 | YYHAGTSRL | 1.69 | 9 |
| ID NO: 383) | | | (SEQ ID NO: | | | (SEQ ID NO: 583) | | |
| | | | 483) | | | | | |
| SLFGMSLMK | 0.99 | 9 | SQVKIPKTI | 1.34 | 9 | KTITVSTGF | 1.69 | 9 |
| (SEQ ID NO: 384) | | | (SEQ ID NO: | | | (SEQ ID NO: 584) | | |
| | | | 484) | | | | | |
| TLAPVRPPLTV | 0.99 | 11 | YLPPVPVSKV | 1.24 | 10 | SFSELVRPF | 1.66 | 9 |
| (SEQ ID NO: 385) | | | (SEQ ID NO: | | | (SEQ ID NO: 585) | | |
| | | | 485) | | | | | |
| SPEIIRQHL | 0.99 | 9 | ILVPKVSGL | 1.22 | 9 | YYKAREMGF | 1.55 | 9 |
| (SEQ ID NO: 386) | | | (SEQ | | | (SEQ ID NO: 586) | | |
| | | | ID NO: 486) | | | | | |
| KFYSKISEY (SEQ | 0.98 | 9 | SRTWSRLSL | 1.21 | 9 | RYQGVEFMSF | 1.52 | 10 |
| ID NO: 387) | | | (SEQ ID NO: 487) | | | (SEQ ID NO: 587) | | |
| AEKYSKNKVW | 0.97 | 10 | YIDDNLRNAL | 1.20 | 10 | FGFPDTSFY | 1.50 | 9 |
| (SEQ ID NO: 388) | | | (SEQ ID NO: 488) | | | (SEQ ID NO: 588) | | |
| APVRPPLTV | 0.97 | 9 | YRDGNPYAV | 1.18 | 9 | AANTGKSLF | 1.44 | 9 |
| (SEQ ID NO: 389) | | | (SEQ ID NO: 489) | | | (SEQ ID NO: 589) | | |
| YYHAGTSRL | 0.97 | 9 | YSKNKVWEV | 1.18 | 9 | SFLTALKRF | 1.43 | 9 |
| (SEQ ID NO: 390) | | | (SEQ ID NO: 490) | | | (SEQ ID NO: 590) | | |
| AETETAHALF | 0.96 | 10 | HQVVPTLAV | 1.16 | 9 | FVNSKSHFW | 1.42 | 9 |
| (SEQ ID NO: 391) | | | (SEQ ID NO: 491) | | | (SEQ ID NO: 591) | | |
| GLYSRTTQQVK | 0.96 | 11 | TKYPLLKLL | 1.15 | 9 | LYTAVSSTW | 1.39 | 9 |
| (SEQ ID NO: 392) | | | (SEQ | | | (SEQ ID NO: 592) | | |
| | | | ID NO: 492) | | | | | |
| SEVYGDTPEW | 0.96 | 10 | SLVEETSFI (SEQ | 1.13 | 9 | QYGSMGVFF | 1.39 | 9 |
| (SEQ ID NO: 393) | | | ID NO: 493) | | | (SEQ ID NO: 593) | | |
| ILVPKVSGL (SEQ | 0.96 | 9 | ERPRKLPQL | 1.13 | 9 | GYIPANTTI | 1.39 | 9 |
| ID NO: 394) | | | (SEQ ID NO: 494) | | | (SEQ ID NO: 594) | | |
| RETIEKLLSK | 0.95 | 10 | IADAGDFYL | 1.13 | 9 | HYTNWTHIY | 1.37 | 9 |
| (SEQ ID NO: 395) | | | (SEQ ID NO: 495) | | | (SEQ ID NO: 595) | | |
| LPSEATVYL | 0.95 | 9 | KQHRDAVQV | 1.13 | 9 | VTTHNNPTF | 1.35 | 9 |
| (SEQ ID NO: 396) | | | (SEQ ID NO: 496) | | | (SEQ ID NO: 596) | | |
| TPVPSVPSTSL | 0.94 | 11 | YHAGTSRLL | 1.12 | 9 | IHLPDPNKF | 1.34 | 9 |
| (SEQ ID NO: 397) | | | (SEQ ID NO: 497) | | | (SEQ ID NO: 597) | | |
| KLLSKLLCV | 0.94 | 9 | AAFGLTPSI | 1.12 | 9 | TIADQILQY | 1.34 | 9 |
| (SEQ ID NO 398) | | | (SEQ | | | (SEQ ID NO: 598 | | |
| | | | ID NO: 498) | | | | | |
| KSLFGMSLMK | 0.94 | 10 | SRTRRETQL | 1.11 | 9 | TTIPFGGAY | 1.32 | 9 |
| (SEQ ID NO: 399) | | | (SEQ ID NO: 499) | | | (SEQ ID NO: 599) | | |
| TVYLPPVPVSK | 0.94 | 11 | FIDAGAPTSV | 1.10 | 10 | KFGFPDTSF | 1.3 | 9 |
| (SEQ ID NO: 400) | | | (SEQ ID NO: 500) | | | (SEQ ID NO: 600) | | |
| EEYDLQFIF (SEQ | 0.94 | 9 | STDTTPAIL | 1.10 | 9 | QAIELQLTL | 1.29 | 9 |
| ID NO: 401) | | | (SEQ ID NO: 501) | | | (SEQ ID NO: 601) | | |
| FELSQMVQW | 0.93 | 9 | TLAPVRPPLTV | 1.09 | 11 | SIADSIKTL | 1.29 | 9 |
| (SEQ ID NO: 402) | | | (SEQ ID NO: 502) | | | (SEQ ID NO: 602) | | |
| RPRKLPQLCTEL | 0.92 | 12 | KQMSMSQWI | 1.08 | 9 | YVARTNIYY | 1.27 | 9 |
| (SEQ ID NO: 403) | | | (SEQ ID NO: 503) | | | (SEQ ID NO: 603) | | |
| TPIPGSRPV | 0.91 | 9 | QAIELQLTL | 1.07 | 9 | LQTITPSTY | 1.26 | 9 |
| (SEQ ID NO 404) | | | (SEQ | | | (SEQ ID NO: 604) | | |
| | | | ID NO: 504) | | | | | |
| GISGHPLLNK | 0.91 | 10 | KHKSAIVTL | 1.06 | 9 | YQGVEFMSF | 1.26 | 9 |
| (SEQ ID NO: 405) | | | (SEQ | | | (SEQ ID NO: 605) | | |
| | | | ID NO: 505) | | | | | |
| TTYKNTNFK | 0.91 | 9 | TLAPVRPPL | 1.05 | 9 | YKNTNFKEY | 1.23 | 9 |
| (SEQ ID NO: 406) | | | (SEQ ID NO: 506) | | | (SEQ ID NO: 606) | | |
| FPLGRKFLL | 0.91 | 9 | IADSIKTL (SEQ | 1.03 | 8 | QYSNEKWTL | 1.23 | 9 |
| (SEQ ID NO: 407) | | | ID NO: 507) | | | (SEQ ID NO: 607) | | |
| KEDPLKKYTF | 0.90 | 10 | ITDQAPSLI (SEQ | 1.01 | 9 | LQYGSMGVF | 1.2 | 9 |
| (SEQ ID NO: 408) | | | ID NO: 508) | | | (SEQ ID NO: 608) | | |
| TPETPATPL | 0.90 | 9 | LRYQGVEFM | 1.01 | 9 | GTLEDTYRF | 1.19 | 9 |
| (SEQ ID NO: 409) | | | (SEQ ID NO: 509) | | | (SEQ ID NO: 609) | | |
| SIADSIKTL | 0.90 | 9 | SLFGMSLMK | 1.01 | 9 | YVHEGIRTYF | 1.19 | 10 |
| (SEQ ID NO: 410) | | | (SEQ ID NO: 510) | | | (SEQ ID NO: 610) | | |
| GPSDPSIVSL | 0.90 | 10 | ITDQAPSL (SEQ | 1.00 | 8 | FLRYQGVEF | 1.18 | 9 |
| (SEQ IDNO: 411) | | | ID NO: 511) | | | (SEQ ID NO: 611) | | |
| AILDINNTV (SEQ | 0.90 | 9 | YLPPVPVSK | 0.98 | 9 | HYYYDLSTI | 1.18 | 9 |
| ID NO: 412) | | | (SEQ ID NO: 512) | | | (SEQ ID NO: 612) | | |
| SPRLKAICI (SEQ | 0.89 | 9 | PSDPSIVSL (SEQ | 0.98 | 9 | ALDGNLVSM | 1.18 | 9 |
| ID NO: 413) | | | ID NO: 513) | | | (SEQ ID NO: 613) | | |
| YLPPVPVSKVV | 0.89 | 11 | ILEDWNFGL | 0.98 | 9 | TAVSSTWHW | 1.17 | 9 |
| (SEQ ID NO: 414) | | | (SEQ ID NO: 514) | | | (SEQ ID NO: 614) | | |
| AEKKQMSMSQW | 0.88 | 11 | LADAKIGML | 0.98 | 9 | LQYGSMGVFF | 1.17 | 10 |
| (SEQ ID NO: 415) | | | (SEQ ID NO: 515) | | | (SEQ ID NO: 615) | | |
| RPVARLGL (SEQ | 0.88 | 8 | QHRDAVQVL | 0.97 | 9 | IYYHAGTSRL | 1.16 | 10 |
| ID NO: 416) | | | (SEQ ID NO: 516) | | | (SEQ ID NO: 616) | | |
| TFELSQMVQW | 0.88 | 10 | KWTLQDVSLEV | 0.97 | 11 | FLIHTHARF | 1.15 | 9 |
| (SEQ ID NO: 417) | | | (SEQ ID NO: 517) | | | (SEQ ID NO: 617) | | |
| KEDPLKKYTFW | 0.87 | 11 | GLYSRTTQQVK | 0.97 | 11 | YYVHEGIRTYF | 1.15 | 11 |
| (SEQ ID NO: 418) | | | (SEQ ID NO: 518) | | | (SEQ ID NO: 618) | | |
| RIHLPDPNK | 0.87 | 9 | FYNPDTQRL | 0.97 | 9 | DQFPLGRKF | 1.14 | 9 |
| (SEQ ID NO: 419) | | | (SEQ ID NO: 519) | | | (SEQ ID NO: 619) | | |
| IPANTTIPF (SEQ ID | 0.86 | 9 | SQVKIPKTITV | 0.95 | 11 | YFPTPSGSM | 1.13 | 9 |
| NO: 420) | | | (SEQ ID NO: 520) | | | (SEQ ID NO: 620) | | |
| ALQAIELQL (SEQ | 0.85 | 9 | RETIEKLLSK | 0.95 | 10 | FYSKISEY | 1.13 | 8 |
| ID NO: 421) | | | (SEQ ID NO: 521) | | | (SEQ ID NO: 621) | | |
| SPTSINNGL | 0.85 | 9 | KSLFGMSLMK | 0.95 | 10 | KYPDYIKMV | 1.11 | 9 |
| (SEQ ID NO: 422) | | | (SEQ ID NO: 522) | | | (SEQ ID NO: 622) | | |
| NLDTASTTL | 0.83 | 9 | GLYSRTTQQV | 0.95 | 10 | GYIPANTTIPF | 1.10 | 11 |
| (SEQ ID NO: 423) | | | (SEQ ID NO: 523) | | | (SEQ ID NO: 623) | | |
| RASATQLYK | 0.83 | 9 | YVHEGIRTY | 0.94 | 9 | FFYLRREQM | 1.09 | 9 |
| (SEQ ID NO: 424) | | | (SEQ ID NO: 524) | | | (SEQ ID NO: 624) | | |
| YVHEGIRTY | 0.82 | 9 | TVYLPPVPVSK | 0.94 | 11 | LYGVSFSEL | 1.09 | 9 |
| (SEQ ID NO: 425) | | | (SEQ ID NO: 525) | | | (SEQ ID NO: 625) | | |
| ALFTAQEAK | 0.81 | 9 | SLWLPSEATV | 0.92 | 10 | SMDYKQTQL | 1.08 | 9 |
| (SEQ ID NO: 426) | | | (SEQ ID NO: 526) | | | (SEQ ID NO: 626) | | |
| RLKAICIEK (SEQ | 0.81 | 9 | GISGHPLLNK | 0.91 | 10 | VSLVEETSF | 1.08 | 9 |
| ID NO: 427) | | | (SEQ ID NO: 527) | | | (SEQ ID NO: 627) | | |
| SLHLTAHTK | 0.81 | 9 | TTYKNTNFK | 0.90 | 9 | DSAPILTAF | 1.08 | 9 |
| (SEQ ID NO: 428) | | | (SEQ ID NO: 528) | | | (SEQ ID NO: 628) | | |
| TEVETQQML | 0.80 | 9 | SVDSAPIL (SEQ | 0.90 | 8 | IADSIKTLL (SEQ | 1.08 | 9 |
| (SEQ ID NO: 429) | | | ID NO: 529) | | | ID NO: 529) | | |
| ILEDWNFGL | 0.80 | 9 | VSEPYGDSL | 0.89 | 9 | LTADVMTYI | 1.07 | 9 |
| (SEQ ID NO: 430) | | | (SEQ ID NO: 530) | | | (SEQ ID NO: 630) | | |
| HPAATHTKAVAL | 0.80 | 12 | YLPPVPVSKVV | 0.89 | 11 | NYFPTPSGSM | 1.06 | 10 |
| (SEQ ID NO: 431) | | | (SEQ ID NO: 531) | | | (SEQ ID NO: 631) | | |
| RPPTATDTL | 0.79 | 9 | VQLKCPPLL | 0.89 | 9 | AFVTTPTKL | 1.06 | 9 |
| (SEQ ID NO: 432) | | | (SEQ ID NO: 532) | | | (SEQ ID NO: 632) | | |
| EEGTGCNGW | 0.79 | 9 | SQYSNEKWTL | 0.87 | 10 | TLQDVSLEV | 1.05 | 9 |
| (SEQ ID NO: 433) | | | (SEQ ID NO: 533) | | | (SEQ ID NO: 633) | | |
| KLRSTAAAL | 0.78 | 9 | YRVFRIHL (SEQ | 0.87 | 8 | TTPTKLITY | 1.04 | 9 |
| (SEQ ID NO: 434) | | | ID NO: 534) | | | (SEQ ID NO: 634) | | |
| RPRKLPQLC | 0.78 | 9 | RIHLPDPNK | 0.87 | 9 | SIKTLLQQY | 1.03 | 9 |
| (SEQ ID NO: 435) | | | (SEQ ID NO: 535) | | | (SEQ ID NO: 635) | | |
| IPGSRPVARL | 0.78 | 10 | TVYLPPVPV | 0.87 | 9 | YYHAGTSRLL | 1.03 | 10 |
| (SEQ ID NO: 436) | | | (SEQ ID NO: 536) | | | (SEQ ID NO: 636) | | |
| ISGHPLLNK | 0.77 | 9 | SRTTQQVKV | 0.87 | 9 | TAASAFRCF | 1.03 | 9 |
| (SEQ ID NO: 437) | | | (SEQ ID NO: 537) | | | (SEQ ID NO: 637) | | |
| RTWSRLSLH | 0.77 | 9 | GQVDYYGLYYV | 0.87 | 11 | FYLHPSYY | 1.03 | 8 |
| (SEQ ID NO: 438) | | | (SEQ ID NO: 538) | | | (SEQ ID NO: 638) | | |
| KEYLRHGEEY | 0.77 | 10 | DQFLSQVKI | 0.87 | 9 | HGYTVEVQF | 1.02 | 9 |
| (SEQ ID NO: 439) | | | (SEQ ID NO: 539) | | | (SEQ ID NO: 639) | | |
| GEEGTGCNGW | 0.76 | 10 | ITDTSTTPV (SEQ | 0.85 | 9 | SKISEYRHY | 1.01 | 9 |
| (SEQ ID NO: 440) | | | ID NO: 540) | | | (SEQ ID NO: 640) | | |
| AISTSETTYK | 0.76 | 10 | SRAAKRRLF | 0.85 | 9 | IYYKAREMGF | 1.01 | 10 |
| (SEQ ID NO: 441) | | | (SEQ ID NO: 541) | | | (SEQ ID NO: 641) | | |
| SLYGTTLEQQYN | 0.75 | 13 | SDPSIVSL (SEQ | 0.84 | 8 | ILVPKVSGL | 1.00 | 9 |
| K SEQ ID NO: 442) | | | ID NO: 542) | | | (SEQ ID NO: 642) | | |
| KTIADQILQY | 0.75 | 10 | AMLAKFKEL | 0.83 | 9 | RSTAAALYW | 1.00 | 9 |
| SEQ ID NO: 443) | | | (SEQ ID NO: 543) | | | (SEQ ID NO: 643) | | |
| IVALHRPAL (SEQ | 0.75 | 9 | RASATQLYK | 0.83 | 9 | NYEEIPMDTF | 1.00 | 10 |
| ID NO: 444) | | | (SEQ ID NO: 544) | | | (SEQ ID NO: 644) | | |
| TPAETGGHFTL | 0.75 | 11 | ILDINNTV (SEQ | 0.83 | 8 | FYLHPSYYML | 1.00 | 10 |
| (SEQ ID NO: 445) | | | ID NO: 545) | | | (SEQ ID NO: 645) | | |
| WPTTPPRPI | 0.75 | 9 | CLYLHIQSL (SEQ | 0.83 | 9 | KQIVMFLRY | 1.00 | 9 |
| (SEQ ID NO: 446) | | | ID NO: 546) | | | (SEQ ID NO: 646) | | |
| SLWLPSEATVYL | 0.74 | 12 | ALFTAQEAK | 0.82 | 9 | LYLHIQSL | 1.00 | 8 |
| (SEQ ID NO: 447) | | | (SEQ ID NO: 547) | | | (SEQ ID NO: 647) | | |
| SLYGTTLEQQY | 0.74 | 11 | MVDTGFGAM | 0.82 | 9 | SFYNPDTQRL | 0.99 | 10 |
| (SEQ ID NO: 448) | | | (SEQ ID NO: 548) | | | (SEQ ID NO: 648) | | |
| KVVDPAFVTTPT K | 0.74 | 13 | SLHLTAHTK | 0.82 | 9 | YSKNKVWEV | 0.99 | 9 |
| (SEQ ID NO: 449) | | | (SEQ ID NO: 549) | | | (SEQ ID NO: 649) | | |
| KWTLQDVSLEV | 0.74 | 11 | YIDDNLRNA | 0.82 | 9 | GQAEPDRAHY | 0.99 | 10 |
| (SEQ ID NO: 450) | | | (SEQ ID NO: 550) | | | (SEQ ID NO: 650) | | |
| GTCPPDIIPK | 0.74 | 10 | RLKAICIEK (SEQ | 0.81 | 9 | VDSAPILTAF | 0.99 | 10 |
| (SEQ ID NO: 451) | | | ID NO: 551) | | | (SEQ ID NO: 651) | | |
| KRASATQLY | 0.74 | 9 | YMLDLQPET | 0.81 | 9 | KRASATQLY | 0.98 | 9 |
| (SEQ ID NO: 452) | | | (SEQ ID NO: 552) | | | (SEQ ID NO: 652) | | |
| TIADQILQY (SEQ | 0.74 | 9 | TLHEYMLDL | 0.79 | 9 | IYYKAREM | 0.98 | 8 |
| ID NO: 453) | | | (SEQ ID NO: 553) | | | (SEQ ID NO: 653) | | |
| SLVEETSFI (SEQ | 0.74 | 9 | YLRREQMFV | 0.78 | 9 | AQIFNKPYW | 0.97 | 9 |
| ID NO: 454) | | | (SEQ ID NO: 554) | | | (SEQ ID NO: 654) | | |
| SEPYGDSLFF | 0.73 | 10 | YGDSLFFYL | 0.78 | 9 | CYSLYGTTL | 0.97 | 9 |
| (SEQ ID NO: 455) | | | (SEQ ID NO: 555) | | | (SEQ ID NO: 655) | | |
| TPAETGGHF | 0.73 | 9 | RQTVLQHSF | 0.78 | 9 | YLPPVPVSKV | 0.97 | 10 |
| (SEQ ID NO: 456) | | | (SEQ ID NO: 556) | | | (SEQ ID NO: 656) | | |
| LPDPNKFGF | 0.72 | 9 | RTWSRLSLH | 0.78 | 9 | GQVDYYGLY | 0.96 | 9 |
| (SEQ ID NO: 457) | | | (SEQ ID NO: 557) | | | (SEQ ID NO: 657) | | |
| KAKPKFTLGK | 0.72 | 10 | KQQLLRREV | 0.77 | 9 | IYNSQYSNEK | 0.96 | 11 |
| (SEQ ID NO: 458) | | | (SEQ ID NO: 558) | | | W (SEQ ID NO: | | |
| | | | | | | 658) | | |
| LLYGAANTGK | 0.72 | 10 | ISGHPLLNK | 0.77 | 9 | IVDDSEIAY | 0.96 | 9 |
| (SEQ ID NO: 459) | | | (SEQ ID NO: 559) | | | (SEQ ID NO: 659) | | |
| STWPTTPPR | 0.72 | 9 | KTIADQILQY | 0.77 | 10 | VTLTYDSEW | 0.95 | 9 |
| (SEQ ID NO: 460) | | | (SEQ ID NO: 560) | | | (SEQ ID NO: 660) | | |
| WEVNLKEKF | 0.72 | 9 | TIDPAEEIEL (SEQ | 0.76 | 10 | EQMFVRHLF | 0.95 | 9 |
| (SEQ ID NO: 461) | | | ID NO: 561) | | | (SEQ ID NO: 661) | | |
| FYLHPSYYM | 0.72 | 9 | AISTSETTYK | 0.76 | 10 | SFNDCTFEL | 0.94 | 9 |
| (SEQ ID NO: 462) | | | (SEQ ID NO: 562) | | | (SEQ ID NO 662) | | |
| HETETPCSQY | 0.72 | 10 | SLYGTTLEQQY | 0.75 | 11 | RHLDKKQRF | 0.94 | 9 |
| (SEQ ID NO: 463) | | | (SEQ ID NO: 563) | | | (SEQ ID NO: 663) | | |
| RPIPKPSPWA | 0.71 | 10 | SLYGTTLEQQYN | 0.75 | 13 | FYNPDTQRLV | 0.93 | 11 |
| (SEQ ID NO: 464) | | | K (SEQ ID NO: | | | W (SEQ ID NO: | | |
| | | | 564) | | | 664) | | |
| KENDGDSLPTF | 0.71 | 11 | SADLDQFPL | 0.75 | 9 | AILDINNTV (SEQ | 0.93 | 9 |
| (SEQ ID NO: 465) | | | (SEQ ID NO: 565) | | | ID NO: 665) | | |
| SPWAPKKHRRL | 0.71 | 11 | SIADSIKTLL (SEQ | 0.75 | 10 | QFPLGRKF | 0.93 | 8 |
| (SEQ ID NO: 466) | | | IDNO: 566) | | | (SEQ ID NO: 666) | | |
| VYLPPVPVSK | 0.71 | 10 | ALQAIELQLTL | 0.75 | 11 | CFVNSKSHF | 0.92 | 9 |
| (SEQ ID NO: 467) | | | (SEQ ID NO: 567) | | | (SEQ ID NO: 667) | | |
| AETGGHFTL | 0.70 | 9 | RDQFLSQVKI | 0.75 | 10 | KHKSAIVTL | 0.91 | 9 |
| (SEQ ID NO: 468) | | | (SEQ ID NO: 568) | | | (SEQ ID NO: 668) | | |

The full dataset for SARS-COV-2 is detailed in the table below.

Top-ranked epitopes from SARS-COV-2 by EWS of three individuals

| Subject: 3819 | | | Subject: 3821 | | | Subject: 10881 | | |
|---|---|---|---|---|---|---|---|---|
| Potency: 1.08 [0.95, 1.90] EWS | | | Potency: 1.62 [1.31, 2.56] EWS | | | Potency: 1.95 [1.88, 3.31] EWS | | |
| Peptide | EWS | Length | Peptide | EWS | Length | Peptide | EWS | Length |
| VYDPLQPEL | 1.90 | 9 | KVDGVVQQL | 2.56 | 9 | KAYNVTQAF | 3.31 | 9 |
| (SEQ ID NO: | | | (SEQ ID NO: | | | (SEQ ID NO: | | |
| 669) | | | 769) | | | 869) | | |
| YLQPRTFLL | 1.78 | 9 | YLQPRTFLL | 2.48 | 9 | KSHKPPISF | 2.90 | 9 |
| (SEQ ID NO: | | | (SEQ ID NO: | | | (SEQ ID NO: | | |
| 670) | | | 770) | | | 870) | | |
| TLDSKTQSL | 1.70 | 9 | TLDSKTQSL | 2.41 | 9 | QSAPHGVVF | 2.70 | 9 |
| (SEQ ID NO: | | | (SEQ ID NO: | | | (SEQ ID NO: | | |
| 671) | | | 771) | | | 871) | | |
| KLFDRYFKY | 1.53 | 9 | TQYNRYLAL | 2.27 | 9 | RTAPHGHVM | 2.69 | 9 |
| (SEQ ID NO: | | | (SEQ ID NO: | | | (SEQ ID NO: | | |
| 672) | | | 772) | | | 872) | | |
| KVDGVVQQ | 1.42 | 9 | TQAPTHLSV | 2.12 | 9 | YVNTFSSTF | 2.57 | 9 |
| L (SEQ ID NO: | | | (SEQ ID NO: | | | (SEQ ID NO: | | |
| 673) | | | 773) | | | 873) | | |
| FLLNKEMYL | 1.38 | 9 | FVDGVPFVV | 2.09 | 9 | YLITPVHVM | 2.57 | 9 |
| (SEQ ID NO: | | | (SEQ ID NO: | | | (SEQ ID NO: | | |
| 674) | | | 774) | | | 874) | | |
| LMIERFVSL | 1.35 | 9 | SQLGGLHLL | 2.05 | 9 | YYTSNPTTF | 2.56 | 9 |
| (SEQ ID NO: | | | (SEQ ID NO: | | | (SEQ ID NO: | | |
| 675) | | | 775) | | | 875) | | |
| FLNRFTTTL | 1.33 | 9 | AQLPAPRTL | 2.04 | 9 | VARDLSLQF | 2.49 | 9 |
| (SEQ ID NO: | | | (SEQ ID NO: | | | (SEQ ID NO: | | |
| 676) | | | 776) | | | 876) | | |
| LLADKFPVL | 1.27 | 9 | FGADPIHSL | 2.01 | 9 | STVFPPTSF | 2.42 | 9 |
| (SEQ ID NO: | | | (SEQ ID NO: | | | (SEQ ID NO: | | |
| 677) | | | 777) | | | 877) | | |
| YLITPVHVM | 1.25 | 9 | TVYSHLLLV | 2.01 | 9 | YYHTTDPSF | 2.41 | 9 |
| (SEQ ID NO: | | | (SEQ ID NO: | | | (SEQ ID NO: | | |
| 678) | | | 778) | | | 878) | | |
| VRFPNITNL | 1.25 | 9 | LMIERFVSL | 1.99 | 9 | LYDKLVSSF | 2.41 | 9 |
| (SEQ ID NO: | | | (SEQ ID NO: | | | (SEQ ID NO: | | |
| 679) | | | 779) | | | 879) | | |
| YYTSNPTTF | 1.24 | 9 | FLNRFTTTL | 1.96 | 9 | VYDPLQPEL | 2.35 | 9 |
| (SEQ ID NO: | | | (SEQ ID NO: | | | (SEQ ID NO: | | |
| 680) | | | 780) | | | 880) | | |
| IRQEEVQEL | 1.21 | 9 | ALWEIQQVV | 1.92 | 9 | VVYRGTTTY | 2.34 | 9 |
| (SEQ ID NO: | | | (SEQ ID NO: | | | (SEQ ID NO: | | |
| 681) | | | 781) | | | 881) | | |
| YITGGVVQL | 1.19 | 9 | YVYSRVKNL | 1.91 | 9 | MSMTYGQQF | 2.27 | 9 |
| (SEQ ID NO: | | | (SEQ ID NO: | | | (SEQ ID NO: | | |
| 682) | | | 782) | | | 882) | | |
| LLLDRLNQL | 1.18 | 9 | HLVDFQVTI | 1.90 | 9 | TANPKTPKY | 2.26 | 9 |
| (SEQ ID NO: | | | (SEQ ID NO: | | | (SEQ ID NO: | | |
| 683) | | | 783) | | | 883) | | |
| VLNDILSRL | 1.17 | 9 | YIDIGNYTV | 1.90 | 9 | KAYKIEELF | 2.24 | 9 |
| (SEQ ID NO: | | | (SEQ ID NO: | | | (SEQ ID NO: | | |
| 684) | | | 784) | | | 884) | | |
| FLLPSLATV | 1.17 | 9 | YLNSTNVTI | 1.87 | 9 | KSYELQTPF | 2.23 | 9 |
| (SEQ ID NO: | | | (SEQ ID NO: | | | (SEQ ID NO: | | |
| 685) | | | 785) | | | 885) | | |
| TMADLVYAL | 1.17 | 9 | VRFPNITNL | 1.86 | 9 | SSLPSYAAF | 2.20 | 9 |
| (SEQ ID NO: | | | (SEQ ID NO: | | | (SEQ ID NO: | | |
| 686) | | | 786) | | | 886) | | |
| VPMEKLKTL | 1.17 | 9 | YVDNSSLTI | 1.85 | 9 | FAYANRNRF | 2.19 | 9 |
| (SEQ ID NO: | | | (SEQ ID NO: | | | (SEQ ID NO: | | |
| 687) | | | 787 | | | 887) | | |
| FGADPIHSL | 1.16 | 9 | RLDKVEAEV | 1.84 | 9 | YYKKDNSYF | 2.18 | 9 |
| (SEQ ID NO: | | | (SEQ ID NO: | | | (SEQ ID NO: | | |
| 688) | | | 788) | | | 888) | | |
| VRDPQTLEI | 1.14 | 9 | YLDAYNMMI | 1.81 | 9 | YFIASFRLF | 2.17 | 9 |
| (SEQ ID NO: | | | (SEQ ID NO: | | | (SEQ ID NO: | | |
| 689) | | | 789) | | | 889) | | |
| YVYSRVKNL | 1.14 | 9 | KVDGVDVEL | 1.77 | 9 | SFYEDFLEY | 2.15 | 9 |
| (SEQ ID NO: | | | (SEQ ID NO: | | | (SEQ ID NO: | | |
| 690) | | | 790) | | | 890) | | |
| YLATALLTL | 1.13 | 9 | VLNDILSRL | 1.76 | 9 | KVDGVVQQL | 2.12 | 9 |
| (SEQ ID NO: | | | (SEQ ID NO: | | | (SEQ ID NO: | | |
| 691) | | | 791) | | | 891) | | |
| VLSDRELHL | 1.13 | 9 | SVVSKVVKV | 1.75 | 9 | LAKDTTEAF | 2.12 | 9 |
| (SEQ ID NO: | | | (SEQ ID NO: | | | (SEQ ID NO: | | |
| 692) | | | 792) | | | 892 | | |
| NLSDRVVFV | 1.12 | 9 | ALSKGVHFV | 1.74 | 9 | VATSRTLSY | 2.12 | 9 |
| (SEQ ID NO: | | | (SEQ ID NO: | | | (SEQ ID NO: | | |
| 693) | | | 793) | | | 893) | | |
| FVDGVPFVV | 1.11 | 9 | RLQSLQTYV | 1.74 | 9 | KQFDTYNLW | 2.09 | 9 |
| (SEQ ID NO: | | | (SEQ ID NO: | | | (SEQ ID NO: | | |
| 694) | | | 794) | | | 894) | | |
| KPNELSRVL | 1.11 | 9 | TRFQTLLAL | 1.74 | 9 | VYYTSNPTTF | 2.08 | 10 |
| (SEQ ID NO: | | | (SEQ ID NO: | | | (SEQ ID NO: | | |
| 695) | | | 795) | | | 895) | | |
| VMYMGTLSY | 1.10 | 9 | GLNDNLLEI | 1.73 | 9 | YFVVKRHTF | 2.06 | 9 |
| (SEQ ID NO: | | | (SEQ ID NO: | | | (SEQ ID NO: | | |
| 696) | | | 796) | | | 896) | | |
| LLYDANYFL | 1.09 | 9 | FLLPSLATV | 1.72 | 9 | LMDGSIIQF | 2.06 | 9 |
| (SEQ ID NO: | | | (SEQ ID NO: | | | (SEQ ID NO: | | |
| 697) | | | 797) | | | 897) | | |
| TLNDLNETL | 1.07 | 9 | KLFIRQEEV | 1.68 | 9 | VYSTGSNVF | 2.05 | 9 |
| (SEQ ID NO: | | | (SEQ ID NO: | | | (SEQ ID NO: | | |
| 698) | | | 798) | | | 898) | | |
| QEYADVFHLY | 1.07 | 10 | TMADLVYAL | 1.68 | 9 | IYNDKVAGF | 2.05 | 9 |
| (SEQ ID NO: | | | (SEQ ID NO: | | | (SEQ ID NO: | | |
| 699) | | | 799) | | | 899) | | |
| TVYSHLLLV | 1.07 | 9 | RQLLFVVEV | 1.68 | 9 | YLQPRTFLL | 2.05 | 9 |
| (SEQ ID NO: | | | (SEQ ID NO: | | | (SEQ ID NO: | | |
| 700) | | | 800) | | | 900) | | |
| SEFSSLPSY | 1.06 | 9 | YLITPVHVM | 1.67 | 9 | MFDAYVNTF | 2.05 | 9 |
| (SEQ ID NO: | | | (SEQ ID NO: | | | (SEQ ID NO: | | |
| 701) | | | 801) | | | 901) | | |
| YLDAYNMMI | 1.06 | 9 | FLLNKEMYL | 1.67 | 9 | YLFDESGEF | 2.03 | 9 |
| (SEQ ID NO: | | | (SEQ ID NO 802) | | | (SEQ ID NO: | | |
| 702) | | | | | | 902) | | |
| RPDTRYVLM | 1.06 | 9 | NRFLYIIKL | 1.65 | 9 | QFAPSASAF | 2.03 | 9 |
| (SEQ ID NO: | | | (SEQ ID NO: | | | (SEQ ID NO: | | |
| 703) | | | 803) | | | 903) | | |
| KIADYNYKL | 1.05 | 9 | KLFDRYFKY | 1.65 | 9 | GYKSVNITF | 2.02 | 9 |
| (SEQ ID NO: | | | (SEQ ID NO: | | | (SEQ ID NO: | | |
| 704) | | | 804 | | | 904 | | |
| FVNEFYAYL | 1.05 | 9 | KLNEEIAII | 1.63 | 9 | FYLTNDVSF | 2.02 | 9 |
| (SEQ ID NO: | | | (SEQ ID NO: | | | (SEQ ID NO: | | |
| 705) | | | 805) | | | 905) | | |
| RLYYDSMSY | 1.04 | 9 | KLLEQWNLV | 1.63 | 9 | VYFLQSINF | 1.97 | 9 |
| (SEQ ID NO: | | | (SEQ ID NO: | | | (SEQ ID NO: | | |
| 706) | | | 806) | | | 906) | | |
| VLLSVLQQL | 1.04 | 9 | IVDEPEEHV | 1.63 | 9 | VASQSIIAY | 1.96 | 9 |
| (SEQ ID NO: | | | (SEQ ID NO: | | | (SEQ ID NO: | | |
| 707) | | | 807) | | | 907) | | |
| ALSKGVHFV | 1.04 | 9 | LLADKFPVL | 1.62 | 9 | AQLPAPRTL | 1.96 | 9 |
| (SEQ ID NO: | | | (SEQ ID NO: | | | (SEQ ID NO: | | |
| 708) | | | 808) | | | 908) | | |
| EEFEPSTQY | 1.04 | 9 | VLLSVLQQL | 1.62 | 9 | VYMPASWVM | 1.95 | 9 |
| (SEQ ID NO: | | | (SEQ ID NO: | | | (SEQ ID NO: | | |
| 709) | | | 809) | | | 909) | | |
| TLVPQEHYV | 1.04 | 9 | KLSYGIATV | 1.61 | 9 | SAKSASVYY | 1.95 | 9 |
| (SEQ ID NO: | | | (SEQ ID NO: | | | (SEQ ID NO: | | |
| 710) | | | 810) | | | 910) | | |
| IPRRNVATL | 1.04 | 9 | FGDDTVIEV | 1.61 | 9 | TTLPVNVAF | 1.94 | 9 |
| (SEQ ID NO: | | | (SEQ ID NO: | | | (SEQ ID NO: | | |
| 711) | | | 811) | | | 911) | | |
| KTFPPTEPK | 1.03 | 9 | YLATALLTL | 1.61 | 9 | VVIPDYNTY | 1.94 | 9 |
| (SEQ ID NO: | | | (SEQ ID NO: | | | (SEQ ID NO: | | |
| 712) | | | 812) | | | 912) | | |
| NYMPYFFTL | 1.03 | 9 | TLVPQEHYV | 1.60 | 9 | SSVELKHFF | 1.93 | 9 |
| (SEQ ID NO: | | | (SEQ ID NO: | | | (SEQ ID NO: | | |
| 713) | | | 813) | | | 913) | | |
| STFNVPMEK | 1.03 | 9 | TLNDLNETL | 1.58 | 9 | SQLGGLHLL | 1.93 | 9 |
| (SEQ ID NO: | | | (SEQ ID NO: | | | (SEQ ID NO: | | |
| 714) | | | 814) | | | 914) | | |
| HPTQAPTHL | 1.03 | 9 | ERSEKSYEL | 1.57 | 9 | VAMPNLYKM | 1.90 | 9 |
| (SEQ ID NO: | | | (SEQ ID NO: | | | (SEQ ID NO: 915) | | |
| 715) | | | 815) | | | | | |
| AEIPKEEVKP | 1.03 | 11 | KIADYNYKL | 1.56 | 9 | NYMPYFFTL | 1.90 | 9 |
| F | | | (SEQ ID NO: | | | (SEQ ID NO: 916) | | |
| (SEQ ID NO: | | | 816) | | | | | |
| 716) | | | | | | | | |
| ALWEIQQVV | 1.03 | 9 | YQPYRVVVL | 1.56 | 9 | MFVKHKHAF | 1.88 | 9 |
| (SEQ ID NO: | | | (SEQ ID NO: | | | (SEQ ID NO: 917) | | |
| 717) | | | 817) | | | | | |
| KPVETSNSF | 1.03 | 9 | YADVFHLYL | 1.56 | 9 | VYIGDPAQL | 1.88 | 9 |
| (SEQ ID NO: | | | (SEQ ID NO: | | | (SEQ ID NO: 918) | | |
| 718) | | | 818) | | | | | |
| VLWAHGFEL | 1.02 | 9 | LLLDRLNQL | 1.55 | 9 | TFFKLVNKF | 1.87 | 9 |
| (SEQ ID NO: | | | (SEQ ID NO: | | | (SEQ ID NO: 919) | | |
| 719) | | | 819) | | | | | |
| VVYRGTTTY | 1.02 | 9 | TLMNVLTLV | 1.53 | 9 | RYKLEGYAF | 1.86 | 9 |
| (SEQ ID NO: | | | (SEQ ID NO: | | | (SEQ ID NO: 920) | | |
| 720) | | | 820) | | | | | |
| GLNDNLLEI | 1.02 | 9 | YLNTLTLAV | 1.53 | 9 | ASRELKVTF | 1.86 | 9 |
| (SEQ ID NO: | | | (SEQ ID NO: | | | (SEQ ID NO: 921) | | |
| 721) | | | 821) | | | | | |
| VPHISRQRL | 1.02 | 9 | YAKPFLNKV | 1.53 | 9 | KHWPQIAQF | 1.85 | 9 |
| (SEQ ID NO: | | | SEQ ID NO: | | | (SEQ ID NO: 922) | | |
| 722) | | | 822) | | | | | |
| VENPHLMG | 1.01 | 9 | YITGGVVQL | 1.52 | 9 | FAVDAAKAY | 1.85 | 9 |
| W | | | (SEQ ID NO: | | | (SEQ ID NO: 923) | | |
| (SEQ ID NO: | | | 823) | | | | | |
| 723) | | | | | | | | |
| QEILGTVSW | 1.01 | 9 | VRIKIVQML | 1.52 | 9 | TTNGDFLHF | 1.83 | 9 |
| (SEQ ID NO: | | | (SEQ ID NO: | | | (SEQ ID NO: 924) | | |
| 724) | | | 824) | | | | | |
| HLVDFQVTI | 1.01 | 9 | SMWALIISV | 1.52 | 9 | LYQPPQTSI | 1.83 | 9 |
| (SEQ ID NO: | | | (SEQ ID NO: | | | (SEQ ID NO: 925) | | |
| 725) | | | 825) | | | | | |
| GEYSHVVAF | 1.01 | 9 | VLSDRELHL | 1.50 | 9 | KALNLGETF | 1.81 | 9 |
| (SEQ ID NO: | | | (SEQ ID NO: | | | (SEQ ID NO: 926) | | |
| 726) | | | 826) | | | | | |
| YLFDESGEFK | 1.01 | 11 | VYDPLQPEL | 1.49 | 9 | AQFAPSASAF | 1.79 | 10 |
| L | | | (SEQ ID NO: | | | (SEQ ID NO: 927) | | |
| (SEQ ID NO: | | | 827) | | | | | |
| 727) | | | | | | | | |
| REHEHEIAW | 1.00 | 9 | MLDMYSVML | 1.48 | 9 | IQLSSYSLF | 1.79 | 9 |
| (SEQ ID NO: | | | (SEQ ID NO: | | | (SEQ ID NO: 928) | | |
| 728) | | | 828) | | | | | |
| ASEFSSLPSY | 1.00 | 10 | FRYMNSQGL | 1.48 | 9 | EYADVFHLY | 1.78 | 9 |
| (SEQ ID NO: | | | (SEQ ID NO: | | | (SEQ ID NO: 929) | | |
| 729) | | | 829) | | | | | |
| KLVNKFLAL | 1.00 | 9 | HLDGEVITF | 1.47 | 9 | YTPSKLIEY | 1.78 | 9 |
| (SEQ ID NO: | | | (SEQ ID NO: | | | (SEQ ID NO: 930) | | |
| 730) | | | 830) | | | | | |
| YLNTLTLAV | 1.00 | 9 | KQLIKVTLV | 1.47 | 9 | YYQLYSTQL | 1.78 | 9 |
| (SEQ ID NO: | | | (SEQ ID NO: | | | (SEQ ID NO: 931) | | |
| 731) | | | 831) | | | | | |
| ILFTRFFYV | 1.00 | 9 | KLDDKDPNF | 1.47 | 9 | ATIPIQASL | 1.78 | 9 |
| (SEQ ID NO: | | | (SEQ ID NO: | | | (SEQ ID NO: 932) | | |
| 732) | | | 832) | | | | | |
| YLNSTNVTI | 1.00 | 9 | IRQEEVQEL | 1.47 | 9 | SIIQFPNTY | 1.77 | 9 |
| (SEQ ID NO: | | | (SEQ ID NO: | | | (SEQ ID NO: 933) | | |
| 733) | | | 833) | | | | | |
| KTFPPTEPKK | 0.99 | 10 | LMDGSIIQF | 1.47 | 9 | MYASAVVLL | 1.77 | 9 |
| (SEQ ID NO: | | | (SEQ ID NO: | | | (SEQ ID NO: 934) | | |
| 734) | | | 834) | | | | | |
| TIKPVTYK | 0.99 | 9 | RLNEVAKNL | 1.47 | 9 | YYRSLPGVF | 1.76 | 9 |
| (SEQ ID NO: | | | (SEQ ID NO: | | | (SEQ ID NO: 935) | | |
| 735) | | | 835) | | | | | |
| VRIQPGQTF | 0.99 | 9 | SLLMPILTL | 1.47 | 9 | KLFDRYFKY | 1.75 | 9 |
| (SEQ ID NO: | | | (SEQ ID NO: | | | (SEQ ID NO: 936) | | |
| 736) | | | 836) | | | | | |
| LLLDDFVEI | 0.99 | 9 | VADAVIKTL | 1.46 | 9 | RFKESPFEL | 1.75 | 9 |
| (SEQ ID NO: | | | (SEQ ID NO: | | | (SEQ ID NO: | | |
| 737) | | | 837) | | | 937) | | |
| YYQLYSTQL | 0.99 | 9 | VLWAHGFEL | 1.45 | 9 | LVSDIDITF | 1.75 | 9 |
| (SEQ ID NO: | | | (SEQ ID NO: | | | (SEQ ID NO: | | |
| 738) | | | 838) | | | 938) | | |
| KTIQPRVEK | 0.99 | 9 | LLLDDFVEI | 1.45 | 9 | YQKVGMQKY | 1.75 | 9 |
| (SEQ ID NO: | | | (SEQ ID NO: | | | (SEQ ID NO: | | |
| 739) | | | 839) | | | 939) | | |
| KRVDWTIEY | 0.98 | 9 | VRSIFSRTL | 1.44 | 9 | RTIKGTHHW | 1.74 | 9 |
| (SEQ ID NO: | | | (SEQ ID NO: | | | (SEQ ID NO: | | |
| 740) | | | 840) | | | 940) | | |
| RLFRKSNLK | 0.98 | 9 | NLSDRVVFV | 1.44 | 9 | ITFDNLKTL | 1.74 | 9 |
| (SEQ ID NO: | | | (SEQ ID NO: | | | (SEQ ID NO: | | |
| 741) | | | 841) | | | 941) | | |
| YPDPSRIL | 0.98 | 8 | VVFLHVTYV | 1.44 | 9 | LMIERFVSL | 1.73 | 9 |
| (SEQ ID NO: | | | (SEQ ID NO: | | | (SEQ ID NO: | | |
| 742) | | | 842) | | | 942) | | |
| ASMPTTIAK | 0.98 | 9 | LQLPQGTTL | 1.43 | 9 | KMFDAYVNTF | 1.72 | 10 |
| (SEQ ID NO: | | | (SEQ ID NO: | | | (SEQ ID NO: | | |
| 743) | | | 843) | | | 943) | | |
| FPRGQGVPI | 0.98 | 9 | SLVKPSFYV | 1.43 | 9 | VMYMGTLSY | .72 | 9 |
| (SEQ ID NO: | | | (SEQ ID NO: | | | (SEQ ID NO: | | |
| 744) | | | 844) | | | 944) | | |
| RTAPHGHVM | 0.98 | 9 | KLVNKFLAL | 1.42 | 9 | SYATHSDKF | 1.71 | 9 |
| (SEQ ID NO: | | | (SEQ ID NO: | | | (SEQ ID NO: | | |
| 745) | | | 845) | | | 945) | | |
| LPYPDPSRIL | 0.98 | 10 | ARYMRSLKV | 1.42 | 9 | KFYGGWHNM | 1.71 | 9 |
| (SEQ ID NO: | | | (SEQ ID NO: | | | (SEQ ID NO: | | |
| 746) | | | 846) | | | 946) | | |
| AVFDKNLYDK | 0.98 | 10 | LLYDANYFL | 1.41 | 9 | SLYVNKHAF | 1.70 | 9 |
| (SEQ ID NO: | | | (SEQ ID NO: | | | (SEQ ID NO: | | |
| 747) | | | 847) | | | 947) | | |
| QELGKYEQY | 0.98 | 9 | IQPGQTFSV | 1.40 | 9 | FGADPIHSL | 1.69 | 9 |
| (SEQ ID NO: | | | (SEQ ID NO: | | | (SEQ ID NO: | | |
| 748) | | | 848) | | | 948) | | |
| FLAHIQWMV | 0.97 | 9 | DADSKIVQL | 1.40 | 9 | SFSASTSAF | 1.68 | 9 |
| (SEQ ID NO: | | | (SEQ ID NO: | | | (SEQ ID NO: | | |
| 749) | | | 849) | | | 949) | | |
| SEVGPEHSL | 0.97 | 9 | EAFEKMVSL | 1.39 | 9 | LALSKGVHF | 1.66 | 9 |
| (SEQ ID NO: | | | (SEQ ID NO: | | | (SEQ ID NO: | | |
| 750) | | | 850) | | | 950) | | |
| KLFAAETLK | 0.97 | 9 | WRNTNPIQL | 1.38 | 9 | RFDNPVLPF | 1.66 | 9 |
| (SEQ ID NO: | | | (SEQ ID NO: | | | (SEQ ID NO: | | |
| 751) | | | 851) | | | 951) | | |
| VVYRGTTTYK | 0.96 | 10 | AMYTPHTVL | 1.38 | 9 | AMYTPHTVL | 1.66 | 9 |
| (SEQ ID NO: | | | (SEQ ID NO: | | | (SEQ ID NO: | | |
| 752) | | | 852) | | | 952) | | |
| TSFGPLVRK | 0.96 | 9 | QQGEVPVSI | 1.38 | 9 | FLNRFTTTL | 1.65 | 9 |
| (SEQ ID NO: | | | (SEQ ID NO: | | | (SEQ ID NO: | | |
| 753) | | | 853) | | | 953) | | |
| SEPVLKGVKL | 0.96 | 10 | LQAENVTGL | 1.37 | 9 | TYFTQSRNL | 1.65 | 9 |
| (SEQ ID NO: | | | (SEQ ID NO: | | | (SEQ ID NO: | | |
| 754) | | | 854) | | | 954) | | |
| KEIKESVQTF | 0.96 | 10 | FQSAVKRTI | 1.37 | 9 | YAFEHIVY | 1.64 | 8 |
| (SEQ ID NO: | | | (SEQ ID NO: | | | (SEQ ID NO: | | |
| 755) | | | 855) | | | 955) | | |
| SQLGGLHLL | 0.96 | 9 | QLSLPVLQV | 1.37 | 9 | LQIPFAMQM | 1.64 | 9 |
| (SEQ ID NO: | | | (SEQ ID NO: | | | (SEQ ID NO: | | |
| 756) | | | 856) | | | 956) | | |
| APRTLLTKGT | 0.96 | 11 | SLDNVLSTF | 1.36 | 9 | AYILFTRFF | 1.64 | 9 |
| L | | | (SEQ ID NO: | | | (SEQ ID NO: | | |
| (SEQ ID NO: | | | 857) | | | 957) | | |
| 757) | | | | | | | | |
| WRNTNPIQL | 0.96 | 9 | FLAHIQWMV | 1.35 | 9 | YYLFDESGEF | 1.63 | 10 |
| (SEQ ID NO: | | | (SEQ ID NO: | | | (SEQ ID NO: | | |
| 758) | | | 858) | | | 958) | | |
| EEAIRHVRAW | 0.96 | 10 | HANEYRLYL | 1.34 | 9 | YSYATHSDKF | 1.63 | 10 |
| (SEQ ID NO: | | | (SEQ DI NO: | | | (SEQ ID NO: | | |
| 759) | | | 859) | | | 959) | | |
| TEKSNIIRGW | 0.96 | 10 | QMAPISAMV | 1.34 | 9 | KFVRIQPGQTF | 1.63 | 11 |
| (SEQ ID NO: | | | (SEQ ID NO: | | | (SEQ ID NO: | | |
| 760) | | | 860) | | | 960) | | |
| RQFHQKLLK | 0.96 | 9 | EQFKKGVQI | 1.33 | 9 | FTSDYYQLY | 1.63 | 9 |
| (SEQ ID NO: | | | (SEQ ID NO: | | | (SEQ ID NO: | | |
| 761) | | | 861) | | | 961) | | |
| APHGHVMVE | 0.95 | 10 | FVNEFYAYL | 1.33 | 9 | VYANGGKGF | 1.62 | 9 |
| L | | | (SEQ ID NO: | | | (SEQ ID NO: | | |
| (SEQ ID NO: | | | 862) | | | 962) | | |
| 762) | | | | | | | | |
| QIYKTPPIK | 0.95 | 9 | YRLFRKSNL | 1.32 | 9 | SVYYTSNPTT | 1.6 | 11 |
| (SEQ ID NO: | | | (SEQ ID NO: | | | F | | |
| 763) | | | 863) | | | (SEQ ID NO: | | |
| | | | | | | 963) | | |
| SVVSKVVKV | 0.95 | 9 | NVFAFPFTI | 1.32 | 9 | YFIKGLNNL | 1.61 | 9 |
| (SEQ ID NO: | | | (SEQ ID NO: | | | (SEQ ID NO: | | |
| 764) | | | 864) | | | 964) | | |
| KENDSKEGF | 0.95 | 9 | ILFTRFFYV | 1.32 | 9 | TLKEILVTY | 1.61 | 9 |
| (SEQ ID NO: | | | (SEQ ID NO: | | | (SEQ ID NO: | | |
| 765) | | | 865) | | | 965) | | |
| KSAGFPFNK | 0.95 | 9 | KLWAQCVQL | 1.32 | 9 | LYLQYIRKL | 1.60 | 9 |
| (SEQ ID NO: | | | (SEQ ID NO: | | | (SEQ ID NO: | | |
| 766 | | | 866) | | | 966) | | |
| MEVTPSGTW | 0.95 | 9 | TLQPVSELL | 1.31 | 9 | KHIDAYKTF | 1.60 | 9 |
| (SEQ ID NO: | | | (SEQ ID NO: | | | (SEQ ID NO: | | |
| 767) | | | 867) | | | 967) | | |
| TLMNVLTLV | 0.95 | 9 | ILDGISQYSL | 1.31 | 10 | RSFIEDLLF | 1.59 | 9 |
| (SEQ ID NO: | | | (SEQ ID NO: 868 | | | (SEQ ID NO: | | |
| 768) | | | | | | 968) | | |

Using these ranked lists, vaccine or T cell therapy development against HPV infection could be done as explained above. The methods involve the identification of a high-density core, which is the 7 amino acids long peptide YLHPSYY (SEQ ID NO: 1084) that is present in highly ranked epitopes of YLHPSYYML (SEQ ID NO: 370) in all three subjects. However, subject 10881 would have four strong epitopes YLHPSYYML (SEQ ID NO: 370), FYLHPSYYM (SEQ ID NO: 582), FYLHPSYY (SEQ ID NO: 638) and FYLHPSYYML (SEQ ID NO: 645) (aggregated EWS=1.87+1.70+1.03+1.00=5.60) compared to the much weaker aggregated EWS of 2.09 (1.37+0.72) for subject 3819 and 1.81 for subject 3821. These data suggest that a 10 amino acids long HPV vaccine antigen of FYLHPSYYML (SEQ ID NO: 645) would induce CD8+ T-cell responses in all three subjects, but the potency of the antigen would be the strongest in subject 10881 and the weakest in subject 3821. The YLHPSYY (SEQ ID NO: 1084) core would be an excellent target for TCR T cell therapy to kill HPV-infected cells in these subjects. Similarly, for SARS-COV-2 the 11 amino acids long peptide SVYYTSNPTTF (SEQ ID NO: 963) could be selected as a vaccine antigen that induces CD8+ T cell responses in all three subjects and the 9 amino acids long core of YYTSNPTTF (SEQ ID NO: 680) as a target for T-cell therapy.

### Determining the Strength of T Cell Responses

Interestingly, the EWS values of the top-ranked epitopes were in a similar range independently of their location in the proteome. A new definition for the strength of the individual's T-cell response can be proposed, namely the average potency of the top ranked epitopes. The strength of T-cell responses can be estimated from the average potency of the top-ranked epitopes, e.g. average EWS of the top-100 epitopes. For example, considering the HBV infection, the virus could induce weak T-cell responses in subject 3819 (Ave EWS=0.81) and strong T cell responses in subject 10881 (Ave EWS=1.32). In another example, considering the SARS-COV-2 data above, subject 3819 displayed weak T-cell responses with an average EWS of 1.1 and a range between 0.95 and 1.90. Subject 3821 had intermediate T-cell responses with an average EWS of 1.62 and a range between 1.31 and 2.56. For subject 10881, the average EWS was 1.95 and it ranged between 1.88 and 3.81. In other words, subject 10881 exhibited the most potent T-cell response based on the mean EWS of the top 100 epitopes. These results cannot be directly confirmed by experimental methods since it is impossible to measure the potency of all putative epitopes of a virus in an individual because the limitation of blood volume that would be required for so many tests.

It is noted that the relative strength of T cell responses to different viruses is similar in individuals. As shown above, the same individuals had weak, intermediate, and potent T-cell responses to Hepatitis B, Human papillomavirus and SARS-COV-2. For example, HBV, HPV and SARS-COV-2 induced strong T cell responses in subject 10881 and weak T cell responses in subject 3819. This data suggests that the HLA-genotype of subjects determine the strength of T-cell responses to viral infections and tumors among other diseases that relates to their clinical response. The strength of T-cell responses against viral infections, estimated by epitope density on the cell surface, is a phenotype determined by the HLA genotype of the individual. Subjects with strong T cell responses have mild or no disease because they rapidly kill sick cells. By contrast, subjects with weak T cell responses may have severe or chronic disease because their T cells are weak to kill sick cells. The computer implemented method invented here is useful to predict the clinical outcome of infectious or malignant diseases via predicting the strength of T-cell responses of individuals. These predictions may influence the preventive measures that the physician or the patient should take to mitigate the risk of severe or chronic diseases.

The results further illustrate that T-cell responses are unique for each individual due to the extreme diversity in HLA-genotypes among individuals. The computer-implemented method described here is the first method that is capable of prioritizing the epitopes of individuals based on an estimation of their density on the cell surface, which is an essential step for the identification of T-cell antigens for the development different medicinal products targeting high-density cell surface molecules. For example, high-density epitopes are the specific and abundant targets of T-cells on the surface of infected cells and tumor cells.

### Example System

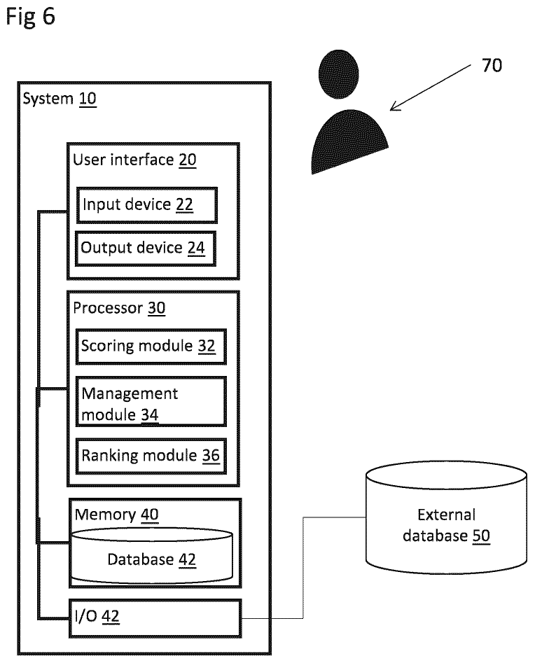

FIG. 6 schematically shows a system 10 for implementing the methods described above. There are standard components of whichever hardware solution is deployed, including for example a user interface 20, a processor 30, memory 40 and an interface 42 for connecting with any external database(s) 50. It will be appreciated that the system may also comprise other standard components which are not depicted for ease of illustration.

The user interface 20 may include an input device 22 such as a touchscreen, keyboard, mouse a voice activated input device or any other suitable device. The user 70 can use the input device 22 to input one or both of the HLA genotype data of the subject to be considered and the protein(s) expressed in the sick cell of the subject. The user interface 20 may also include an output device 24 such any suitable display on which information may be displayed to a user 70. The output device 24 may display the output of the computer implemented method described above. For example, the output may be a ranked list of T-cell antigens of a subject individual optionally with a value for the potency of the T-cell antigens. Where appropriate, the output may be a personalised vaccine or a suggested treatment plan for the individual.

The processor 30 may process the received inputs to generate the desired outputs as described above. For example, the specific processing may be completed in dedicated modules, e.g. a scoring module 32 which generates the scores as described above and a ranking module 36 which ranks the scores. The scoring and ranking modules may use any suitable technology, for example the ranking module may include an AI core which implements a diagnostics algorithm to predict the potency of T-cell responses based on subject's data. The modules, particularly the scoring module 32, may access the database 42 for scores associated with specific T-cell antigens and cores. This database may be stored locally in the memory 40. Alternatively, the data may be stored in an external database 50 and the machine learning module 32 is able to communicate with such an external database 52.

The processor 30 also comprises a management module 34. Such a management module may be included to manage workflow and to track subject data (for example, to make sure there is CDISC compliant data structure and storage). The management module 34 may be used to manage vaccine manufacturing and delivery. The management module 34 may also be configured to generate subject specific reports (e.g. T-cell antigens diagnostics, potency of T cell responses). The management module 34 may also thus communicate with the internal database 42 and/or the external database 50. For both internal and external databases, it will be appreciated that the data may be split across more than one database and may be stored in any suitable storage, e.g. the cloud for the external database. The external databases may be remote (i.e. in a different location) to the system.

Terms such as ‘component’, ‘module’, ‘processor’ or ‘unit’ used herein may include, but are not limited to, a hardware device, such as circuitry in the form of discrete or integrated components, graphics processing units (GPUs), a Field Programmable Gate Array (FPGA) or Application Specific Integrated Circuit (ASIC), which performs certain tasks or provides the associated functionality. In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. Although the example embodiments have been described with reference to the components, modules and units discussed herein, such functional elements may be combined into fewer elements or separated into additional elements.

### Database

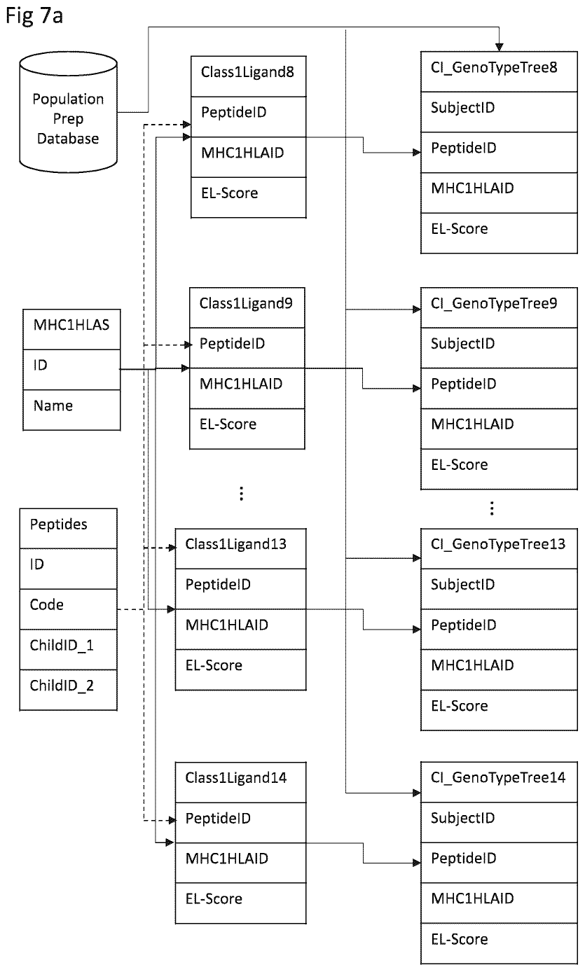

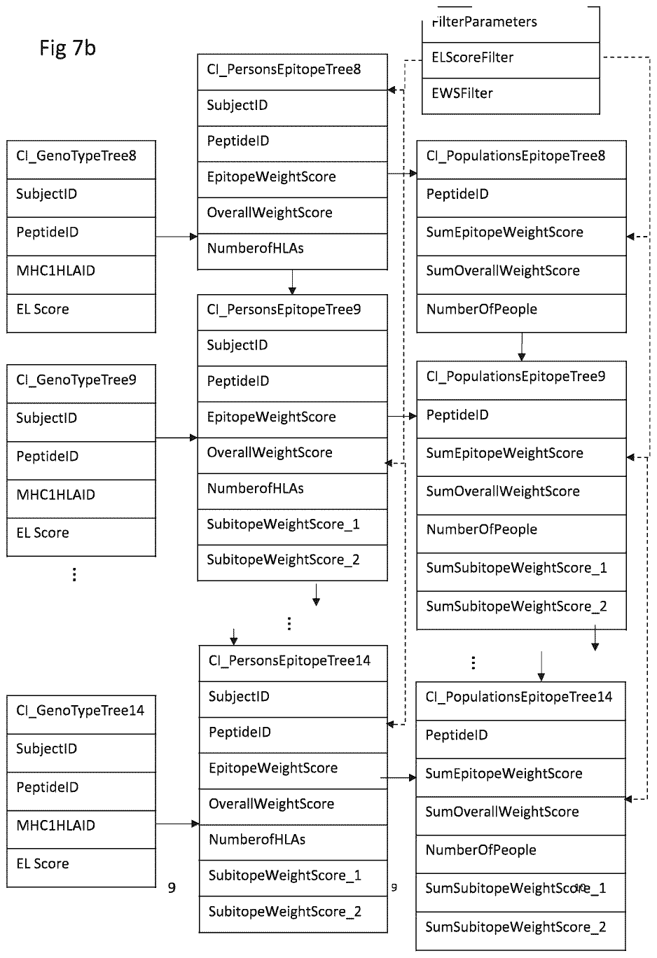

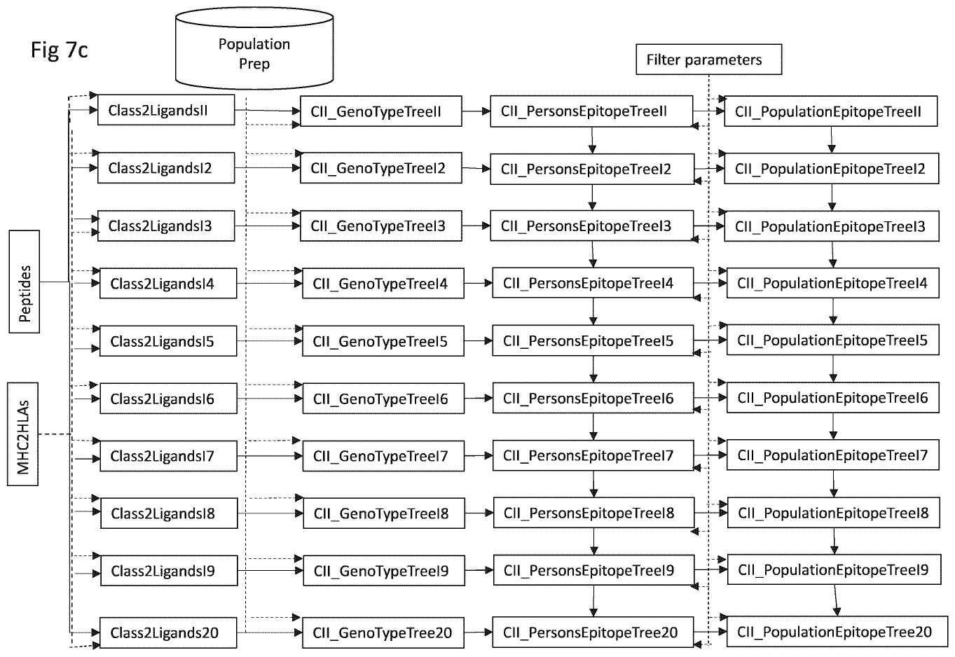

As explained above, various scores may be calculated or determined as part of the VERDI method. To facilitate the determination of these scores, various databases can be used to store information. This allows the scores to be calculated in a prompt timescale. For example, FIG. 7a, shows an example epitope database which contains the sequence of all putative epitopes (between 8 and 20 amino acids in length) from proteins (e.g. all proteins expressed from the SARS COV-2 genome). Epitope databases from different proteins relevant to different diseases are typically stored separately. The HLA data may be separated into HLA Class I databases and HLA Class II databases, each comprising the data which represents the sequence of the HLA allele (e.g. the 4 to 8 digits described below). The HLA database of FIG. 7a is entitled MHC1HLAs and includes the ID and the name of each class I HLA allele. The population database shown in FIG. 7a may include the ID of the individual, their 6 HLA class I alleles and their 12 HLA class II alleles.

The database entitled Peptides includes the ID of each peptide, the amino acid sequence (code) for each peptide, the ID of the left sub-epitope and the ID of the right sub-epitope. The left and right sub-epitopes are each one amino acid shorter than the original peptides. For example, for the sequence TLDSKTQSL (SEQ ID NO: 672), the left sub-epitope is TLDSKTQS (SEQ ID NO: 1115) and the right sub-epitope is LDSKTQSL (SEQ ID NO: 1116). The database of peptides may be based on a single protein of interest, e.g. the SARS-COV-2 protein. The protein may be cut or segmented into a plurality of smaller sequences and the database may include all these smaller sequences which may be termed putative epitopes. For the SARS-COV-2 genome that encodes 29 proteins, there may be around 70,000 or 100,000 smaller sequences which are derivable from the original viral proteins, and which are to be paired with the HLA class I or HLA class II alleles respectively. Putative epitopes can also originate from distinct protein products translated from alternatively spliced regions or alternative reading frames.

As explained above, the EL score may be calculated for each known pair of HLA allele and peptide. Each score may be stored together with the ID of the peptide and the ID of the HLA allele as shown in FIG. 7a and an example database is entitled “Class1Ligandx” and may be termed a ligand data set. Just four of these datasets “Class1Ligandx” are shown in FIG. 7a to illustrate the detail within each database and also the flow of data between the databases. In summary, each of these intermediate datasets is created by pairing a peptide from the Peptides database having x amino acids with one of the Class-I HLA from the MHC1HLAs database and generating a score for each paired peptide and Class-I HLA.

Once the data for an individual is obtained, the EL-score for each paired sequence and HLA allele (both class I and class II) for each individual may be obtained. As illustrated in FIG. 7a, the EL-score may be obtained from the appropriate ligand databases which have been built as described above. The EL scores may be stored for individuals, for example in second intermediate data sets entitled “CL_GenoTypeTreex”. Each dataset contains the ID of the subject, the IDs of the paired peptide and the Class-I HLA and the score for each of the paired peptide and the Class-I HLAs. As previously, x is a number between 8 and 14 in this example for HLA class I alleles. A similar database is also generated for the HLA class II alleles. These databases may be considered to be subject ligand databases.

FIG. 7b shows how the subject ligand datasets may be used to obtain a set of output datasets which are labelled “Cl-PersonsEpitopeTreex” where x is the number between 8 and 14 for class I and 11 to 20 for class II. The table labelled “Cl-PersonsEpitopeTreex”contains the efficacy of each peptide for each subject and may thus be termed a personal epitope potency database.

The table labelled “Cl-PersonsEpitopeTreex” contains the ID (anonymised) for the subject and the ID of the epitope for which potency is of interest. The capacity of an epitope to activate T-cells depends on the number of epitopes on the cell surface signaling simultaneously and/or consecutively to TCRs. The density of each epitope on the cell surface may be quantified using the EpitopeWeightScore (EWS) which is described above. The EWS is the number of contributing HLA-epitope pairs, each weighted by the associated EL-score (or associated/contributing EL scores (ELs)). The EWS may also be weighted by the expression level and stability of the different HLA alleles. For example, as described in “Variations in HLA-B cell surface expression, half-life and extracellular antigen receptivity” by Yarzabek et al published in eLife Immunology and Inflammation in July 2018, HLA-B*0.8-01 allele has high cell-surface expression and high cell surface stability in lymphocytes compared to other alleles (HLA-B*51-01).

The EL scores used to calculate the EWS may be taken from the tables CI_GenoTypeTreex and thus as shown in FIG. 7b, there is an arrow showing data flow from CI_GenoTypeTree8 to Cl-PersonsEpitopeTree8 (and so on). The table labelled “Cl-PersonsEpitopeTreex” also contains the number of HLAs for which the peptide has an EL score above the EL threshold. Similarly, as shown in FIG. 7b, data can flow from the tables labelled “Cl-PersonsEpitopeTreex” which contain the Epitope WeightScores for the peptides which are one shorter than the peptide being considered. In other words, the EWS from the earlier tables may be used to calculate the subitope scores.

The table labelled “Cl-PopulationEpitopeTreex” contains the efficacy of each peptide for the overall population and may thus be termed an overall peptide efficacy database. Together with the input population data, the data from the table labelled “Cl-PersonsEpitopeTreex” may be used to create the data in the table labelled “Cl-PopulationEpitopeTreex”. This table contains the ID of the peptide for which efficacy is of interest. The table contains various scores which are summed across the entire training population and thus the table also contains the number of people which contribute to the sum.

For the shortest peptides, e.g. 8 length in this example, the scores include the SumEpitopeWeightScore (SEWS) which is the sum of the EpitopeWeightScore (EWS) for the peptide for all subjects in the population and the SumOverallWeightScore (SOWS) which is calculated as set out below. For the longer peptides, these sums are also present together with the additional scores: the SumSubitope WeightScore_1 (SSWS1) which is the sum of the SubitopeWeightScore_1 (SWS1) for the peptide for all subjects in the population and the SumSubitope WeightScore_2 (SSWS2) which is the sum of the Subitope WeightScore_1 (SWS2) for the peptide for all subjects in the population. These calculations may be done using:

$$SEWS ( x ) = {\sum }_{l = 1}^{q} {EWS}_{l} ( x ) \\ SSWS 1 ( x ) = {\sum }_{l = 1}^{q} SWS {1}_{l} ( x ) \\ SSWS 2 ( x ) = {\sum }_{l = 1}^{q} SWS {2}_{l} ( x ) \\ SOWS ( x ) = {\sum }_{l = 1}^{q} {OWS}_{l} ( x )$$

where l is between 1 and q with q being the number of subjects in the population and (x) indicates that these are calculated for each peptide.

FIG. 7b also shows that the data in these output tables may be adjusted by using filter parameters. These include an ELScoreFilter and an EWSFilter. As explained above, only the EL scores which are above an EL threshold may be summed to calculate the EWS and the ELScoreFilter may be used to set this EL threshold. The EL threshold is set to filter out the peptides that unlikely bind to an HLA (peptides that very unlikely to be transported to the cell surface). When the EL-score is between 0 and 1, an EL-threshold of 0 means that all scores are summed. Similarly, an EWS threshold may be set within the EWSFilter. The EWSFilter is set to filter out epitopes that unlikely induce T-cell responses (low amount on the cell surface to activate T-cells). As explained above, this threshold may be set based on the objectives how many peptides needs to be included.

FIGS. 7a and 7b show the detail of the tables for each of the class-I HLAs being considered. The same data flows and resulting data tables may also be created for the analysis of the class-II HLAs. FIG. 7c combines the information from FIGS. 7a and 7b to show the data flows but omits the details of the data within each table for clarity. It will be appreciated that the data in each table is the same as for FIGS. 7a and 7b except for the use of II rather than I to show that class-II HLAs are being considered. Similarly, FIGS. 7a and 7b can be combined with the same data flow shown in FIG. 7c. As in FIGS. 7a and 7b, the tables in FIG. 7c are labelled “Class2Ligandx”, “CII_GenoTypeTreex”, “CII_PersonsEpitopeTreex”, “CII_PopulationEpitopeTreex” where x is the number of amino acids in each peptide. In FIGS. 7a and 7b, x varied between 8 and 14 and in FIG. 7c, x varies between 11 and 20.

### Validation Model Training and Inference

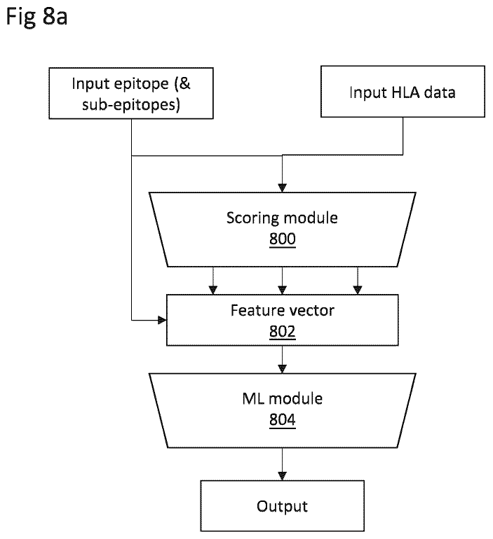

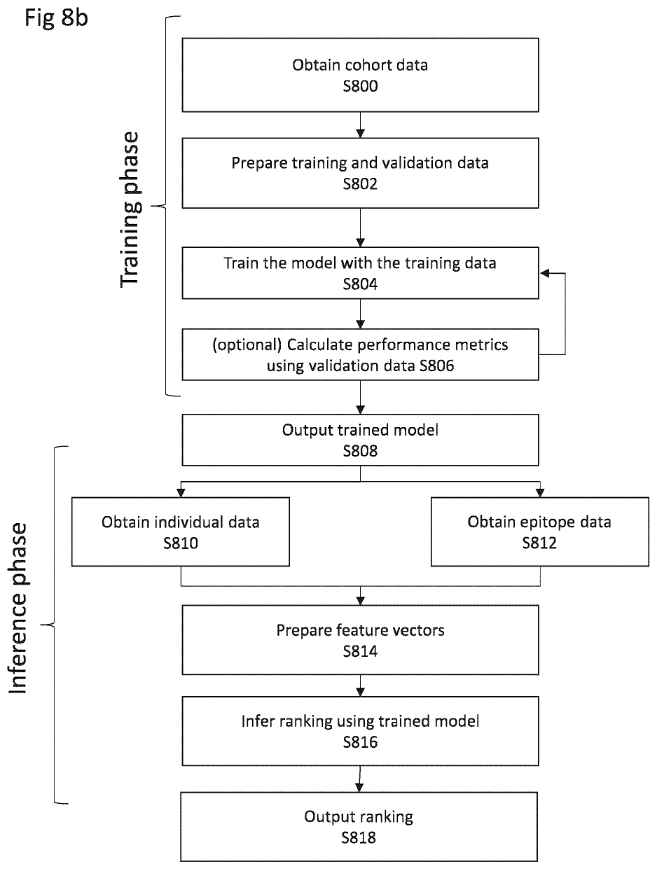

As shown in FIG. 5a, the first step is to obtain a ranked list of epitopes. As describe above, the ranking may be based on the EWS score. To further validate the methods described above, a validation system which is schematically shown in FIG. 8a was developed. As shown, there are two separate inputs: the HLA genotype data for a test subject and an input epitope (with its sub-epitopes). The separate inputs are input as a pair to a scoring module 800. The scoring module generates a plurality of scores for each pair as explained above in relation to FIG. 4. Several arrows are indicated but it will be appreciated that three is merely illustrative and more typically when considering HLA class I molecules, six scores may be calculated. For example, each score may be derived from an EL score generated by the NetMCHPan4.1 or similar module. These scores may be output to a feature vector 802.

Features relating to the original input epitope may also be input to the feature vector. One feature may be termed “log-fold change” which quantifies the potency of the epitope compared to no epitope or a baseline indicating the expansion of antigen-specific T-cells after an infection in the body. Other features may include for example the dominant core for each of the HLA-A, HLA B and HLA-C class I alleles. The core is the pattern or sequence of amino acids in the epitope which is recognized by the TCR.

The feature vector is then input to a machine learning module 804 which has been trained as explained below. The machine learning module 804 may use any suitable technique, for example gradient boosting decision tree (GBDT), RIDGE regression, logistic regression and a balanced random forest technique (described for example, in “Stochastic gradient boosting,” by Jerome H Friedman published in Computational Statistics and Data Analysis, vol. 38, no. 4, pp. 367-378, 2002, “Kernel ridge regression in Empirical inference” by Vovk V. in Springer; 2013. p. 105-16, “Logistic Regression: Relating Patient Characteristics to Outcomes” by Tolles et al in JAMA.2016; 316 (5): 533-534. doi: 10.1001/jama.2016.765 or https://statistics.berkeley.edu/sites/default/files/tech-reports/666.pdf). The machine learning module 804 is used to predict the probability that the input epitope is immunogenic for the individual from whom the HLA genotype data was obtained. The process is repeated for multiple input epitopes which may be ranked based on their probability. The output which is reported is the specificity of each epitope (namely the sequence of amino acids in the epitope) and the potency of each epitope (e.g. a probability value of between 0 to 100 that the epitope will induce a T-cell response) The top-ranked epitopes define the T-cell antigen repertoire for an individual and can be used to predict the response of an individual to a specific illness such as SARS-CoV-2 or cancer.

FIG. 8b illustrates a process for training such a machine learning model as well as the use of the model in the interference stage. In a first stage S800 of the training phase, the cohort data which is being used to train the model is obtained. The cohort data may include the complete HLA genotype of individuals. An example of such data has been gathered together by ImmPort and for the present techniques, the ImmPort database (e.g. using https://www.immport.org/shared/home) was used to access the complete HLA genotype of individuals in the European SDY614 and US SDY28 population. The cohort data also comprises data which evaluates the potency of T-cell responses of individuals to particular illnesses.

As an example, the model may be trained to accurately rank SARS-COV-2 specific T-cell antigens by potency. Examples of such cohort data which are suitable for training a model to make such a prediction include the ABF data described in “SARS-COV-2 genome wide T-cell epitope mapping reveals immunodominance and substantial CD8⁺ T cell activation in COVID-19 patients” by Saini et al published in Sci Immunol 2021 Apr. 14. The ABF cohort comprises 79 individuals characterized with a 4-digit genotype of 6HLA class I alleles representing the sequence of the epitope-binding pocket of the HLA. Another example of cohort data is the Adaptive data described in “Magnitude and Dynamics of the T-Cell Response to SARS-COV-2 Infection at Both Individual and Population Levels” by Snyder et al published in MedRxiv in 2020. The Adaptive data comprises 114 individuals characterized with a 4-digit genotype.

The next step S802 is to prepare the training and validation data from the cohort data. The ABF cohort data may be used as training and cross-validation data. The set of clinical outcomes which are recorded for this data includes healthy, high-risk healthy, outpatient and hospitalized. When preparing the data, epitopes may first be selected to pair with the individuals' data. The epitopes which are selected are those which have a strong binding to one or more dominant HLA class I alleles for the individuals. The binding strength may be calculated using any suitable technique, e.g. using the NetMHCpan 4.1 prediction which was described above in relation to calculating the EWS score. The scores which are used in the feature vector for the inference stage are also obtained for each subject and each selected peptide. T-cell responses may also be measured with labelled peptide MHC-I multimers to quantify the potency of CD8⁺ T-cell antigens as “log-fold change (LFC)” compared to no antigen or baseline indicating the expansion of antigen-specific T-cells after SARS-CoV-2 infection in the body. The “log-fold change” value may be included in the training data. All the published potency data of the epitopes per HLA-allele matched individuals was also obtained. 2,204 epitopes may be selected and the potency data of an average of 920 [209-1452] epitopes may be obtained. Merely as an example of the data which is generated at this step, the table below shows examples of 10 peptides for two individuals: one who was hospitalized and the other who was healthy. It is noted that the first 10 peptides for these two individuals are not the same.

Training data based on ABF cohort

| ID | Peptide | LFC | p | EWS_A | EWS_B | EWS_C | SWS_A | SWS_B | SWS_C | EL_Max |
|---|---|---|---|---|---|---|---|---|---|---|
| AP020 1 | ADLDDFSKQL | 0 | 0 | 0.43795 | 0.00287 | 0.00488 | 0.20439 | 0.06577 | 0.04083 | 0.43742 |
| | (SEQ ID NO: | | | | | | | | | |
| | 969) | | | | | | | | | |
| AP020 1 | AFPFTIYSL | 0 | 0 | 0.60717 | 0.02471 | 0.16018 | 0.01223 | 1.24204 | 0.00778 | 0.59309 |
| | (SEQ ID NO: | | | | | | | | | |
| | 970) | | | | | | | | | |
| AP020 1 | AHAEETRKL | 0 | 0 | 0.01894 | 0.01312 | 0.20139 | 0.00055 | 0.07715 | 0.05133 | 0.19299 |
| | (SEQ ID NO: | | | | | | | | | |
| | 971) | | | | | | | | | |
| AP020 1 | ALLADKFPVL | 0 | 0 | 0.49288 | 0.0049 | 0.00564 | 1.95057 | 0.41905 | 0.48676 | 0.49145 |
| | (SEQ ID NO: | | | | | | | | | |
| | 972) | | | | | | | | | |
| AP020 1 | ALVYFLQSI | 0 | 0 | 0.57459 | 0.00606 | 0.02414 | 0.00432 | 0.21684 | 0.02284 | 0.57381 |
| | (SEQ ID NO: | | | | | | | | | |
| | 973) | | | | | | | | | |
| AP020 1 | ALWEIQQVV | 0 | 0 | 0.98417 | 0.02962 | 0.12551 | 0.85967 | 0.04618 | 0.00922 | 0.98246 |
| | (SEQ ID NO: | | | | | | | | | |
| | 974) | | | | | | | | | |
| AP020 1 | ANPKTPKYKF | 0 | 0 | 0.04313 | 0.43724 | 0.00166 | 0.0161 | 0.7811 | 0.02457 | 0.4317 |
| | (SEQ ID NO: | | | | | | | | | |
| | 975) | | | | | | | | | |
| AP020 1 | APFLYLYAL | 0 | 0 | 0.00255 | 0.95826 | 0.00658 | 0.00168 | 0.103 | 0.00013 | 0.64646 |
| | (SEQ ID NO: | | | | | | | | | |
| | 976) | | | | | | | | | |
| AP020 1 | APHGVVFL | 0 | 0 | 0.00061 | 0.76929 | 0.00392 | 0 | 0 | 0 | 0.57212 |
| | (SEQ ID NO: | | | | | | | | | |
| | 977) | | | | | | | | | |
| AP020 1 | APHGVVFLHV | 0 | 0 | 0.00129 | 0.36514 | 0.00242 | 0.00341 | 1.01004 | 0.02207 | 0.21838 |
| | (SEQ ID NO: | | | | | | | | | |
| | 978) | | | | | | | | | |
| BC10 | AADLDDFSKQL | 0 | 0 | 0.33319 | 0.00356 | 0.03438 | 0.63855 | 0.01728 | 0.51329 | 0.33319 |
| | (SEQ ID NO: | | | | | | | | | |
| | 979) | | | | | | | | | |
| BC10 | ALLADKFPVL | 0 | 0 | 0.49145 | 0.03154 | 0.004 | 1.91578 | 0.32491 | 0.46371 | 0.49145 |
| | (SEQ ID NO: | | | | | | | | | |
| | 980) | | | | | | | | | |
| BC10 | ALLAVFQSA | 0 | 0 | 0.55856 | 0.01402 | 0.00159 | 0.01419 | 0.00109 | 0.00012 | 0.55856 |
| | (SEQ ID NO: | | | | | | | | | |
| | 981) | | | | | | | | | |
| BC10 | ALQDAYYRA | 0 | 0 | 0.53492 | 0.0028 | 0.00248 | 0.00134 | 0.0003 | 0.00062 | 0.53492 |
| | (SEQ ID NO: | | | | | | | | | |
| | 982) | | | | | | | | | |
| BC10 | ALRKVPTDNY | 0 | 0 | 0.0007 | 0.7012 | 0.00612 | 0.00011 | 0.09329 | 0.13022 | 0.69991 |
| | (SEQ ID NO: | | | | | | | | | |
| | 983) | | | | | | | | | |
| BC10 | ALTCFSTQF | 0 | 0 | 0.00882 | 0.24754 | 0.0072 | 0.00013 | 0.00947 | 0.00081 | 0.24648 |
| | (SEQ ID NO: | | | | | | | | | |
| | 984) | | | | | | | | | |
| BC10 | ALWEIQQVV | 0 | 0 | 0.98246 | 0.05262 | 0.04982 | 0.85777 | 0.00454 | 0.03457 | 0.98246 |
| | (SEQ ID NO: | | | | | | | | | |
| | 985) | | | | | | | | | |
| BC10 | AMDEFIERY | 0 | 0 | 0.16011 | 0.49283 | 0.35678 | 0.00303 | 0.00387 | 0.0068 | 0.4911 |
| | (SEQ ID NO: | | | | | | | | | |
| | 986) | | | | | | | | | |
| BC10 | APISAMVRM | 0 | 0 | 0.00109 | 0.76739 | 0.03833 | 3.9E−05 | 0.01404 | 0.00047 | 0.76242 |
| | (SEQ ID NO: | | | | | | | | | |
| | 987) | | | | | | | | | |
| BC10 | APKEIIFL | 0 | 0 | 0.00041 | 0.52002 | 0.01206 | 0 | 0 | 0 | 0.51973 |
| | (SEQ ID NO: | | | | | | | | | |
| | 988) | | | | | | | | | |

| | | Genome | | | | |
|---|---|---|---|---|---|---|
| ID | Peptide | Index | Condition | Dom HLA | DomCore | |
| AP020 1 | ADLDDFSKQL | 29053 | Hospitalized | HLA-A*02:01 | ALDDFSKQL | |
| | (SEQ ID NO: | | | | (SEQ ID NO: 1085) | |
| | 969) | | | | | |
| AP020 1 | AFPFTIYSL | 29140 | Hospitalized | HLA-A*24:02 | AFPFTIYSL | |
| | (SEQ ID | | | | (SEQ ID NO: 970) | |
| | NO: 970) | | | | | |
| AP020 1 | AHAEETRKL | 4138 | Hospitalized | HLA-C*07:02 | AHAEETRKL | |
| | (SEQ ID | | | | (SEQ ID NO: 971) | |
| | NO: 971) | | | | | |
| AP020 1 | ALLADKFPVL | 18733 | Hospitalized | HLA-A*02:01 | ALLDKFPVL | |
| | (SEQ ID | | | | (SEQ ID NO: 972) | |
| | NO: 972) | | | | | |
| AP020 1 | ALVYFLQSI | 25435 | Hospitalized | HLA-A*02:01 | ALVYFLQSI | |
| | (SEQ ID | | | | (SEQ ID NO: 97) | |
| | NO: 973) | | | | | |
| AP020 1 | ALWEIQQVV | 12280 | Hospitalized | HLA-A*02:01 | ALWEIQQVV | |
| | (SEQ ID | | | | (SEQ ID NO: 974) | |
| | NO: 974) | | | | | |
| AP020 1 | ANPKTPKYKF | 10069 | Hospitalized | HLA-B*07:02 | APKTPKYKF | |
| | (SEQ ID | | | | (SEQ ID NO: 1086) | |
| | NO: 975) | | | | | |
| AP020 1 | APFLYLYAL | 25414 | Hospitalized | HLA-B*07:02 | APFLYLYAL | |
| | (SEQ ID | | | | (SEQ ID NO: 976) | |
| | NO: 976) | | | | | |
| AP020 1 | APHGVVFL | 24454 | Hospitalized | HLA-B*07:02 | APHXGVVFL | |
| | (SEQ ID | | | | (SEQ ID NO: 1087) | |
| | NO: 977) | | | | | |
| AP020 1 | APHGVVFLHV | 24454 | Hospitalized | HLA-B*07:02 | APHGVVLHV | |
| | (SEQ ID | | | | (SEQ ID NO: 1088) | |
| | NO: 978) | | | | | |
| BC10 | AADLDDFSKQL | 29050 | Healthy | HLA-A*02:01 | ALDDFSKQL | |
| | (SEQ ID | | | | (SEQ ID NO: 1089) | |
| | NO: 979) | | | | | |
| BC10 | ALLADKFPVL | 18733 | Healthy | HLA-A*02:01 | ALLDKFPVL | |
| | (SEQ ID | | | | (SEQ ID NO: 1090) | |
| | NO: 980) | | | | | |
| BC10 | ALLAVFQSA | 25258 | Healthy | HLA-A*02:01 | ALLAVFQSA | |
| | (SEQ ID | | | | (SEQ ID NO: 981) | |
| | NO: 981) | | | | | |
| BC10 | ALQDAYYRA | 5080 | Healthy | HLA-A*02:01 | ALQDAYYRA | |
| | (SEQ ID | | | | (SEQ ID NO: 982) | |
| | NO: 982) | | | | | |
| BC10 | ALTCFSTQF | 27169 | Healthy | HLA-B*15:01 | ALTCFSTQF | |
| | (SEQ ID | | | | (SEQ ID NO: 984) | |
| | NO: 984) | | | | | |
| BC10 | ALWEIQQVV | 12280 | Healthy | HLA-A*02:01 | ALWEIQQVV | |
| | (SEQ ID | | | | (SEQ ID NO: 985) | |
| | NO: 985) | | | | | |
| BC10 | AMDEFIERY | 20005 | Healthy | HLA-B*15:01 | AMDEFIERY | |
| | (SEQ ID | | | | (SEQ ID NO: 986) | |
| | NO: 986) | | | | | |
| BC10 | APISAMVRM | 7123 | Healthy | HLA-B*07:02 | APISAMVRM | |
| | (SEQ ID | | | | (SEQ ID NO: 987) | |
| | NO: 987) | | | | | |
| BC10 | APKEIIFL | 2203 | Healthy | HLA-B*07:02 | APKEIIFXL | |
| | (SEQ ID | | | | (SEQ ID NO: 1092) | |
| | NO: 988) | | | | | |
| | | | | | EWS | |
| ID | Peptide | DomCoreA | DomCoreB | DomCoreC | total | EWS |
| AP020 1 | ADLDDF | ALDDFSKQL | ALDDFSKQL | ALDDFSKQ | 0.756691 | 0.4457 |
| | SKQL | (SEQ ID NO: | (SEQ ID | L | | |
| | (SEQ ID | 1085) | NO: 1085) | (SEQ ID NO: | | |
| | NO: 969) | | | 1085) | | |
| AP020 1 | AFPFTIY | AFPFTIYSL | AFPFTIYSL | AFPFTIYSL | 2.0541 | 0.79205 |
| | SL | (SEQ ID NO: | (SEQ ID | (SEQ ID NO: | | |
| | (SEQ ID | 970) | NO: 970) | 970) | | |
| | NO: 970) | | | | | |
| AP020 1 | AHAEET | AHAEETRKL | AHAEETRKL | AHAEETRK | 0.36247 | 0.23345 |
| | RKL | (SEQ ID NO: | (SEQ ID | L | | |
| | (SEQ ID | 971) | NO: 971) | (SEQ ID NO: | | |
| | NO: 971) | | | 971) | | |
| AP020 1 | ALLADK | ALLDKFPVL | ALADKFPVL | ALADKFPV | 3.359796 | 0.50342 |
| | FPVL | (SEQ ID NO: | (SEQ ID | L | | |
| | (SEQ ID | 972) | NO: 1093) | (SEQ ID NO: | | |
| | NO: 972) | | | 1093) | | |
| AP020 1 | ALVYFL | ALVYFLQSI | ALVYFLQSI | ALVYFLQSI | 0.84878 | 0.60478 |
| | QSI | (SEQ ID NO: | (SEQ ID | (SEQ ID NO: | | |
| | (SEQ ID | 973) | NO: 973) | 973) | | |
| | NO: 973) | | | | | |
| AP020 1 | ALWEIQ | ALWEIQQVV | ALWEIQQVV | ALWEIQQV | 2.054364 | 1.1393 |
| | QVV | (SEQ ID NO: | (SEQ ID | V | | |
| | (SEQ ID | 974) | NO: 974) | (SEQ ID NO: | | |
| | NO: 974) | | | 974) | | |
| AP020 1 | ANPKTP | ANPKTPYKF | APKTPKYKF | ANPKTPYKF | 1.303788 | 0.48203 |
| | KYKF | (SEQ ID NO: | (SEQ ID | (SEQ ID NO: | | |
| | (SEQ ID | 975) | NO: 1086) | 975) | | |
| | NO: 975) | | | | | |
| AP020 1 | APFLYLY | APFLYLYAL | APFLYLYAL | APFLYLYAL | 1.072195 | 0.96739 |
| | AL | (SEQ ID NO: | (SEQ ID | (SEQ ID NO: | | |
| | (SEQ ID | 976) | NO: 976) | 976) | | |
| | NO: 976) | | | | | |
| AP020 1 | APHGVV | APHXGVVFL | APHXGVVFL | APHXGVVF | 0.773821 | 0.77382 |
| | FL | (SEQ ID NO: | (SEQ ID | L | | |
| | (SEQ ID | 1087) | NO: 1087) | (SEQ ID NO: | | |
| | NO: 977) | | | 1087) | | |
| AP020 1 | APHGVV | APHGVVLHV | APHGVVLHV | APHGVVLH | 1.404367 | 0.36886 |
| | FLHV | (SEQ ID NO: | (SEQ ID | V | | |
| | (SEQ ID | 1088) | NO: 1088) | (SEQ ID NO: | | |
| | NO: 978) | | | 1088) | | |
| BC10 | AADLDD | ALDDFSKQL | AADDFSKQL | ALDDFSKQ | 1.540239 | 0.37113 |
| | FSKQ | (SEQ ID NO: | (SEQ ID | L | | |
| | L | 1089) | NO: 1094) | (SEQ ID NO: | | |
| | (SEQ ID | | | 1089) | | |
| | NO: 979) | | | | | |
| BC10 | ALLADK | ALLDKFPVL | ALADKFPVL | ALADKFPV | 3.231395 | 0.52699 |
| | FPVL | (SEQ ID NO: | (SEQ ID | L | | |
| | (SEQ ID | 1090) | NO: 1095) | (SEQ ID NO: | | |
| | NO: 980) | | | 1095) | | |
| BC10 | ALLAVF | ALLAVFQSA | ALLAVFQSA | ALLAVFQSA | 0.589569 | 0.57417 |
| | QSA | (SEQ ID NO: | (SEQ ID | (SEQ ID NO: | | |
| | (SEQ ID | 981) | NO: 981) | 981) | | |
| | NO: 981) | | | | | |
| BC10 | ALQDAY | ALQDAYYRA | ALQDAYYRA | ALQDAYYR | 0.542454 | 0.5402 |
| | YRA | (SEQ ID NO: | (SEQ ID | A | | |
| | (SEQ ID | 982) | NO: 982) | (SEQ ID NO: | | |
| | NO: 982) | | | 982) | | |
| BC10 | ALRKVP | ALRKPTDNY | ALRKPTDNY | ALRKVPTD | 0.931627 | 0.70801 |
| | TDNY | (SEQ ID NO: | (SEQ ID | NY (SEQ ID | | |
| | (SEQ ID | 983) | NO: 983) | NO: 983) | | |
| | NO: 983) | | | | | |
| BC10 | ALTCFST | ALTCFSTQF | ALTCFSTQF | ALTCFSTQF | 0.273959 | 0.26356 |
| | QF | (SEQ ID NO: | (SEQ ID | (SEQ ID NO: | | |
| | (SEQ ID | 984) | NO: 984) | 984) | | |
| | NO: 984) | | | | | |
| BC10 | ALWEIQ | ALWEIQQVV | ALWEIQQVV | ALWEIQQV | 1.98179 | 1.0849 |
| | QVV | (SEQ ID NO: | (SEQ ID | V | | |
| | (SEQ ID | 985) | NO: 985) | (SEQ ID NO: | | |
| | NO: 985) | | | 985) | | |
| BC10 | AMDEFI | AMDEFIERY | AMDEFIERY | AMDEFIERY | 1.023412 | 1.00971 |
| | ERY | (SEQ ID NO: | (SEQ ID | (SEQ ID NO: | | |
| | (SEQ ID | 986) | NO: 986) | 986) | | |
| | NO: 986) | | | | | |
| BC10 | APISAM | APISAMVRM | APISAMVRM | APISAMVR | 0.821359 | 0.80681 |
| | VRM | (SEQ ID NO: | (SEQ ID | M | | |
| | (SEQ ID | 987) | NO: 987) | (SEQ ID NO: | | |
| | NO: 987) | | | 987) | | |
| BC10 | APKEIIF | APKEIIFXL | APKEIIFXL | APKXEIIFL | 0.532492 | 0.53249 |
| | L (SEQ | (SEQ ID NO: | (SEQ ID | (SEQ ID NO: | | |
| | ID NO: | 1092) | NO: 1092) | 988) | | |
| | 988) | | | | | |

The Adaptive cohort data may be used as validation data. The set of clinical outcomes which are recorded for this data includes Covid-19 acute, Covid-19 non-acute, Covid-19 convalescent, Covid-19 exposed and healthy (no known exposure). When preparing the data, epitopes may first be selected to pair with the individuals' data as for the ABF data. Using the NetMHCpan 4.1 prediction again, in this example 545 distinct HLA class I binding epitopes may be predicted. An average of 169 [18-240] epitopes per individual were tested with the TCR sequencing method that quantifies the potency of T-cell antigens as “hits” compared to the no antigen control. A “hit” shows the number of copies of each TCR sequence that measures the expansion of an antigen-specific T-cell clone after SARS-COV-2 infection in the individual. Since several experiments tested pools of peptides, we postulated that the T-cell responses were dominated by one epitope in the pool (highlighted in bold) after the strongest binding affinity to the dominant HLA-allele of the subject, resulting in an average of 51 [5 to 121] tested epitopes per subject. Merely as an example of the data which is generated at this step, the table below shows examples of 10 peptides for two individuals: one who was Covid-19 acute and the other who was healthy. It is noted that the first 10 peptides for these two individuals are not the same.

Data for Adaptive cohort

| | | | | Start | End | | |
|---|---|---|---|---|---|---|---|
| | | | | Index in | Index in | | |
| Subject | Amino_Acids | Cohort | Hits | Genomey | Genomey | ID | value |
| 1005699 | AFLLFLVLI (SEQ ID NO: | COVID- | 4 | 27756 | 27830 | 9612_ | IELSLIDFYL |
| | 1134), FLAFLLFLV (SEQ | 19-Acute | | | | 10 | (SEQ ID NO: |
| | ID NO: 1135), | | | | | | 1009) |
| | FYLCFLAFL | | | | | | |
| | (SEQ ID NO: 1136), | | | | | | |
| | FYLCFLAFLL (SEQ ID | | | | | | |
| | NO: 1137), IDFYLCFLAF | | | | | | |
| | (SEQ ID NO: 1138), | | | | | | |
| | IELSLIDFYL (SEQ ID | | | | | | |
| | NO: 1009), LIDFYLCFL | | | | | | |
| | (SEQ ID NO: | | | | | | |
| | 1139), LLFLVLIML (SEQ | | | | | | |
| | ID NO: 1140), | | | | | | |
| | MIELSLIDFY | | | | | | |
| | (SEQ ID NO: 1141), | | | | | | |
| | SLIDFYLCFL (SEQ ID | | | | | | |
| | NO: 1142), | | | | | | |
| | YLCFLAFLL | | | | | | |
| | (SEQ ID NO: 1143) | | | | | | |
| 1005699 | APHGVVFL (SEQ ID NO: | COVID- | 1 | 24728 | 24766 | 8147_ | VVFLHVTYV |
| | 1144), APHGVVFLHV | 19-Acute | | | | 9 | (SEQ ID NO: |
| | (SEQ ID NO: 1145), | | | | | | 1010) |
| | GVVFLHVTY | | | | | | |
| | (SEQ ID NO: 1146), | | | | | | |
| | VVFLHVTYV (SEQ ID | | | | | | |
| | NO: 1010) | | | | | | |
| 1005699 | ASQSIIAYTM (SEQ ID | COVID- | 23 | 23615 | 23659 | 7774_ | VASQSIIAY |
| | NO: 1147), | 19-Acute | | | | 9 | (SEQ ID NO: |
| | RSVASQSII (SEQ ID | | | | | | 1011) |
| | NO: 1148), SIIAYTMSL | | | | | | |
| | (SEQ ID NO: 1149), | | | | | | |
| | SQSIIAYTM (SEQ ID NO: | | | | | | |
| | 1150), VASQSIIAY (SEQ | | | | | | |
| | ID NO: 1011) | | | | | | |
| 1005699 | AYILFTRFFYV | COVID- | 3 | 7253 | 7285 | 2329_ | AYILFTRFFYV |
| | (SEQ ID NO: 1012) | 19-Acute | | | | 11 | (SEQ ID NO: |
| | | | | | | | 1012) |
| 1005699 | CELYHYQECV (SEQ ID | COVID- | 12 | 27418 | 27465 | 8963_ | ITLATCELY |
| | NO: 1151), | 19-Acute | | | | 9 | (SEQ ID NO: |
| | ITLATCELY (SEQ ID | | | | | | 1013) |
| | NO: 1013), LITLATCELY | | | | | | |
| | (SEQ ID NO: 1152), | | | | | | |
| | TLATCELYHY | | | | | | |
| | (SEQ ID NO: 1153) | | | | | | |
| 1005699 | CNDPFLGVY (SEQ ID | COVID- | 1 | 21965 | 21997 | 7221_ | FCNDPFLGVY |
| | NO: 1154), | 19-Acute | | | | 10 | (SEQ ID NO: |
| | CNDPFLGVYY (SEQ | | | | | | 1014) |
| | ID NO: 1155), | | | | | | |
| | FCNDPFLGVY | | | | | | |
| | (SEQ ID NO: 1014) | | | | | | |
| 1005699 | CTFEYVSQPF (SEQ ID | COVID- | 1 | 22058 | 22093 | 7255_ | FEYVSQPFL |
| | NO: 1156), | 19-Acute | | | | 9 | (SEQ ID NO: |
| | EYVSQPFLM (SEQ | | | | | | 1015) |
| | ID NO: 1157), | | | | | | |
| | FEYVSQPFL | | | | | | |
| | (SEQ ID NO: 1015) | | | | | | |
| 1005699 | DLFMRIFTI (SEQ ID | COVID- | 1 | 25393 | 25422 | 8354_ | DLFMRIFTI |
| | NO: 1016), | 19-Acute | | | | 9 | (SEQ ID NO: |
| | MDLFMRIFTI | | | | | | 1016) |
| | (SEQ ID NO: 1158) | | | | | | |
| 1005699 | ELIRQGTDY (SEQ ID | COVID- | 2 | 29138 | 29176 | 9469_ | ELIRQGTDY |
| | NO: 1017), | 19-Acute | | | | 9 | (SEQ ID NO: |
| | QELIRQGTDY | | | | | | 1017) |
| | (SEQ ID NO: 1159), | | | | | | |
| | QELIRQGTDYKHW | | | | | | |
| | (SEQ ID NO: 1160) | | | | | | |
| 1005699 | FAYANRNRF (SEQ ID | COVID- | 1 | 26625 | 26663 | 8723_ | FAYANRNRF |
| | NO: 1018), | 19-Acute | | | | 9 | (SEQ ID NO: |
| | LQFAYANRNR (SEQ | | | | | | 1018) |
| | ID NO: 1161), | | | | | | |
| | YANRNRFLY | | | | | | |
| | (SEQ ID NO: 1162) | | | | | | |
| 1327414 | AFLLFLVLI (SEQ ID NO: | Healthy | 24 | 27756 | 27830 | 9611_ | MIELSLIDFY |
| | 1163), FLAFLLFLV (SEQ | | | | | 10 | (SEQ ID NO: |
| | ID NO: 1164), | | | | | | 1019) |
| | FYLCFLAFL | | | | | | |
| | (SEQ ID NO: 1165), | | | | | | |
| | FYLCFLAFLL (SEQ ID | | | | | | |
| | NO: 1166), | | | | | | |
| | IDFYLCFLAF (SEQ | | | | | | |
| | ID NO: 1167), | | | | | | |
| | IELSLIDFYL (SEQ ID | | | | | | |
| | NO: 1168), | | | | | | |
| | LIDFYLCFL (SEQ ID NO: | | | | | | |
| | 1169), LLFLVLIML (SEQ | | | | | | |
| | ID NO: 1170), | | | | | | |
| | MIELSLIDFY | | | | | | |
| | (SEQ ID NO: 1019), | | | | | | |
| | SLIDFYLCFL (SEQ ID | | | | | | |
| | NO: 1171), | | | | | | |
| | YLCFLAFLL | | | | | | |
| | (SEQ ID NO: 1172) | | | | | | |
| 1327414 | AFPFTIYSL (SEQ ID NO: | Healthy | 19 | 29558 | 29605 | 9586_ | NVFAFPFTIY |
| | 1173), GYINVFAFPF | | | | | 10 | (SEQ ID NO: |
| | (SEQ ID NO: 1174), | | | | | | 1020) |
| | INVFAFPFTI (SEQ ID | | | | | | |
| | NO: 1175), | | | | | | |
| | MGYINVFAF (SEQ ID | | | | | | |
| | NO: 1176), | | | | | | |
| | NVFAFPFTI (SEQ ID | | | | | | |
| | NO: 1177), NVFAFPFTIY | | | | | | |
| | (SEQ ID NO: 1020), | | | | | | |
| | YINVFAFPF | | | | | | |
| | (SEQ ID NO: 1178) | | | | | | |
| 1327414 | AIMTRCLAV | Healthy | 1 | 18860 | 18886 | 6198_ | AIMTRCLAV |
| | (SEQ ID NO: 1001) | | | | | 9 | (SEQ ID NO: |
| | | | | | | | 1021) |
| 1327414 | ALNTLVKQL (SEQ ID | Healthy | 1 | 24428 | 24460 | 8045_ | ALNTLVKQL |
| | NO: 1022), | | | | | 9 | (SEQ ID NO: |
| | AQALNTLVK | | | | | | 1022) |
| | (SEQ ID NO: 1179) | | | | | | |
| 1327414 | APAHISTI (SEQ ID NO: | Healthy | 5 | 19895 | 26325 | 8635_ | SVLLFLAFV |
| | 1180), LIVNSVLLFL | | | | | 9 | (SEQ ID NO: |
| | (SEQ ID | | | | | | 1023) |
| | NO: 1181), LLFLAFVVFL | | | | | | |
| | (SEQ ID NO: 1182), | | | | | | |
| | SVLLFLAFV | | | | | | |
| | (SEQ ID NO: 1023) | | | | | | |
| 1327414 | APHGVVFL (SEQ ID NO: | Healthy | 8 | 24728 | 24766 | 8147_ | VVFLHVTYV |
| | 1183), APHGVVFLHV | | | | | 9 | (SEQ ID NO: |
| | (SEQ ID NO: 1184), | | | | | | 1024) |
| | GVVFLHVTY | | | | | | |
| | (SEQ ID NO: 1185), | | | | | | |
| | VVFLHVTYV | | | | | | |
| | (SEQ ID NO: 1024) | | | | | | |
| 1327414 | APKEIIFL (SEQ ID NO: | Healthy | 1 | 2468 | 2506 | 736_1 | KEIIFLEGETL |
| | 1186), KEIIFLEGETL | | | | | 1 | (SEQ ID NO: |
| | (SEQ ID NO: 1025) | | | | | | 1025) |
| 1327414 | ASQSIIAYTM (SEQ ID | Healthy | 4 | 23615 | 23659 | 7774_ | VASQSIIAY |
| | NO: 1187), | | | | | 9 | (SEQ ID NO: |
| | RSVASQSII (SEQ ID | | | | | | 1026) |
| | NO: 1188), SIIAYTMSL | | | | | | |
| | (SEQ ID NO: 1189), | | | | | | |
| | SQSIIAYTM (SEQ ID NO: | | | | | | |
| | 1190), VASQSIIAY | | | | | | |
| | (SEQ ID NO: 1026) | | | | | | |
| 1327414 | AYILFTRFFYV | Healthy | 1 | 7253 | 7285 | 2329_ | AYILFTRFFYV |
| | (SEQ ID NO: 1007) | | | | | 11 | (SEQ ID NO: |
| | | | | | | | 1027) |
| 1327414 | CELYHYQECV (SEQ ID | Healthy | 1 | 27418 | 27465 | 8963_ | ITLATCELY |
| | NO: 1191), | | | | | 9 | (SEQ ID NO: |
| | ITLATCELY (SEQ ID | | | | | | 1028) |
| | NO: 1028), LITLATCELY | | | | | | |
| | (SEQ ID NO: 1192), | | | | | | |
| | TLATCELYHY | | | | | | |
| | (SEQ ID NO: 1193) | | | | | | |

The Adaptive data only reported immunogenic epitopes (T-cell antigens) and there are no results of the experiments with no T-cell responses. Accordingly, the “hits” variable is used above and can be used to rank epitopes for each individual. For each selected peptide in the group, the scores which are used in the feature vector for the inference stage are also obtained for each subject and each selected peptide.

(Cont'd) Data for Adaptive cohort

| ID | Peptide | EWS_A | EWS_B | EWS_C | SWS_A | SWS_B | SWS_C | EL_Max |
|---|---|---|---|---|---|---|---|---|
| 1005699 | IELSLIDFYL | 0.00177 | 0.18304 | 7.6E−05 | 0.02065 | 0.20127 | 0.00716 | 0.17284 |
| | (SEQ ID NO: | | | | | | | |
| | 1009) | | | | | | | |
| 1005699 | VVFLHVTYV | 0.18795 | 0.36684 | 0.51368 | 0.05239 | 0.09107 | 0.21392 | 0.36009 |
| | (SEQ ID NO: | | | | | | | |
| | 1010) | | | | | | | |
| 1005699 | VASQSIIAY | 0.07924 | 0.06444 | 1.64194 | 0.00358 | 0.00867 | 0.06901 | 0.86889 |
| | (SEQ ID NO: | | | | | | | |
| | 1011) | | | | | | | |
| 1005699 | AYILFTRFFYV | 0.00338 | 0.00142 | 0.00012 | 0.47114 | 0.24104 | 0.15596 | 0.0018 |
| | (SEQ ID NO: | | | | | | | |
| | 1012) | | | | | | | |
| 1005699 | ITLATCELY | 0.05294 | 0.0079 | 0.03374 | 0.00105 | 0.01129 | 0.0072 | 0.05068 |
| | (SEQ ID NO: | | | | | | | |
| | 1013) | | | | | | | |
| 1005699 | FCNDPFLGVY | 0.00223 | 0.00202 | 0.18935 | 0.00284 | 0.05285 | 0.15412 | 0.16466 |
| | (SEQ ID NO: | | | | | | | |
| | 1014) | | | | | | | |
| 1005699 | FEYVSQPFL | 0.00317 | 1.32465 | 0.04418 | 0.00192 | 0.77808 | 0.00721 | 0.91035 |
| | (SEQ ID NO: | | | | | | | |
| | 1015) | | | | | | | |
| 1005699 | DLFMRIFTI | 0.09938 | 0.25574 | 0.01063 | 0.00362 | 0.03738 | 0.00091 | 0.24912 |
| | (SEQ ID NO: | | | | | | | |
| | 1016) | | | | | | | |
| 1005699 | ELIRQGTDY | 0.00227 | 0.00088 | 0.01074 | 0.00018 | 0.00047 | 0.0064 | 0.00928 |
| | (SEQ ID NO: | | | | | | | |
| | 1017) | | | | | | | |
| 1005699 | FAYANRNRF | 0.07722 | 0.25763 | 1.79427 | 0.04306 | 0.00717 | 0.02058 | 0.91575 |
| | (SEQ ID NO: | | | | | | | |
| | 1018) | | | | | | | |
| 1327414 | MIELSLIDFY | 0.11622 | 0.32239 | 0.00016 | 0.32025 | 1.61622 | 0.00202 | 0.21644 |
| | (SEQ ID NO: | | | | | | | |
| | 1019) | | | | | | | |
| 1327414 | NVFAFPFTIY | 0.67771 | 0.01222 | 0.00757 | 1.79374 | 0.1051 | 0.75772 | 0.67581 |
| | (SEQ ID NO: | | | | | | | |
| | 1020) | | | | | | | |
| 1327414 | AIMTRCLAV | 0.15849 | 0.00017 | 0.01054 | 0.00219 | 4.6E−05 | 0.00034 | 0.15737 |
| | (SEQ ID NO: | | | | | | | |
| | 1021) | | | | | | | |
| 1327414 | ALNTLVKQL | 0.66142 | 0.01005 | 0.03583 | 0.00347 | 0.00078 | 0.00137 | 0.6574 |
| | (SEQ ID NO: | | | | | | | |
| | 1022) | | | | | | | |
| 1327414 | SVLLFLAFV | 0.20913 | 7.5E−05 | 0.00068 | 0.03008 | 0.00434 | 0.00498 | 0.20794 |
| | (SEQ ID NO: | | | | | | | |
| | 1023) | | | | | | | |
| 1327414 | VVFLHVTYV | 0.76748 | 0.00088 | 0.11367 | 0.33866 | 0.05741 | 0.14615 | 0.74167 |
| | (SEQ ID NO: | | | | | | | |
| | 1024) | | | | | | | |
| 1327414 | KEIIFLEGETL | 0.02527 | 0.11749 | 0.00015 | 0.14698 | 0.01055 | 0.03264 | 0.10924 |
| | (SEQ ID NO: | | | | | | | |
| | 1025) | | | | | | | |
| 1327414 | VASQSIIAY | 0.64192 | 0.16664 | 1.00943 | 0.04348 | 0.01705 | 0.206 | 0.9081 |
| | (SEQ ID NO: | | | | | | | |
| | 1026) | | | | | | | |
| 1327414 | AYILFTRFFYV | 0.12464 | 0.00021 | 0.0001 | 3.343 | 0.03347 | 0.12177 | 0.11659 |
| | (SEQ ID NO: | | | | | | | |
| | 1027) | | | | | | | |
| 1327414 | ITLATCELY | 0.57609 | 0.00419 | 0.05212 | 0.09648 | 0.00195 | 0.03677 | 0.57572 |
| | (SEQ ID NO: | | | | | | | |
| | 1028) | | | | | | | |

Additional potency data may also be obtained for each identified peptide as shown in the table below. The dominant core for each of the A, B and C loci are also obtained but for ease of display are not shown (they are the same for these peptides as the overall dominant core):

(Cont'd) Data for Adaptive cohort

| | | Genome | Dom | | | EWS | |
|---|---|---|---|---|---|---|---|
| ID | Peptide | Index | HLA | DomCore | HitEpi | Total | EWS |
| 1005699 | IELSLIDFYL | 27373 | HLA- | IELSLIFYL | IELSLIDFYL | 0.413967 | 0.18489 |
| | (SEQ ID NO: | | B*40:02 | (SEQ ID NO. | (SEQ ID NO: | | |
| | 1009) | | | 1096) | 1009) | | |
| 1005699 | VVFLHVTYV | 24466 | HLA- | VVFLHVTYV | VVFLHVTYV | 1.425845 | 1.06846 |
| | (SEQ ID NO: | | B*52:01 | (SEQ ID NO: | (SEQ ID NO: | | |
| | 1010) | | | 1010) | 1010) | | |
| 1005699 | VASQSIIAY | 23347 | HLA- | VASQSIIAY | VASQSIIAY | 1.866881 | 1.78561 |
| | (SEQ ID NO: | | C*12:02 | (SEQ ID NO: | (SEQ ID NO: | | |
| | 1011) | | | 1011) | 1011) | | |
| 1005699 | AYILFTRFFYV | 6988 | HLA- | ALFTRFFYV | AYILFTRFFYV | 0.873063 | 0.00492 |
| | (SEQ ID NO: | | A*33:01 | (SEQ ID NO: | (SEQ ID NO: | | |
| | 1012) | | | 1097) | 1012) | | |
| 1005699 | ITLATCELY | 27034 | HLA- | ITLATCELY | ITLATCELY | 0.114109 | 0.09457 |
| | (SEQ ID NO: | | A*32:01 | (SEQ ID NO: | (SEQ ID NO: | | |
| | 1013) | | | 1013) | 1013) | | |
| 1005699 | FCNDPFLGVY | 21691 | HLA- | FCNDPFLVY | FCNDPFLGVY | 0.403413 | 0.19361 |
| | (SEQ ID NO: | | C*12:02 | (SEQ ID NO: | (SEQ ID NO: | | |
| | 1014) | | | 1098) | 1014) | | |
| 1005699 | FEYVSQPFL | 21790 | HLA- | FEYVSQPFL | FEYVSQPFL | 2.159204 | 1.372 |
| | (SEQ ID NO: | | B*40:02 | (SEQ ID NO: | (SEQ ID NO: | | |
| | 1015) | | | 1015) | 1015) | | |
| 1005699 | DLFMRIFTI | 25111 | HLA- | DLFMRIFTI | DLFMRIFTI | 0.407647 | 0.36575 |
| | (SEQ ID NO: | | B*52:01 | (SEQ ID NO: | (SEQ ID NO: | | |
| | 1016) | | | 1016) | 1016) | | |
| 1005699 | ELIRQGTDY | 28729 | HLA- | ELIRQGTDY | ELIRQGTDY | 0.020934 | 0.01388 |
| | (SEQ ID NO: | | C*12:02 | (SEQ ID NO: | (SEQ ID NO: | | |
| | 1017) | | | 1017) | 1017) | | |
| 1005699 | FAYANRNRF | 26266 | HLA- | FAYANRNRF | FAYANRNRF | 2.199924 | 2.12912 |
| | (SEQ ID NO: | | C*12:02 | (SEQ ID NO: | (SEQ ID NO: | | |
| | 1018) | | | 1018) | 1018) | | |
| 1327414 | MIELSLIDFY | 27370 | HLA- | MELSLIDFY | MIELSLIDFY | 2.377257 | 0.43877 |
| | (SEQ ID NO: | | B*44:03 | (SEQ ID NO: | (SEQ ID NO: | | |
| | 1019) | | | 1099) | 1019) | | |
| 1327414 | NVFAFPFTIY | 29131 | HLA- | NVFAFPFTY | NVFAFPFTIY | 3.354067 | 0.6975 |
| | (SEQ ID NO: | | A*29:02 | (SEQ ID NO: | (SEQ ID NO: | | |
| | 1020) | | | 1100) | 1020) | | |
| 1327414 | AIMTRCLAV | 18595 | HLA- | AIMTRCLAV | AIMTRCLAV | 0.171778 | 0.1692 |
| | (SEQ ID NO: | | A*02:01 | (SEQ ID NO: | (SEQ ID NO: | | |
| | 1021) | | | 1021) | 1021) | | |
| 1327414 | ALNTLVKQL | 24160 | HLA- | ALNTLVKQL | ALNTLVKQL | 0.712913 | 0.7073 |
| | (SEQ ID NO: | | A*02:01 | (SEQ ID NO: | (SEQ ID NO: | | |
| | 1022) | | | 1022) | 1022) | | |
| 1327414 | SVLLFLAFV | 25978 | HLA- | SVLLFLAFV | SVLLFLAFV | 0.249276 | 0.20988 |
| | (SEQ ID NO: | | A*02:01 | (SEQ ID NO: | (SEQ ID NO: | | |
| | 1023) | | | 1023) | 1023) | | |
| 1327414 | VVFLHVTYV | 24466 | HLA- | VVFLHVTYV | VVFLHVTYV | 1.424247 | 0.88203 |
| | (SEQ ID NO: | | A*02:01 | (SEQ ID NO: | (SEQ ID NO: | | |
| | 1024) | | | 1024) | 1024) | | |
| 1327414 | KEIIFLEGETL | 2209 | HLA- | KEFLEGETL | KEIIFLEGETL | 0.33307 | 0.1429 |
| | (SEQ ID NO: | | B*44:03 | (SEQ ID NO: | (SEQ ID NO: | | |
| | 1025) | | | 1101) | 1025) | | |
| 1327414 | VASQSIIAY | 23347 | HLA- | VASQSIIAY | VASQSIIAY | 2.084511 | 1.81798 |
| | (SEQ ID NO: | | C*16:01 | (SEQ ID NO: | (SEQ ID NO: | | |
| | 1026) | | | 1026) | 1026) | | |
| 1327414 | AYILFTRFFYV | 6988 | HLA- | ALFTRFFYV | AYILFTRFFYV | 3.623186 | 0.12495 |
| | (SEQ ID NO: | | A*02:01 | (SEQ ID NO: | (SEQ ID NO: | | |
| | 1027) | | | 1102) | 1027) | | |
| 1327414 | ITLATCELY | 27034 | HLA- | ITLATCELY | ITLATCELY | 0.767597 | 0.6324 |
| | (SEQ ID NO: | | A*29:02 | (SEQ ID NO: | (SEQ ID NO: | | |
| | 1028) | | | 1028) | 1028) | | |

Once the training and validation data is prepared, the model can then be trained with the training data at step S804. As shown at step S806, performance or evaluation metrics for the trained model can be optionally computed using the validation (i.e. test) data. There may be several loops of steps S804 and S806 as indicated in FIG. 8b depending on the training method chosen. Merely as an example, a five-fold cross-validation procedure may be used, and this splits the clinical data into five separate random folds of 80% and 20% training and test data. The splits may be stratified to maintain the ratios of immunogenic and non-immunogenic antigens in each subject. The grouped data may also not be shared between the training and test data sets.

The evaluation metrics may include one or both of population and individual metrics. Population metrics are metrics which are computed across the dataset and consider each data point in the dataset regardless of the individual. Such metrics provide useful comparative information on model performance as described in more detail in the model validation section below. Examples of suitable population metrics include the receiver operating characteristic curve (ROC) and the precision recall curve (PR) which are calculated using known techniques. The PR curve may be considered to be a better alternative than the ROC in this class imbalanced scenario and may provide a more accurate perspective on the performance of the binary classification compared to a random model. The metric which is reported may be the area under the ROC (AUC-ROC) and the area under the PR curve (AUC-PR), both averaged across all data points.

Individual metrics are metrics which are specific to an individual and are more consistent with the diagnostic function of the present method. At the individual level, we are interested in identifying the Top-N epitopes with the highest T-cell response, where N is defined according to the number of epitopes of interest per individual. For the ABF dataset, N may be set to 20 and for the Adaptive dataset, N may be set to 3. The lower number for the Adaptive dataset reflects the fact that it contains fewer characterized epitopes per individual. For each Top-N cases, one performance metric that can be calculated is the ranked accuracy A_{R}, which may be calculated using:

$${A}_{R} = \frac{{TP}_{R}}{N}$$

where TP_{R} is the number of top ranked antigens in the ranking generated by the model which also appear in the ranking generated by using the ground-truth potency measure in each dataset for each individual. Thus the ranked accuracy can be interpreted as the fraction of the top-N antigens that were predicted correctly for an individual.

In addition to (or as an alternative to) ranked accuracy, the precision P and recall R for each individual may be calculated using

$$P = \frac{TP}{TP + FP} \\ R = \frac{TP}{TP + FN}$$

where TP is the number of true positives, FP is the number of false positives and FN is the number of false negatives in the epitopes of interest for an individual. In other words, TP and TN (true negatives) are the correct predictions.

In the Adaptive dataset, only positive values are reported. For this case, a different metric which is a common metric in positive unlabeled learning may be used. The positive unlabeled metric PU may be calculated using

$$PU = \frac{R \cdot R}{PR ( {y}^{′} = 1 )}$$

where R is the recall for the Top-N ranked epitopes and PR (y′=1) corresponds to the fraction of the positive predictions made by the model. This metric approximates the f-score when true negatives are not available. A model with a higher PU is preferred. More information on the PU metric is found in reference 9 (Jaskie).

Once the model has been trained, it is output at step S808 for use in predicting the T-cell response of an individual. As explained with reference to FIG. 2a, the inputs to the computer-implemented method are the individual data and the epitope data which are shown as being obtained at steps S810 and S812 respectively. The data may be obtained simultaneously as indicated or in any order. The next step is to prepare the feature vector at step S814. The feature vector comprises as much of the information in the columns for the training data as possible. The feature vector may thus comprise some or all of the following scores which are calculated as described above: average EWS score, the total EWS score, the EWS score for each loci (e.g. A, B and C for HLA class I), the SWS score for each loci. The feature vector may also comprise information relevant to the epitope being considered, including some or all of the overall dominant core, the dominant core for each loci, the genome index, the start and end index, the number of hits, and the dominant HLA. Once the feature vector is prepared, the ranking for each epitope for an individual can be inferred using the trained model at step S816. The ranking may then output to a user, e.g. on a display screen, at step S818 or output to another system for further processing, e.g. to generate a suggestion for a vaccine or TCR therapy.

### Results—Prediction of SARS-COV-2 Specific T-Cell Responses in an Individual

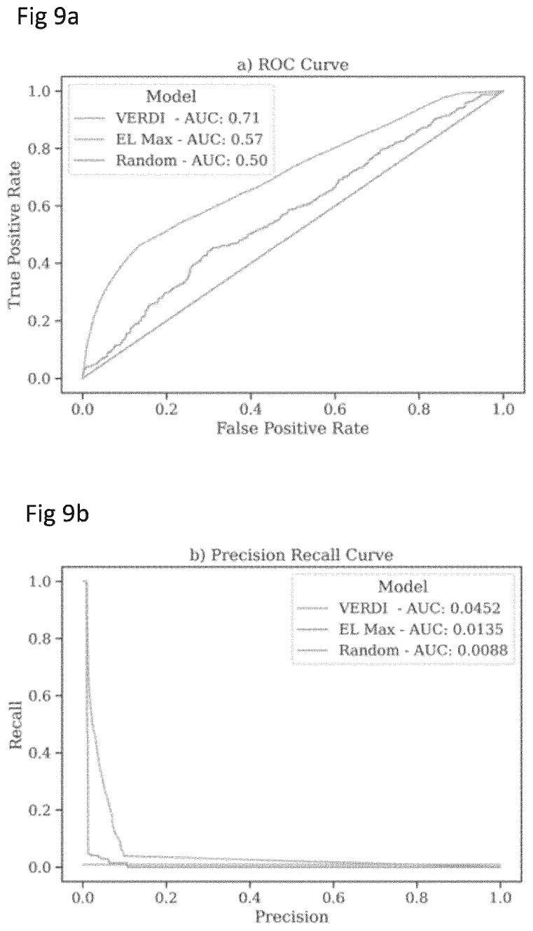

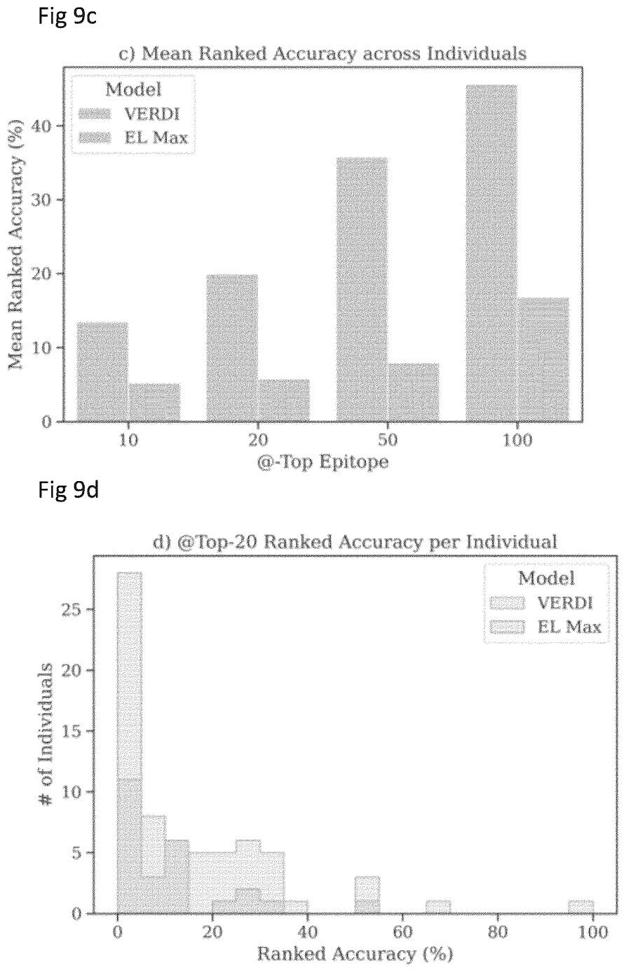

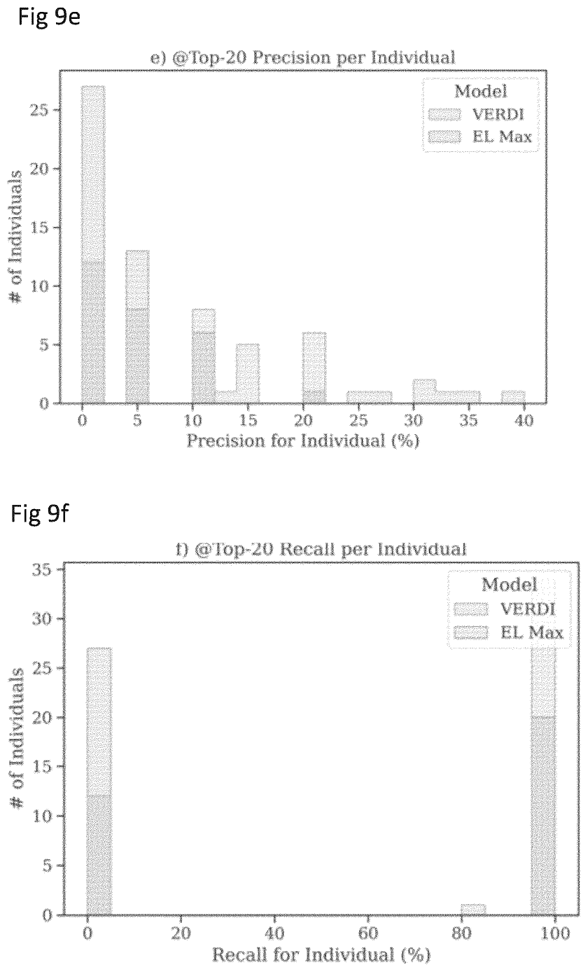

The ranking which is predicted by the trained model was compared with the ranking which is predicted by random selection (termed random) and the ranking which is predicted by considering the maximum EL score alone (termed EL Max model). FIG. 9a plots the ROC which shows the true positive rate against the false positive rate for each of the three predictive methods and the area under the curve (AUC) values are shown. The AUC of the current method (termed VERDI) is 0.71 and is better than both the EL Max model and the random selection which are 0.57 and 0.5 respectively. Indeed, the EL Max model appears to be only slightly better than random guessing. This result is also confirmed in FIG. 9b which plots the PR curve showing Recall against Precision. Again, the current method (termed VERDI) is the best with an area under curve value of 0.0452 which is significantly higher than those for El Max model and random guessing at 0.0135 and 0.0088 respectively.

Turning to individual performance metrics, FIG. 9c is a bar chart comparing the mean ranked accuracy by the current method and the EL-Max model for the top-10, top-20, top-50 and top-100 epitopes for each individual averaged across all individuals. The current method (VERDI) significantly outperforms the EL-Max model with the latter only correctly predicting fewer than 10% of test epitopes in the top-10, top-20 and top-50 which would produce a detectable T-cell response in HLA-allele matched test subjects. By contrast, the current method was able to predict up to 40% of the T-cell antigens when considering all epitopes. These results validate the performance of the proposed method in predicting the specificity and potency of T-cell antigens at the individual level.

FIGS. 9d to 9f compare the accuracy, precision and recall metrics for the top-20 ranked epitopes (T-cell antigen candidates) across individuals. The comparison process using just the EL Max score has limited performance for most individuals across all these metrics. On the other hand, the proposed method has adequate performance for most individuals, albeit there is a large spread across individuals. For precision and accuracy calculations, we use the same decision threshold but we explored other thresholds for the model, all yielding similar results.

To evaluate the clinical performance of the proposed method outside the ABF cohort, the Adaptive dataset was used. The Adaptive dataset was collected independently by Adaptive Biotechnologies for the development of the first T-cell response diagnostic test. The Adaptive dataset encompasses different epitopes, different cohorts of individuals living in a different part of the world (US not Denmark) and different methods to quantify T-cells (TCR sequencing versus multimer staining). The Adaptive dataset also tests a different number of SARS-COV-2 epitopes per subject: an average of 51 [ranging between 5 and 121] versus an average of 920 [ranging from 209 to 1452] for the ABF dataset.

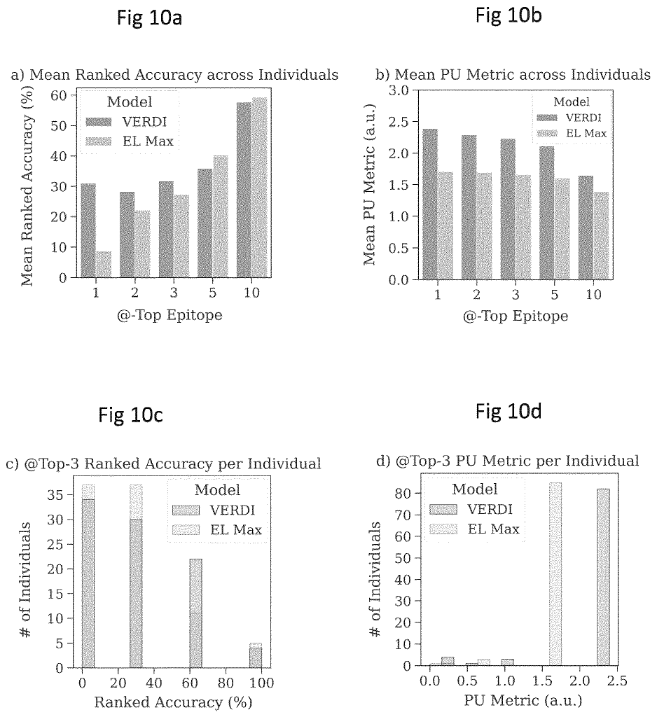

FIGS. 10a to 10d show the validation results using the Adaptive dataset. Using the proposed method, all 70,000 putative epitopes for SARS-COV-2 were ranked for each individual in the Adaptive dataset. The Adaptive dataset only reported experiments with epitopes that induced T-cell responses (T-cell antigens) and thus FIGS. 10a to 10d focus on the mean ranked accuracy metrics across individuals. FIG. 10a plots the mean ranked accuracy for all individuals for the top-1, top-2, top-3, top-5 and top-10 ranked epitopes using the proposed method (VERDI) and the EL Max model for comparison. Smaller numbers of ranked epitopes are evaluated than the rankings generated for the ABF dataset because fewer epitopes per individual were tested by the Adaptive dataset. As shown in FIG. 10a, the proposed method had an accuracy of approximately 30% for the top-1, top-2, top-3 ranking. The proposed method also outperforms the EL Max model except for top-10 ranking. The Adaptive dataset reported only the positive T-cell responses on epitopes predicted to be likely immunogenic and consequently an excess of false positives will not be adequately penalised. Thus, the El Max model which generates a large number of false positives appears to outperform the proposed method for larger N rankings.

FIG. 10b plots the mean PU metric for all individuals for the top-1, top-2, top-3, top-5 and top-10 ranked epitopes using the proposed method (VERDI) and the EL Max model for comparison. The PU metric is used to correct for false positives. As shown in FIG. 8b, the proposed method is superior to the El Max model for all top-N rankings listed.

FIGS. 10c and 10d focus on the top-3 ranking. FIG. 10c plots the number of individuals against the ranked accuracy and FIG. 8d plots the number of individuals against the PU metric. As shown in FIG. 10c, the proposed method predicts at least 1 of the 3 most potent T-cell antigens identified by the FDA approved T-cell response diagnostic for most subjects. FIG. 10d illustrates that the proposed method has a good predictive performance in most individuals. FIGS. 10a to 10d shows that the proposed method is a better model to predict the specificity and potency of T-cell responses than the EL max model, even on this challenging Adaptive dataset.

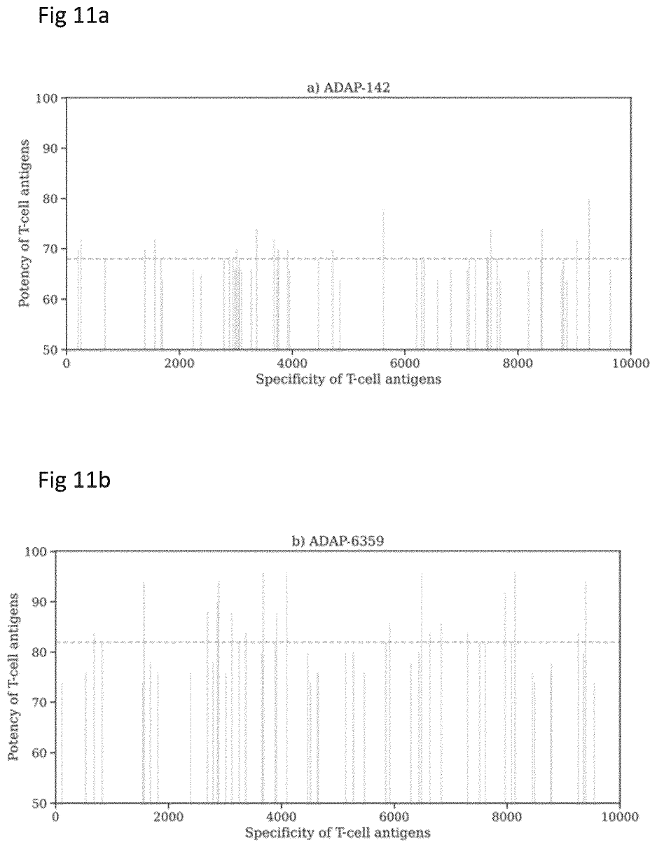

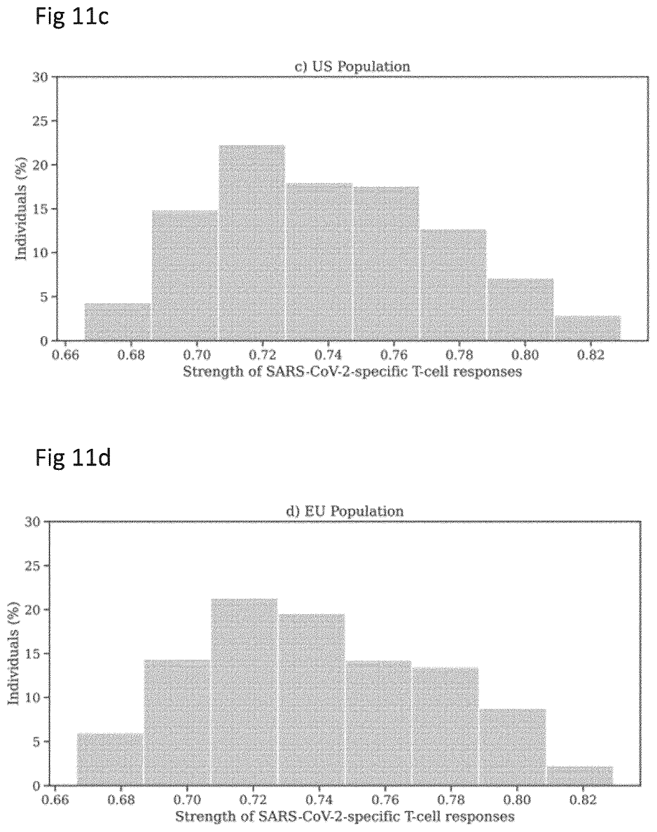

Individuals typically respond to an average of 30 epitopes (see for example reference 17 (Tarke). Accordingly, outputting the top-50 most potent epitopes from SARS-COV-2 for an individual should adequately characterize the SARS-COV-2 response for that individual. FIGS. 11a and 11b plots the top-50 ranked epitopes as 9-mer cores for two 101nonymized individuals from the Adaptive dataset labelled with the IDs ADAP-142 and ADAP-6359 respectively. FIGS. 11a and 11b plot the potency (likelihood of T-cell response) against specificity (indicated as the location in the proteome). The HLA genotype data for each individual is shown in the table below:

| Subject ID | ADAP-142 | ADAP-6359 |
|---|---|---|
| HLA_A_1_ID | HLA-A*24:02 | HLA-A*02:01 |
| HLA_A_2_ID | HLA-A*24:02 | HLA-A*24:02 |
| HLA_B_1_ID | HLA-B*15:35 | HLA-B*07:02 |
| HLA_B_2_ID | HLA-B*40:02 | HLA-B*07:02 |
| HLA_C_1_ID | HLA-C*08:01 | HLA-C*07:02 |
| HLA_C_2_ID | HLA-C*15:02 | HLA-C*07:02 |

FIGS. 11a and 11b show that the spread of T-cell antigens through the SARS-COV-2 proteome is extremely variable. This result is repeated for each of the 2346 individuals evaluated. There is only a small subset of T-cell antigens which are shared by a subset of the individuals and no single T-cell antigen was immunogenic in all individuals. However, although there is variation in the specificity of the T-cell antigens, for each individual the potency is fairly consistent. The dotted line on each of FIGS. 11a and 11b illustrates the average (i.e. mean) potency of the top-50 ranked antigens which can be defined as the strength of the individual's response. For the individual ADAP-142, the average potency is 68% and minimum and maximum potency values are 64% and 80% respectively. For the individual ADAP-6359, the average potency is 82% and minimum and maximum potency values are 74% and 96% respectively. The individual ADAP-142 has a weak T-cell response with no ranked T-cell antigens having over 80% potency. By contrast, individual ADAP-6359 has a strong T-cell response with no ranked T-cell antigens having over 80% potency.

It is important to diagnose the strength of the SARS-COV-2 specific T-cell responses because this is associated with the outcome of infection and vaccine protection (for example as described in reference 3 (Toss)). FIGS. 11e and 11d plot the number of individuals (as a percentage) in a population against average potency across the top-50 ranked epitopes. FIGS. 11c and 11d both resemble a Gaussian distribution in the separate US and EU populations. Similar results are expected with the 12 HLA class II molecules. This suggests that the strength of T-cell response to a SARS-COV-2 infection is inherited as an HLA-genotype dependent heritable phenotype. We propose that subjects at the left tail on the Gaussian curve with weak T-cell responses experience symptomatic COVID-19 and need additional intervention.

In individuals with strong T-cell responses, we propose that a few potent antigens quickly and vigorously stimulate T-cells to proliferate and kill infected cells (e.g. eight T-cell antigens with greater than 90% potency). After rapidly clearing the infection, a fraction of the activated T-cells establishes the memory pool, and the others undergo apoptosis. In contrast, in individuals with less potent T-cell antigens, SARS-COV-2 induces broad T-cell responses. Less potent antigens activate T-cells more slowly and proliferation is languorous which prolongs the time to elimination of infected cells, for example 50 T-cells with between 64% to 80% potency as for the individual shown in FIG. 11a. Until the virus is replicating, additional antigens activate T-cells leading to a broader T-cell repertoire. The consequence of weak T-cell responses is the expression of inhibitory molecules (PD1, Tim3) that promote viral persistence. Individuals with weak T-cell responses likely experience high viral load and severe Covid-19 (as suggested in reference 19 (Cevik). The extreme heterogeneity in the specificity and potency of T-cell antigens explains the diverse clinical findings amongst SARS-COV-2 infected individuals, including the expansion, maintenance and exhaustion of T-cell responses.

Healthy individuals like ADAP-6359, with potent T-cell responses against sick cells may not need vaccines to kill infected cells. However, healthy individuals like ADAP-6359 might need a vaccine containing antigens that boost T-cell responses to kill infected cells. After T cell vaccination ADAP-6359 will have long-lasting memory cells that can quicky proliferate after virus infection and destroy infected cells. Therefore, high-ranked epitopes can be used to characterize the potency of the T cell responses of an individual against sick cells. T cell antigens may also be selected for vaccine development to induce potent T-cell responses in HLA genotype matched recipients. These vaccines are effective to attack sick cells presenting the same epitopes, and useful for the prevention and treatment of SARS-COV-2, other viral infections, cancer and few other diseases.

Personalised Vaccines and their Uses

In the following passages, different aspects of the disclosure relating to personalized vaccines and their uses are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N. Y. (2012).

Peptide synthesis and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

In the present invention, unless otherwise specified, scientific and technical terms used herein have the meanings commonly understood by those skilled in the art. In addition, the cell culture, molecular genetics, nucleic acid chemistry, and immunology laboratory operation steps used herein are all routine steps widely used in the corresponding fields. Meanwhile, for a better understanding of the present invention, definitions and explanations of related terms are provided below.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of peptide, dose, time, temperature, enzymatic activity or other biological activity and the like, is meant to encompass variations of ±20%, ±10%, ±5%, ±1%, ±0.5%, or even ±0.1% of the specified amount.

The term “multiple” may be defined as “at least two”, or “two or more”, or “a plurality”.

### Limitations of Currently Investigated Personalized Vaccines

Existing personalized peptide vaccines in clinical trials consist of a mixture of multiple peptides coding an epitope and adjuvants. Sometime, the epitopes are expressed by mRNA, DNA or viral vectors. The currently used process of developing a personalized vaccine is time-consuming and expensive. It involves the identification of unique mutations, predicting which will produce an immune response, manufacturing the vaccine, and finally administering it to the patient. Personalized peptide vaccines often utilize overlapping long-peptides or predicted peptides that bind to specific HLA alleles. However, the process of predicting which epitope will produce an immune response is not accurate, since none of the predicted epitope induce both CD8 and CD4 T-cell responses against a target proteins. Despite a personalized vaccine include several predicted epitopes of the patient, not all patients will have a strong immune response to the vaccine. Furthermore, the production of personalized vaccines presents significant logistical and manufacturing challenges. For peptide vaccines it is related to peptide solubility, peptide interactions, quality control, and potential side effects from adjuvants. Consequently, personalized vaccines may not be effective and accessible to all patients. The inventors have sought to address these limitations.

### Personalized Vaccines Designed by the VERDI System and Computer Implemented Methods

The invention set forth herein is a personalised VERDI Vaccine designed by the system and the computer implemented method invented here. This is advantageous because it is a single peptide coding at least one cell surface antigen that comprises a set of highly ranked epitopes presented on the cell surface by HLA class I and/or HLA class II molecules of the recipient and can induce simultaneously CD8 and CD4 T cell responses in the individual. The personalised vaccine presented here is both patient specific and disease agnostic. The vaccine may be used in the treatment of conditions such as, for example, cancer, viral infection, and bacterial infection.

Efficacy against an infectious disease or a cancer that can evolve over time is achieved by administering a set of personalized VERDI Vaccines that target different antigens expressed in the unhealthy cells (at the same time and subsequently) but, unlike current vaccines, only a single dose of each vaccine is required. Adjuvants are optional but not required for the personalise vaccines of the present invention.

The VERDI personalized vaccine, as described here, is a novel invention that can take various forms. One preferred embodiment of this invention includes a single synthetic peptide chain that encodes interchangeable CD8 and CD4 antigens. These antigens consist of a set of highly ranked epitopes presented by the subject's HLA molecules on the cell surface. These antigens are derived from proteins expressed in target cells, such as tumor cells or infected cells.

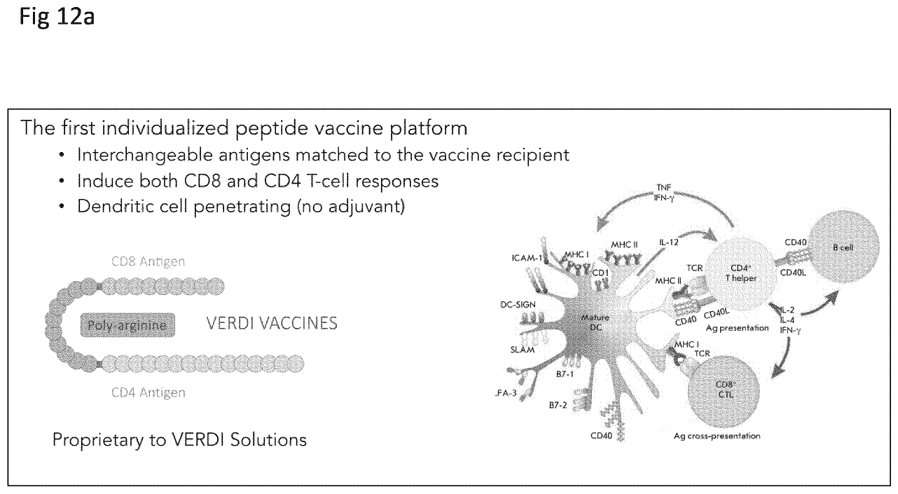

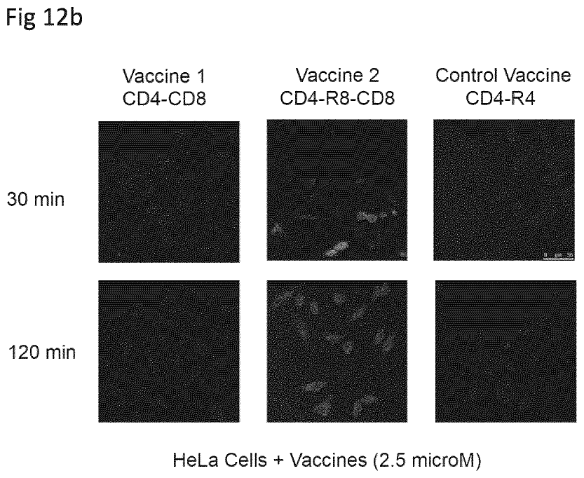

A unique feature of this vaccine is the use of a polyarginine bridge (composed of a peptide with 8 or more arginine units) that covalently links the CD8 and CD4 antigens within the peptide chain (as illustrated in FIGS. 12a, 13a, 14a). With a length of 26-40 amino acids, the VERDI Vaccine provides a personalized and innovative solution for inducing effective T-cell responses with natural peptides.

The polyarginine cell-penetrating peptide plays a pivotal role in the composition of the VERDI vaccine. It acts as an immunologically inert bridge, aiding the uptake of the VERDI vaccine into antigen-presenting dendritic cells and promoting the subsequent induction of potent T-cell responses.

The introduction of this novel peptide vaccine platform represents a significant advancement in personalized cancer vaccines. It addresses the limitations of existing peptide vaccines in terms of (i) antigen uptake, (ii) simultaneous induction of potent CD8 and CD4 T cell responses against a target protein by a single peptide, and (iii) rapid pharmaceutical preparation of the vaccine from two synthetic peptides using the Diels-Alder reaction.

Further preferred aspects and embodiments of the invention will now be described in detail.

According to one aspect of the invention, there is provided a personalised vaccine or treatment composition prepared according to any of the computer implemented methods set out above.

In a further aspect of the invention, the personalised vaccine or treatment composition comprises at least one peptide antigen derived from a protein expressed in the unhealthy cells in a subject wherein the peptide antigen is comprised a set of overlapping epitopes capable of being displayed by the subject's HLA class I and/or class II molecules, induces a CD4 and/or CD8 T cell response, and shares a common sequence with the plurality of epitopes capable of being displayed by the subject's HLA class I and/or class II genotype and, optionally, a cell penetrating peptide.

In one embodiment, the cell penetrating peptide may be positioned at the N-terminus of the at least one peptide antigen. In another embodiment, the cell penetrating peptide may be positioned at the C-terminus of the at least one peptide antigen. In embodiments comprising at least two or more peptide antigens, the cell penetrating peptide may be positioned at the N-terminus of the peptide formed by the at least two antigens, at the C-terminus of the peptide formed by the at least two antigens, or positioned between the at least two antigens. In some embodiments comprising more than two antigens, the vaccine may comprise more than one cell penetrating peptide.

In one embodiment, the at least one peptide antigen may comprise one T cell epitope. In another embodiment, the at least one peptide antigen may comprise at least two T cell epitopes. The at least two T cell epitopes may be arranged contiguously, separated by another peptide region or regions, or overlapping. In one embodiment, the antigen may comprise multiple highly ranked epitopes presented by the subject HLA molecules which is advantageous as this may assist with inducing a CD4 and/or CD8 T cell response.

In one embodiment, the personalised vaccine or treatment composition comprises at least two antigens, wherein the at least two antigens comprise at least one antigen which induces a CD4 T-cell response and at least one antigen which induces a CD8 T-cell response. The antigenic peptide of the present invention takes into account the importance of both CD8 and CD4 T-cell responses against a protein expressed in the unhealthy cells. The diagnosed highly ranked CD8 and CD4 epitopes of an individual, carefully matched with the HLA genotype and disease-specific proteins of the recipients, are included in the peptide antigens selected for the vaccine composition. Therefore, the induction of both CD8 and CD4 T-cell responses in an individual against a sick cell represents a substantial improvement in the efficacy of current T-cell vaccines.

The peptide antigen of the present invention is advantageous because it can induce the CD8 and CD4 T-cell responds together against the sick cells, resulting in improved efficacy of the personalized vaccines. Current vaccine peptides are not designed to induce both CD8 and CD4 T-cell responses against a protein expressed in the target cells, despite the help signals of CD4+ T-cells to CD8+ T-cells during priming to optimize the magnitude and quality of the CTL responses. In addition, sometimes CD4+ T-cells can also kill tumor cells and infected cells which are presenting the specific epitopes recognized by the TCR on the cell surface.

In one embodiment, the at least one peptide antigen contains a set of epitopes having “core” recognized by the TCR of both a CD4 and a CD8 T-cells of the subject. It has been previously recognized that epitopes may be “promiscuous”, that is to say they may bind to more than one HLA class I or HLA class II molecules. However, it is different in this invention since our system and computer-implemented method analyse both HLA-epitope and TCR-epitope recognition on the cell surface at the individual level. In one embodiment, the at least one peptide antigen may contain 1-40 highly ranked epitopes presented by the subject's HLA class I molecules and/or 1-40 highly ranked epitopes presented by the subject's HLA class II molecules on the cell surface.

In one embodiment, in which the personalised vaccine comprises at least two peptide antigens, the cell penetrating peptide is positioned between the at least two antigens. In one aspect of the invention, there is provided an antigenic peptide, comprising the amino acid sequences of at least two peptide antigens and a cell penetrating peptide. In one embodiment of this aspect, one of the antigens can activate CD4⁺ helper T-cells and the other antigen can activate CD8⁺ cytotoxic T-cells and the cell penetrating peptide is positioned between the CD4 antigen amino acid sequence and the CD8 antigen amino acid sequence (FIGS. 12a, 13a, 14a).

A further advantage of the present invention is that the personalized vaccine consists or comprises a single synthetic peptide chain. This eliminates the issues of poor solubility, precipitation, and peptide reactions, making the vaccine preparation and quality control process more efficient and reliable. Current peptide vaccines can comprise 8-20 peptides and the mixture of these different peptides can have different solubilities, meaning that some may precipitate. Further, peptides in the mixture can react with each other. This makes quality control of the peptide mixture time-consuming and challenging, e.g., identity is difficult to confirm because they do not separate in the HPLC column.

Current peptide vaccines will also comprise adjuvants included in the vaccines to increase immunogenicity (e.g. by providing T cell help to CD8 T cell responses and antibody responses) which can cause mild to moderate local side effects (pain, erythema, edema, swelling, heat, redness, etc.) that may occasionally become severe, require treatment, and repeated vaccinations could be poorly tolerated. The personalized peptide vaccine of the present invention is advantageous because it does not rely on adjuvants, reducing the potential for side effects associated with their use and does not require repeated injection due to the simultaneous induction of potent CD8 and CD4 T cell responses. In some embodiments, the present invention does not comprise any adjuvants.

### Cell Penetrating Peptides (CPP)

As used herein, the term “cell-penetrating peptide” refers to a peptide capable of performing transmembrane delivery into a cell of a molecule of interest to which it is attached. The cell penetrating peptide ensures that the vaccine quickly enters into cells, including antigen-presenting dendritic cells, and activates T cell responses. For example, the cell-penetrating peptide of the present application is capable of performing transmembrane delivery into a cell of a biological molecule of interest (e.g., a peptide of interest or nucleic acid of interest) to which it is attached. In the present application, the cell-penetrating peptide can be attached to a biological molecule of interest (e.g., a peptide of interest or a nucleic acid of interest) through covalent or non-covalent linkage.

For example, the cell-penetrating peptide of the present application can be attached to a peptide of interest by covalent linkage (optionally via a linker, for example, a peptide linker). Thus, in certain embodiments, the cell-penetrating peptide of the present application can be optionally fused to a peptide of interest via a peptide linker. Methods for conjugating a peptide molecule to a peptide of interest or nucleic acid of interest are known in the art, for example, using various known bifunctional linkers.

In addition, the cell-penetrating peptide of the present application can be attached to a biological molecule of interest (e.g., a peptide of interest) in a non-covalent manner. Thus, in certain embodiments, the cell-penetrating peptide of the present application can be attached to a biological molecule of interest (e.g., a peptide of interest) through a specific intermolecular interaction/specific binding (e.g., interaction/binding between antigen and antibody; interaction/binding between DNA binding domain and DNA molecule).

The present invention is not limited to any particular CPP or sequence. However, the following specific sequences may be preferable and advantageous because of their sequence, function, tissue specificity, or mode of action.

In one embodiment, the CPP is HIV-TAT comprising the sequence of GRKKRRQRRRPQ (SEQ ID NO. 1029). In one embodiment the CPP is 8 polyarginine comprising the sequence of RRRRRRRR (SEQ ID NO.1030). In one embodiment, the CPP is 9 polyarginine comprising the sequence of RRRRRRRRR (SEQ ID NO. 1031). In one embodiment, the CPP is Penetratin comprising RQIKIWFQNRRMKWKK (SEQ ID NO 1032). In one embodiment, the CPP is KLAL comprising a sequence of KLALKLALKALKAALKLA (SEQ ID NO. 1033). In one embodiment, the CPP is VP-22 comprising the sequence of DAATATRGRSAASRPTERPRAPARSASRPRRPVD (SEQ ID NO.1034). In one embodiment, the CPP is MPG comprising the sequence of GALFLGFLGAAGSTMGAWSQPKKKRKV (SEQ ID NO. 1035). In one embodiment, the CPP is KADY comprising the sequence of Ac-GLWRALWRLLRSLWRLLWKAcysteamide (SEQ ID NO.1036). In one embodiment, the CPP is pVEC comprising the sequence of LLIILRRRIRKQAHAHSK-NH2 (SEQ ID NO.1037). In one embodiment, the CPP is M-918 comprising MVTVLFRRLRIRRASGPPRVRV-NH2 (SEQ ID NO. 1038). In one embodiment, the CPP is KALA comprising WEAKLAKALAKALAKHLAKALAKALKACEA (SEQ ID NO. 1039). In one embodiment, the CPP is PEP-1 comprising Ac-KETWWETWWTEWSQPKKKRKC-cya (SEQ ID NO.1040). In one embodiment, the CPP is EB1 comprising LIKLWSHLIHIWFQNRRLKWKKK (SEQ ID NO. 1041). In one embodiment, the CPP is Transportan comprising a sequence of GWTLNSAGYLLGKINLKALAALAKKIL (SEQ ID NO.1042). In one embodiment, the CPP is p-Antp comprising a sequence of RQIKIWFQNRRMKWKK (SEQ ID NO. 1043). In one embodiment, the CPP is hCT (18-32) comprising a sequence of KFHTFPQTAIGVGAP-NH2 (SEQ ID NO. 1044). In one embodiment, the CPP is KLA comprising a sequence of KLALKLALKALKAALKLA (SEQ ID NO.1045). In one embodiment, the CPP is AGR which is a cancer/tissue specific CPP for prostate carcinoma and comprises the sequence of CAGRRSAYC (SEQ ID NO. 1046). In one embodiment, the CPP is LyP-2 which is a cancer/tissue specific CPP for skin or cervix tumour and comprising the sequence of CNRRTKAGC (SEQ ID NO. 1047). In one embodiment, the CPP is REA which is a cancer/tissue specific CPP for prostate, cervix, or breast carcinoma and comprising the sequence of CREAGRKAC (SEQ ID NO.148). In one embodiment, the CPP is LSD which is a cancer/tissue specific CPP for melanoma or osteocarcinoma and comprising the sequence of CLSDGKRKC (SEQ ID NO. 1049). In one embodiment, the CPP is HN-1 which is a cancer/tissue specific CPP for head and neck squamous cell carcinoma and comprising the sequence of TSPLNIHNGQKL (SEQ ID NO.1050). In one embodiment, the CPP is CTP which is a cancer/tissue specific CPP for cardiac myocytes and comprising the sequence of APWHLSSQYSRT (SEQ ID NO. 1051). In one embodiment, the CPP is HAP-1 which is a cancer/tissue specific CPP for synovial tissue and comprising the sequence of SFHQFARATLAS (SEQ ID NO. 1052). In one embodiment, the CPP is 293P-1 which is a cancer/tissue specific CPP for Keratocyte growth factor and comprising the sequence of SNNNVRPIHIWP (SEQ ID NO.1053).

In one preferred embodiment, the cell penetrating peptide is an immunologically inert cell penetrating peptide. Immunologically inert cell penetrating peptides are advantageous because they will not cause an adverse immunological reaction which may impede a T cell epitope response or cause a T cell epitope response to the CPP rather than the intended antigens in the vaccine.

One example of an immunologically inert cell penetrating peptide is an at least 8-mer polyarginine. The at least 8-mer polyarginine maybe poly-8-arginine or poly-9-arginine. In one embodiment, the at least 8-mer polyarginine may comprise tetrazine.

### Preparation and Manufacture of Personalised Peptide Vaccines

In one embodiment, the CD4 antigen amino acid sequence is a subject specific amino acid sequence and/or the CD8 antigen amino acid sequence is a subject specific amino acid sequence. For example, in one embodiment, the CD4 antigen amino acid sequence and/or the CD8 antigen amino acid sequence is specific to a subject's HLA class I or class II genotype and/or proteins expressed specifically in the sick cells.

In one embodiment, the CD4 antigen, the CD8 antigen, and the cell penetrating peptide are covalently conjugated. In one embodiment, the CD4 antigen contains highly ranked epitopes that are 9-20 amino acids long. In one embodiment, the CD8 antigen contains highly ranked epitopes that are 8-15 amino acids long.

According to one aspect of the invention, there is provided a pharmaceutical composition comprising the personalized peptide vaccine of an aspect of the invention. In one embodiment, the composition further comprises a pharmaceutically acceptable excipient.

According to one aspect of the invention, there is provided a use of a peptide antigen according to an aspect of the invention or a pharmaceutical composition according to an aspect of the invention as a vaccine. In one embodiment, the CD4 antigen amino acid sequence and the CD8 antigen amino acid sequence is a tumor associated antigen. A tumor associated antigens may be viral antigens expressed specifically in the patient's tumor, cancer testis antigens expressed specifically in the patient's tumor, and overexpressed antigens in tumor cells compared to healthy cells. Highly ranked epitopes derived from these tumor-specific antigens are transported by the patient's HLAs to the cell surface and “cores” of these epitopes are recognized by T cells involved in the cellular immune responses.

The antigens derived using the methods set out herein may be included in other delivery system, but mRNA, DNA, viral vector-based vaccines need to be produced industrially and are not therefore affordable and accessible personalized vaccines.

The personalized VERDI Vaccines in the present invention has been designed such that it may be prepared for use in a pharmacy or doctor's office. The single peptide antigen in the vaccine may be synthesized and purified in two separate parts and combined prior to administration. The present invention also allows for personalized peptide vaccines with combinations of different antigens to be efficiently prepared. The vaccine may comprise at least two peptides which comprises or consists of at least one antigen and a portion of a CPP. Upon combination of the at least two peptides, the vaccine is created such that it comprises at least two antigens separated by a complete CPP between the said at least two antigens. Alternatively, the single peptide antigen in the vaccine is synthesized and purified and mixed with one or more excipient prior to administration. This is an innovative new approach to preparing a personalized peptide vaccine for a single individual for a single immunization. It is advantageous because it is not only excellent pharmaceutical quality but also as safe as any peptide vaccines, cost effective, easy to make, and time saving.

The present invention therefore relates to a method of preparing a personalised vaccine or treatment composition comprising preparing a first amino acid sequence and preparing a second amino acid sequence, wherein the first amino acid sequences comprises a first peptide antigen which induces a CD4 and/or CD8 T-cell response and at least a portion of a cell penetrating peptide, wherein the second amino acid sequences comprises a second peptide antigen which induces a CD4 and/or CD8 T-cell response and at least a portion of a cell penetrating peptide, and covalently linking the first and second amino acid sequences to form a personalised vaccine comprising a cell penetrating peptide positioned between the first antigen and the second antigen.

In one preferred embodiment, both the first and second antigens may be linked (covalently or non-covalently) to four arginine and are furnished with a tetrazine or a norbornene moiety. These chemical modifications that makes them amenable for covalent reactions employing the inverse electron demand Diels-Alder reaction.

A general specification for the synthetic peptides is established since the vaccines are individualized and the CD4 and CD8 antigens are interchangeable, specific for the vaccine recipient. Lyophilized peptides for the first and second antigens linked to the four arginine may be conjugated to form the finalized personalized peptide vaccine product.

To get the conjugated vaccine product the peptides of the first and second antigens linked to the four arginine may be dissolved in Phosphate-buffered saline (PBS) at a concentration of 1 mg/mL, and the reaction mixture is heated to 40° C. for 1-4 hours. The reaction can be followed by the decolorization of the purple color of the tetrazine moiety. This rapid chemical reaction ensures the conjugation of two peptide antigens in a fast and efficient biorthogonal way.

### Protocol for In Vitro Vaccination of HLA-Genotyped Human Subjects

According to an aspect of the invention, there is provided a method for administering a peptide antigen to a subject, comprising administering the peptide antigen according to an aspect of the invention or a pharmaceutical composition according to an aspect of the invention to the subject. In one embodiment, the antigenic peptide according to an aspect of the invention or a pharmaceutical composition according to an aspect of the invention is administered to the subject at an effective amount.

According to an aspect of the invention, there is provided a method for inducing antigen-specific immunity in a subject comprising administering the peptide antigen according to an aspect of the invention or a pharmaceutical composition according to an aspect of the invention to the subject.

Directive 2010/63/BU requires integrating the 3Rs principles and welfare standards for the treatment of animals in all aspects of the development, manufacture, and testing of medicines. The 3R principles encourage the reduction, refinement, and replacement of animal testing in the development of medicines. Inventors have actively pursued alternatives to animal experiments and, for the first time, successfully identified an in vitro human vaccination model that replaces the need for animal testing.

The use of in vitro human vaccination holds significant advantages over animal models when it comes to the development of personalized vaccines. Unlike animal models, in vitro human vaccination provides a more accurate prediction of the antigen-specific T-cell responses induced by personalized vaccines in an individual, taking into account the specific sets of HLA class I and class II molecules present in that individual. This personalized approach is crucial because T-cell responses vary among individuals and are regulated by all human HLA molecules, which consist of six HLA class I and twelve HLA class II molecules. Animal models, such as HLA-transgenic mice, fall short in mimicking the complex genetic background of human subjects and therefore cannot reliably predict antigen-specific T-cell responses in individuals.

By employing an in vitro human vaccination model, it is possible to overcome the limitations of animal models and gain a more accurate understanding of the personalized vaccines' impact on T-cell responses in humans. This approach aligns with the principles of personalized medicine, where individual variations in immune responses play a critical role.

The in vitro method for assessing the efficacy of personalized vaccination in human subjects, which replaces animal testing, is outlined in detail as follows.

In one embodiment, the in vitro method for assessing the efficacy of vaccination of human subjects comprises the steps of:
a) preparing monocyte-derived dendritic cells (DC) from HLA-genotyped individuals;
b) incubating DC with a peptide antigen, the one to be included in the personalized peptide vaccine;
c) co-culturing the DC with autologous PBMC or isolated T cells in T cell medium;
d) testing antigen-specific CD8 and CD4 T-cell responses and/or performing CD8 and CD4 T-cell proliferation assays.

This approach makes it possible to tailor vaccines to the specific immune characteristics of individuals.

Such study conducted on the vaccine composition provides compelling evidence for its potential as the first individualized peptide vaccine platform. This innovative vaccine exhibits several novel features that contribute to its effectiveness in activating both CD8 and CD4 T-cell responses. The inclusion of both CD8 and CD4 T-cell antigens enables individualization based on the unique HLA class I and class II alleles expressed by each individual. The uptake of the vaccine by dendritic cells ensures efficient processing and presentation of antigens, leading to robust T-cell responses. Importantly, the vaccine composition, devoid of adjuvants, offers a safe and natural peptide-based vaccine solution. Overall, the vaccine holds great promise for personalized cancer immunotherapy, offering new avenues for improved patient outcomes and revolutionizing the field of personalized vaccination.

### Treatment Using the Personalized VERDI Vaccine of the Present Invention

The personalised VERDI vaccines represent an entirely new way to treat disease and are bespoke for each individual. Following administration, the personalized vaccine induces both CD8 and CD4 T-cell responses against the unhealthy cells of the subject. This is significant because CD4 helper T-cells play a crucial role in supporting dendritic cell maturation and the induction of CD8 cytotoxic T-cell responses and antibody responses. Inclusion of both CD8 and CD4 T-cell antigens in the vaccine ensures individualization, as the T-cell responses against antigens derived from unhealthy cells, e.g. cancer or infected cells, are determined by the unique HLA class I and class II alleles expressed by each patient.

Another important feature of the vaccine is its high uptake by cells within just 30 minutes. This efficient loading of dendritic cells with the vaccine peptide enables the processing of the vaccine to epitopes and the rapid saturation both HLA class I and class II molecules, leading to superior antigen presentation to T cells and the induction of CD8 and CD4 T-cell responses, respectively.

The VERDI personalised vaccines do not require adjuvants to be administered at the same time as the vaccine or subsequently. This is highly advantageous when treating a subject because of the known side effects often associated with such adjuvants. The VERDI personalised vaccines are therefore the safest vaccine platforms ever developed.

The vaccine may be administered intramuscularly, intravenously, intratracheally, intrabursally, intraperitoneally, subcutaneously, or intraocularly. In one embodiment, the vaccine is administered at an effective dose. In a further embodiment, the vaccine is administered in a single effective dose. In one embodiment, the subject is a mammal. In a further embodiment, the subject is a human.

According to one aspect of the invention, there is provided a method of treating or preventing a disease, comprising administering the personalised vaccine or treatment composition to the subject. In one embodiment, the personalised vaccine or treatment composition is administered in conjunction with an additional therapeutic agent and wherein said administration is concurrently or sequentially.

In one embodiment, the at least two personalised vaccine or treatment compositions comprising different peptide antigens are administered to the subject concurrently or sequentially.

In one embodiment, the personalised vaccine or treatment composition is administered with an adjuvant and wherein administration is concurrently or sequentially.

In one embodiment, the disease is cancer and/or viral infection and/or autoimmune disease.

In a further aspect of the invention, there is provided a method for inducing antigen-specific immunity in a subject comprising administering the personalised vaccine or treatment composition to the subject.

In a further aspect of the invention, there is provided a personalised vaccine or treatment composition for use in the prevention or treatment of a disease.

Presently available therapeutic and preventive vaccines use repeated doses of the vaccine to increase immune responses. In one embodiment of the present invention, only a single vaccine in a single dose is required to be administered to the patient as therapeutic vaccine. A single dose of any one vaccine is preferable because repeated vaccination with multiple doses may exhausts T cell responses. This is similar to what is found in chronic infection and cancer and limits the ability of the body to fight disease and is one reason why the present invention is advantageous over current therapeutic vaccines.

In a further embodiment, if a further vaccine dose is required, then the vaccine is directed to a different target. This is the benefit of personalised vaccines because they can be prepared to specific target antigens from an unhealthy cell. For example, if the unhealthy cell is a cancer cell, then a first doses of personalized vaccines can be prepared and administered using antigens expressed in the cancer cells that may be determined by transcriptome analysis of the tumor biopsy. If a new tumor(s) is diagnosed in the patient, or the vaccines cannot entirely eliminate the tumor, then a further tumor sample can be taken, and new vaccines designed and used for the treatment of the patient. This is repeated until the patient has detectable tumor. Such flexible and curative method of treatment invented here is only possible with personalized VERDI Vaccines.

In one embodiment, the disease is cancer, and the cancer is selected from a list comprising adenoid cystic carcinoma, adrenal gland tumor. amyloidosis, anal cancer, appendix cancer, astrocytoma, ataxia-telangiectasia, Beckwith-Wiedemann syndrome, bile duct cancer (cholangiocarcinoma), Birt-Hogg-Dubé syndrome, bladder cancer, bone cancer (sarcoma of bone), brain stem glioma, brain tumor, breast cancer, carney complex, central nervous system tumors, cervical cancer, colorectal cancer, Cowden syndrome, craniopharyngioma, desmoid tumor, desmoplastic infantile ganglioglioma, ependymoma, esophageal cancer, ewing sarcoma, eye cancer, eyelid cancer, familial adenomatous polyposis, familial malignant melanoma, familial pancreatic cancer, gallbladder cancer, gastrointestinal stromal tumor, germ cell tumor, gestational trophoblastic disease, head and neck cancer, diffuse gastric cancer, leiomyomatosis, renal cell cancer, mixed polyposis syndrome, pancreatitis, papillary renal carcinoma, juvenile polyposis syndrome, kidney cancer, laryngeal cancer, hypopharyngeal cancer, leukemia, lymphoblastic cancer, lymphocytic cancer, acute myeloid cancer, b-cell prolymphocytic leukemia, hairy cell leukemia, eosinophilic leukemia, Li-Fraumeni syndrome, liver cancer, lung cancer, non-small cell carcinoma, small cell carcinoma, Hodgkin lymphoma, Non-Hodgkin lymphoma, Lynch syndrome, mastocytosis, medulloblastoma, melanoma, meningioma, mesothelioma, multiple endocrine neoplasia type 1, multiple endocrine neoplasia type 2, multiple myeloma, MUTYH (or MYH)-associated polyposis, myelodysplastic syndromes, nasal cavity cancer, and paranasal sinus cancer, nasopharyngeal cancer, neuroblastoma, neuroendocrine tumor of the gastrointestinal tract, neuroendocrine tumor of the lung, neuroendocrine tumor of the pancreas, neuroendocrine tumors, neurofibromatosis type 1, neurofibromatosis type 2, nevoid basal cell carcinoma syndrome, oral cancer, oropharyngeal cancer, osteosarcoma, ovarian cancer, fallopian tube cancer, peritoneal cancer, pancreatic cancer, parathyroid cancer, penile cancer, Peutz-Jeghers syndrome, pheochromocytoma, paraganglioma, pituitary gland tumor, pleuropulmonary blastoma, prostate cancer, retinoblastoma, rhabdomyosarcoma, salivary gland cancer, Kaposi sarcoma, soft tissue sarcomas, skin cancer, small bowel cancer, stomach cancer, testicular cancer, thymoma carcinoma, thymic carcinoma, thyroid cancer, tuberous sclerosis complex, uterine cancer, vaginal cancer, Von Hippel-Lindau syndrome, vulvar cancer, Waldenstrom macroglobulinemia (lymphoplasmacytic lymphoma), Werner syndrome, Wilms tumor, and xeroderma pigmentosum. In one embodiment, the personalized VERDI vaccines can be administered in conjunction with any drugs and biologicals, either in combination or sequentially, based on the physician's discretion. It is similar to prophylactic vaccines, such as the COVID-19 vaccines, which are administered in addition to the therapies that the patient is receiving. Specifically, the personalized VERDI vaccines may be administered in combination with at least one anti-cancer therapeutic.

In one embodiment, the anti-cancer therapeutic is selected from a list comprising alkylating agents, cytotoxic antibiotics, antimetabolites, antiangiogenics, histone deacetylase inhibitors, hormones, protein kinase inhibitors, growth factors, CAR T-cells, taxanes, topoisomerase inhibitors, vinca alkaloids, polyclonal antibodies, monoclonal antibodies or fragments thereof, or immune checkpoint inhibitors.

Progression of the disease being treated by the vaccine may be monitored post administration in numerous ways of which the skilled person would be well aware. For example, detection of biomarkers, X-ray scans, MRI scans, CT scans, polymerase chain reaction (PCR), reverse transcription-polymerase chain reaction (RT-PCR) tests, branched DNA (bDNA) tests, nucleic acid sequence-based amplification (NASBA) tests, bacterial culturing and many other known techniques.

### Personalized VERDI Vaccine Kits

Preparation of a single dose of personalized peptide vaccine before administration may require a personalized VERDI Vaccine kit.

A kit comprising the personalised vaccine or treatment composition, optionally, wherein the kit further comprising a pharmaceutically acceptable excipient, and, further optionally, including instructions for use.

A kit comprising the several items required to prepare the personalised vaccine composition for the individual comprising: two synthetic peptides wherein each synthetic peptide comprising at least one antigen and further comprising at least a portion of a cell-penetrating peptide. The personalized vaccine preparation before administration includes the covalent linkage of the two synthetic peptides that results in reconstitution of the function of the cell-penetrating peptide

According to a further aspect of the invention, there is provided a kit comprising the personalized peptide antigen composition and the other ingredients and tools required for the preparation of the personalized vaccines in the pharmacy and administration of the personalized vaccine. For example, excipients, adjuvants, syringes, adaptors, sterile filters, plastic ware, instructions for use, etc. This list is not limited to these components and further components of the it may be apparent to the skilled person.

The invention is further defined in the following numbered aspects:
1. A computer-implemented method for identifying, for a subject, at least one antigen which is expected to induce a T-cell response to attack unhealthy cells in the subject, the method comprising:
receiving subject data identifying multiple major histocompatibility complex, MHC, molecules for the subject;
receiving sequence data for a plurality of epitopes within a protein which is expressed in the unhealthy cells, wherein each epitope is an amino acid sequence within the protein;
obtaining for each epitope in the plurality of epitopes, a potency score which is indicative of the likelihood that each of the identified multiple MHC molecules displays the epitope on their surface;
generating a ranked list of epitopes based on the determined potency scores; identifying at least one antigen by selecting at least one epitope which is a highly ranked in the ranked list; and
outputting at least one of the ranked list and sequence data for the identified at least one antigen.
2. The method of aspect 1, further comprising
selecting multiple epitopes which are highly ranked in the ranked list,
identifying the subitopes of each of the selected epitopes using a directed graph network, and
identifying each subitope which is common to more than one of the selected epitopes.
3. The method of aspect 2, further comprising identifying the at least one antigen by selecting a common subitope which is highly ranked in the ranked list.
4. The method of aspect 2 or aspect 3, further comprising identifying the at least one antigen by selecting a longest common subitope.
5. The method of any one of aspects 2 to 4, further comprising identifying a shortest common subitope as a target sequence.
6. The method according to any one of the preceding aspects, wherein obtaining a potency score comprises calculating an epitope weight score for each epitope in the plurality of epitopes by
selecting an epitope from the plurality of epitopes,
obtaining, for each of the identified MHC molecules, a probability score which is indicative of the likelihood that each MHC molecule transports the selected epitope; and calculating the epitope weight score for the selected epitope by aggregating at least some of the probability scores.
7. The method of aspect 6, wherein the probability score is an eluted ligand score which is indicative of the likelihood that the epitope can be eluted from a given MHC molecule.
8. The method of aspect 7, wherein the epitope weight score is calculated from:

$${EWS}_{( x )} = {\sum }_{i = 1}^{n} {w}_{i} * {ELS}_{( x , i )} for all {w}_{i} * {ELS}_{( x , i )} > ELT$$

where x is a peptide, i is between 1 and n with n being the number of identified MHC molecules, ELS_{(x,i)} is the probability score, i.e. eluted ligand score for each MHC molecule and epitope pair, ELT is an eluted ligand threshold and wᵢ is a weighting parameter.
9. The method of any one of the preceding aspects, when dependent on any one of aspects 2 to 5, further comprising calculating at least one additional score for each epitope which is indicative of whether the subitopes of each epitopes are capable of triggering the same T-cell response.
10. The method of aspect 9, further comprising calculating, for each epitope in the plurality of epitopes, at least one of:
a left subitope score which is based on the epitope weight score for the subitopes in a left subgraph of the directed graph network for the epitope, and
a right subitope score which is based on the epitope weight score for the subitopes in a right subtree of the directed graph network for the epitope.
11. The method of aspect 10, wherein the left subitope score (SWS1) and the right subitope score (SWS2) may be calculated from:

$$SWS {1}_{( {x}_{i} )} = {EWS}_{( {lx}_{i - 1} )} + SWS {1}_{( {lx}_{i - 1} )} + SWS {2}_{( {lx}_{i - 1} )} \\ SWS {2}_{( {x}_{i} )} = {EWS}_{( {rx}_{i - 1} )} + SWS {2}_{( {rx}_{i - 1} )}$$

where xᵢ is an epitope with the length of i, where i has a range of between a to b amino acids, lxᵢ₋₁ is the left sub-epitope of xᵢ and rxᵢ₋₁ is the right sub-epitope of x; and the initial values are SWS1(xₐ)=0, and SWS2(xₐ)=0.
12. The method of any one of aspects 9 to 11, further comprising calculating, for each epitope in the plurality of epitopes, an overall weight score OWS which combines the epitope weight score with the at least one additional score.
13. The method of aspect 12, when dependent on aspect 11, wherein the overall weight score OWS is calculated from:

$$OWS = EWS + {w}_{i} * SWS 1 + {v}_{i} * SWS 2 when EWS exceeds an EWS \\ threshold otherwise OWS = SWS 1 + SWS 2$$

where EWS is the epitope weight score, SWS1 is the left subitope score, SWS2 is the right subitope score and wᵢ and vᵢ are weights.
14. The method of any one of the preceding aspects, wherein the subject is a human and wherein the subject data identifies at least part of the human leukocyte antigen, HLA-genotype for the subject.
15. The method of aspect 14, wherein the subject data identifies at least one of a set of HLA class I molecules and a set of HLA class II molecules.
16. The method of aspect 15, wherein when the subject data identifies the set of HLA class I molecules, each of the plurality of epitopes is an amino acid sequence of between 8 to 14 amino acids.
17. The method of aspect 15 or aspect 16, wherein when the subject data identifies the set of HLA class II molecules, each of the plurality of epitopes is an amino acid sequence of at least 9 amino acids.
18. The method of any one of aspects 15 to 17, wherein the method comprises obtaining for each epitope in the plurality of epitopes, a first potency score which is indicative of the likelihood that each of the identified molecules in the set of HLA class I molecules displays the epitope on their surface; and
obtaining for each epitope in the plurality of epitopes, a second potency score which is indicative of the likelihood that each of the identified molecules in the set of HLA class II molecules displays the epitope on their surface.
19. The method of aspect 18, comprising:
generating a first ranked list of epitopes based on the obtained first potency scores; and
generating a second ranked list of epitopes based on the obtained first potency scores.
20. The method of any preceding aspect, further comprising
determining an aggregated score using at least some of the potency scores from the highly ranked epitopes and
predicting the strength of the subject's response to the illness based on the aggregated score.
21. A method of stratifying a group of subjects for vaccination by
determining an aggregated score as set out in aspect 20,
comparing the aggregated score to a strength threshold, and
classifying subjects having aggregated scores below the strength threshold as a priority for preventive and treatment approaches like vaccination and T cell therapy.
22. A method of designing a personalized vaccine to induce a T-cell response against a protein expressed in the sick cells, the method comprising selecting one or more of the antigens identified by any one of aspects 1 to 21 for incorporation in the vaccine.
23. A method of designing a personalized vaccine to induce a T-cell response to attack unhealthy cells in a subject, the method comprising
receiving subject data identifying multiple major histocompatibility complex, MHC, molecules for the subject, wherein the subject data identifies at least one of a set of HLA class I molecules and a set of HLA class II molecules;
receiving sequence data for a plurality of epitopes within a protein expressed in the unhealthy cells wherein each epitope is an amino acid sequence within the protein;
obtaining for each epitope in the plurality of epitopes, a first potency score which is indicative of the likelihood that each of the identified set of HLA class I molecules displays the epitope on their surface;
generating a first ranked list of epitopes based on the determined first potency scores;
selecting multiple epitopes which are highly ranked in the first ranked list;
identifying the subitopes of each of the selected epitopes using a directed graph network;
identifying each subitope which is common to more than one of the selected epitopes;
identifying at least one first antigen by selecting at least one subitope which is itself a highly ranked epitope in the first ranked list; and
outputting the identified at least one first antigen for the personalized vaccine.
24. The method according to aspect 23, further comprising
obtaining for each epitope in the plurality of epitopes, a second potency score which is indicative of the likelihood that each of the identified set of HLA class II molecules displays the epitope on their surface;
generating a second ranked list of epitopes based on the determined second potency scores;
selecting multiple epitopes which are highly ranked in the second ranked list;
identifying the subitopes of each of the selected epitopes using a directed graph network;
identifying each subitope which is common to more than one of the selected epitopes;
identifying at least one second antigen by selecting at least one subitope which is itself a highly ranked epitope in the second ranked list; and
outputting the identified at least one second antigen for the personalized vaccine.
25. The method according to aspect 24, further comprising
determining whether the identified first antigen is a highly ranked epitope in the second ranked list, and
when the first antigen is a highly ranked epitope in the second ranked list, designing the personalized vaccine based on the first antigen.
26. A method of designing a general-purpose vaccine, the method comprising:
designing a plurality of personalized vaccines as set out in aspects 23 to 25,
selecting at least one antigen which is more frequently used in the personalized vaccine and
including the at least one selected antigen in the general-purpose vaccine.
27. A method of predicting a response to the personalized or general-purpose vaccine designed using any one of the aspects 23 to 26, the method of predicting comprising:
receiving subject data identifying multiple major histocompatibility complex, MHC, molecules for the subject;
receiving sequence data for each epitope within the vaccine, wherein each epitope is an amino acid sequence;
obtaining for each epitope in the vaccine, a potency score which is indicative of the likelihood that each of the identified MHC molecules displays the epitope on their surface; and
predicting the subject's response to the vaccine based on the potency score.
28. A computer-implemented method of identifying targets for T-cell therapy derived from a protein expressed in unhealthy cells, the method comprising
receiving subject data identifying multiple major histocompatibility complex, MHC, molecules for the subject;
receiving sequence data for a plurality of epitopes within the protein, wherein each epitope is an amino acid sequence within the protein;
obtaining for each epitope in the plurality of epitopes, a potency score which is indicative of the likelihood that each of the identified multiple MHC molecules displays the epitope on their surface;
selecting multiple epitopes which are highly ranked in the ranked list, identifying the subitopes of each of the selected
epitopes using a directed graph network,
identifying each subitope which is common to more than one of the selected epitopes;
determining a length of the sequence each identified subitope and
outputting a subitope with the shortest length as the target.
29. A method of predicting a response to the T-cell therapy designed using aspect 28, the method of predicting comprising:
receiving subject data identifying multiple major histocompatibility complex, MHC, molecules for the subject;
receiving sequence data for each epitope which comprises the output target, wherein each epitope is an amino acid sequence;
obtaining for each epitope, a potency score which is indicative of the likelihood that each of the identified MHC molecules displays the epitope on their surface;
   and
predicting the subject's response to the T-cell therapy based on the potency score.
30. The method of any preceding aspect, wherein the protein is the SARS-CoV-2 protein which is associated with Covid-19.
31. The method of any one of aspects 1 to 30, wherein the protein is expressed in a tumor cell, e.g. a protein expressed in metastatic breast cancer.
32. A personalised vaccine or treatment composition prepared according to the method of any one of aspects 1 to 31 and, optionally, a cell penetrating peptide.
33. A personalised vaccine or treatment composition, comprising:
at least one peptide antigen derived from a protein expressed in or on an unhealthy cell or a pathogen in a subject wherein the peptide is capable of being displayed by the subject's HLA class I and/or class II genotype, induces a CD4 and/or CD8 T cell response and shares a common sequence with a plurality of peptide antigens capable of being displayed by the subject's HLA class I and/or class II genotype and a cell penetrating peptide.
34. The personalised vaccine or treatment composition according to aspect 32 or aspect 33, wherein the cell penetrating peptide is an immunologically inert cell penetrating peptide.
35. The personalised vaccine or treatment composition according to aspect 33 or 34, wherein the immunologically inert cell penetrating peptide is at least 8-mer polyarginine.
36. The personalised vaccine or treatment composition according to any one of the preceding aspects, wherein the personalised vaccine or treatment composition comprises at least two antigens, wherein the at least two antigens comprise at least one antigen which induces a CD4 T-cell response and at least one antigen which induces a CD8 T-cell response.
37. The personalised vaccine or treatment composition according to any one of aspects 33 to 35, wherein the personalised vaccine or treatment composition comprises at least two antigens, wherein at least one antigen or at least two antigens induce both a CD4 T-cell response and a CD8 T-cell response.
38. The personalised vaccine or treatment composition according to aspect 36 or aspect 37, wherein the cell penetrating peptide is positioned between the at least two antigens.
39. A method of treating or preventing a disease, comprising administering the personalised vaccine or treatment composition according to any of aspects 33 to 38 to the subject.
40. The method of treatment according to aspect 39, wherein the personalised vaccine or treatment composition is administered in conjunction with an additional therapeutic agent and wherein said administration is concurrently or sequentially.
41. The method of treatment according to any one of aspect 39 and aspect 40, wherein at least two personalised vaccine or treatment compositions comprising different peptide antigens are administered to the subject concurrently or sequentially.
42. The method of treatment according to any one of aspects 39 to 41, wherein the personalised vaccine or treatment composition is administered with an adjuvant and wherein administration is concurrently or sequentially.
43. The method of treatment according to any one of aspects 39 to 42 wherein the disease is cancer or viral infection.
44. A method for inducing antigen-specific immunity in a subject comprising administering the personalised vaccine or treatment composition according to any of aspects 1 to 38 to the subject.
45. A personalised vaccine or treatment composition according to any of aspects 33 to 38 for use in the prevention or treatment of a disease.
46. A method of preparing a personalised vaccine or treatment composition comprising preparing a first amino acid sequence and preparing a second amino acid sequence, wherein the first amino acid sequences comprises a first peptide antigen which induces a CD4 and/or CD8 T-cell response and at least a portion of a cell penetrating peptide, wherein the second amino acid sequences comprises a second peptide antigen which induces a CD4 and/or CD8 T-cell response and at least a portion of a cell penetrating peptide, and covalently linking the first and second amino acid sequences to form a personalised vaccine comprising a cell penetrating peptide positioned between the first antigen and the second antigen.
47. A kit comprising the personalised vaccine or treatment composition according to any of aspects 33 to 38, optionally, wherein the kit further comprising a pharmaceutically acceptable excipient, and, further optionally, including instructions for use.
48. The kit according to aspect 47 further comprising a first amino acid sequence comprising a first peptide antigen which induces a CD4 and/or CD8 T-cell response and at least a portion of a cell penetrating peptide and a the second amino acid sequences comprises a second peptide antigen which induces a CD4 and/or CD8 T-cell response and at least a portion of a cell penetrating peptide, and means for covalently linking the first and second amino acid sequences to form a personalised vaccine comprising a cell penetrating peptide positioned between the first antigen and the second antigen.
49. The it according to aspect 48, wherein the means for covalently linking the first and second amino acid sequences to form a personalised vaccine is a phosphate-buffered saline (PBS) solution.

All of the features contained herein may be combined with any of the above aspects in any combination.

The invention is further illustrated in the following non-binding examples.

### EXAMPLES OF PERSONALISED VACCINES ACCORDING TO THE INVENTION

### Example 1. Cellular Uptake of the Vaccine Composed with Model Antigens

Study objectives: The objective of the study is to demonstrate effective cellular uptake of vaccine composition.

Model Vaccine design: Two well-characterized T-cell antigens were selected as model antigens: CD8 antigen (from pp65 CMV): NLVPMVATV (SEQ ID NO: 1054); CD4 antigen (from Tetanus toxin): QYIKANSKFIGITE (SEQ ID NO: 1055).

Two peptide vaccines were designed compositions using the CD4 and CD8 model antigens: Vaccine 1 (control): KSS-QYIKANSKFIGITE-AAA-LNVPMVATV (SEQ ID NO: 1117); Vaccine 2 (Vaccine): KSS-QYIKANSKFIGITE-RRRRRRRR-NVPMVATV (SEQ ID NO: 1118)

Synthesis of the study vaccines: Fluorescently-labelled and control vaccines were synthesized for cellular uptake studies:

Labelled Vaccine (CD4-CD8)

(SEQ ID NO: 1057)
Fluoresc-KSSQYIKANSKFIGITEAAALNVPMVATV-NH2
Labelled Vaccine conjugate (CD4-R8-CD8):
(SEQ ID NO: 1103)
Fluoresc-KSSQYIKANSKFIGITERRRRRRRRLNVPMVATV-NH2

### 3. Labelled Control Vaccine (CD4-R4)

(SEQ ID NO: 1058)
Fluoresc-KSSQYIKANSKFIGITERRRR-norbornene-NH
4. Not labelled Control Vaccine conjugate
(CD4-R8-CD8):
(SEQ ID NO: 1056)
KSSQYIKANSKFIGITERRRRRRRRLNVPMVATV-NH2

Methods: In this experiment, the uptake of two fluorescent-labeled peptide vaccines was examined using a fluorescent microscope in cultured cells. HeLa cells were grown on cover slides for one day and then treated with peptide vaccines. Cells were fixed with 4% paraformaldehyde, washed with PBS, and analyzed with fluorescent microscopy. The non-labeled negative control vaccine is not shown, because as expected it did not show any fluorescence.

Results: FIG. 12b illustrates the cellular uptake of the vaccine and control vaccines in HeLa cells after 30 minutes and 120 minutes of incubation. Cellular uptake was visualized using fluorescence microscopy. The results clearly depict a substantial increase in the uptake of the vaccine. vaccine compared to the control vaccines. This enhanced cellular delivery highlights the potential efficacy of the vaccine in immunization.

Conclusion: Enhanced Cellular Uptake of vaccine. The experiments revealed notable observations regarding the uptake of the vaccine (CD4-R8-CD8) compared to peptide vaccines (CD4-CD8) lacking the R8 component. The results indicated that the vaccine exhibited rapid uptake into both the cytoplasm and nucleus of the cells within 30 minutes. Subsequently, the vaccine showed a diffuse distribution throughout the cytoplasm and nucleus after 120 minutes. In contrast, the peptide vaccines lacking the R8 component demonstrated poorly detectable cellular uptake within the same 120-minute timeframe and did not exhibit significant accumulation within the cells. These findings led to the conclusion that the cellular uptake of the vaccine is considerably faster in comparison to the control peptide vaccines.

The improved cellular uptake of the vaccine suggests enhanced efficiency in loading the peptide antigens onto the HLA molecules within the cells. This efficient loading process is crucial for the subsequent immune response triggered by the T cells, emphasizing the potential of the vaccine to effectively induce immune reactions and generate robust T-cell responses against target antigens.

### Example 2. Cellular Uptake of Personalized Vaccine Composed with Human Papilloma Virus (HPV) Antigens

Study objectives: The objective of the study is to quantify the cellular uptake of HPV-specific personalized vaccine.

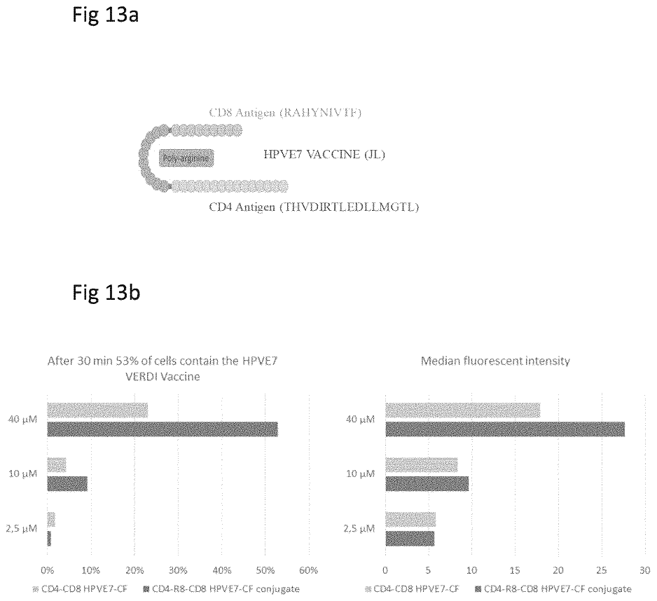

Vaccine design: The inventors have designed HPV-specific vaccine matching with an individualised HLA-genotype of a subject (FIG. 13a). This vaccine contains a CD8 and a CD4 antigen derived from the HPV E7 protein expressed by high-risk types of HPV-16. E7 is known for its oncogenic properties and plays a key role in HPV-associated cervical cancer and other HPV-related malignancies. It interacts with host cell proteins, including tumor suppressor proteins like pRb, leading to dysregulation of the cell cycle and promotion of cell proliferation. The HPV E7 protein is considered a potential target for therapeutic interventions and diagnostic approaches aimed at combating HPV-associated diseases.

Synthesis of the study vaccines: Fluorescently-labeled HPV-specific vaccines were synthesized and the control vaccine (no polyarginine) for cellular uptake studies:

### Labeled HPV-Specific Vaccine Conjugate (CD4-R8-CD8):

(SEQ ID NO: 1104):
Fluoresc-THVDIRTLEDLLMGTL-RRRRRRRR-RAHYNIVTF-NH2

### Labeled Control Vaccine (CD4-CD8):

Fluoresc-THVDIRTLEDLLMGTL-RAHYNIVTF-NH2 (SEQ ID NO: 1105)

Methods: HeLa cells were seeded at a density of 200,000 cells per well in 6-well plates and allowed to grow for one day. On the second day, the cells were treated with the peptides at three different concentrations (2.5 μM, 10 μM, and 40 μM) for two different incubation times (15 minutes and 30 minutes). After the treatment, the cells were detached using trypsin, washed, and resuspended in PBS. The cells were then analyzed using a FACS Canto flow cytometer. A total of 30,000 events were acquired, and the percentage of fluorescent cells and fluorescent intensity (geometric mean and median) were determined through flow analysis.

Results: FIG. 13b shows quantification of cellular uptake of personalized HPV-specific vaccine (blue) compared to control (red) after 30 minutes incubation with human cells. The cellular uptake was assessed by flow cytometry, and the intensity of fluorescence was measured as an indicator of vaccine uptake by the cells. The results demonstrate a significantly higher uptake of the vaccine (blue) compared to the control (red), indicating its enhanced cellular delivery and potential efficacy in HPV-specific immunization.

Conclusions: Effective Cellular Uptake of HPV-specific Personalized Vaccine. The experimental findings demonstrated a considerably higher uptake within 30 minutes of the vaccine compared to the control peptide vaccines lacking the R8 component. This observation is particularly relevant for peptide vaccination because the peptides used in such vaccines are known to degrade rapidly after injection. The experiments revealed that a concentration of 40 μM was optimal for the formulating the vaccine, resulting in more than 50% of the cells taking up the vaccine within just 30 minutes. This rapid and efficient cellular uptake is crucial for peptide vaccination as it ensures timely delivery of the antigens to the target cells.

The fluorescence intensity values were analyzed using the median, which represents the middle value in a sorted list of intensity values. By utilizing the median, the analysis considered the distribution of values and was less affected by extreme values or outliers compared to the mean. This approach provided a robust measure of central tendency, suitable for distributions with different shapes, including skewed or non-normal distributions. Notably, the median analysis revealed that approximately 30% more peptide accumulated in each cell when using the vaccine compared to the control vaccines. This finding further emphasizes the enhanced cellular uptake and potential efficacy of the vaccine in loading the epitopes to the patient's HLA molecules.

### Example 3. Cellular Uptake of the Personalized Vaccine Composed with AKAP-4 Tumor-Specific Antigens

Study objectives: The objective of the study is to quantify the cellular uptake of personalized AKAP-4-specific vaccine.

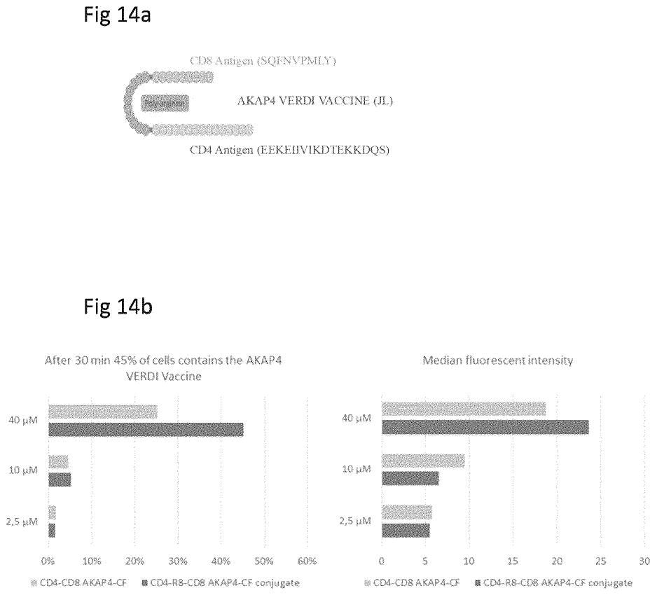

Vaccine design: AKAP-4-specific vaccines were designed by matching with the HLA genotype of a specific individual (FIG. 14a). This vaccine contains a CD8 and a CD4 antigen derived from the AKAP-4 protein that is expressed in aggressive ovarian, lung, colorectal, pancreatic, and prostate cancers. AKAP-4 is an exceptional target for T cell therapy and cancer vaccines due to its specific expression patterns in cancer cells and absence of expression in healthy cells. By targeting AKAP-4, these therapeutic approaches hold promise in enhancing anti-tumor immune responses, potentially leading to improved outcomes for cancer patients.

Synthesis of the study vaccines: Fluorescently-labelled AKAP-4-specific vaccine were synthesised and the control vaccine (no polyarginine) for cellular uptake studies:

### Labelled HPV-Specific Vaccine Conjugate (CD4-R8-CD8);

(SEQ ID NO: 1106)
Fluoresc-EEKEIIVIKDTEKKDQS-RRRRRRRR-SQFNVPMLY-NH2
Labelled control vaccine (CD4-CD8):
(SEQ ID NO: 1107)
Fluoresc-EEKEIIVIKDTEKKDQS-SQFNVPMLY-NH2

Methods HeLa cells were seeded at a density of 200,000 cells per well in 6-well plates and allowed to grow for one day. On the second day, the cells were treated with the peptides at three different concentrations (2.5 μM, 10 μM, and 40 μM) for two different incubation times (15 minutes and 30 minutes). After the treatment, the cells were detached using trypsin, washed, and resuspended in PBS. The cells were then analyzed using a FACS Canto flow cytometer. A total of 30,000 events were acquired, and the percentage of fluorescent cells and fluorescent intensity (geometric mean and median) were determined through flow analysis.

Results: As shown in FIG. 14b, quantification of cellular uptake of personalized AKAP-4-specific vaccine (blue) compared to control (red). The cellular uptake was assessed by flow cytometry, and the intensity of fluorescence was measured as an indicator of vaccine uptake by the cells. The results demonstrate a significantly higher uptake of the vaccine (blue) compared to the control (red), indicating its enhanced cellular delivery and potential efficacy in AKAP-specific immunization.

Conclusions: Effective Cellular Uptake of AKAP-4-specific Personalized Vaccine. The experimental results further confirmed the significantly higher uptake of the vaccine, which includes AKAP4-specific antigens, compared to the control peptide vaccines without the R8 component. Notably, the experiments revealed that a concentration of 40 UM was found to be optimal for formulating the vaccine, resulting in 45% of the cells efficiently taking up the vaccine within just 30 minutes. Interestingly, the HPV-specific vaccine showed an even higher cellular uptake of 53% of the cells, possibly due to the CD8 component starting with an “R.” This led to the formation of an R9 bridge, potentially further increasing cellular uptake.

### Example 4. Dendritic Cell Uptake of the Vaccine Composed with Model Antigens

Study objectives: The objective of the study is to demonstrate improved uptake of vaccine composition in dendritic cells compared to control vaccine.

Vaccine design: Two well-characterized T-cell antigens were used as model antigens: 6

CD8 antigen (from pp65 CMV)
(SEQ ID NO: 1054)
NLVPMVATV
CD4 antigen (from Tetanus toxin)
(SEQ ID NO: 1055)
QYIKANSKFIGITE

Synthesis of the study vaccine: Fluorescent-labelled vaccine were synthesized and the control vaccine (no polyarginine) for cellular uptake studies:

### Labelled Model Vaccine Conjugate (CD4-R8-CD8):

(SEQ ID NO: 1108)
Fluoresc-KSS-QYIKANSKFIGITE-RRRRRRRR-LNVPMVATV

### Labelled Control Vaccine (CD4-CD8):

(SEQ ID NO: 1109)
Fluoresc-KSS-QYIKANSKFIGITE-AAA-LNVPMVATV

### Labelled Control Vaccine (CD4-R4):

(SEQ ID NO: 1110)
Fluoresc-KSSQYIKANSKFIGITERRRR-norbornene-NH

Methods: Monocyte-derived dendritic cells were generated by isolating monocytes from peripheral blood using density gradient centrifugation. The monocytes were then cultured in complete medium supplemented with IL-4 and GM-CSF for [indicate the duration] to promote their differentiation into dendritic cells. After [mention the duration], the differentiated dendritic cells were grown directly on cover slides. The cells were treated with peptide vaccines and fixed with 4% paraformaldehyde. Following fixation, the cells were washed with PBS and analyzed using fluorescent microscopy.

Immature DCs were treated with 2.5 or 10 microMfluorescent-peptide vaccines in duplicate. Two wells of untreated DCs were used as a negative control. 0.3 mL (2×105 cells) was removed and washed 3× with 5 mL PBS to remove surface-bound peptides and fluorophores. The washed cells were resuspended in 3 ml PBS (ca.2×105 cells). 1.5 ml (1×105 cells) was centrifuged in a table-top centrifuge and resuspended in 0.1 ml (ca.1×106 cells/mL) of Live Cell Imaging Solution (Invitrogen) before imaging.

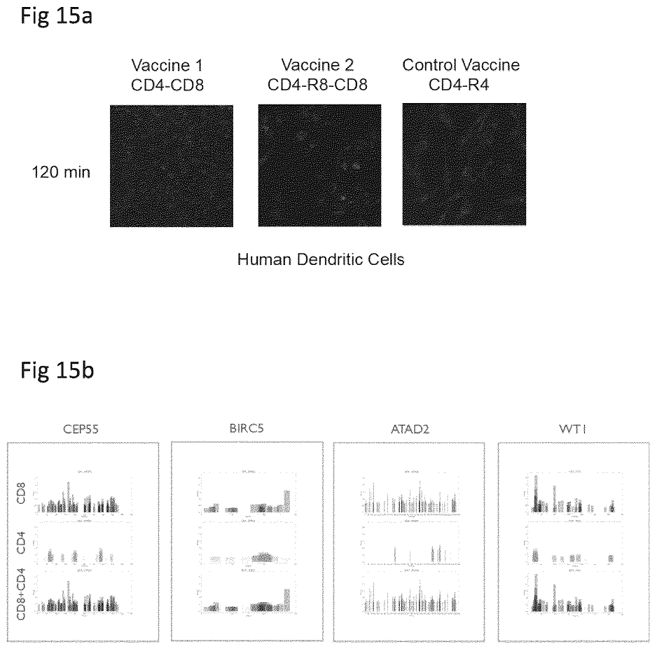

Results: FIG. 15a illustrates the cellular uptake of the vaccine and control vaccines in dendritic cells after 120 minutes of incubation. Cellular uptake was visualized using fluorescence microscopy. The results clearly depict a substantial increase in the uptake of the vaccine compared to the control vaccines. This enhanced dendritic cell delivery of vaccines similarly to HeLa cells and suggest the potential efficacy of the vaccine in immunization.

Conclusions: Effective uptake of the vaccine by human primary dendritic cells. Vaccine exhibited exceptional uptake not only by HeLa cells but also by human primary dendritic cells suggesting the following mechanism for induction of immune responses. After the injected vaccine are taken up to dendritic cells, the vaccine-derived epitopes saturate the patient's HLA molecules. These epitopes presented on the surface of dendritic cells at high density capable to induce both CD8 and CD4 T cell responses. The efficient uptake of the vaccine by dendritic cells ensures the generation of robust T cell responses.

The efficient uptake of the vaccine by dendritic cells not only highlights its effectiveness but also raises the exciting possibilities injecting natural peptide without adjuvant for future vaccine strategies. The ability to harness the inherent immunological capabilities of peptides and dendritic cells holds tremendous potential for revolutionizing the field of personalized vaccination. This discovery opens doors to exploring novel approaches that exploit the unique properties of vaccine to maximize the safety and efficacy.

### Example 5. Personalized VERDI Vaccine for Patient with Advanced Ovarian Leiomyosarcoma

The cancer patient suffers in advanced ovarian leiomyosarcoma (OLMS) with lung metastasis that is treated with surgical resection and checkpoint-inhibitor based immunotherapy (OPDIVO, nivolumab).

Sequencing of tumor and blood samples of the patient: Paraffin embedded tumor sample (FFPE) were available from the surgical resection of the tumor. Full transcriptome sequencing from the patient's FFPE samples were performed by two contractors: Lexogen (Vienna Biocenter, AT) and Ibioscience (Pécs, HU). Complete 4-digits HLA genotyping were performed from blood sample at the Universitätsklinik für Transfusionsmedizin und Zelltherapie (AKH, Vienna, AT).

TABLE
Results of the HLA sequencing (genotyping) of the patient with OMLS

| | | | | | |
|---|---|---|---|---|---|
| 6 Class | HLA_A_1 | HLA-A*26:01 | 12 Class | HLA_DPA1_1 | HLA-DPA1*01:03 |
| I Alleles | HLA_A_2 | HLA-A*66:01 | II Alleles | HLA_DPA1_2 | HLA-DPA1*01:03 |
| | HLA_B_1 | HLA-B*38:01 | | HLA_DPB1_1 | HLA-DPB1*04:01 |
| | HLA_B_2 | HLA-B*41:02 | | HLA_DPB1_2 | HLA-DPB1*04:01 |
| | HLA_C_1 | HLA-C*12:03 | | HLA_DQA1_1 | HLA-DQA1*03:01 |
| | HLA_C_2 | HLA-C*17:03 | | HLA_DQA1_2 | HLA-DQA1*05:05 |
| | | | | HLA_DQB1_1 | HLA-DQB1*03:01 |
| | | | | HLA_DQB1_2 | HLA-DQB1*03:02 |
| | | | | HLA_DRB1_1 | HLA-DRB1*04:02 |
| | | | | HLA_DRB1_2 | HLA-DRB1*13:03 |
| | | | | HLA_DRBX_1 | HLA-DRB3*01:01 |
| | | | | HLA_DRBX_2 | HLA-DRB4*01:03 |

Results of the vaccine target identification in the tumour by transcriptome analyses: The table below is a summary of the outcomes of the transcriptome analysis of the OMLS patient tumor biopsy, which pinpointed 12 distinct proteins. Predominantly comprising cancer testis antigens, alongside a handful of previously employed overexpressed proteins, these targets have historically demonstrated safety in cancer vaccine therapies for the treatment of cancer patients.

| | Ibioscience | Lexogen | Relative Expression |
|---|---|---|---|
| TARGETS | TPM | TPM | Target/1000 GAPDH |
| GAPDH (control) | 257 | 369 | 1000 |
| ATAD2 | 11 | 19 | 56 |
| BIRC5 | 6 | 8 | 24 |
| CEP55 | 6 | 8 | 26 |
| CNOT9 | 6 | 14 | 34 |
| EPCAM | 8 | 10 | 31 |
| KNL1 | 5 | 11 | 27 |
| ODF2 | 7 | 17 | 40 |
| PBK | 3 | 6 | 16 |
| SPAG1 | 3 | 7 | 23 |
| SPAG9 | 20 | 44 | 104 |
| TTK | 4 | 6 | 16 |
| WT1 | 3 | 6 | 13 |

Relative Expression of the vaccine target proteins is determined by analyzing the transcriptome of the patient's tumor sample. The expression level is quantified as TPM (Transcripts Per Million) of the TARGET relative to the housekeeping gene GAPDH (Glyceraldehyde-3-Phosphate Dehydrogenase) in the tumor sample. The formula used for calculation is TPM of the TARGET/TPM of GAPDH*1000. This relative expression value provides insights into the abundance of the TARGET in the tumor compared to the reference GAPDH.

Results of the predictive immune diagnosis test supporting VERDI Vaccine design: The predictive diagnosis of the epitope repertoire that participate in the tumour-specific immune responses of the OMLS patient is illustrated in FIG. 15b. The predictive diagnosis highlights a pivotal stage in our process. Utilizing the system and computer implemented method of the invention, which has been trained and validated using clinical data, highly ranked epitope selection emerges as a crucial step. This predictive diagnosis comprehensively evaluates all possible epitopes originating from the target proteins that is expressed in the patient's tumor. Its significance lies in its role as a cornerstone for the precise design of personalized peptide vaccines by VERDI. This technology forms the bedrock upon which our innovative approach to personalized vaccine rests.

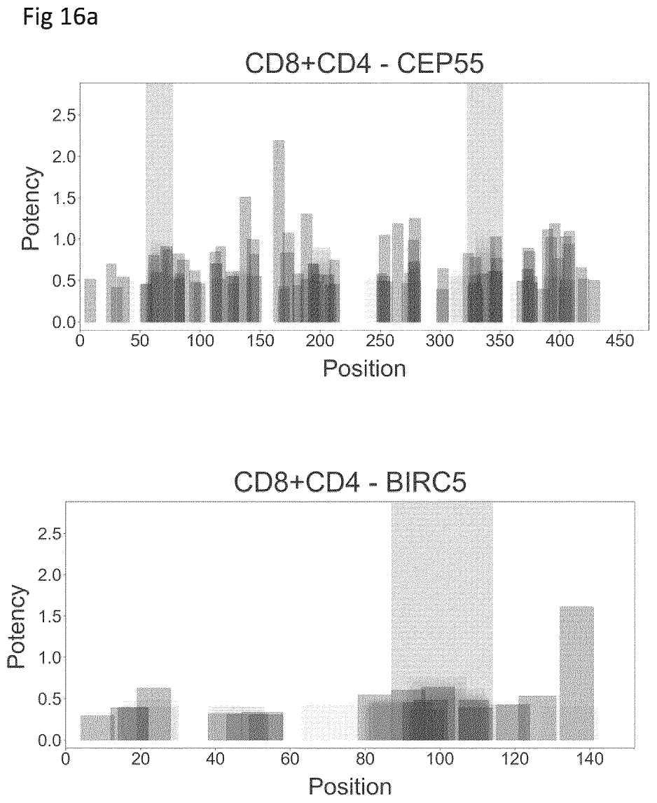

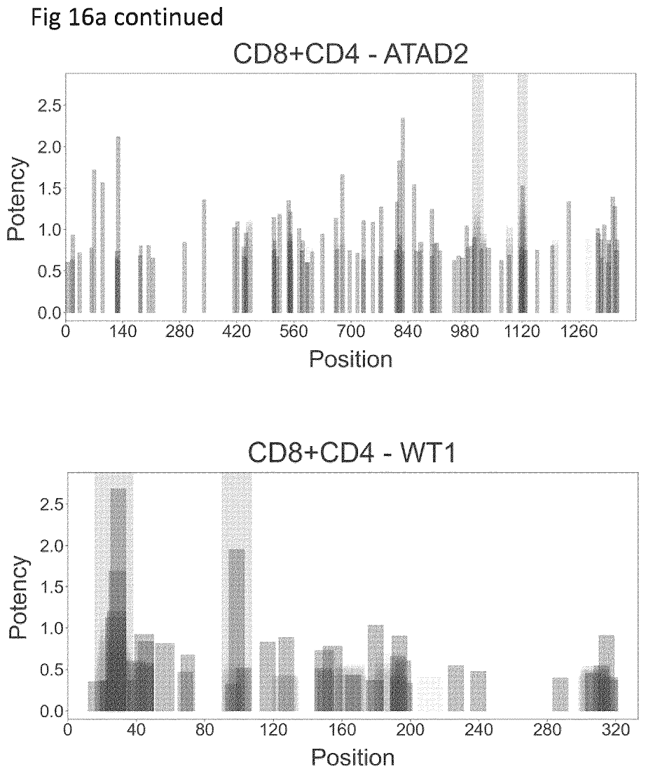

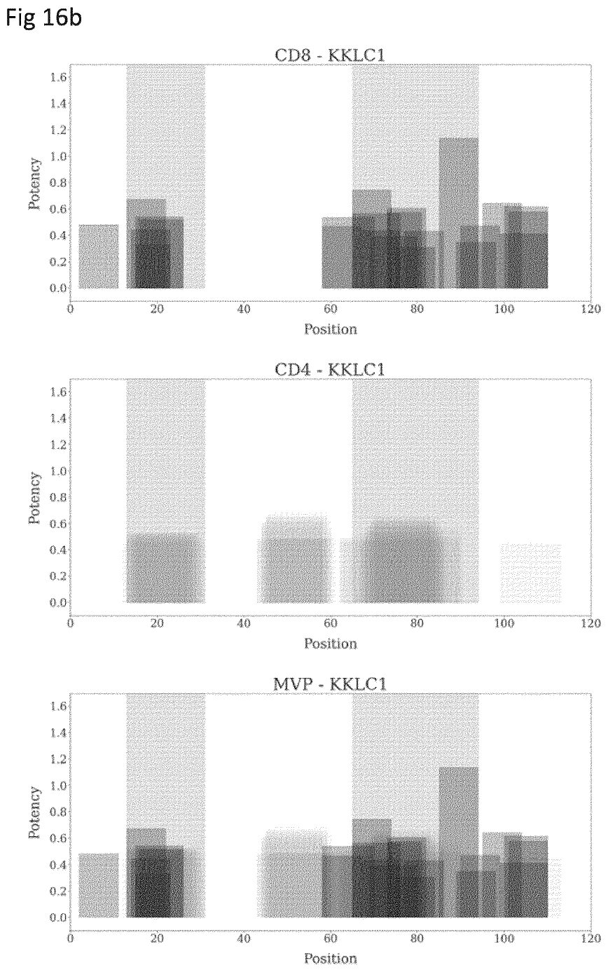

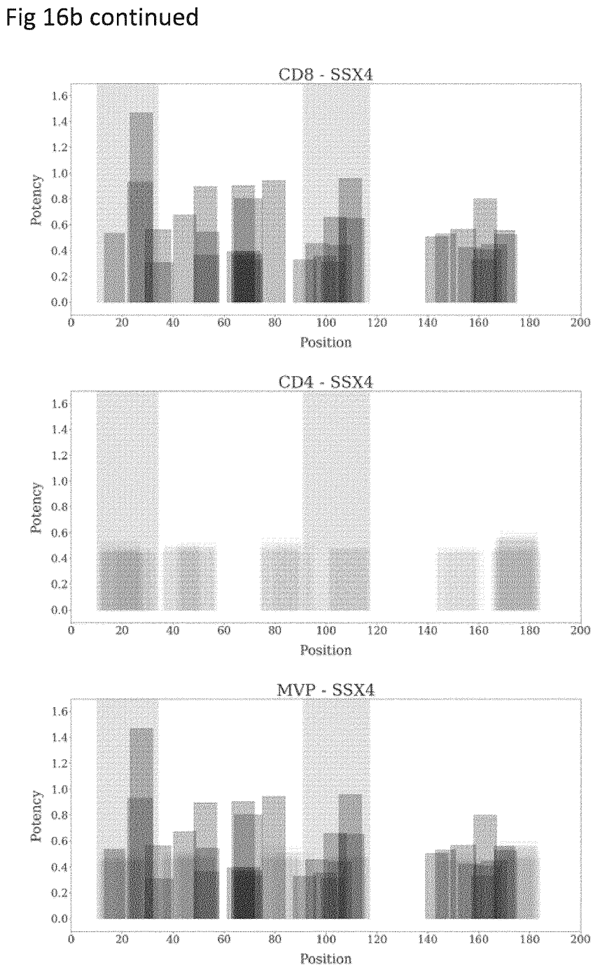

Results of the identification of candidate peptides for vaccine design: FIG. 16a shows the process of peptide antigen selection that consists of a set of highly ranked epitopes presented by the patient's HLAs on the cell surface, a pivotal phase in the creation of potent VERDI Vaccines. The antigen selection is based on identification of peptide that contains a set of overlapping highly ranked epitopes presented by class I and class II HLAs of the patent on the cell surface. Each meticulously crafted VERDI Vaccine is strategically engineered to trigger robust CD8 and CD4 T-cell responses, targeting specific proteins expressed within the patient's tumor cells.

Once identified, the selected peptides undergo rigorous safety testing, a pivotal safeguard against potential autoimmunity. Peptides that do not meet the stringent safety criteria, or contain sequences that could hinder automatic synthesis, are conscientiously eliminated from the roster of candidate vaccines. This curation ensures that only the most promising and structurally viable candidates progress to the next stage of development, exemplifying our unwavering commitment to patient safety and the pursuit of ground-breaking personalized vaccination.

TABLE
Results of the vaccine design by VERDI: Report to the physician

| | | Rel. | | | |
|---|---|---|---|---|---|
| UID | Target | Exp. | Peptide Sequence | CD4 | CD8 |
| AT_VERDI001_D1_1 | ATAD2 | 56 | H-RGCSS SKYAP SYYHV | 27 | 4 |
| | | | MPKQN STL-NH2 (SEQ | | |
| | | | ID NO: 1059) | | |
| AT_VERDI001_D1_2 | ATAD2 | | H-RLAID KRFRV FTKPV | 29 | 2 |
| | | | DPDEV PDYVT VI-NH2 | | |
| | | | (SEQ ID NO: 1060) | | |
| AT_VERDI001_D1_3 | BIRC5 | 24 | H-SVKKQ FEELT LGEFL | 42 | 7 |
| | | | KLDRE RAKNK IA-NH2 | | |
| | | | (SEQ ID NO: 1061) | | |
| AT_VERDI001_D1_4 | CEP55 | 26 | H-RQRLL EKIRV LEAEK | 24 | 4 |
| | | | EKNAY QL-NH2 (SEQ ID | | |
| | | | NO: 1062) | | |
| AT_VERDI001_D1_5 | CEP55 | | H-RSEEL LSQVQ FLYTS | 24 | 13 |
| | | | LLKQQ EEQTR VALL- | | |
| | | | NH2 (SEQ ID NO: 1063) | | |
| AT_VERDI001_D1_6 | EPCAM | 31 | H-PGQ TLIYY VDEKA | 38 | 5 |
| | | | PEFSM QGL-NH2 (SEQ ID | | |
| | | | NO: 1064) | | |
| AT_VERDI001_D1_7 | KNL1 | 27 | H-NDLA YANDF ASEYY | 46 | 2 |
| | | | LESEG QPLSA-NH2 (SEQ | | |
| | | | ID NO: 1065) | | |
| AT_VERDI001_D1_8 | KNL1 | | H-DKRYR KIVDV NFQSL | 16 | 3 |
| | | | LDEDQ APPSS LLV-NH2 | | |
| | | | (SEQ ID NO: 1066) | | |
| AT_VERDI001_D1_9 | ODF2 | 40 | H-KEKGD LELEI IVLND | 23 | 4 |
| | | | RVTDL VNQ-NH2 (SEQ | | |
| | | | ID NO: 1067) | | |
| AT_VERDI001_D1_10 | ODF2 | | H-RSSTP PLHVH VDENT | 17 | 2 |
| | | | PVHVH I-NH2 (SEQ ID | | |
| | | | NO: 1068) | | |
| AT_VERDI001_D1_11 | SPAG1 | 23 | H-SQGNE LFRSG QFAEA | 17 | 2 |
| | | | AGKYS A-NH2 (SEQ ID | | |
| | | | NO: 1069) | | |
| AT_VERDI001_D1_12 | JIP4 | 104 | H-ENLDS VFAQD QEHQV | 11 | 8 |
| | | | ELELL-NH2 (SEQ ID NO: | | |
| | | | 1070) | | |
| AT_VERDI001_D1_13 | TTK | 16 | H-VLNEK KQIYA IKYVN | 35 | 3 |
| | | | LEEAD NQTL-NH2 (SEQ | | |
| | | | ID NO: 1071) | | |
| AT_VERDI001_D1_14 | WT1 | 13 | H-AIRNQ GYSTV TFDGT | 41 | 5 |
| | | | PSYGH TP-NH2 | | |
| | | | (SEQ ID NO: 1072) | | |
| AT_VERDI001_D1_15 | PBK | 17 | H-AALGT RPPIN MEELD | 38 | 2 |
| | | | ESYQK VIEL-NH2 | | |
| | | | (SEQ ID NO: 1073) | | |

### Notes to the Table:

Most of the VERDI Vaccines targets Cancer Testis Antigens (CTAs). CTAs are proteins found in both testicular cells and certain cancer cells. These antigens are not typically expressed in normal adult tissues, except for the testis. However, their expression become activated in cancer cells, making them potential targets for cancer vaccines. CTAs can induce a robust immune response against cancer, helping the immune system recognize and attack tumor cells. Personalized vaccines designed by VERDI Solutions target CTAs which expression in the patient's tumor cells are confirmed by transcriptome analysis of paraffin-embedded tumor sample. Other targets are overexpressed antigens investigated previously in several clinical trials in cancer patients.

Relative Expression of the vaccine target proteins is determined by analyzing the transcriptome of the patient's tumor sample. The expression level is quantified as TPM (Transcripts Per Million) of the TARGET relative to the housekeeping gene GAPDH (Glyceraldehyde-3-Phosphate Dehydrogenase) in the tumor sample. The formula used for calculation is TPM of the TARGET/TPM of GAPDH*1000. This relative expression value provides insights into the abundance of the TARGET in the tumor compared to the reference GAPDH.

CD4 and CD8 are indicative to the potency of tumor-specific T-cell responses induced by the personalized peptide vaccine. Both CD4 helper T cells and CD8 cytotoxic T cells play vital roles in orchestrating a robust and effective immune response against cancer. The numbers reported represent the diversity of T-cell clones that may be activated by the peptides present in the vaccine. These peptides are fragments of the target protein, which is already expressed in the patient's tumor sample. By including these specific peptides in the vaccine, inventors aim to stimulate a broad spectrum of T-cell responses, engaging both CD4 and CD8 T cells to work in tandem.

Summary of the predicted safety and efficacy according the methods invented here: The 15 personalized vaccines designed for AT-VERDI001 patient include peptides that likely induce both CD8 and CD4 T-cell responses in the patient. Safety by design: (1) All the peptides represent a fraction of the target protein which is already expressed in the patient's tumor sample. (2) We exclude autoimmunity by testing whether any of the 8 amino

acid long fragments of the candidate peptides show homology with any other human proteins. (3) peptide vaccines (different sequences) have been shown excellent safety and tolerability profile in thousands of patients including cancer patients. In a meta-analysis including 500 patients, only 1.2% of the vaccinated patients suffer from vaccine-related serious adverse events. Efficacy by design: (1) Target selection from the patient's tumor sample using transcriptome analysis makes most likely that the target antigen express in the tumor despite RNA expression is not linearly associate with protein expression. (2) VERDI's unique predictive diagnostic test ensures that the vaccine peptides induce tumor-specific immune responses. (3) Each peptide vaccine has the potential to induce several CD8 and CD8 T-cell clones against the target protein which increase the chance that the potent immune response can attack the patient tumor cells.

The active pharmaceutical ingredient of personalized vaccine regimen designed by VERDI for patient AT-VERDI001 encompasses a collection of 15 peptides, each strategically designed to elicit both CD8 and CD4 T-cell responses within the patient's immune system.

Safety remains a paramount consideration throughout this process, manifested through a meticulous design approach:
1. All peptides constituting these vaccines are derived from segments of the target protein already present in the patient's tumor sample, ensuring a harmonious alignment between vaccine components and the patient's own biological makeup.
2. To further safeguard against potential autoimmunity, a rigorous screening protocol is executed. This entails assessing if any 8-amino acid fragments of the candidate peptides display homology with other human proteins, eliminating the risk of unintended immune responses.
3. A robust foundation of safety is fortified by extensive clinical validation.

Thousands of patients, including those undergoing cancer treatment, have received peptide vaccines with different sequences, collectively demonstrating an excellent safety and tolerability profile (43). A comprehensive meta-analysis, encompassing 500 patients, revealed a mere 1.2% occurrence of vaccine-related serious adverse events (44).

The efficacy of our approach is intricately woven into every facet of vaccine design:
1. Target selection is precisely curated from the patient's own tumour sample through transcriptome analysis. This approach significantly enhances the likelihood of the chosen target antigen being expressed within the tumour environment, bridging the gap between RNA and protein expression dynamics.
2. VERDI's unique predictive diagnostic test stands as a pivotal hallmark, confirming the capability of vaccine peptides to induce tumour-specific immune responses.
3. Each individual peptide vaccine possesses the innate capacity to stimulate multiple CD8 and CD4 T-cell clones, an aspect that augments the likelihood of a potent immune response capable of launching an effective attack against the patient's tumour cells.

In essence, our personalized vaccine regimen represents a combination of safety and efficacy, underpinned by a multifaceted design strategy that leverages advanced scientific insights to enhance the prospects of successful immune intervention.

### Example 6. Personalized Peptide Vaccine Design for the Murcia Patient with Metastatic Signet Ring Cell Adenocarcinoma

Signet ring cell cancer, a very rare and aggressive variant of adenocarcinoma, presents unique diagnostic and therapeutic challenges. It typically originates in the gastrointestinal tract and is characterized by the presence of cells with abundant intracytoplasmic mucin that pushes the nucleus to the periphery, giving the cells a “signet ring” appearance. Signet ring cell carcinomas are reported to be more aggressive than other histological subtypes of colorectal carcinoma and are usually detected at a more advanced stage due to its endophytic/infiltrative growth pattern. Here we illustrate the diagnostic challenges and therapeutic considerations associated with this rare and aggressive malignancy. Since no option is currently available to expand tumor specific T cell responses in patients with signet ring carcinoma, this case report focuses on the utilization of personalized VERDI vaccines as adjuvant treatment of this malignancy.

Clinical history: The patient is a 70-year-old male, who had a history of well-controlled type 2 diabetes on metformin treatment, a previous smoking habit, prostatic syndrome under alpha-blocker treatment, cholelithiasis, and a left renal ureteral colic diagnosis. In February 2023, he was admitted for the evaluation of bone lytic lesions, which were causing pain in the sacrococcygeal and left lumbar regions. Clinical examination at admission was unremarkable.

Oncological history: The patient was diagnosed with stage IV Signet Ring Cell Adenocarcinoma (SRCAC) of unknown origin with multiple bone metastases. He was admitted to the hospital on Feb. 22, 2023, for the investigation of bone lytic lesions. The patient had presented two months earlier with pain in the sacro-coccygeal region, radiating to the left lumbar region, which worsened on standing and improved with rest. Laboratory tests revealed elevated glucose levels (118), alkaline phosphatase (711), CEA (2.8), and Ca 19.9. (2,199) The PSA level was within reference range. PET-CT scan demonstrated hypermetabolic lesions in bone structures and the prostate parenchyma, along with adrenal nodules. Biopsy of several bone lesion confirmed stage IV signet ring cell carcinoma with multiple metastases. Gastroscopy and colonoscopy indicated chronic gastritis, duodenitis, and colonic diverticulosis, but no neoplasia. Biopsies indicated with moderate atrophic chronic gastritis, severe activity of inflammation caused by Helicobacter pylori and extensive intestinal and gastric metaplasia. Echo-endoscopy showed no significant abnormalities except for gallbladder lithiasis. Molecular studies showed no loss of MLH1 or MSH2 expression, HER2 negative, CPS PDL-1 and TPS negative (0). DPyD normal metabolizer.

In March 2023 the patient underwent a diagnostic laparoscopy and peritoneal implant biopsies, confirming peritoneal adenocarcinoma with signet ring cells. Based on the molecular analysis performed using OncoDeep test, there were no specific genetic or molecular changes detected in the tissue sample that would make the cancer suitable for targeted treatments. The absence of any alterations limited the availability of targeted therapy options for this patient.

Treatment: In March 2023 the patient commenced first-line treatment with FOLFOX6m. In May 2023, after four cycles of treatment PET CT detected omental and mesenteric carcinomatosis with slight metabolic increase without clear hypermetabolic lesions. CA19.9 tumor marker increased in two months from 2,199 to 4,390 suggesting disease progression.

In May 29, 2023, the patient initiated second-line treatment consisting of Paclitaxel and Ramucirumab in response to peritoneal progression. With no available industrial treatment options for curing the patient, the medical team reached out to VERDI Solution to design personalized vaccines. Upon the patient's provision of written informed consent, VERDI Solutions obtained the patient's HLA genotype and clinical data to design personalized vaccines. Subsequently, the Murcia patient underwent a regimen of 10 personalized VERDI vaccines, concurrently administered with Paclitaxel and Ramucirumab.

Scheduling the adjuvant VERDI vaccinations presented a treatment challenge. A 20 mg dose of Dexamethasone was administered as premedication before Paclitaxel and Ramucirumab to mitigate potential life-threatening infusion-related reactions. Dexamethasone, a corticosteroid, has the potential to suppress the immune system by reducing inflammation, raising concerns about its impact on vaccine efficacy. After meticulous evaluation, the medical team decided to commence Paclitaxel and Ramucirumab treatment on Jul. 3, 2023. The first four vaccines were administered on Jul. 13, 2023, with the remaining six given on Jul. 27, 2023. The chemotherapy was postponed until Aug. 1, 2023, to optimize treatment sequencing.

Personalised VERDI vaccines: VERDI's solution for cancer patients involves designing at least 10 personalized vaccines, matching their individual immunogenetic profile and tumour characteristics. To enable this, the inventors has developed and validated the predictive diagnostic test that predicts highly ranked epitopes from proteins that participate in the T cell responses of the individual. Using the result of this test VERDI can assists physicians in designing personalized peptide vaccines that most likely effective to destroy the patient's tumor cells.

The VERDI test forecasts the patient's epitope repertoire responsible for eliciting tumor-specific immune responses, leveraging two input data obtained from sequencing blood and tumor specimen: 4-digit HLA genotype and tumor specific proteins. The HLA genotype was readily available for the Murcia patient: HLA-A*24:02, HLA-A*31:01, HLA-B*08:01, HLA-B*51:01, HLA-C*05:01, HLA-C*07:01, HLA-DPA1*01:03, HLA-DPA1*01:03, HLA-DPB1*03:01, HLA-DPB1*04:01, HLA-DQA1*01:03, HLA-DQA1*05:01, HLA-DQB1*02:01, HLA-DQB1*06:03, HLA-DRB1*03:01, HLA-DRB1*13:01, HLA-DRB3*01:01, HLA-DRB3*01:01. However, due to the unavailability of tumor samples from the patient, the selection of tumor-specific proteins was guided by data derived from peer-reviewed literature. Given that the patient had an active Helicobacter pylori infection, and considering the likely gastric origin of the tumor, our focus shifted to identifying Cancer Testis Antigens (CTAs) associated with gastric cancer induced by H. pylori. Inventors identified KK-LC-1 as an exceptionally promising target, as it demonstrates expression in approximately 80% of gastric cancers linked to H. pylori infection (45). Additionally, our selection included GTGIB and SSX4, CTAs expressed in 24% and 16% of H. pylori-positive gastric cancers, respectively, based on findings from the same publication. Furthermore, the inclusion of five additional CTAs was guided by a comprehensive transcriptome analysis of 375 gastric cancer specimens (46). This selection process allowed us to assemble a panel of tumor-specific proteins with potential to express in the tumor of the patient.

The second step involves entering the HLA and CTA data into the VERDI Test to predict the epitopes that are most likely to induce CD8 and CD4 T cell responses against the Murcia patient's Signet Ring Cell Adenocarcinoma (SRCAC). The VERDI Test provides a selection of the predicted immunogenic epitopes, including their sequences and the predicted potency of the T cell responses they may trigger, typically ranging from 0.4 to 2. This information can be visually represented using a comb diagram, which illustrates the predicted antigen-specific T cell responses in the Murcia patient (FIG. 16b). Notably, it reveals fluctuations between immunologically active and inactive regions within the proteins and demonstrates the independent induction of CD4 and CD8 responses by immunologically active regions.

The third step encompasses the design of the personalized VERDI vaccines, guided by the outcomes of the VERDI Test. Our approach prioritizes both efficacy and safety through deliberate design. To ensure vaccine efficacy, the peptides included in the VERDI vaccine are selected to induce robust CD8 and CD4 T cell responses against antigens expressed in the Murcia patient's tumor. Safety considerations are addressed by excluding any potential for autoimmunity. Given the impossibility to control the safety and efficacy of personalized VERDI vaccines in other patients, our vaccine design comprises vaccines that are most likely to induce potent T cell responses in the Murcia patient, with some of them being probable tumor targets while others are less likely to target the patient's tumor. As positive controls, inventors selected two peptides from KKLC1, as it was highly likely that this antigen was expressed in the Murcia patient's tumor. Conversely, for negative controls, inventors designed two similarly immunogenic peptides from SSX4, as its expression in H. pylori positive SRCAC was improbable. To address tumor heterogeneity, inventors included immunogenic peptides from an additional six CTAs that are likely to be expressed in gastric cancers. The selection criteria for vaccine peptides aimed to maximize the number of epitopes recognized by the same TCR. Then inventors excluded vaccines that could cause autoimmunity by subjecting all candidate vaccines to an immunogenicity investigation in a digital twin. Consequently, inventors designed 10 peptide vaccines for the Murcia patient, each containing a single peptide ranging from 18 to 29 amino acids in length (FIG. 16b).

The table below is a list of Target Antigens, Peptide Sequences, and Immunological Characterization of the Personalized VERDI Vaccines Administered to the Murcia Patient as Adjuvant Immunization against his H. pylori Positive Signet Ring Cell Adenocarcinoma.

TABLE x
Peptide antigens of 1st generation personalized vaccines designed for the
Murcia patient.

| | | | # of | | | |
|---|---|---|---|---|---|---|
| | | | Epitopes | | Potency AVG [MIN; MAX] | |
| | Target | VERDI Vaccines | CD4 | CD8 | CD4 | CD8 |
| C1 | KKLC1 | ILNNFPHSIARQKRILVNLSMVENKLVEL | 22 | 9 | 1.0 | 0.6 |
| | | (SEQ ID NO: 1074) | | | [0.9; 1.3] | [0.3; 1.1] |
| C2 | KKLC1 | ALIVFWKYRRFQRNTGEM | 11 | 5 | 1.0 | 0.5 |
| | | (SEQ ID NO: 1075) | | | [0.9; 1.1] | [0.3; 0.7] |
| C3 | CTG1B | TVSGNILTIRLTAADHRQLQLS | 25 | 7 | 1.1 | 0.5 |
| | | (SEQ ID NO: 1076) | | | [0.8; 1.5] | [0.3; 1.5] |
| C4 | SSX4 | PRDDAQISEKLRKAFDDIAKYFSK | 13 | 3 | 0.9 | 1.0 |
| | | (SEQ ID NO: 1077) | | | [0.9; 1.1] | [0.5; 1.5] |
| C5 | SSX4 | NQVERPQMTFGSLQRIFPKIMPKKPA | 4 | 7 | 1.0 | 0.6 |
| | | (SEQ ID NO: 1078) | | | [0.9; 1.0] | [0.3; 1.0] |
| C6 | VCX2 | SDPKKKTTKVAKKGKAVRRGRR | 11 | 7 | 1. | 0.6 |
| | | (SEQ ID NO: 1079) | | | [1.3; 1.4] | [0.3; 1.5] |
| C8 | MAGA3 | TFPDLESEFQAALSRKVAELVHFL | 3 | 5 | 1.7 | 0.7 |
| | | (SEQ ID NO: 1080) | | | [1.5; 1.9] | [0.6; 0.9] |
| C9 | MAGA6 | KASDSLQLVFGIELMEVDPIGHVYIF | 4 | 9 | 1.6 | 0.6 |
| | | (SEQ ID NO: 1081) | | | [1.5; 1.6] | [0.3; 1.0] |
| C10 | MAGA4 | KVLEHVVRVNARVRIAYPSLREAAL | 37 | 6 | 1.9 | 0.8 |
| | | (SEQ ID NO: 1082) | | | [1.5; 2.7] | [0.5; 1.4] |
| C11 | MAGAC | WGPRALVETSYVKVLHHLL | 12 | 5 | 1.2 | 1.0 |
| | | (SEQ ID NO: 1083) | | | [1.1; 1.4] | [0.7; 1.5] |

Inventors have decided to utilize the peptide vaccine platform because its exceptional safety and tolerability feature that has been demonstrated in thousands of patients. In addition, personalized VERDI vaccines that comprise a peptide solution emulsified in Montanide adjuvant (Seppic, France) can be prepared by pharmacists or physicians making this treatment accessible to patients.

Safety findings: The treatment including the vaccinations were excellently tolerated, with no observed side effects. However, on Sep. 1, 2023, a thrombotic event presented a diagnostic challenge due to its atypical region and absence of prior venous catheter placement. The event was characterized by pronounced dilation of the left jugular vein, with a notable absence of contrast filling, extending from its cranial exit point at the jugular foramen to its bifurcation point at the left brachiocephalic venous trunk. Accompanying this were peripheral inflammatory changes consistent throughout the cervical tract, multiple reactive-appearing millimetric lymph nodes, and notable bulging against the ipsilateral sternocleidomastoid muscle, causing a slight indentation of the ipsilateral piriform sinus.

Upon review of the patient's medical history and the literature, it was hypothesized that the thrombosis was most likely associated with the underlying cancer (SRCAC) or potentially the angiogenesis inhibitor treatment, rather than the peptide vaccination. This hypothesis was supported by the patient's elevated platelet count, which exceeded 350,000, fitting the criteria for prophylactic anticoagulation according to Khorana's score. SRCAC has been reported to present initially with Internal Jugular Vein (IV) thrombosis, potentially due to a hypercoagulable state related to the malignancy. Additionally, the angiogenesis inhibitor, Ramucirumab, has been associated with a slightly higher incidence of Grade 3 or higher arterial thromboembolic events. However, these events are primarily arterial in nature, and the patient's thrombosis was venous. Regarding the vaccination, thrombosis side effects are very rare and are primarily associated with vaccinations using adenovirus vectors and thrombocytopenia. As such, it seems unlikely that the peptide vaccination contributed to the thrombosis.

Immunological outcomes: An integral part of this case is the immunological response observed after the administration of personalized VERDI vaccines to the Murcia patient. T cell responses were assessed using the QuantiFERON® ELISA (QFN) assay, designed to detect human interferon-gamma (IFN-γ) in plasma following an overnight peptide stimulation of whole blood cells (Qiagen, USA). The QFN test offers a high degree of sensitivity, with a detection limit as low as 0.065 IU/ml. The quantity of IFN-γ serves as a measure of the VERDI vaccine-specific T-cells, encompassing both CD4 and CD8 T cell responses (49, 50).

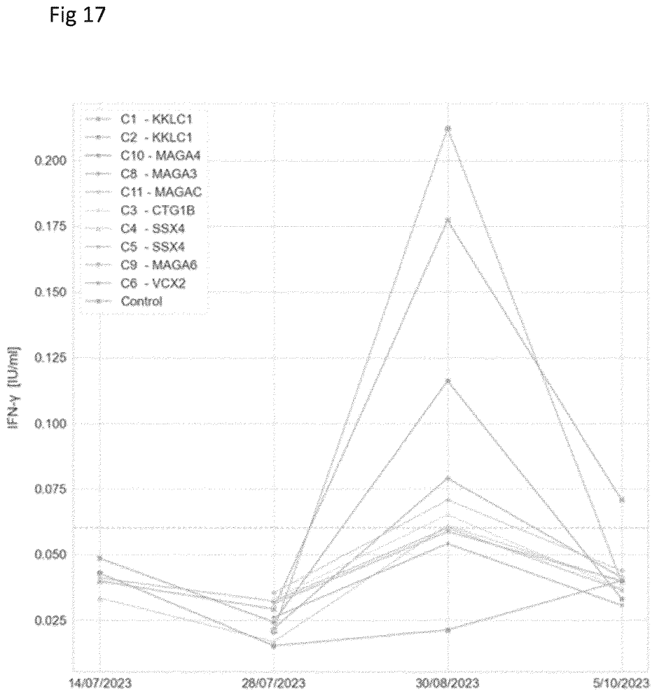

T cell responses were identified against four specific peptides out of the ten VERDI vaccines administered (FIG. 17). Notably, peptides C1 and C2, originating from the KK-LC-1 antigen and serving as positive controls, consistently induced T-cell responses following a single vaccination. In contrast, the two negative control VERDI vaccines targeting SSX4 consistently yielded responses around the QFN test's detection limit. These results strongly suggested the presence of KK-LC-1-expressing tumor cells in the Murcia patient, capable of in vivo stimulation of KKLC1-specific T cells, resulting in a boost effect. Conversely, the absence of detectable responses against the two SSX4 peptides provided robust evidence that this target was not expressed in the patient's tumor. Additionally, positive T cell responses were observed against MAGEA4 and MAGA3, indicating the potential of these vaccines to enhance immune responses against the tumor cells. The inclusion of internal positive and negative controls within our study not only shed light on potential tumor targets but also supported our hypothesis regarding the safety, immunogenicity, and effectiveness of the different VERDI vaccines as adjuvant treatment for SRCAC.

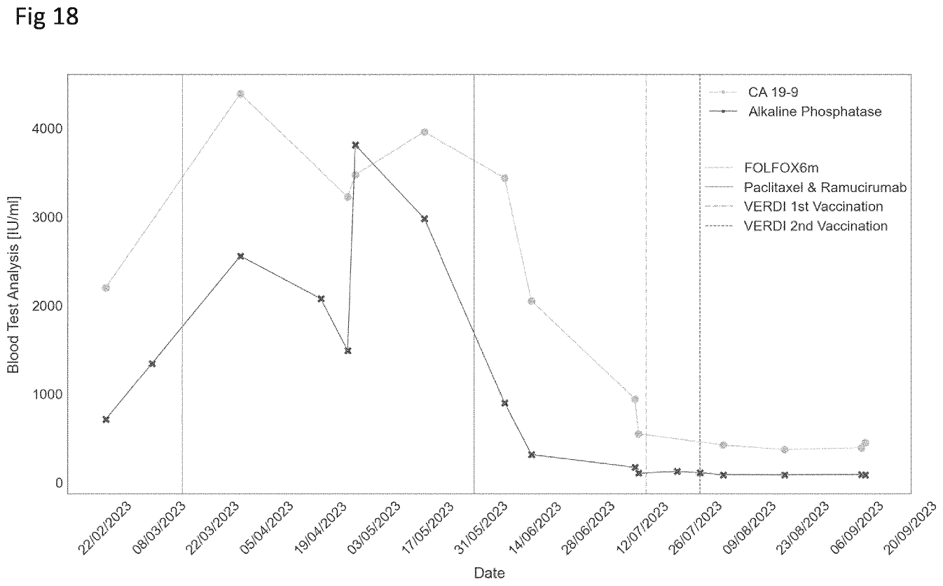

Clinical Progress: The patient's clinical condition remained promising throughout the treatment course. The noteworthy decrease in Carbohydrate antigen 19-9 (CA 19-9) levels during his 2^{nd} line therapy from 4,390 ng/mL on May 29, 2023, to 372 ng/mL on Sep. 7, 2023, signifies a positive response to the implemented cancer treatment regimen (FIG. 18). This substantial reduction in CA 19-9, a recognized biomarker in pancreatic and gastrointestinal cancers, strongly suggests an effective control of the disease and a reduction in tumor activity. The data underscores the significance of regular biomarker monitoring in tracking disease progression and gauging the therapeutic response.

We received results from PET-CT scan for an assessment of the tumor's response. The examination conducted on Sep. 19, 2023, in comparison with the previous scan from Jul. 17, 2023. The findings revealed known peritoneal carcinomatosis with involvement of the omentum and mesentery. The metabolic evaluation, while partially limited due to heterogeneous intestinal hypermetabolism, demonstrated stability when compared to the previous study. Noteworthy aspects included the absence of pathological focal deposits in the liver, unchanged bilateral adrenal nodular thickening, and an enlarged prostate without clear hypermetabolic foci. No evidence of abdominal or pelvic adenopathy was detected, and there were no suspicious lung nodules. In terms of bone involvement, there was a mild metabolic increase in some lesions compared to the previous study. No macroscopic malignant disease was evident in other locations throughout the body.

A remarkable observation unfolded in the divergence between the trends observed in the PET-CT and the biomarkers. While the former displayed a tendency to remain stable with some focal variations, the latter exhibited a dramatic improvement, indicating an amelioration and significant decrease in tumor-induced bone damage (illustrated by a remarkable 35-fold reduction in Alkaline Phosphatase) and a decline in gastrointestinal tumor activity (with CA 19-9 decreasing almost 10-fold) (FIG. 18). The decline in Alkaline Phosphatase commenced after the second line of chemotherapy and continued post-vaccination. Notably, the turning point for the gastrointestinal antigen CA 19-9 was particularly noteworthy after vaccination. Considering these shifts, we contemplated the possibility of pseudoprogression, a phenomenon that mimics disease progression but is, in fact, a hypermetabolic “flare-phenomenon” induced by T-cell tumor infiltration. It's worth noting that patients experiencing pseudoprogression often exhibit an objective response in the subsequent stages of therapy.

Next, our objective was to validate the pseudoprogression through pathological analysis. In the event of identifying tumor tissue, our strategy was to design additional VERDI Vaccines, utilizing targets selected from the tumor transcriptome analysis. To obtain a sample, we opted for a CT-guided puncture of pathological areas in the peritoneum, deeming it more efficient than obtaining biopsies from bony areas, where metastases were present. A noteworthy challenge arose in the form of complicating interventional procedures due to the administration of Ramucirumab, which hindered proper wound healing. Two Right Peritoneal Biopsies (RPB) were obtained on Oct. 4, 2023. Pathological examination of the biopsy showed 80% signet ring cells, significant mucoid secretion, and the absence of lymphocytes in hematoxylin staining. Vaccine-induced T-cell responses were also absent in the peripheral blood obtained the same day, suggesting the absence of specific target cells in the tumor that could be recognized by T-cells (FIG. 17).

Transcriptome analysis of the tumor biopsies (next Table) confirmed the absence of tumor cells expressing any of the targeted CTAs by the personalized vaccines despite there are a 96% probability that at least one of the target CTAs are expressed in gastric cancer cells. These results strongly suggest that vaccine-induced T-cells killed the specific target cells in the Murcia patient's tumor. These results provide evidence for the efficacy of the personalized VERDI Vaccine. Single immunization with personalized VERDI Vaccine can completely destroy the targeted tumor cells in a metastatic cancer patient.

TABLE
Evidence for the killing of the targeted tumour cells by personalized
VERDI Vaccine treatment and identification of new targets for 2^{nd}
generation personalized vaccine design
Results of the tanscriptome analyses of the Murcia patient's peritoneal
tumour biopsies (Oct. 4, 2023)

| | Relative | |
|---|---|---|
| Targets | expression * | Annotation |
| KK-LC-1 | 0 | CTA, overexpressed in gastric cancers including H. |
| | | Pylori+ signet ring cell carcinoma, associated with poor |
| | | prognosis |
| MAGEA1 | 0 | CTA, a peptide vaccine was well-tolerated and induced |
| | | MAGE-A1-specific CD8+ T cell responses in patients |
| | | with advanced melanoma |
| MAGEA3 | 0 | CTA, used in a recombinant vaccine tested in non-small |
| | | cell lung cancer and melanoma, but not yet in gastric |
| | | cancer |
| MAGEA4 | 0 | CTA, a peptide vaccine was safe and immunogenic in |
| | | patients with various solid tumors, including esophageal, |
| | | lung, and head and neck cancers |
| MAGEA6 | 0 | CTA, itself has not been the primary target of a vaccine, |
| | | it is often co-expressed with other MAGE-A antigens |
| MAGAC | 0 | CTA, member of MAGE family |
| NY-ESO-1 | 0 | CTA, one of the most immunogenic one, successfully |
| | | targeted in clinical trials for gastric cancer |
| VCX2 | 0 | CTA, overexpressed in gastric cancers, expression of |
| | | VCX2 inversely correlate with promoter methylation and |
| | | its expression could be activated by treatment with a |
| | | DNA methyltransferase inhibitor and histone deacetylase |
| | | inhibitor. |
| SSX4 | 0 | CTA, member of the SSX family are widely co- |
| | | expressed in various tumors including gastric cancers, |
| | | associated with poor prognosis |
| BLTP2 | 95 | It is overexpressed in human gastric cancer and silencing |
| | | reduces the proliferation, migration, and invasion of |
| | | gastric cancer cells |
| SPAG9 | 70 | CTA, induces humoral immune response, promotes cell |
| | | proliferation. A DNA vaccine targeting SPAG9 has been |
| | | tested in patient with cervical cancer |
| WT1 | 59 | Highly prioritized cancer vaccine target according to |
| | | NCI. Clinical trials of with WT1-targeted peptides and T |
| | | cells have confirmed its safety, immunogenicity, and |
| | | clinical efficacy. |
| KDM5B | 28 | Overexpressed in several cancers and suppresses anti- |
| | | tumour immunity by epigenetic silencing of |
| | | retroelements |
| GPATCH2 | 19 | Suppressing its expression results in growth suppression |
| | | of cancer cells |
| ODF2 | 19 | CTA, epitopes derived from ODF2 induced peptide |
| | | specific CD8+ T cells in gastric cancer models |
| ATAD2 | 9 | CTA, highly expressed in gastric cancers, including |
| | | those caused by H. pylori |

The transcriptome analysis of the Murcia patient was used to identify proteins expressed in the patient's tumor cells and not expressed in healthy cells (cancer testic antigens). All the putative 8-20 amino acids long epitopes were created from these proteins and used for designing the 2nd generation of personalized vaccines according to the invention. The next Table illustrate the characterization of the 2nd generation personalized vaccines. These antigens have different length and amino acid sequences. All selected peptide antigen contains multiple highly ranked epitopes (13-66) presented on the cell surface by the patient's HLA class I and class II molecules. The range of the potency score is indicated for the selected epitopes. Epitopes presented by the patient's HLA class I and class II molecules are indicated to induce CD8 and CD4 responses, respectively. As described in the invention, the potency of each epitope in the vaccines was calculated and indicated to inform the physician about the predicted efficacy of the personalised vaccines in the recipient.

TABLE
Peptide antigens of 2nd generation personalized vaccines designed for the
Murcia patient.

| | | | # of | | | |
|---|---|---|---|---|---|---|
| | Relative | | Epitopes | | Potency AVG [MIN; MAX] | |
| Targets | expression | VERDI Vaccines | CD4 | CD8 | CD4 | CD8 |
| BLTP2 | 95 | TGIPVVKPRQLIATDDAVPL | 46 | 4 | 3.58 | 0.86 |
| | | GPGKGV SEQ ID NO: 1119 | | | [2; 4.81] | [0.55; 1.61] |
| BLTP2 | 95 | AFVAEDKFITLAAESVSLS | 17 | 2 | 2.42 | 1.3 |
| | | SEQ ID NO: 1120 | | | [1.89; 3.11] | [0.61; 1.98] |
| BLTP2 | 95 | QCRDLLRATVFPETVPSL | 11 | 2 | 2.33 | 1.35 |
| | | SEQ ID NO: 1121 | | | [1.9; 2.83] | [1.18; 1.52] |
| SPAG9 | 70 | RNQYKERLMELQEAVRWT | 11 | 7 | 1.8 | 0.69 |
| | | EMIRASREN SEQ ID NO: | | | [1.53; 2.11] | [0.42; 1.77] |
| | | 1122 | | | | |
| SPAG9 | 70 | VGDGVWVSIRLDSTLRLY | 57 | 9 | 2.49 | 0.52 |
| | | HAHTYQHL SEQ ID NO: | | | [1.53; 3.67] | [0.32; 0.74] |
| | | 1123 | | | | |
| SPAG9 | 70 | LENLDSVFAQDQEHQVELE | 30 | 2 | 2.27 | 0.96 |
| | | LL SEQ ID NO: 1124 | | | [1.62; 3.02] | [0.63; 1.29] |
| WT1 | 59 | LLLRTPYSSDNLYQMTSQL | 16 | 4 | 1.4 | 0.55 |
| | | SEQ ID NO: 1125 | | | [1; 1.94] | [0.33; 0.72] |
| WT1 | 59 | AQYRIHTHGVFRGIQDVRR | 48 | 14 | 1.24 | 0.52 |
| | | VPGVAPTLVR SEQ ID NO: | | | [1; 1.84] | [0.3; 0.94] |
| | | 1126 | | | | |
| KBM5B | 28 | DVDKLHFTPRIQRLNELEA | 7 | 7 | 1.73 | 0.7 |
| | | QTRVKL SEQ ID NO: 1127 | | | [1.64; 1.84] | [0.37; 1.38] |
| KBM5B | 28 | KLRYRYTLDDLYPMMNAL | 7 | 6 | 1.7 | 0.73 |
| | | KL SEQ ID NO: 1128 | | | [1.61; 1.8] | [0.38; 1.48] |
| ODF2 | 19 | VMKTRLEADEVAAQLER | 13 | 4 | 2.44 | 0.71 |
| | | SEQ ID NO: 1129 | | | [1.51; 3.5] | [0.39; 1.38] |
| ODF2 | 19 | RSSTPPLHVHVDENTPVHV | 36 | 6 | 2.37 | 0.53 |
| | | HIK SEQ ID NO: 1130 | | | [1.55; 3.27] | [0.31; 0.92] |
| ATAD2 | 9 | SLADVDPMQLDSSVRFDS | 43 | 6 | 2.75 | 0.69 |
| | | VGGLSNHI SEQ ID NO: | | | [1.98; 4.21] | [0.4; 1.25] |
| | | 1131 | | | | |
| ATAD2 | 9 | SERQLRLLFDQAYQMRPSII | 18 | 7 | 2.54 | 0.65 |
| | | FF SEQ ID NO: 1132 | | | [2.02; 3.07] | [0.41; 0.94] |
| GPATCH2 | | PLWHESDFAVDNVGNRTL | 36 | 3 | 2.32 | 0.45 |
| | | RRRR SEQ ID NO: 1133 | | | [1.57; 3.1] | [0.36; 0.6] |

Conclusion: This study unveils a milestone in the treatment of metastatic SRCAC, an exceedingly rare and aggressive colon cancer subtype with a five-year survival rate of 36% (51). The intricacies of the Murcia patient's case have not only shed light on the multifaceted nature of this condition but have also introduced a ground-breaking approach to personalized cancer care, courtesy of the VERDI System. The VERDI Test, a new diagnostic tool, stands as the keystone of this innovation. It not only predicts peptide fragments capable of eliciting robust CD4 and CD8 T cell responses but also empowers clinicians to craft personalized peptide vaccines (VERDI vaccines) tailored to destroy the unique tumor cells of each patient. In the context of this aggressive adenocarcinoma, integrating personalized VERDI vaccines into the patient's treatment regimen represents a significant advancement.

Summary: In conclusion, the findings from these studies highlight the potential of the vaccine as an innovative approach to personalized vaccination. The ability to elicit both CD8 and CD4 T-cell responses, along with its individualized nature and efficient dendritic cell uptake, sets the vaccine apart as a promising strategy in the field of personalized cancer immunotherapy. By harnessing the power of natural peptides and dendritic cells, the vaccine offers new possibilities for enhancing patient outcomes and revolutionizing the landscape of cancer treatment.

### Example 7. Exemplary Method of Preparing a VERDI Personalised Vaccine

VERDI Vaccines may be prepared from “Raw Peptide Materials” utilizing inverse electron demand Diels-Alder (IEDA) conjugation. The lyophilized Raw Peptide Materials, equipped with a tetrazine or a norbornene moiety, are dissolved in Phosphate-buffered saline (PBS) in 1 mg mL concentration. The reaction mixture is heated to 40° C. for 1 h to obtain the conjugated VERDI Vaccine product (API). The VERDI Vaccine is sterilized by filtration and filled into a syringe for subcutaneous injection. The reaction is followed by the decolorization of the purple colour of the tetrazine moiety. This rapid chemical reaction ensures the conjugation of two peptide antigens in a fast and efficient biorthogonal way.

TABLE
Preparation of VERDI vaccine

| Rx VERDI | |
|---|---|
| VACCINES | Preparation Process |
| Raw Peptide Material | 1 mg/vial lyophilized Raw Peptide Materials with CoA10 |
| shipping and storage | shipped at room temperature |
| | Raw Peptide Material should be stored in a box in a |
| | freezer at −20° C. |
| VERDI Vaccine | From the Raw Peptide Material box stored at −20° C. and |
| preparation | labelled |
| | with the name of the patient select the two vials of |
| | lyophilized peptides |
| | that meet the medical prescription of the VERDI Vaccine # |
| | Incubate the two vials at room temperature for 30 minutes |
| | Add to each vial 10 microliter DMSO - vortex 10 second |
| | Add to each vial 0.5 mL PBS - vortex 10 seconds |
| | Control by visual inspection: No precipitation |
| | Mix the content of the 2 vials by pipetting all the 0.5 mL |
| | solution to |
| | the other vial (peptide concentration: 1 mg/ml) - vortex 3 |
| | seconds |
| | Incubate the mixture at 40° C. for 1 hour |
| | Acceptance criteria: decolorization of the purple colour of |
| | the mixture |
| | Sterilize the peptide solution by filtering through a |
| | syringe filter* |
| | into a sterile 1 mL sterile plastic vial |
| | Control by visual inspection: Intact sterile filter after |
| | filtration |
| | Fill 0.5 mL peptide solution into the labelled 1 mL sterile |
| | syringe |
| | Control by visual inspection: Clear to yellow solution |
| | Deliver at room temperature to the physician for prompt |
| | injection |

The Raw Materials are lyophilized peptides, supplied by VERDI Solutions GmbH with a Certificate of Analysis and full analytical documentation to the pharmacy for VERDI Vaccine preparation.
All the starting materials meet the highest possible grade, TSE contamination is excluded (Name and address of the vendor)
All the solvents used during the synthesis and the purification steps are Ph. Eur. reagent grade and impede the growth of bacteria
The water used during the purification step is type-I purified water Raw Peptide Material synthesis and purification.

The peptide chain is elongated on TentaGel R RAM resin (0.19 mmol g⁻¹) with a Rink amide linker on a 0.1 mmol scale manually with Fmoc protection scheme. The coupling is performed in two steps.
1. 3 equivalents of Fmoc-protected amino acid, 3 equivalents of the uronium coupling agent O-(7-azabenzotriazol-1-yl)-N,N,N′,N′-tetramethyluronium hexafluorophosphate (HATU) and 6 equivalents of N,N-diisopropylethylamine (DIPEA) is used in N,N-dimethylformamide (DMF) as a solvent with shaking for 3 h.
2. The second coupling is performed with 1 equivalent of amino acid, 1 equivalent of HATU, and 2 equivalents of DIPEA.

After the coupling steps, the resin is washed 3 times with DMF, once with MeOH and 3 times with DCM. No truncated sequences were observed under these coupling conditions. Deprotection was performed with 2% DBU and 2% piperidine in DMF in two steps, with reaction times of 5 and 15 min. The cleavage is performed with TFA/water/dl-dithiothreitol (DTT)/TIS (90:5:2.5:2.5) at 0° C. for 1 h.

The purification is carried out by RP-HPLC, using a Phenomenex Luna C18100 Å 10 μm column (10 mm×250 mm). The HPLC apparatus was made by JASCO. The solvent system: 0.1% TFA in water; 0.1% TFA in 80% acetonitrile in water; a linear gradient was used during 60 min, at a flow rate of 4.0 mL min-1, with detection at 206 nm.

The purities of the fractions are determined by analytical RP-HPLC-MS using an Agilent 1200HPLC system equipped with a Bruker HCT II ion trap MS with a Phenomenex Luna C18100 Å 5 μm column (4.6 mm×250 mm) 123 and the pure fractions are pooled and lyophilized. The purified peptides are characterized by MS, Bruker HCT II ion trap mass spectrometer equipped with an electrospray ion source.

Although a few preferred embodiments of the present invention have been shown and described, it will be appreciated by those skilled in the art that various changes and modifications might be made without departing from the scope of the invention, as defined in the appended claims. The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive. Each feature disclosed in this specification (including any accompanying claims, abstract and drawings) may be replaced by alternative features serving the same, equivalent, or similar purpose, unless expressly stated otherwise. Thus, unless expressly stated otherwise, each feature disclosed is one example only of a generic series of equivalent or similar features.

### Example 8: Industrial Applicability of the Invention for the Treatment of Cancer Patients

The invention offers a novel approach to personalized cancer treatment, challenging traditional pharmaceutical models. Our innovative solution involves the development of personalized vaccines tailored to each cancer patient's unique genetic profile and tumor antigen characteristics.

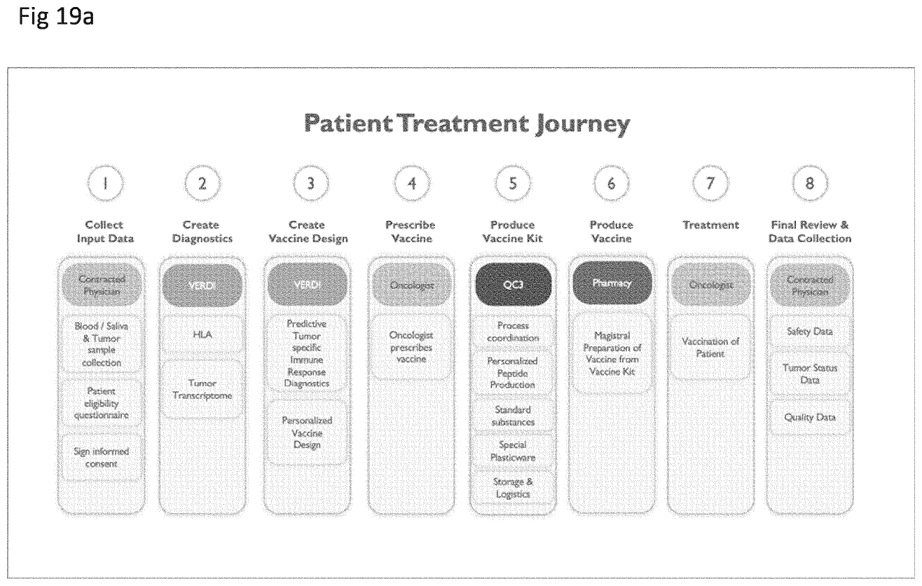

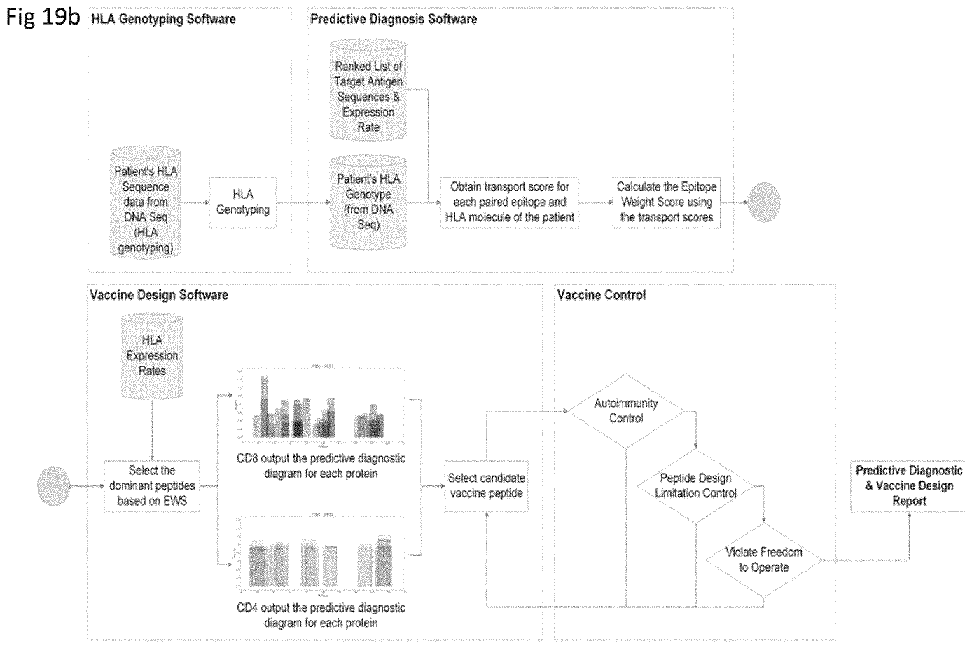

Our process starts tumor biopsy-based transcriptome sequencing and 4-digit HLA genotype data collection, both readily available commercially in Europe, facilitating seamless implementation. Using these patient-specific dataset VERDI offers to physicians a cloud-based predictive diagnostic of tumor-specific T cell responses and personalized peptide vaccines design services (FIG. 19a).

VERDI's vaccine design software operates as a clinical decision support tool, offering evidence-based personalized vaccine recommendations to physicians. The objective evidence is generated by our clinically validated predictive diagnostic tool from the patient's HLA genotype and tumor mRNA data. It supports decision-making by providing a ranked list of personalized vaccines based on patient-specific data, empowering healthcare professionals to integrate informed choices into patient care alongside their expertise and other treatment options.

The prescribed peptide vaccines are prepared as a magistral preparation in pharmacies, ensuring accessibility and affordability for patients. Our “Efficacy by Design” approach ensures the high likelihood that the personalized vaccines designed by VERDI elicit tumor-specific T cell responses, enhancing the efficacy of the traditional treatment, and increasing the likelihood of long-term remission.

Our unique and breakthrough technology includes a predictive diagnostic software that utilizes machine learning. This software is trained and validated with clinical data to accurately predict individual-level T-cell responses and create a ranked list of epitopes with the highest probability of inducing immune responses. Consequently, our personalized VERDI vaccines are designed based on objective clinical evidence, ensuring an optimized approach to each individual's immune responses (FIG. 19b).

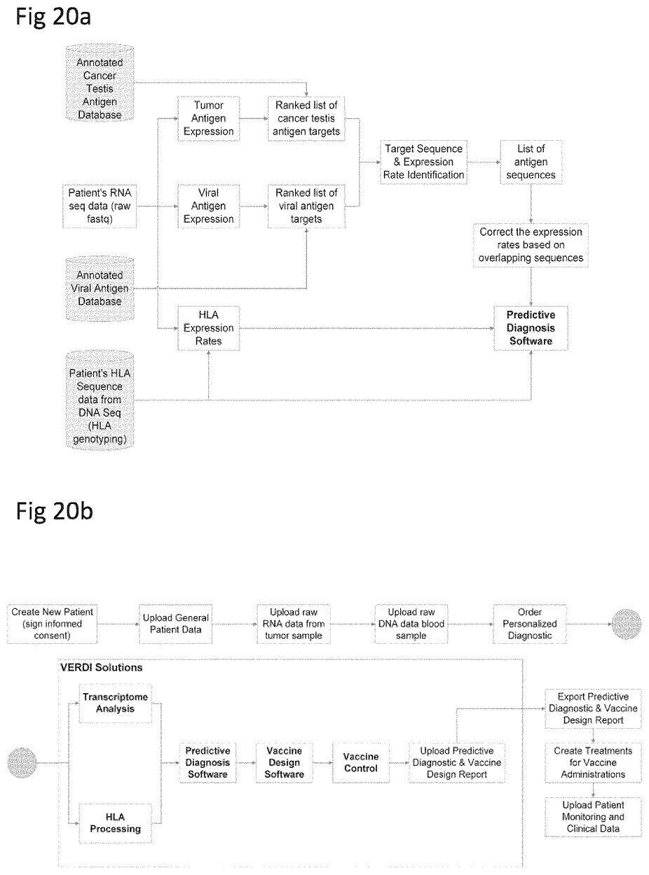

To enhance the quality of input data for predictive diagnosis, own transcriptome analysis software processes RNASEQ data extracted from tumor biopsies, skillfully ranking the patient's tumor-specific antigens to their relative expression levels. This ensures that the design of VERDI vaccines can incorporate considerations for sufficiently expressed tumor antigens (FIG. 20a).

Certified users can securely access the predictive diagnostic and vaccine design results through our user-friendly web-based application. This platform not only ensures efficiency but also prioritizes data security, adhering to GDPR and other relevant regulations. Our commitment to privacy and compliance is seamlessly integrated into every aspect of the user experience, providing a reliable and responsible service to our customers (FIG. 20b).

Current innovative medical products are a) available only to a fraction of eligible patients and b) and those can only be accessed through clinical trials from centrally produced drugs of big pharma. The “state of art” one-fits-all drugs in clinical trials are conducted on a population of patients who are most likely going to respond positively to the investigational drug.

Our business innovation is that ALL cancer patients can already NOW receive our personalized vaccines produced at local pharmacies through magistral exemption of the drug law. That way we can generate real world evidence before even starting the clinical trials and reach hundreds of patients at least before the clinical trial to significantly speed up the EMA market approval.

The timing for bringing this innovation to the market is ideal NOW due to several key factors:

Trend: Just as we can see our unique reflections in a mirror, our DNA also reveals our individuality. In an era where individualism is celebrated, why does medicine still follow a one-size-fits-all strategy? It's time for a shift towards personalized healthcare.

Immunotherapy Revolution: The ongoing revolution in immunotherapy has underscored the limitations of existing solutions, paving the way for personalized approaches like ours.

Advancements in Vaccine Development: Major vaccine providers are investing in the development of personalized RNA vaccines, heralding a new era in therapeutic vaccine development.

Growing Interest in Personalized Cancer Therapies: Novel initiatives, such as the Center for Personalized Medicine in Germany, reflect a burgeoning interest in personalized cancer therapies.

Common Sense Oncology Initiative: Leading oncologists worldwide have initiated reformative efforts in cancer trials and care, aligning perfectly with VERDI's mission.

Urgent Medical Need: The fact that the first cancer patients have already been treated with a personalized vaccine designed by VERDI underscores the urgent medical need and acceptance by stakeholders.

COVID pandemic: The pandemic has increased society's knowledge and acceptance of vaccines, creating a favorable environment for personalized vaccine therapies for cancer patients.

Advances in AI: The significant advancements in AI, as highlighted by our evaluators, further support the timely introduction of our AI-powered solution enabling personalized cancer care.

Societal Needs: The urgency to develop our proposed project is justified not only in terms of societal needs but also in alignment with current scientific and technological trends.

Our business model is largely scalable due to the fact that the components needed for the magistral preparation of our personalized vaccines are NOT produced in house. Instead, our personalized vaccines are produced in local pharmacies, whereas our vaccine kits with the components are locally sourced from biotech companies. This fully eliminates dependency on big-pharma companies.

With our Web Application we manage the entire patient treatment journey, commencing from diagnostics and extending through vaccine design, prescription, personalized production at the nearest or swiftest provider, central source delivery, pharmacy kit distribution, magistral preparation at the pharmacy, culminating in the patient's treatment at the oncologist's office. Each stakeholder can track every stage of the process, akin to tracking a package with DHL, UPS, or FedEx. This is the foundation of the scalability of our business.

1. A computer-implemented method for identifying in a subject at least one antigen, which is expected to induce a T-cell response to attack unhealthy cells in the subject, the method comprising:
receiving subject data identifying multiple major histocompatibility complex (MHC) molecules for the subject;
receiving sequence data for a plurality of epitopes within a protein which that is expressed in the unhealthy cells, wherein each epitope of the plurality of epitopes is an amino acid sequence within the protein;
obtaining, for each epitope of the plurality of epitopes, a potency score indicative of a likelihood that each of the identified multiple MHC molecules displays an epitope on their surface;
generating a ranked list of epitopes based on the determined potency scores;
identifying at least one antigen by selecting at least one epitope that is a highly ranked in the ranked list; and
outputting at least one of the ranked list and sequence data for the identified at least one antigen.

2. The method of claim 1 further comprising:
selecting multiple epitopes that are highly ranked in the ranked list;
identifying one or more subitopes of each of the selected epitopes using a directed graph network; and
identifying each subitope of the one or more subitopes that is common to more than one of the selected epitopes.

3. The method of claim 2, further comprising identifying the at least one antigen by selecting a common subitope that is highly ranked in the ranked list.

4. The method of claim 2, further comprising identifying the at least one antigen by selecting a longest common subitope.

5. The method of claim 2, further comprising identifying a shortest common subitope as a target sequence.

6. The method of claim 1, wherein the step of obtaining the potency score comprises calculating an epitope weight score for each epitope of the plurality of epitopes, wherein the step of calculating the epitope weight score comprises:
selecting an epitope from the plurality of epitopes,
obtaining, for each of the identified MHC molecules, a probability score that is indicative of a likelihood that each MHC molecule transports the selected epitope; and
calculating the epitope weight score for the selected epitope by aggregating at least some of the probability scores.

7. The method of claim 6, wherein the probability score is an eluted ligand score that is indicative of a likelihood that the epitope can be eluted from a given MHC molecule.

8. The method of claim 6, wherein the epitope weight score is calculated from:
$${EWS}_{( x )} = {\sum }_{i = 1}^{n} {w}_{i} * {ELS}_{( x , i )} for all {w}_{i} * {ELS}_{( x , i )} > ELT$$
Where x is a peptide, i is between 1 and n with n being the number of identified MHC molecules, ELS(x,i) is the probability score, i.e. eluted ligand score for each MHC molecule and epitope pair, ELT is an eluted ligand threshold and wi is a weighting parameter.

9. The method of claim 2, further comprising calculating at least one additional score for each epitope of the plurality of epitopes, the additional score being indicative of whether the one or more subitopes of each epitope are capable of triggering an identical T-cell response.

10. A method of designing a personalized vaccine to induce a T-cell response to attack unhealthy cells in a subject, the method comprising
receiving subject data identifying multiple major histocompatibility complex (MHC) molecules for the subject, wherein the subject data identifies at least one of a set of HLA class I molecules and a set of HLA class II molecules;
receiving sequence data for a plurality of epitopes within a protein expressed in the unhealthy cells, wherein each epitope is an amino acid sequence within the protein;
obtaining, for each epitope of the plurality of epitopes, a first potency score that is indicative of a likelihood that each of the identified set of HLA class I molecules displays an epitope on their surface;
generating a first ranked list of epitopes based on the first potency score for each epitope of the plurality of epitopes;
selecting multiple epitopes that are highly ranked in the first ranked list;
identifying subitopes of each of the selected multiple epitopes using a directed graph network;
identifying each subitope that is common to more than one of the selected multiple epitopes;
identifying at least one first antigen by selecting at least one subitope that is itself a highly ranked epitope in the first ranked list; and
outputting the identified at least one first antigen for the personalized vaccine.

11. The method according to claim 10, further comprising:
obtaining for each epitope of the plurality of epitopes, a second potency score that is indicative of a likelihood that each of the identified set of HLA class II molecules displays the epitope on their surface;
generating a second ranked list of epitopes based on the determined second potency scores;
selecting multiple epitopes that are highly ranked in the second ranked list;
identifying subitopes of each of the selected multiple epitopes using a directed graph network;
identifying each subitope that is common to more than one of the selected multiple epitopes;
identifying at least one second antigen by selecting at least one subitope that is itself a highly ranked epitope in the second ranked list; and
outputting the identified at least one second antigen for the personalized vaccine.

12. The method according to claim 11, further comprising:
determining if the identified first antigen is a highly ranked epitope in the second ranked list; and
in response to the first antigen being a highly ranked epitope in the second ranked list, designing the personalized vaccine based on the first antigen.

13. The method according to claim 11, wherein the first or second antigen comprises multiple epitopes that are highly ranked in the respective first or second ranked lists, capable of being displayed by the subject's MHC class I and MHC class II molecules on the cell surface, induce CD4 and CD8 T-cell responses, and share at least one common sequence capable of triggering an identical T-cell response.

14-22. (canceled)

23. A method of treating a patient with cancer or infectious disease with a personalized vaccine that induces immune responses against unhealthy cells, the method comprising:
a) obtaining at least one sample from a subject, the sample containing the unhealthy cells;
b) analyzing the at least one sample to identify an expression of major histocompatibility complex (MHC) class I and MHC class II molecules in the subject and at least one protein expressed in the unhealthy cells;
c) generating a ranked list of epitopes from the at least one protein based on an ability of the epitopes to be displayed by the MHC molecules of the subject and to induce CD4 and CD8 T-cell responses;
d) selecting multiple highly-ranked epitopes from the ranked list that share at least one common sequence capable of triggering a T-cell response;
e) incorporating the selected highly-ranked epitopes to prepare a personalized vaccine; and
f) administering the personalized vaccine to the subject.

24. The method of claim 23, wherein the ranked list of epitopes is generated by:
receiving subject data identifying MHC molecules for the subject, wherein the subject data identifies at least one of a set of HLA class I molecules and a set of HLA class II molecules;
receiving sequence data for a plurality of epitopes within the at least one protein expressed in the unhealthy cells, wherein each epitope is an amino acid sequence within the at least one protein;
obtaining, for each epitope of the plurality of epitopes, a potency score that is indicative of a likelihood that each of the identified set of HLA class I molecules displays an epitope on their surface; and
using the potency score for each epitope of the plurality of epitopes to generate the ranked list of epitopes.

25. The method of claim 24, wherein the personalized vaccine comprises a peptide antigen derived from the at least one protein expressed in the unhealthy cells, the peptide antigen comprising multiple highly-ranked epitopes that are highly ranked in the ranked list.

26. The method of claim 25, wherein the multiple highly-ranked epitopes are capable of being displayed on a cell surface of the MHC class I and MHC class II molecules and induce CD4 and CD8 T-cell responses, the multiple highly-ranked epitopes sharing at least one common sequence capable of triggering an identical T-cell response.

27. The method of claim 24, wherein the step of obtaining the potency score comprises calculating an epitope weight score for each epitope of the plurality of epitopes, wherein the step of calculating the epitope weight score comprises:
selecting an epitope from the plurality of epitopes,
obtaining, for each of the identified MHC molecules, a probability score that is indicative of a likelihood that each MHC molecule transports the selected epitope; and
calculating the epitope weight score for the selected epitope by aggregating at least some of the probability scores.

28. The method of claim 27, wherein the probability score is an eluted ligand score that is indicative of a likelihood that the epitope can be eluted from a given MHC molecule.

29. The method of claim 23, wherein the personalized vaccine is designed based on an antigen, wherein the antigen is identified by:
selecting multiple epitopes that are highly ranked in the ranked list;
identifying subitopes of each of the selected multiple epitopes using a directed graph network;
identifying each subitope that is common to more than one of the selected multiple epitopes; and
identifying the antigen by selecting at least one subitope that is itself a highly ranked epitope in the ranked list.


SEQUENCE LISTING (1133 sequences)

SEQ ID NO: 1 - protein, 9 aa, Homo sapiens
YVDEKAPEF

SEQ ID NO: 2 - protein, 9 aa, Homo sapiens
SVDPAQLEF

SEQ ID NO: 3 - protein, 9 aa, Homo sapiens
MPPFPPPSL

SEQ ID NO: 4 - protein, 9 aa, Homo sapiens
APSDPVSVL

SEQ ID NO: 5 - protein, 9 aa, Homo sapiens
KPIPASVVL

SEQ ID NO: 6 - protein, 9 aa, Homo sapiens
SLDEELHLL

SEQ ID NO: 7 - protein, 9 aa, Homo sapiens
SPEPLQALL

SEQ ID NO: 8 - protein, 9 aa, Homo sapiens
FFFERIMKY

SEQ ID NO: 9 - protein, 11 aa, Homo sapiens
FPMPPFPPPSL

SEQ ID NO: 10 - protein, 9 aa, Homo sapiens
EVDPTGHSF

SEQ ID NO: 11 - protein, 9 aa, Homo sapiens
TPSRGPPQL

SEQ ID NO: 12 - protein, 15 aa, Homo sapiens
KKDFRAMKDLAQQIN

SEQ ID NO: 13 - protein, 15 aa, Homo sapiens
MKVLEYIANANGRDP

SEQ ID NO: 14 - protein, 15 aa, Homo sapiens
VKGYELAVTQHQQLA

SEQ ID NO: 15 - protein, 16 aa, Homo sapiens
VEFLLIKNANANAVNK

SEQ ID NO: 16 - protein, 17 aa, Homo sapiens
SLPQKYKQVTNGQGENK

SEQ ID NO: 17 - protein, 15 aa, Homo sapiens
SPSVVASLPLDQSDE

SEQ ID NO: 18 - protein, 15 aa, Homo sapiens
VPVKGYELAVTQHQQ

SEQ ID NO: 19 - protein, 15 aa, Homo sapiens
KPAIEMQNSVPNKAF

SEQ ID NO: 20 - protein, 15 aa, Homo sapiens
DDPLYGISVSLSSED

SEQ ID NO: 21 - protein, 15 aa, Homo sapiens
DVAYYFEKDVKGESL

SEQ ID NO: 22 - protein, 15 aa, Homo sapiens
KKDFRAMKDLAQQIN

SEQ ID NO: 23 - protein, 13 aa, Homo sapiens
KKDFRAMKDLAQQ

SEQ ID NO: 24 - protein, 14 aa, Homo sapiens
KKDFRAMKDLAQQI

SEQ ID NO: 25 - protein, 16 aa, Homo sapiens
MKKDFRAMKDLAQQIN

SEQ ID NO: 26 - protein, 14 aa, Homo sapiens
MKKDFRAMKDLAQQ

SEQ ID NO: 27 - protein, 15 aa, Homo sapiens
MKKDFRAMKDLAQQI

SEQ ID NO: 28 - protein, 16 aa, Homo sapiens
KKDFRAMKDLAQQINL

SEQ ID NO: 29 - protein, 17 aa, Homo sapiens
KMKKDFRAMKDLAQQIN

SEQ ID NO: 30 - protein, 15 aa, Homo sapiens
KMKKDFRAMKDLAQQ

SEQ ID NO: 31 - protein, 16 aa, Homo sapiens
KMKKDFRAMKDLAQQI

SEQ ID NO: 32 - protein, 17 aa, Homo sapiens
MKKDFRAMKDLAQQINL

SEQ ID NO: 33 - protein, 14 aa, Homo sapiens
KDFRAMKDLAQQIN

SEQ ID NO: 34 - protein, 16 aa, Homo sapiens
EKMKKDFRAMKDLAQQ

SEQ ID NO: 35 - protein, 18 aa, Homo sapiens
EKMKKDFRAMKDLAQQIN

SEQ ID NO: 36 - protein, 17 aa, Homo sapiens
KKDFRAMKDLAQQINLS

SEQ ID NO: 37 - protein, 13 aa, Homo sapiens
KDFRAMKDLAQQI

SEQ ID NO: 38 - protein, 18 aa, Homo sapiens
KMKKDFRAMKDLAQQINL

SEQ ID NO: 39 - protein, 17 aa, Homo sapiens
EKMKKDFRAMKDLAQQI

SEQ ID NO: 40 - protein, 12 aa, Homo sapiens
KDFRAMKDLAQQ

SEQ ID NO: 41 - protein, 15 aa, Homo sapiens
IPAFAAAYFESLLEK

SEQ ID NO: 42 - protein, 15 aa, Homo sapiens
EDRFYNNHAFEEQEP

SEQ ID NO: 43 - protein, 16 aa, Homo sapiens
NIPAFAAAYFESLLEK

SEQ ID NO: 44 - protein, 15 aa, Homo sapiens
KEEVAAVKIQAAFRG

SEQ ID NO: 45 - protein, 14 aa, Homo sapiens
PAFAAAYFESLLEK

SEQ ID NO: 46 - protein, 16 aa, Homo sapiens
EDRFYNNHAFEEQEPP

SEQ ID NO: 47 - protein, 17 aa, Homo sapiens
DNIPAFAAAYFESLLEK

SEQ ID NO: 48 - protein, 16 aa, Homo sapiens
IPAFAAAYFESLLEKR

SEQ ID NO: 49 - protein, 14 aa, Homo sapiens
KEEVAAVKIQAAFR

SEQ ID NO: 50 - protein, 16 aa, Homo sapiens
VEDRFYNNHAFEEQEP

SEQ ID NO: 51 - protein, 15 aa, Homo sapiens
NIPAFAAAYFESLLE

SEQ ID NO: 52 - protein, 15 aa, Homo sapiens
EKEEVAAVKIQAAFR

SEQ ID NO: 53 - protein, 16 aa, Homo sapiens
EKEEVAAVKIQAAFRG

SEQ ID NO: 54 - protein, 15 aa, Homo sapiens
PAFAAAYFESLLEKR

SEQ ID NO: 55 - protein, 17 aa, Homo sapiens
NIPAFAAAYFESLLEKR

SEQ ID NO: 56 - protein, 14 aa, Homo sapiens
IPAFAAAYFESLLE

SEQ ID NO: 57 - protein, 13 aa, Homo sapiens
PAFAAAYFESLLE

SEQ ID NO: 58 - protein, 15 aa, Homo sapiens
NIPAFAAAYFESLLE

SEQ ID NO: 59 - protein, 9 aa, Homo sapiens
YVDEKAPEF

SEQ ID NO: 60 - protein, 15 aa, Homo sapiens
DVAYYFEKDVKGESL

SEQ ID NO: 61 - protein, 9 aa, Homo sapiens
HLYSHPIIL

SEQ ID NO: 62 - protein, 9 aa, Homo sapiens
RFYPKVTKY

SEQ ID NO: 63 - protein, 9 aa, Homo sapiens
SRPPVGSSL

SEQ ID NO: 64 - protein, 10 aa, Homo sapiens
FRPTTGRTSL

SEQ ID NO: 65 - protein, 8 aa, Homo sapiens
RPPNAPIL

SEQ ID NO: 66 - protein, 9 aa, Homo sapiens
HYFQTRHYL

SEQ ID NO: 67 - protein, 9 aa, Homo sapiens
FYPKVTKYL

SEQ ID NO: 68 - protein, 9 aa, Homo sapiens
FAVPNLQSL

SEQ ID NO: 69 - protein, 9 aa, Homo sapiens
SLYADSPSV

SEQ ID NO: 70 - protein, 9 aa, Homo sapiens
HPAAMPHLL

SEQ ID NO: 71 - protein, 9 aa, Homo sapiens
RPTTGRTSL

SEQ ID NO: 72 - protein, 9 aa, Homo sapiens
NPHWKTPSF

SEQ ID NO: 73 - protein, 8 aa, Homo sapiens
RPPVGSSL

SEQ ID NO: 74 - protein, 10 aa, Homo sapiens
APLPIHTAEL

SEQ ID NO: 75 - protein, 9 aa, Homo sapiens
QILPKVLHK

SEQ ID NO: 76 - protein, 9 aa, Homo sapiens
SERPVFPCW

SEQ ID NO: 77 - protein, 9 aa, Homo sapiens
VPTDHGAHL

SEQ ID NO: 78 - protein, 9 aa, Homo sapiens
TPSFPNIHL

SEQ ID NO: 79 - protein, 10 aa, Homo sapiens
RPFSGSLGTL

SEQ ID NO: 80 - protein, 9 aa, Homo sapiens
FLLSLGIHL

SEQ ID NO: 81 - protein, 8 aa, Homo sapiens
FLGPLLVL

SEQ ID NO: 82 - protein, 10 aa, Homo sapiens
RPTTGRTSLY

SEQ ID NO: 83 - protein, 9 aa, Homo sapiens
QPTPLSPPL

SEQ ID NO: 84 - protein, 11 aa, Homo sapiens
VPTDHGAHLSL

SEQ ID NO: 85 - protein, 9 aa, Homo sapiens
AESRLVVDF

SEQ ID NO: 86 - protein, 13 aa, Homo sapiens
RLPFRPTTGRTSL

SEQ ID NO: 87 - protein, 11 aa, Homo sapiens
APLPIHTAELL

SEQ ID NO: 88 - protein, 10 aa, Homo sapiens
TLWKAGILYK

SEQ ID NO: 89 - protein, 9 aa, Homo sapiens
GLYSSTVPV

SEQ ID NO: 90 - protein, 9 aa, Homo sapiens
LRNTHPQAM

SEQ ID NO: 91 - protein, 9 aa, Homo sapiens
AAYPAVSTF

SEQ ID NO: 92 - protein, 10 aa, Homo sapiens
SLPQEHIIQK

SEQ ID NO: 93 - protein, 9 aa, Homo sapiens
YMDDVVLGA

SEQ ID NO: 94 - protein, 10 aa, Homo sapiens
RQPTPLSPPL

SEQ ID NO: 95 - protein, 10 aa, Homo sapiens
RLPFRPTTGR

SEQ ID NO: 96 - protein, 10 aa, Homo sapiens
HQILPKVLHK

SEQ ID NO: 97 - protein, 9 aa, Homo sapiens
HLPDRVHFA

SEQ ID NO: 98 - protein, 11 aa, Homo sapiens
SPQAQGILQTL

SEQ ID NO: 99 - protein, 12 aa, Homo sapiens
LPFRPTTGRTSL

SEQ ID NO: 100 - protein, 8 aa, Homo sapiens
SPSVPSHL

SEQ ID NO: 101 - protein, 10 aa, Homo sapiens
SCLHQSPVRK

SEQ ID NO: 102 - protein, 9 aa, Homo sapiens
GLLGWSPQA

SEQ ID NO: 103 - protein, 8 aa, Homo sapiens
GPSLYSIL

SEQ ID NO: 104 - protein, 9 aa, Homo sapiens
HLHQDIIKK

SEQ ID NO: 105 - protein, 11 aa, Homo sapiens
AHQILPKVLHK

SEQ ID NO: 106 - protein, 9 aa, Homo sapiens
RETTHSASF

SEQ ID NO: 107 - protein, 11 aa, Homo sapiens
AAYPAVSTFEK

SEQ ID NO: 108 - protein, 9 aa, Homo sapiens
LLYQTFGRK

SEQ ID NO: 109 - protein, 9 aa, Homo sapiens
LPRLADEGL

SEQ ID NO: 110 - protein, 11 aa, Homo sapiens
ADDPSRGRLGL

SEQ ID NO: 111 - protein, 9 aa, Homo sapiens
CLHQSPVRK

SEQ ID NO: 112 - protein, 8 aa, Homo sapiens
FPSVRDLL

SEQ ID NO: 113 - protein, 9 aa, Homo sapiens
FLLAQFTSA

SEQ ID NO: 114 - protein, 9 aa, Homo sapiens
TPTGWGLVM

SEQ ID NO: 115 - protein, 10 aa, Homo sapiens
KTPSFPNIHL

SEQ ID NO: 116 - protein, 9 aa, Homo sapiens
WPKFAVPNL

SEQ ID NO: 117 - protein, 11 aa, Homo sapiens
IPMGVGLSPFL

SEQ ID NO: 118 - protein, 10 aa, Homo sapiens
TPSRVTGGVF

SEQ ID NO: 119 - protein, 8 aa, Homo sapiens
ILSPFLPL

SEQ ID NO: 120 - protein, 11 aa, Homo sapiens
ASCLHQSPVRK

SEQ ID NO: 121 - protein, 9 aa, Homo sapiens
IPRTPSRVT

SEQ ID NO: 122 - protein, 10 aa, Homo sapiens
YPVARQRPGL

SEQ ID NO: 123 - protein, 10 aa, Homo sapiens
TVNAHQILPK

SEQ ID NO: 124 - protein, 10 aa, Homo sapiens
IPMGVGLSPF

SEQ ID NO: 125 - protein, 11 aa, Homo sapiens
YYPEHLVNHYF

SEQ ID NO: 126 - protein, 10 aa, Homo sapiens
AVPTDHGAHL

SEQ ID NO: 127 - protein, 11 aa, Homo sapiens
RPPVGSSLQSK

SEQ ID NO: 128 - protein, 9 aa, Homo sapiens
GEEIRLKVF

SEQ ID NO: 129 - protein, 10 aa, Homo sapiens
VPNLQSLTNL

SEQ ID NO: 130 - protein, 11 aa, Homo sapiens
HTLWKAGILYK

SEQ ID NO: 131 - protein, 10 aa, Homo sapiens
LSRPPVGSSL

SEQ ID NO: 132 - protein, 10 aa, Homo sapiens
RLGLSRPLLR

SEQ ID NO: 133 - protein, 13 aa, Homo sapiens
HLYSHPIILGFRK

SEQ ID NO: 134 - protein, 9 aa, Homo sapiens
RLADEGLNR

SEQ ID NO: 135 - protein, 8 aa, Homo sapiens
HPAAMPHL

SEQ ID NO: 136 - protein, 9 aa, Homo sapiens
SPYSWEQDL

SEQ ID NO: 137 - protein, 8 aa, Homo sapiens
YPKVTKYL

SEQ ID NO: 138 - protein, 11 aa, Homo sapiens
GSLPQEHIIQK

SEQ ID NO: 139 - protein, 10 aa, Homo sapiens
YPKVTKYLPL

SEQ ID NO: 140 - protein, 8 aa, Homo sapiens
RPLLRLPF

SEQ ID NO: 141 - protein, 9 aa, Homo sapiens
KYTSFPWLL

SEQ ID NO: 142 - protein, 10 aa, Homo sapiens
SERPVFPCWW

SEQ ID NO: 143 - protein, 10 aa, Homo sapiens
LPKVLHKRTL

SEQ ID NO: 144 - protein, 8 aa, Homo sapiens
LPIHTAEL

SEQ ID NO: 145 - protein, 9 aa, Homo sapiens
VSIPWTHKV

SEQ ID NO: 146 - protein, 11 aa, Homo sapiens
RTSLYADSPSV

SEQ ID NO: 147 - protein, 9 aa, Homo sapiens
RAFPHCLAF

SEQ ID NO: 148 - protein, 8 aa, Homo sapiens
KPYYPEHL

SEQ ID NO: 149 - protein, 8 aa, Homo sapiens
MENITSGF

SEQ ID NO: 150 - protein, 8 aa, Homo sapiens
SRDLVVSY

SEQ ID NO: 151 - protein, 9 aa, Homo sapiens
KPYYPEHLV

SEQ ID NO: 152 - protein, 10 aa, Homo sapiens
SPTYKAFLCK

SEQ ID NO: 153 - protein, 11 aa, Homo sapiens
RVTGGVFLVDK

SEQ ID NO: 154 - protein, 9 aa, Homo sapiens
DPSRGRLGL

SEQ ID NO: 155 - protein, 11 aa, Homo sapiens
KVFVLGGCRHK

SEQ ID NO: 156 - protein, 10 aa, Homo sapiens
FNPHWKTPSF

SEQ ID NO: 157 - protein, 10 aa, Homo sapiens
YYPEHLVNHY

SEQ ID NO: 158 - protein, 9 aa, Homo sapiens
LYSSTVPVF

SEQ ID NO: 159 - protein, 11 aa, Homo sapiens
MPHLLVGSSGL

SEQ ID NO: 160 - protein, 12 aa, Homo sapiens
SPPLRNTHPQAM

SEQ ID NO: 161 - protein, 9 aa, Homo sapiens
HLYSHPIIL

SEQ ID NO: 162 - protein, 9 aa, Homo sapiens
FAVPNLQSL

SEQ ID NO: 163 - protein, 9 aa, Homo sapiens
SLYADSPSV

SEQ ID NO: 164 - protein, 8 aa, Homo sapiens
LLDTASAL

SEQ ID NO: 165 - protein, 9 aa, Homo sapiens
VSIPWTHKV

SEQ ID NO: 166 - protein, 9 aa, Homo sapiens
SRPPVGSSL

SEQ ID NO: 167 - protein, 9 aa, Homo sapiens
YQHFRRLLL

SEQ ID NO: 168 - protein, 9 aa, Homo sapiens
EQFVGPLTV

SEQ ID NO: 169 - protein, 9 aa, Homo sapiens
SRNLYVSLL

SEQ ID NO: 170 - protein, 9 aa, Homo sapiens
RQQGRSWSI

SEQ ID NO: 171 - protein, 9 aa, Homo sapiens
LRNTHPQAM

SEQ ID NO: 172 - protein, 9 aa, Homo sapiens
GLYSSTVPV

SEQ ID NO: 173 - protein, 9 aa, Homo sapiens
AAYPAVSTF

SEQ ID NO: 174 - protein, 8 aa, Homo sapiens
ARFSWLSL

SEQ ID NO: 175 - protein, 9 aa, Homo sapiens
FLLSLGIHL

SEQ ID NO: 176 - protein, 8 aa, Homo sapiens
IGDPALNM

SEQ ID NO: 177 - protein, 9 aa, Homo sapiens
RVTGGVFLV

SEQ ID NO: 178 - protein, 9 aa, Homo sapiens
QLDPARDVL

SEQ ID NO: 179 - protein, 9 aa, Homo sapiens
KQYLNLYPV

SEQ ID NO: 180 - protein, 9 aa, Homo sapiens
RFYPKVTKY

SEQ ID NO: 181 - protein, 9 aa, Homo sapiens
YMDDVVLGA

SEQ ID NO: 182 - protein, 9 aa, Homo sapiens
QHFRRLLLL

SEQ ID NO: 183 - protein, 9 aa, Homo sapiens
RQRPGLCQV

SEQ ID NO: 184 - protein, 9 aa, Homo sapiens
HYFQTRHYL

SEQ ID NO: 185 - protein, 8 aa, Homo sapiens
FLGPLLVL

SEQ ID NO: 186 - protein, 11 aa, Homo sapiens
YADSPSVPSHL

SEQ ID NO: 187 - protein, 9 aa, Homo sapiens
GLLGWSPQA

SEQ ID NO: 188 - protein, 9 aa, Homo sapiens
VRRAFPHCL

SEQ ID NO: 189 - protein, 9 aa, Homo sapiens
QILPKVLHK

SEQ ID NO: 190 - protein, 8 aa, Homo sapiens
RQLLWFHI

SEQ ID NO: 191 - protein, 10 aa, Homo sapiens
SQFSRGNYRV

SEQ ID NO: 192 - protein, 9 aa, Homo sapiens
HLPDRVHFA

SEQ ID NO: 193 - protein, 8 aa, Homo sapiens
LLDYQGML

SEQ ID NO: 194 - protein, 9 aa, Homo sapiens
LRFTSARRM

SEQ ID NO: 195 - protein, 11 aa, Homo sapiens
KCEQFVGPLTV

SEQ ID NO: 196 - protein, 9 aa, Homo sapiens
GLSRPLLRL

SEQ ID NO: 197 - protein, 11 aa, Homo sapiens
RTSLYADSPSV

SEQ ID NO: 198 - protein, 8 aa, Homo sapiens
TRHYLHTL

SEQ ID NO: 199 - protein, 9 aa, Homo sapiens
RAFPHCLAF

SEQ ID NO: 200 - protein, 10 aa, Homo sapiens
TLWKAGILYK

SEQ ID NO: 201 - protein, 10 aa, Homo sapiens
HQILPKVLHK

SEQ ID NO: 202 - protein, 9 aa, Homo sapiens
HQLDPAFRA

SEQ ID NO: 203 - protein, 10 aa, Homo sapiens
SLPQEHIIQK

SEQ ID NO: 204 - protein, 8 aa, Homo sapiens
ILSPFLPL

SEQ ID NO: 205 - protein, 9 aa, Homo sapiens
VHFASPLHV

SEQ ID NO: 206 - protein, 10 aa, Homo sapiens
RLPFRPTTGR

SEQ ID NO: 207 - protein, 10 aa, Homo sapiens
SCLHQSPVRK

SEQ ID NO: 208 - protein, 10 aa, Homo sapiens
AQFTSAICSV

SEQ ID NO: 209 - protein, 9 aa, Homo sapiens
QTFGRKLHL

SEQ ID NO: 210 - protein, 9 aa, Homo sapiens
FLLAQFTSA

SEQ ID NO: 211 - protein, 9 aa, Homo sapiens
HLHQDIIKK

SEQ ID NO: 212 - protein, 11 aa, Homo sapiens
AHQILPKVLHK

SEQ ID NO: 213 - protein, 11 aa, Homo sapiens
RLADEGLNRRV

SEQ ID NO: 214 - protein, 9 aa, Homo sapiens
SRVTGGVFL

SEQ ID NO: 215 - protein, 11 aa, Homo sapiens
AAYPAVSTFEK

SEQ ID NO: 216 - protein, 9 aa, Homo sapiens
ALMPLYACI

SEQ ID NO: 217 - protein, 9 aa, Homo sapiens
LLYQTFGRK

SEQ ID NO: 218 - protein, 8 aa, Homo sapiens
GLSPTVWL

SEQ ID NO: 219 - protein, 9 aa, Homo sapiens
VLHKRTLGL

SEQ ID NO: 220 - protein, 9 aa, Homo sapiens
CLHQSPVRK

SEQ ID NO: 221 - protein, 9 aa, Homo sapiens
SLQAVPWVA

SEQ ID NO: 222 - protein, 9 aa, Homo sapiens
FYPKVTKYL

SEQ ID NO: 223 - protein, 9 aa, Homo sapiens
AVTNFLLSL

SEQ ID NO: 224 - protein, 11 aa, Homo sapiens
ASCLHQSPVRK

SEQ ID NO: 225 - protein, 9 aa, Homo sapiens
GLSRYVARL

SEQ ID NO: 226 - protein, 9 aa, Homo sapiens
LLDDEAGPL

SEQ ID NO: 227 - protein, 10 aa, Homo sapiens
TVNAHQILPK

SEQ ID NO: 228 - protein, 8 aa, Homo sapiens
SRNLYVSL

SEQ ID NO: 229 - protein, 9 aa, Homo sapiens
LSYQHFRRL

SEQ ID NO: 230 - protein, 11 aa, Homo sapiens
HTLWKAGILYK

SEQ ID NO: 231 - protein, 8 aa, Homo sapiens
GTDNSVVL

SEQ ID NO: 232 - protein, 8 aa, Homo sapiens
ILTIPQSL

SEQ ID NO: 233 - protein, 11 aa, Homo sapiens
RPPVGSSLQSK

SEQ ID NO: 234 - protein, 10 aa, Homo sapiens
RLGLSRPLLR

SEQ ID NO: 235 - protein, 9 aa, Homo sapiens
LLAQFTSAI

SEQ ID NO: 236 - protein, 13 aa, Homo sapiens
HLYSHPIILGFRK

SEQ ID NO: 237 - protein, 9 aa, Homo sapiens
RLADEGLNR

SEQ ID NO: 238 - protein, 10 aa, Homo sapiens
RVHFASPLHV

SEQ ID NO: 239 - protein, 9 aa, Homo sapiens
LQAVPWVAL

SEQ ID NO: 240 - protein, 11 aa, Homo sapiens
GSLPQEHIIQK

SEQ ID NO: 241 - protein, 11 aa, Homo sapiens
FADATPTGWGL

SEQ ID NO: 242 - protein, 9 aa, Homo sapiens
YSHPIILGF

SEQ ID NO: 243 - protein, 9 aa, Homo sapiens
FADATPTGW

SEQ ID NO: 244 - protein, 8 aa, Homo sapiens
QHFRRLLL

SEQ ID NO: 245 - protein, 9 aa, Homo sapiens
NLYVSLLLL

SEQ ID NO: 246 - protein, 10 aa, Homo sapiens
TSLYADSPSV

SEQ ID NO: 247 - protein, 9 aa, Homo sapiens
KLHLYSHPI

SEQ ID NO: 248 - protein, 9 aa, Homo sapiens
FTPPHGGLL

SEQ ID NO: 249 - protein, 9 aa, Homo sapiens
KRWGYSLNF

SEQ ID NO: 250 - protein, 12 aa, Homo sapiens
KKCEQFVGPLTV

SEQ ID NO: 251 - protein, 10 aa, Homo sapiens
YQTFGRKLHL

SEQ ID NO: 252 - protein, 9 aa, Homo sapiens
RVSWPKFAV

SEQ ID NO: 253 - protein, 8 aa, Homo sapiens
SLDVSAAF

SEQ ID NO: 254 - protein, 11 aa, Homo sapiens
RVTGGVFLVDK

SEQ ID NO: 255 - protein, 10 aa, Homo sapiens
SPTYKAFLCK

SEQ ID NO: 256 - protein, 9 aa, Homo sapiens
IQSKQAFTF

SEQ ID NO: 257 - protein, 11 aa, Homo sapiens
KVFVLGGCRHK

SEQ ID NO: 258 - protein, 8 aa, Homo sapiens
LQAGFFLL

SEQ ID NO: 259 - protein, 9 aa, Homo sapiens
DRVHFASPL

SEQ ID NO: 260 - protein, 10 aa, Homo sapiens
ARFYPKVTKY

SEQ ID NO: 261 - protein, 9 aa, Homo sapiens
AAYPAVSTF

SEQ ID NO: 262 - protein, 9 aa, Homo sapiens
RAFPHCLAF

SEQ ID NO: 263 - protein, 9 aa, Homo sapiens
YSHPIILGF

SEQ ID NO: 264 - protein, 9 aa, Homo sapiens
RFYPKVTKY

SEQ ID NO: 265 - protein, 9 aa, Homo sapiens
SSSGHAVEF

SEQ ID NO: 266 - protein, 9 aa, Homo sapiens
LYSSTVPVF

SEQ ID NO: 267 - protein, 9 aa, Homo sapiens
FAVPNLQSL

SEQ ID NO: 268 - protein, 9 aa, Homo sapiens
FASPLHVAW

SEQ ID NO: 269 - protein, 9 aa, Homo sapiens
TFSPTYKAF

SEQ ID NO: 270 - protein, 9 aa, Homo sapiens
HLYSHPIIL

SEQ ID NO: 271 - protein, 9 aa, Homo sapiens
IQSKQAFTF

SEQ ID NO: 272 - protein, 9 aa, Homo sapiens
HYFQTRHYL

SEQ ID NO: 273 - protein, 10 aa, Homo sapiens
KAAYPAVSTF

SEQ ID NO: 274 - protein, 11 aa, Homo sapiens
RKAAYPAVSTF

SEQ ID NO: 275 - protein, 8 aa, Homo sapiens
MQWNSTTF

SEQ ID NO: 276 - protein, 9 aa, Homo sapiens
QAFTFSPTY

SEQ ID NO: 277 - protein, 9 aa, Homo sapiens
FYPKVTKYL

SEQ ID NO: 278 - protein, 8 aa, Homo sapiens
AYPAVSTF

SEQ ID NO: 279 - protein, 9 aa, Homo sapiens
VSIPWTHKV

SEQ ID NO: 280 - protein, 10 aa, Homo sapiens
FTFSPTYKAF

SEQ ID NO: 281 - protein, 11 aa, Homo sapiens
YYPEHLVNHYF

SEQ ID NO: 282 - protein, 9 aa, Homo sapiens
LSLDVSAAF

SEQ ID NO: 283 - protein, 9 aa, Homo sapiens
RSQSERPVF

SEQ ID NO: 284 - protein, 9 aa, Homo sapiens
KYTSFPWLL

SEQ ID NO: 285 - protein, 9 aa, Homo sapiens
SLFTAVTNF

SEQ ID NO: 286 - protein, 8 aa, Homo sapiens
FFLLTRIL

SEQ ID NO: 287 - protein, 8 aa, Homo sapiens
FFPSVRDL

SEQ ID NO: 288 - protein, 10 aa, Homo sapiens
LYSHPIILGF

SEQ ID NO: 289 - protein, 8 aa, Homo sapiens
WYWGPSLY

SEQ ID NO: 290 - protein, 9 aa, Homo sapiens
LYQTFGRKL

SEQ ID NO: 291 - protein, 10 aa, Homo sapiens
VFADATPTGW

SEQ ID NO: 292 - protein, 8 aa, Homo sapiens
LYSILSPF

SEQ ID NO: 293 - protein, 10 aa, Homo sapiens
HFASPLHVAW

SEQ ID NO: 294 - protein, 9 aa, Homo sapiens
SYQHFRRLL

SEQ ID NO: 295 - protein, 10 aa, Homo sapiens
YYPEHLVNHY

SEQ ID NO: 296 - protein, 9 aa, Homo sapiens
AMQWNSTTF

SEQ ID NO: 297 - protein, 9 aa, Homo sapiens
STTDLEAYF

SEQ ID NO: 298 - protein, 9 aa, Homo sapiens
SHLPDRVHF

SEQ ID NO: 299 - protein, 8 aa, Homo sapiens
MWYWGPSL

SEQ ID NO: 300 - protein, 8 aa, Homo sapiens
FYPKVTKY

SEQ ID NO: 301 - protein, 9 aa, Homo sapiens
FADATPTGW

SEQ ID NO: 302 - protein, 10 aa, Homo sapiens
KQAFTFSPTY

SEQ ID NO: 303 - protein, 10 aa, Homo sapiens
RQRPGLCQVF

SEQ ID NO: 304 - protein, 9 aa, Homo sapiens
STSNPLGFF

SEQ ID NO: 305 - protein, 9 aa, Homo sapiens
RSWSIRAGF

SEQ ID NO: 306 - protein, 9 aa, Homo sapiens
SLYADSPSV

SEQ ID NO: 307 - protein, 9 aa, Homo sapiens
NYRVSWPKF

SEQ ID NO: 308 - protein, 9 aa, Homo sapiens
NTHPQAMQW

SEQ ID NO: 309 - protein, 8 aa, Homo sapiens
YSSTVPVF

SEQ ID NO: 310 - protein, 9 aa, Homo sapiens
NWILRGTSF

SEQ ID NO: 311 - protein, 9 aa, Homo sapiens
SFVYVPSAL

SEQ ID NO: 312 - protein, 9 aa, Homo sapiens
LSTSNPLGF

SEQ ID NO: 313 - protein, 8 aa, Homo sapiens
FLGPLLVL

SEQ ID NO: 314 - protein, 9 aa, Homo sapiens
KSVQHLESL

SEQ ID NO: 315 - protein, 10 aa, Homo sapiens
ARFYPKVTKY

SEQ ID NO: 316 - protein, 9 aa, Homo sapiens
FLLSLGIHL

SEQ ID NO: 317 - protein, 11 aa, Homo sapiens
RAFPHCLAFSY

SEQ ID NO: 318 - protein, 9 aa, Homo sapiens
VLSRKYTSF

SEQ ID NO: 319 - protein, 8 aa, Homo sapiens
SWWTSLNF

SEQ ID NO: 320 - protein, 11 aa, Homo sapiens
VFNPHWKTPSF

SEQ ID NO: 321 - protein, 10 aa, Homo sapiens
RRAFPHCLAF

SEQ ID NO: 322 - protein, 10 aa, Homo sapiens
ASFCGSPYSW

SEQ ID NO: 323 - protein, 9 aa, Homo sapiens
TVPVFNPHW

SEQ ID NO: 324 - protein, 9 aa, Homo sapiens
VFPCWWLQF

SEQ ID NO: 325 - protein, 11 aa, Homo sapiens
FFPDHQLDPAF

SEQ ID NO: 326 - protein, 10 aa, Homo sapiens
STVPVFNPHW

SEQ ID NO: 327 - protein, 9 aa, Homo sapiens
RLVVDFSQF

SEQ ID NO: 328 - protein, 8 aa, Homo sapiens
YFQTRHYL

SEQ ID NO: 329 - protein, 9 aa, Homo sapiens
SFCGSPYSW

SEQ ID NO: 330 - protein, 10 aa, Homo sapiens
HQSPVRKAAY

SEQ ID NO: 331 - protein, 9 aa, Homo sapiens
SAGPCALRF

SEQ ID NO: 332 - protein, 11 aa, Homo sapiens
SASFCGSPYSW

SEQ ID NO: 333 - protein, 8 aa, Homo sapiens
SFLPSDFF

SEQ ID NO: 334 - protein, 10 aa, Homo sapiens
RFYPKVTKYL

SEQ ID NO: 335 - protein, 9 aa, Homo sapiens
GYPALMPLY

SEQ ID NO: 336 - protein, 9 aa, Homo sapiens
RETTHSASF

SEQ ID NO: 337 - protein, 9 aa, Homo sapiens
QAQGILQTL

SEQ ID NO: 338 - protein, 8 aa, Homo sapiens
HYFQTRHY

SEQ ID NO: 339 - protein, 8 aa, Homo sapiens
SQFSRGNY

SEQ ID NO: 340 - protein, 9 aa, Homo sapiens
GLYSSTVPV

SEQ ID NO: 341 - protein, 10 aa, Homo sapiens
RNTHPQAMQW

SEQ ID NO: 342 - protein, 9 aa, Homo sapiens
FTPPHGGLL

SEQ ID NO: 343 - protein, 10 aa, Homo sapiens
KSVQHLESLF

SEQ ID NO: 344 - protein, 11 aa, Homo sapiens
KHSSSGHAVEF

SEQ ID NO: 345 - protein, 9 aa, Homo sapiens
NHYFQTRHY

SEQ ID NO: 346 - protein, 9 aa, Homo sapiens
SLLLLYQTF

SEQ ID NO: 347 - protein, 8 aa, Homo sapiens
SQSERPVF

SEQ ID NO: 348 - protein, 8 aa, Homo sapiens
SRDLVVSY

SEQ ID NO: 349 - protein, 12 aa, Homo sapiens
PYYPEHLVNHYF

SEQ ID NO: 350 - protein, 9 aa, Homo sapiens
ILRGTSFVY

SEQ ID NO: 351 - protein, 9 aa, Homo sapiens
YMDDVVLGA

SEQ ID NO: 352 - protein, 9 aa, Homo sapiens
RANTANPDW

SEQ ID NO: 353 - protein, 9 aa, Homo sapiens
KRWGYSLNF

SEQ ID NO: 354 - protein, 10 aa, Homo sapiens
FAAPFTQCGY

SEQ ID NO: 355 - protein, 10 aa, Homo sapiens
HSSSGHAVEF

SEQ ID NO: 356 - protein, 9 aa, Homo sapiens
LQLIMPARF

SEQ ID NO: 357 - protein, 9 aa, Homo sapiens
YQHFRRLLL

SEQ ID NO: 358 - protein, 9 aa, Homo sapiens
GLLGWSPQA

SEQ ID NO: 359 - protein, 11 aa, Homo sapiens
VHFASPLHVAW

SEQ ID NO: 360 - protein, 11 aa, Homo sapiens
LTNLLSSNLSW

SEQ ID NO: 361 - protein, 20 aa, synthetic construct
NEQDLVREEAQKMSSLLPTM

SEQ ID NO: 362 - protein, 20 aa, synthetic construct
SVYADQVNIDYLMNRPQNLR

SEQ ID NO: 363 - protein, 20 aa, synthetic construct
FTSSRMSSFNRHMKTHTSEK

SEQ ID NO: 364 - protein, 20 aa, synthetic construct
TLPPAWQPFLKDHRISTFKN

SEQ ID NO: 365 - protein, 20 aa, synthetic construct
QEESFSPTAMDAIFGSLSDE

SEQ ID NO: 366 - protein, 20 aa, synthetic construct
RLDQLLRHVMNPLETLSITN

SEQ ID NO: 367 - protein, 20 aa, synthetic construct
SGSSYFVLANGHILPNSENA

SEQ ID NO: 368 - protein, 20 aa, synthetic construct
SDTKDYFMSKTLGIGRLKRP

SEQ ID NO: 369 - protein, 9 aa, Homo sapiens
FYNPDTQRL

SEQ ID NO: 370 - protein, 9 aa, Homo sapiens
YLHPSYYML

SEQ ID NO: 371 - protein, 9 aa, Homo sapiens
SMDYKQTQL

SEQ ID NO: 372 - protein, 9 aa, Homo sapiens
YRDGNPYAV

SEQ ID NO: 373 - protein, 9 aa, Homo sapiens
RPIPKPSPW

SEQ ID NO: 374 - protein, 9 aa, Homo sapiens
VPSVPSTSL

SEQ ID NO: 375 - protein, 10 aa, Homo sapiens
YLPPVPVSKV

SEQ ID NO: 376 - protein, 9 aa, Homo sapiens
ALDGNLVSM

SEQ ID NO: 377 - protein, 9 aa, Homo sapiens
TLQDVSLEV

SEQ ID NO: 378 - protein, 9 aa, Homo sapiens
TLAPVRPPL

SEQ ID NO: 379 - protein, 9 aa, Homo sapiens
YLPPVPVSK

SEQ ID NO: 380 - protein, 9 aa, Homo sapiens
SRIGNKQTL

SEQ ID NO: 381 - protein, 9 aa, Homo sapiens
AETETAHAL

SEQ ID NO: 382 - protein, 9 aa, Homo sapiens
YRFKKHCTL

SEQ ID NO: 383 - protein, 8 aa, Homo sapiens
RPRKLPQL

SEQ ID NO: 384 - protein, 9 aa, Homo sapiens
SLFGMSLMK

SEQ ID NO: 385 - protein, 11 aa, Homo sapiens
TLAPVRPPLTV

SEQ ID NO: 386 - protein, 9 aa, Homo sapiens
SPEIIRQHL

SEQ ID NO: 387 - protein, 9 aa, Homo sapiens
KFYSKISEY

SEQ ID NO: 388 - protein, 10 aa, Homo sapiens
AEKYSKNKVW

SEQ ID NO: 389 - protein, 9 aa, Homo sapiens
APVRPPLTV

SEQ ID NO: 390 - protein, 9 aa, Homo sapiens
YYHAGTSRL

SEQ ID NO: 391 - protein, 10 aa, Homo sapiens
AETETAHALF

SEQ ID NO: 392 - protein, 11 aa, Homo sapiens
GLYSRTTQQVK

SEQ ID NO: 393 - protein, 10 aa, Homo sapiens
SEVYGDTPEW

SEQ ID NO: 394 - protein, 9 aa, Homo sapiens
ILVPKVSGL

SEQ ID NO: 395 - protein, 10 aa, Homo sapiens
RETIEKLLSK

SEQ ID NO: 396 - protein, 9 aa, Homo sapiens
LPSEATVYL

SEQ ID NO: 397 - protein, 11 aa, Homo sapiens
TPVPSVPSTSL

SEQ ID NO: 398 - protein, 9 aa, Homo sapiens
KLLSKLLCV

SEQ ID NO: 399 - protein, 10 aa, Homo sapiens
KSLFGMSLMK

SEQ ID NO: 400 - protein, 11 aa, Homo sapiens
TVYLPPVPVSK

SEQ ID NO: 401 - protein, 9 aa, Homo sapiens
EEYDLQFIF

SEQ ID NO: 402 - protein, 9 aa, Homo sapiens
FELSQMVQW

SEQ ID NO: 403 - protein, 12 aa, Homo sapiens
RPRKLPQLCTEL

SEQ ID NO: 404 - protein, 9 aa, Homo sapiens
TPIPGSRPV

SEQ ID NO: 405 - protein, 10 aa, Homo sapiens
GISGHPLLNK

SEQ ID NO: 406 - protein, 9 aa, Homo sapiens
TTYKNTNFK

SEQ ID NO: 407 - protein, 9 aa, Homo sapiens
FPLGRKFLL

SEQ ID NO: 408 - protein, 10 aa, Homo sapiens
KEDPLKKYTF

SEQ ID NO: 409 - protein, 9 aa, Homo sapiens
TPETPATPL

SEQ ID NO: 410 - protein, 9 aa, Homo sapiens
SIADSIKTL

SEQ ID NO: 411 - protein, 10 aa, Homo sapiens
GPSDPSIVSL

SEQ ID NO: 412 - protein, 9 aa, Homo sapiens
AILDINNTV

SEQ ID NO: 413 - protein, 9 aa, Homo sapiens
SPRLKAICI

SEQ ID NO: 414 - protein, 11 aa, Homo sapiens
YLPPVPVSKVV

SEQ ID NO: 415 - protein, 11 aa, Homo sapiens
AEKKQMSMSQW

SEQ ID NO: 416 - protein, 8 aa, Homo sapiens
RPVARLGL

SEQ ID NO: 417 - protein, 10 aa, Homo sapiens
TFELSQMVQW

SEQ ID NO: 418 - protein, 11 aa, Homo sapiens
KEDPLKKYTFW

SEQ ID NO: 419 - protein, 9 aa, Homo sapiens
RIHLPDPNK

SEQ ID NO: 420 - protein, 9 aa, Homo sapiens
IPANTTIPF

SEQ ID NO: 421 - protein, 9 aa, Homo sapiens
ALQAIELQL

SEQ ID NO: 422 - protein, 9 aa, Homo sapiens
SPTSINNGL

SEQ ID NO: 423 - protein, 9 aa, Homo sapiens
NLDTASTTL

SEQ ID NO: 424 - protein, 9 aa, Homo sapiens
RASATQLYK

SEQ ID NO: 425 - protein, 9 aa, Homo sapiens
YVHEGIRTY

SEQ ID NO: 426 - protein, 9 aa, Homo sapiens
ALFTAQEAK

SEQ ID NO: 427 - protein, 9 aa, Homo sapiens
RLKAICIEK

SEQ ID NO: 428 - protein, 9 aa, Homo sapiens
SLHLTAHTK

SEQ ID NO: 429 - protein, 9 aa, Homo sapiens
TEVETQQML

SEQ ID NO: 430 - protein, 9 aa, Homo sapiens
ILEDWNFGL

SEQ ID NO: 431 - protein, 12 aa, Homo sapiens
HPAATHTKAVAL

SEQ ID NO: 432 - protein, 9 aa, Homo sapiens
RPPTATDTL

SEQ ID NO: 433 - protein, 9 aa, Homo sapiens
EEGTGCNGW

SEQ ID NO: 434 - protein, 9 aa, Homo sapiens
KLRSTAAAL

SEQ ID NO: 435 - protein, 9 aa, Homo sapiens
RPRKLPQLC

SEQ ID NO: 436 - protein, 10 aa, Homo sapiens
IPGSRPVARL

SEQ ID NO: 437 - protein, 9 aa, Homo sapiens
ISGHPLLNK

SEQ ID NO: 438 - protein, 9 aa, Homo sapiens
RTWSRLSLH

SEQ ID NO: 439 - protein, 10 aa, Homo sapiens
KEYLRHGEEY

SEQ ID NO: 440 - protein, 10 aa, Homo sapiens
GEEGTGCNGW

SEQ ID NO: 441 - protein, 10 aa, Homo sapiens
AISTSETTYK

SEQ ID NO: 442 - protein, 13 aa, Homo sapiens
SLYGTTLEQQYNK

SEQ ID NO: 443 - protein, 10 aa, Homo sapiens
KTIADQILQY

SEQ ID NO: 444 - protein, 9 aa, Homo sapiens
IVALHRPAL

SEQ ID NO: 445 - protein, 11 aa, Homo sapiens
TPAETGGHFTL

SEQ ID NO: 446 - protein, 9 aa, Homo sapiens
WPTTPPRPI

SEQ ID NO: 447 - protein, 12 aa, Homo sapiens
SLWLPSEATVYL

SEQ ID NO: 448 - protein, 11 aa, Homo sapiens
SLYGTTLEQQY

SEQ ID NO: 449 - protein, 13 aa, Homo sapiens
KVVDPAFVTTPTK

SEQ ID NO: 450 - protein, 11 aa, Homo sapiens
KWTLQDVSLEV

SEQ ID NO: 451 - protein, 10 aa, Homo sapiens
GTCPPDIIPK

SEQ ID NO: 452 - protein, 9 aa, Homo sapiens
KRASATQLY

SEQ ID NO: 453 - protein, 9 aa, Homo sapiens
TIADQILQY

SEQ ID NO: 454 - protein, 9 aa, Homo sapiens
SLVEETSFI

SEQ ID NO: 455 - protein, 10 aa, Homo sapiens
SEPYGDSLFF

SEQ ID NO: 456 - protein, 9 aa, Homo sapiens
TPAETGGHF

SEQ ID NO: 457 - protein, 9 aa, Homo sapiens
LPDPNKFGF

SEQ ID NO: 458 - protein, 10 aa, Homo sapiens
KAKPKFTLGK

SEQ ID NO: 459 - protein, 10 aa, Homo sapiens
LLYGAANTGK

SEQ ID NO: 460 - protein, 9 aa, Homo sapiens
STWPTTPPR

SEQ ID NO: 461 - protein, 9 aa, Homo sapiens
WEVNLKEKF

SEQ ID NO: 462 - protein, 9 aa, Homo sapiens
FYLHPSYYM

SEQ ID NO: 463 - protein, 10 aa, Homo sapiens
HETETPCSQY

SEQ ID NO: 464 - protein, 10 aa, Homo sapiens
RPIPKPSPWA

SEQ ID NO: 465 - protein, 11 aa, Homo sapiens
KENDGDSLPTF

SEQ ID NO: 466 - protein, 11 aa, Homo sapiens
SPWAPKKHRRL

SEQ ID NO: 467 - protein, 10 aa, Homo sapiens
VYLPPVPVSK

SEQ ID NO: 468 - protein, 9 aa, Homo sapiens
AETGGHFTL

SEQ ID NO: 469 - protein, 9 aa, Homo sapiens
SMDYKQTQL

SEQ ID NO: 470 - protein, 9 aa, Homo sapiens
YRFKKHCTL

SEQ ID NO: 471 - protein, 9 aa, Homo sapiens
ALDGNLVSM

SEQ ID NO: 472 - protein, 9 aa, Homo sapiens
YLHPSYYML

SEQ ID NO: 473 - protein, 9 aa, Homo sapiens
TLQDVSLEV

SEQ ID NO: 474 - protein, 9 aa, Homo sapiens
NLDTASTTL

SEQ ID NO: 475 - protein, 9 aa, Homo sapiens
VKIPKTITV

SEQ ID NO: 476 - protein, 9 aa, Homo sapiens
SRIGNKQTL

SEQ ID NO: 477 - protein, 9 aa, Homo sapiens
AILDINNTV

SEQ ID NO: 478 - protein, 9 aa, Homo sapiens
KLLSKLLCV

SEQ ID NO: 479 - protein, 9 aa, Homo sapiens
YRFVTSQAI

SEQ ID NO: 480 - protein, 9 aa, Homo sapiens
ALQAIELQL

SEQ ID NO: 481 - protein, 9 aa, Homo sapiens
SIADSIKTL

SEQ ID NO: 482 - protein, 9 aa, Homo sapiens
IADSIKTLL

SEQ ID NO: 483 - protein, 9 aa, Homo sapiens
KVSGLQYRV

SEQ ID NO: 484 - protein, 9 aa, Homo sapiens
SQVKIPKTI

SEQ ID NO: 485 - protein, 10 aa, Homo sapiens
YLPPVPVSKV

SEQ ID NO: 486 - protein, 9 aa, Homo sapiens
ILVPKVSGL

SEQ ID NO: 487 - protein, 9 aa, Homo sapiens
SRTWSRLSL

SEQ ID NO: 488 - protein, 10 aa, Homo sapiens
YIDDNLRNAL

SEQ ID NO: 489 - protein, 9 aa, Homo sapiens
YRDGNPYAV

SEQ ID NO: 490 - protein, 9 aa, Homo sapiens
YSKNKVWEV

SEQ ID NO: 491 - protein, 9 aa, Homo sapiens
HQVVPTLAV

SEQ ID NO: 492 - protein, 9 aa, Homo sapiens
TKYPLLKLL

SEQ ID NO: 493 - protein, 9 aa, Homo sapiens
SLVEETSFI

SEQ ID NO: 494 - protein, 9 aa, Homo sapiens
ERPRKLPQL

SEQ ID NO: 495 - protein, 9 aa, Homo sapiens
IADAGDFYL

SEQ ID NO: 496 - protein, 9 aa, Homo sapiens
KQHRDAVQV

SEQ ID NO: 497 - protein, 9 aa, Homo sapiens
YHAGTSRLL

SEQ ID NO: 498 - protein, 9 aa, Homo sapiens
AAFGLTPSI

SEQ ID NO: 499 - protein, 9 aa, Homo sapiens
SRTRRETQL

SEQ ID NO: 500 - protein, 10 aa, Homo sapiens
FIDAGAPTSV

SEQ ID NO: 501 - protein, 9 aa, Homo sapiens
STDTTPAIL

SEQ ID NO: 502 - protein, 11 aa, Homo sapiens
TLAPVRPPLTV

SEQ ID NO: 503 - protein, 9 aa, Homo sapiens
KQMSMSQWI

SEQ ID NO: 504 - protein, 9 aa, Homo sapiens
QAIELQLTL

SEQ ID NO: 505 - protein, 9 aa, Homo sapiens
KHKSAIVTL

SEQ ID NO: 506 - protein, 9 aa, Homo sapiens
TLAPVRPPL

SEQ ID NO: 507 - protein, 8 aa, Homo sapiens
IADSIKTL

SEQ ID NO: 508 - protein, 9 aa, Homo sapiens
ITDQAPSLI

SEQ ID NO: 509 - protein, 9 aa, Homo sapiens
LRYQGVEFM

SEQ ID NO: 510 - protein, 9 aa, Homo sapiens
SLFGMSLMK

SEQ ID NO: 511 - protein, 8 aa, Homo sapiens
ITDQAPSL

SEQ ID NO: 512 - protein, 9 aa, Homo sapiens
YLPPVPVSK

SEQ ID NO: 513 - protein, 9 aa, Homo sapiens
PSDPSIVSL

SEQ ID NO: 514 - protein, 9 aa, Homo sapiens
ILEDWNFGL

SEQ ID NO: 515 - protein, 9 aa, Homo sapiens
LADAKIGML

SEQ ID NO: 516 - protein, 9 aa, Homo sapiens
QHRDAVQVL

SEQ ID NO: 517 - protein, 11 aa, Homo sapiens
KWTLQDVSLEV

SEQ ID NO: 518 - protein, 11 aa, Homo sapiens
GLYSRTTQQVK

SEQ ID NO: 519 - protein, 9 aa, Homo sapiens
FYNPDTQRL

SEQ ID NO: 520 - protein, 11 aa, Homo sapiens
SQVKIPKTITV

SEQ ID NO: 521 - protein, 10 aa, Homo sapiens
RETIEKLLSK

SEQ ID NO: 522 - protein, 10 aa, Homo sapiens
KSLFGMSLMK

SEQ ID NO: 523 - protein, 10 aa, Homo sapiens
GLYSRTTQQV

SEQ ID NO: 524 - protein, 9 aa, Homo sapiens
YVHEGIRTY

SEQ ID NO: 525 - protein, 11 aa, Homo sapiens
TVYLPPVPVSK

SEQ ID NO: 526 - protein, 10 aa, Homo sapiens
SLWLPSEATV

SEQ ID NO: 527 - protein, 10 aa, Homo sapiens
GISGHPLLNK

SEQ ID NO: 528 - protein, 9 aa, Homo sapiens
TTYKNTNFK

SEQ ID NO: 529 - protein, 8 aa, Homo sapiens
SVDSAPIL

SEQ ID NO: 530 - protein, 9 aa, Homo sapiens
VSEPYGDSL

SEQ ID NO: 531 - protein, 11 aa, Homo sapiens
YLPPVPVSKVV

SEQ ID NO: 532 - protein, 9 aa, Homo sapiens
VQLKCPPLL

SEQ ID NO: 533 - protein, 10 aa, Homo sapiens
SQYSNEKWTL

SEQ ID NO: 534 - protein, 8 aa, Homo sapiens
YRVFRIHL

SEQ ID NO: 535 - protein, 9 aa, Homo sapiens
RIHLPDPNK

SEQ ID NO: 536 - protein, 9 aa, Homo sapiens
TVYLPPVPV

SEQ ID NO: 537 - protein, 9 aa, Homo sapiens
SRTTQQVKV

SEQ ID NO: 538 - protein, 11 aa, Homo sapiens
GQVDYYGLYYV

SEQ ID NO: 539 - protein, 9 aa, Homo sapiens
DQFLSQVKI

SEQ ID NO: 540 - protein, 9 aa, Homo sapiens
ITDTSTTPV

SEQ ID NO: 541 - protein, 9 aa, Homo sapiens
SRAAKRRLF

SEQ ID NO: 542 - protein, 8 aa, Homo sapiens
SDPSIVSL

SEQ ID NO: 543 - protein, 9 aa, Homo sapiens
AMLAKFKEL

SEQ ID NO: 544 - protein, 9 aa, Homo sapiens
RASATQLYK

SEQ ID NO: 545 - protein, 8 aa, Homo sapiens
ILDINNTV

SEQ ID NO: 546 - protein, 9 aa, Homo sapiens
CLYLHIQSL

SEQ ID NO: 547 - protein, 9 aa, Homo sapiens
ALFTAQEAK

SEQ ID NO: 548 - protein, 9 aa, Homo sapiens
MVDTGFGAM

SEQ ID NO: 549 - protein, 9 aa, Homo sapiens
SLHLTAHTK

SEQ ID NO: 550 - protein, 9 aa, Homo sapiens
YIDDNLRNA

SEQ ID NO: 551 - protein, 9 aa, Homo sapiens
RLKAICIEK

SEQ ID NO: 552 - protein, 9 aa, Homo sapiens
YMLDLQPET

SEQ ID NO: 553 - protein, 9 aa, Homo sapiens
TLHEYMLDL

SEQ ID NO: 554 - protein, 9 aa, Homo sapiens
YLRREQMFV

SEQ ID NO: 555 - protein, 9 aa, Homo sapiens
YGDSLFFYL

SEQ ID NO: 556 - protein, 9 aa, Homo sapiens
RQTVLQHSF

SEQ ID NO: 557 - protein, 9 aa, Homo sapiens
RTWSRLSLH

SEQ ID NO: 558 - protein, 9 aa, Homo sapiens
KQQLLRREV

SEQ ID NO: 559 - protein, 9 aa, Homo sapiens
ISGHPLLNK

SEQ ID NO: 560 - protein, 10 aa, Homo sapiens
KTIADQILQY

SEQ ID NO: 561 - protein, 10 aa, Homo sapiens
TIDPAEEIEL

SEQ ID NO: 562 - protein, 10 aa, Homo sapiens
AISTSETTYK

SEQ ID NO: 563 - protein, 11 aa, Homo sapiens
SLYGTTLEQQY

SEQ ID NO: 564 - protein, 13 aa, Homo sapiens
SLYGTTLEQQYNK

SEQ ID NO: 565 - protein, 9 aa, Homo sapiens
SADLDQFPL

SEQ ID NO: 566 - protein, 10 aa, Homo sapiens
SIADSIKTLL

SEQ ID NO: 567 - protein, 11 aa, Homo sapiens
ALQAIELQLTL

SEQ ID NO: 568 - protein, 10 aa, Homo sapiens
RDQFLSQVKI

SEQ ID NO: 569 - protein, 9 aa, Homo sapiens
YVHEGIRTY

SEQ ID NO: 570 - protein, 9 aa, Homo sapiens
VVFTFPNEF

SEQ ID NO: 571 - protein, 9 aa, Homo sapiens
RAHYNIVTF

SEQ ID NO: 572 - protein, 9 aa, Homo sapiens
FYNPDTQRL

SEQ ID NO: 573 - protein, 10 aa, Homo sapiens
YYVHEGIRTY

SEQ ID NO: 574 - protein, 9 aa, Homo sapiens
KFYSKISEY

SEQ ID NO: 575 - protein, 9 aa, Homo sapiens
FTFPNEFPF

SEQ ID NO: 576 - protein, 9 aa, Homo sapiens
YLHNRLVVF

SEQ ID NO: 577 - protein, 9 aa, Homo sapiens
KSAIVTLTY

SEQ ID NO: 578 - protein, 9 aa, Homo sapiens
KSIGAKVHY

SEQ ID NO: 579 - protein, 9 aa, Homo sapiens
YLHPSYYML

SEQ ID NO: 580 - protein, 9 aa, Homo sapiens
FYLRREQMF

SEQ ID NO: 581 - protein, 9 aa, Homo sapiens
RQTVLQHSF

SEQ ID NO: 582 - protein, 9 aa, Homo sapiens
FYLHPSYYM

SEQ ID NO: 583 - protein, 9 aa, Homo sapiens
YYHAGTSRL

SEQ ID NO: 584 - protein, 9 aa, Homo sapiens
KTITVSTGF

SEQ ID NO: 585 - protein, 9 aa, Homo sapiens
SFSELVRPF

SEQ ID NO: 586 - protein, 9 aa, Homo sapiens
YYKAREMGF

SEQ ID NO: 587 - protein, 10 aa, Homo sapiens
RYQGVEFMSF

SEQ ID NO: 588 - protein, 9 aa, Homo sapiens
FGFPDTSFY

SEQ ID NO: 589 - protein, 9 aa, Homo sapiens
AANTGKSLF

SEQ ID NO: 590 - protein, 9 aa, Homo sapiens
SFLTALKRF

SEQ ID NO: 591 - protein, 9 aa, Homo sapiens
FVNSKSHFW

SEQ ID NO: 592 - protein, 9 aa, Homo sapiens
LYTAVSSTW

SEQ ID NO: 593 - protein, 9 aa, Homo sapiens
QYGSMGVFF

SEQ ID NO: 594 - protein, 9 aa, Homo sapiens
GYIPANTTI

SEQ ID NO: 595 - protein, 9 aa, Homo sapiens
HYTNWTHIY

SEQ ID NO: 596 - protein, 9 aa, Homo sapiens
VTTHNNPTF

SEQ ID NO: 597 - protein, 9 aa, Homo sapiens
IHLPDPNKF

SEQ ID NO: 598 - protein, 9 aa, Homo sapiens
TIADQILQY

SEQ ID NO: 599 - protein, 9 aa, Homo sapiens
TTIPFGGAY

SEQ ID NO: 600 - protein, 9 aa, Homo sapiens
KFGFPDTSF

SEQ ID NO: 601 - protein, 9 aa, Homo sapiens
QAIELQLTL

SEQ ID NO: 602 - protein, 9 aa, Homo sapiens
SIADSIKTL

SEQ ID NO: 603 - protein, 9 aa, Homo sapiens
YVARTNIYY

SEQ ID NO: 604 - protein, 9 aa, Homo sapiens
LQTITPSTY

SEQ ID NO: 605 - protein, 9 aa, Homo sapiens
YQGVEFMSF

SEQ ID NO: 606 - protein, 9 aa, Homo sapiens
YKNTNFKEY

SEQ ID NO: 607 - protein, 9 aa, Homo sapiens
QYSNEKWTL

SEQ ID NO: 608 - protein, 9 aa, Homo sapiens
LQYGSMGVF

SEQ ID NO: 609 - protein, 9 aa, Homo sapiens
GTLEDTYRF

SEQ ID NO: 610 - protein, 10 aa, Homo sapiens
YVHEGIRTYF

SEQ ID NO: 611 - protein, 9 aa, Homo sapiens
FLRYQGVEF

SEQ ID NO: 612 - protein, 9 aa, Homo sapiens
HYYYDLSTI

SEQ ID NO: 613 - protein, 9 aa, Homo sapiens
ALDGNLVSM

SEQ ID NO: 614 - protein, 9 aa, Homo sapiens
TAVSSTWHW

SEQ ID NO: 615 - protein, 10 aa, Homo sapiens
LQYGSMGVFF

SEQ ID NO: 616 - protein, 10 aa, Homo sapiens
IYYHAGTSRL

SEQ ID NO: 617 - protein, 9 aa, Homo sapiens
FLIHTHARF

SEQ ID NO: 618 - protein, 11 aa, Homo sapiens
YYVHEGIRTYF

SEQ ID NO: 619 - protein, 9 aa, Homo sapiens
DQFPLGRKF

SEQ ID NO: 620 - protein, 9 aa, Homo sapiens
YFPTPSGSM

SEQ ID NO: 621 - protein, 8 aa, Homo sapiens
FYSKISEY

SEQ ID NO: 622 - protein, 9 aa, Homo sapiens
KYPDYIKMV

SEQ ID NO: 623 - protein, 11 aa, Homo sapiens
GYIPANTTIPF

SEQ ID NO: 624 - protein, 9 aa, Homo sapiens
FFYLRREQM

SEQ ID NO: 625 - protein, 9 aa, Homo sapiens
LYGVSFSEL

SEQ ID NO: 626 - protein, 9 aa, Homo sapiens
SMDYKQTQL

SEQ ID NO: 627 - protein, 9 aa, Homo sapiens
VSLVEETSF

SEQ ID NO: 628 - protein, 9 aa, Homo sapiens
DSAPILTAF

SEQ ID NO: 629 - protein, 9 aa, Homo sapiens
IADSIKTLL

SEQ ID NO: 630 - protein, 9 aa, Homo sapiens
LTADVMTYI

SEQ ID NO: 631 - protein, 10 aa, Homo sapiens
NYFPTPSGSM

SEQ ID NO: 632 - protein, 9 aa, Homo sapiens
AFVTTPTKL

SEQ ID NO: 633 - protein, 9 aa, Homo sapiens
TLQDVSLEV

SEQ ID NO: 634 - protein, 9 aa, Homo sapiens
TTPTKLITY

SEQ ID NO: 635 - protein, 9 aa, Homo sapiens
SIKTLLQQY

SEQ ID NO: 636 - protein, 10 aa, Homo sapiens
YYHAGTSRLL

SEQ ID NO: 637 - protein, 9 aa, Homo sapiens
TAASAFRCF

SEQ ID NO: 638 - protein, 8 aa, Homo sapiens
FYLHPSYY

SEQ ID NO: 639 - protein, 9 aa, Homo sapiens
HGYTVEVQF

SEQ ID NO: 640 - protein, 9 aa, Homo sapiens
SKISEYRHY

SEQ ID NO: 641 - protein, 10 aa, Homo sapiens
IYYKAREMGF

SEQ ID NO: 642 - protein, 9 aa, Homo sapiens
ILVPKVSGL

SEQ ID NO: 643 - protein, 9 aa, Homo sapiens
RSTAAALYW

SEQ ID NO: 644 - protein, 10 aa, Homo sapiens
NYEEIPMDTF

SEQ ID NO: 645 - protein, 10 aa, Homo sapiens
FYLHPSYYML

SEQ ID NO: 646 - protein, 9 aa, Homo sapiens
KQIVMFLRY

SEQ ID NO: 647 - protein, 8 aa, Homo sapiens
LYLHIQSL

SEQ ID NO: 648 - protein, 10 aa, Homo sapiens
SFYNPDTQRL

SEQ ID NO: 649 - protein, 9 aa, Homo sapiens
YSKNKVWEV

SEQ ID NO: 650 - protein, 10 aa, Homo sapiens
GQAEPDRAHY

SEQ ID NO: 651 - protein, 10 aa, Homo sapiens
VDSAPILTAF

SEQ ID NO: 652 - protein, 9 aa, Homo sapiens
KRASATQLY

SEQ ID NO: 653 - protein, 8 aa, Homo sapiens
IYYKAREM

SEQ ID NO: 654 - protein, 9 aa, Homo sapiens
AQIFNKPYW

SEQ ID NO: 655 - protein, 9 aa, Homo sapiens
CYSLYGTTL

SEQ ID NO: 656 - protein, 10 aa, Homo sapiens
YLPPVPVSKV

SEQ ID NO: 657 - protein, 9 aa, Homo sapiens
GQVDYYGLY

SEQ ID NO: 658 - protein, 11 aa, Homo sapiens
IYNSQYSNEKW

SEQ ID NO: 659 - protein, 9 aa, Homo sapiens
IVDDSEIAY

SEQ ID NO: 660 - protein, 9 aa, Homo sapiens
VTLTYDSEW

SEQ ID NO: 661 - protein, 9 aa, Homo sapiens
EQMFVRHLF

SEQ ID NO: 662 - protein, 9 aa, Homo sapiens
SFNDCTFEL

SEQ ID NO: 663 - protein, 9 aa, Homo sapiens
RHLDKKQRF

SEQ ID NO: 664 - protein, 11 aa, Homo sapiens
FYNPDTQRLVW

SEQ ID NO: 665 - protein, 9 aa, Homo sapiens
AILDINNTV

SEQ ID NO: 666 - protein, 8 aa, Homo sapiens
QFPLGRKF

SEQ ID NO: 667 - protein, 9 aa, Homo sapiens
CFVNSKSHF

SEQ ID NO: 668 - protein, 9 aa, Homo sapiens
KHKSAIVTL

SEQ ID NO: 669 - protein, 9 aa, Homo sapiens
VYDPLQPEL

SEQ ID NO: 670 - protein, 9 aa, Homo sapiens
YLQPRTFLL

SEQ ID NO: 671 - protein, 9 aa, Homo sapiens
TLDSKTQSL

SEQ ID NO: 672 - protein, 9 aa, Homo sapiens
KLFDRYFKY

SEQ ID NO: 673 - protein, 9 aa, Homo sapiens
KVDGVVQQL

SEQ ID NO: 674 - protein, 9 aa, Homo sapiens
FLLNKEMYL

SEQ ID NO: 675 - protein, 9 aa, Homo sapiens
LMIERFVSL

SEQ ID NO: 676 - protein, 9 aa, Homo sapiens
FLNRFTTTL

SEQ ID NO: 677 - protein, 9 aa, Homo sapiens
LLADKFPVL

SEQ ID NO: 678 - protein, 9 aa, Homo sapiens
YLITPVHVM

SEQ ID NO: 679 - protein, 9 aa, Homo sapiens
VRFPNITNL

SEQ ID NO: 680 - protein, 9 aa, Homo sapiens
YYTSNPTTF

SEQ ID NO: 681 - protein, 9 aa, Homo sapiens
IRQEEVQEL

SEQ ID NO: 682 - protein, 9 aa, Homo sapiens
YITGGVVQL

SEQ ID NO: 683 - protein, 9 aa, Homo sapiens
LLLDRLNQL

SEQ ID NO: 684 - protein, 9 aa, Homo sapiens
VLNDILSRL

SEQ ID NO: 685 - protein, 9 aa, Homo sapiens
FLLPSLATV

SEQ ID NO: 686 - protein, 9 aa, Homo sapiens
TMADLVYAL

SEQ ID NO: 687 - protein, 9 aa, Homo sapiens
VPMEKLKTL

SEQ ID NO: 688 - protein, 9 aa, Homo sapiens
FGADPIHSL

SEQ ID NO: 689 - protein, 9 aa, Homo sapiens
VRDPQTLEI

SEQ ID NO: 690 - protein, 9 aa, Homo sapiens
YVYSRVKNL

SEQ ID NO: 691 - protein, 9 aa, Homo sapiens
YLATALLTL

SEQ ID NO: 692 - protein, 9 aa, Homo sapiens
VLSDRELHL

SEQ ID NO: 693 - protein, 9 aa, Homo sapiens
NLSDRVVFV

SEQ ID NO: 694 - protein, 9 aa, Homo sapiens
FVDGVPFVV

SEQ ID NO: 695 - protein, 9 aa, Homo sapiens
KPNELSRVL

SEQ ID NO: 696 - protein, 9 aa, Homo sapiens
VMYMGTLSY

SEQ ID NO: 697 - protein, 9 aa, Homo sapiens
LLYDANYFL

SEQ ID NO: 698 - protein, 9 aa, Homo sapiens
TLNDLNETL

SEQ ID NO: 699 - protein, 10 aa, Homo sapiens
QEYADVFHLY

SEQ ID NO: 700 - protein, 9 aa, Homo sapiens
TVYSHLLLV

SEQ ID NO: 701 - protein, 9 aa, Homo sapiens
SEFSSLPSY

SEQ ID NO: 702 - protein, 9 aa, Homo sapiens
YLDAYNMMI

SEQ ID NO: 703 - protein, 9 aa, Homo sapiens
RPDTRYVLM

SEQ ID NO: 704 - protein, 9 aa, Homo sapiens
KIADYNYKL

SEQ ID NO: 705 - protein, 9 aa, Homo sapiens
FVNEFYAYL

SEQ ID NO: 706 - protein, 9 aa, Homo sapiens
RLYYDSMSY

SEQ ID NO: 707 - protein, 9 aa, Homo sapiens
VLLSVLQQL

SEQ ID NO: 708 - protein, 9 aa, Homo sapiens
ALSKGVHFV

SEQ ID NO: 709 - protein, 9 aa, Homo sapiens
EEFEPSTQY

SEQ ID NO: 710 - protein, 9 aa, Homo sapiens
TLVPQEHYV

SEQ ID NO: 711 - protein, 9 aa, Homo sapiens
IPRRNVATL

SEQ ID NO: 712 - protein, 9 aa, Homo sapiens
KTFPPTEPK

SEQ ID NO: 713 - protein, 9 aa, Homo sapiens
NYMPYFFTL

SEQ ID NO: 714 - protein, 9 aa, Homo sapiens
STFNVPMEK

SEQ ID NO: 715 - protein, 9 aa, Homo sapiens
HPTQAPTHL

SEQ ID NO: 716 - protein, 11 aa, Homo sapiens
AEIPKEEVKPF

SEQ ID NO: 717 - protein, 9 aa, Homo sapiens
ALWEIQQVV

SEQ ID NO: 718 - protein, 9 aa, Homo sapiens
KPVETSNSF

SEQ ID NO: 719 - protein, 9 aa, Homo sapiens
VLWAHGFEL

SEQ ID NO: 720 - protein, 9 aa, Homo sapiens
VVYRGTTTY

SEQ ID NO: 721 - protein, 9 aa, Homo sapiens
GLNDNLLEI

SEQ ID NO: 722 - protein, 9 aa, Homo sapiens
VPHISRQRL

SEQ ID NO: 723 - protein, 9 aa, Homo sapiens
VENPHLMGW

SEQ ID NO: 724 - protein, 9 aa, Homo sapiens
QEILGTVSW

SEQ ID NO: 725 - protein, 9 aa, Homo sapiens
HLVDFQVTI

SEQ ID NO: 726 - protein, 9 aa, Homo sapiens
GEYSHVVAF

SEQ ID NO: 727 - protein, 11 aa, Homo sapiens
YLFDESGEFKL

SEQ ID NO: 728 - protein, 9 aa, Homo sapiens
REHEHEIAW

SEQ ID NO: 729 - protein, 10 aa, Homo sapiens
ASEFSSLPSY

SEQ ID NO: 730 - protein, 9 aa, Homo sapiens
KLVNKFLAL

SEQ ID NO: 731 - protein, 9 aa, Homo sapiens
YLNTLTLAV

SEQ ID NO: 732 - protein, 9 aa, Homo sapiens
ILFTRFFYV

SEQ ID NO: 733 - protein, 9 aa, Homo sapiens
YLNSTNVTI

SEQ ID NO: 734 - protein, 10 aa, Homo sapiens
KTFPPTEPKK

SEQ ID NO: 735 - protein, 8 aa, Homo sapiens
TIKPVTYK

SEQ ID NO: 736 - protein, 9 aa, Homo sapiens
VRIQPGQTF

SEQ ID NO: 737 - protein, 9 aa, Homo sapiens
LLLDDFVEI

SEQ ID NO: 738 - protein, 9 aa, Homo sapiens
YYQLYSTQL

SEQ ID NO: 739 - protein, 9 aa, Homo sapiens
KTIQPRVEK

SEQ ID NO: 740 - protein, 9 aa, Homo sapiens
KRVDWTIEY

SEQ ID NO: 741 - protein, 9 aa, Homo sapiens
RLFRKSNLK

SEQ ID NO: 742 - protein, 8 aa, Homo sapiens
YPDPSRIL

SEQ ID NO: 743 - protein, 9 aa, Homo sapiens
ASMPTTIAK

SEQ ID NO: 744 - protein, 9 aa, Homo sapiens
FPRGQGVPI

SEQ ID NO: 745 - protein, 9 aa, Homo sapiens
RTAPHGHVM

SEQ ID NO: 746 - protein, 10 aa, Homo sapiens
LPYPDPSRIL

SEQ ID NO: 747 - protein, 10 aa, Homo sapiens
AVFDKNLYDK

SEQ ID NO: 748 - protein, 9 aa, Homo sapiens
QELGKYEQY

SEQ ID NO: 749 - protein, 9 aa, Homo sapiens
FLAHIQWMV

SEQ ID NO: 750 - protein, 9 aa, Homo sapiens
SEVGPEHSL

SEQ ID NO: 751 - protein, 9 aa, Homo sapiens
KLFAAETLK

SEQ ID NO: 752 - protein, 10 aa, Homo sapiens
VVYRGTTTYK

SEQ ID NO: 753 - protein, 9 aa, Homo sapiens
TSFGPLVRK

SEQ ID NO: 754 - protein, 10 aa, Homo sapiens
SEPVLKGVKL

SEQ ID NO: 755 - protein, 10 aa, Homo sapiens
KEIKESVQTF

SEQ ID NO: 756 - protein, 9 aa, Homo sapiens
SQLGGLHLL

SEQ ID NO: 757 - protein, 11 aa, Homo sapiens
APRTLLTKGTL

SEQ ID NO: 758 - protein, 9 aa, Homo sapiens
WRNTNPIQL

SEQ ID NO: 759 - protein, 10 aa, Homo sapiens
EEAIRHVRAW

SEQ ID NO: 760 - protein, 10 aa, Homo sapiens
TEKSNIIRGW

SEQ ID NO: 761 - protein, 9 aa, Homo sapiens
RQFHQKLLK

SEQ ID NO: 762 - protein, 10 aa, Homo sapiens
APHGHVMVEL

SEQ ID NO: 763 - protein, 9 aa, Homo sapiens
QIYKTPPIK

SEQ ID NO: 764 - protein, 9 aa, Homo sapiens
SVVSKVVKV

SEQ ID NO: 765 - protein, 9 aa, Homo sapiens
KENDSKEGF

SEQ ID NO: 766 - protein, 9 aa, Homo sapiens
KSAGFPFNK

SEQ ID NO: 767 - protein, 9 aa, Homo sapiens
MEVTPSGTW

SEQ ID NO: 768 - protein, 9 aa, Homo sapiens
TLMNVLTLV

SEQ ID NO: 769 - protein, 9 aa, Homo sapiens
KVDGVVQQL

SEQ ID NO: 770 - protein, 9 aa, Homo sapiens
YLQPRTFLL

SEQ ID NO: 771 - protein, 9 aa, Homo sapiens
TLDSKTQSL

SEQ ID NO: 772 - protein, 9 aa, Homo sapiens
TQYNRYLAL

SEQ ID NO: 773 - protein, 9 aa, Homo sapiens
TQAPTHLSV

SEQ ID NO: 774 - protein, 9 aa, Homo sapiens
FVDGVPFVV

SEQ ID NO: 775 - protein, 9 aa, Homo sapiens
SQLGGLHLL

SEQ ID NO: 776 - protein, 9 aa, Homo sapiens
AQLPAPRTL

SEQ ID NO: 777 - protein, 9 aa, Homo sapiens
FGADPIHSL

SEQ ID NO: 778 - protein, 9 aa, Homo sapiens
TVYSHLLLV

SEQ ID NO: 779 - protein, 9 aa, Homo sapiens
LMIERFVSL

SEQ ID NO: 780 - protein, 9 aa, Homo sapiens
FLNRFTTTL

SEQ ID NO: 781 - protein, 9 aa, Homo sapiens
ALWEIQQVV

SEQ ID NO: 782 - protein, 9 aa, Homo sapiens
YVYSRVKNL

SEQ ID NO: 783 - protein, 9 aa, Homo sapiens
HLVDFQVTI

SEQ ID NO: 784 - protein, 9 aa, Homo sapiens
YIDIGNYTV

SEQ ID NO: 785 - protein, 9 aa, Homo sapiens
YLNSTNVTI

SEQ ID NO: 786 - protein, 9 aa, Homo sapiens
VRFPNITNL

SEQ ID NO: 787 - protein, 9 aa, Homo sapiens
YVDNSSLTI

SEQ ID NO: 788 - protein, 9 aa, Homo sapiens
RLDKVEAEV

SEQ ID NO: 789 - protein, 9 aa, Homo sapiens
YLDAYNMMI

SEQ ID NO: 790 - protein, 9 aa, Homo sapiens
KVDGVDVEL

SEQ ID NO: 791 - protein, 9 aa, Homo sapiens
VLNDILSRL

SEQ ID NO: 792 - protein, 9 aa, Homo sapiens
SVVSKVVKV

SEQ ID NO: 793 - protein, 9 aa, Homo sapiens
ALSKGVHFV

SEQ ID NO: 794 - protein, 9 aa, Homo sapiens
RLQSLQTYV

SEQ ID NO: 795 - protein, 9 aa, Homo sapiens
TRFQTLLAL

SEQ ID NO: 796 - protein, 9 aa, Homo sapiens
GLNDNLLEI

SEQ ID NO: 797 - protein, 9 aa, Homo sapiens
FLLPSLATV

SEQ ID NO: 798 - protein, 9 aa, Homo sapiens
KLFIRQEEV

SEQ ID NO: 799 - protein, 9 aa, Homo sapiens
TMADLVYAL

SEQ ID NO: 800 - protein, 9 aa, Homo sapiens
RQLLFVVEV

SEQ ID NO: 801 - protein, 9 aa, Homo sapiens
YLITPVHVM

SEQ ID NO: 802 - protein, 9 aa, Homo sapiens
FLLNKEMYL

SEQ ID NO: 803 - protein, 9 aa, Homo sapiens
NRFLYIIKL

SEQ ID NO: 804 - protein, 9 aa, Homo sapiens
KLFDRYFKY

SEQ ID NO: 805 - protein, 9 aa, Homo sapiens
KLNEEIAII

SEQ ID NO: 806 - protein, 9 aa, Homo sapiens
KLLEQWNLV

SEQ ID NO: 807 - protein, 9 aa, Homo sapiens
IVDEPEEHV

SEQ ID NO: 808 - protein, 9 aa, Homo sapiens
LLADKFPVL

SEQ ID NO: 809 - protein, 9 aa, Homo sapiens
VLLSVLQQL

SEQ ID NO: 810 - protein, 9 aa, Homo sapiens
KLSYGIATV

SEQ ID NO: 811 - protein, 9 aa, Homo sapiens
FGDDTVIEV

SEQ ID NO: 812 - protein, 9 aa, Homo sapiens
YLATALLTL

SEQ ID NO: 813 - protein, 9 aa, Homo sapiens
TLVPQEHYV

SEQ ID NO: 814 - protein, 9 aa, Homo sapiens
TLNDLNETL

SEQ ID NO: 815 - protein, 9 aa, Homo sapiens
ERSEKSYEL

SEQ ID NO: 816 - protein, 9 aa, Homo sapiens
KIADYNYKL

SEQ ID NO: 817 - protein, 9 aa, Homo sapiens
YQPYRVVVL

SEQ ID NO: 818 - protein, 9 aa, Homo sapiens
YADVFHLYL

SEQ ID NO: 819 - protein, 9 aa, Homo sapiens
LLLDRLNQL

SEQ ID NO: 820 - protein, 9 aa, Homo sapiens
TLMNVLTLV

SEQ ID NO: 821 - protein, 9 aa, Homo sapiens
YLNTLTLAV

SEQ ID NO: 822 - protein, 9 aa, Homo sapiens
YAKPFLNKV

SEQ ID NO: 823 - protein, 9 aa, Homo sapiens
YITGGVVQL

SEQ ID NO: 824 - protein, 9 aa, Homo sapiens
VRIKIVQML

SEQ ID NO: 825 - protein, 9 aa, Homo sapiens
SMWALIISV

SEQ ID NO: 826 - protein, 9 aa, Homo sapiens
VLSDRELHL

SEQ ID NO: 827 - protein, 9 aa, Homo sapiens
VYDPLQPEL

SEQ ID NO: 828 - protein, 9 aa, Homo sapiens
MLDMYSVML

SEQ ID NO: 829 - protein, 9 aa, Homo sapiens
FRYMNSQGL

SEQ ID NO: 830 - protein, 9 aa, Homo sapiens
HLDGEVITF

SEQ ID NO: 831 - protein, 9 aa, Homo sapiens
KQLIKVTLV

SEQ ID NO: 832 - protein, 9 aa, Homo sapiens
KLDDKDPNF

SEQ ID NO: 833 - protein, 9 aa, Homo sapiens
IRQEEVQEL

SEQ ID NO: 834 - protein, 9 aa, Homo sapiens
LMDGSIIQF

SEQ ID NO: 835 - protein, 9 aa, Homo sapiens
RLNEVAKNL

SEQ ID NO: 836 - protein, 9 aa, Homo sapiens
SLLMPILTL

SEQ ID NO: 837 - protein, 9 aa, Homo sapiens
VADAVIKTL

SEQ ID NO: 838 - protein, 9 aa, Homo sapiens
VLWAHGFEL

SEQ ID NO: 839 - protein, 9 aa, Homo sapiens
LLLDDFVEI

SEQ ID NO: 840 - protein, 9 aa, Homo sapiens
VRSIFSRTL

SEQ ID NO: 841 - protein, 9 aa, Homo sapiens
NLSDRVVFV

SEQ ID NO: 842 - protein, 9 aa, Homo sapiens
VVFLHVTYV

SEQ ID NO: 843 - protein, 9 aa, Homo sapiens
LQLPQGTTL

SEQ ID NO: 844 - protein, 9 aa, Homo sapiens
SLVKPSFYV

SEQ ID NO: 845 - protein, 9 aa, Homo sapiens
KLVNKFLAL

SEQ ID NO: 846 - protein, 9 aa, Homo sapiens
ARYMRSLKV

SEQ ID NO: 847 - protein, 9 aa, Homo sapiens
LLYDANYFL

SEQ ID NO: 848 - protein, 9 aa, Homo sapiens
IQPGQTFSV

SEQ ID NO: 849 - protein, 9 aa, Homo sapiens
DADSKIVQL

SEQ ID NO: 850 - protein, 9 aa, Homo sapiens
EAFEKMVSL

SEQ ID NO: 851 - protein, 9 aa, Homo sapiens
WRNTNPIQL

SEQ ID NO: 852 - protein, 9 aa, Homo sapiens
AMYTPHTVL

SEQ ID NO: 853 - protein, 9 aa, Homo sapiens
QQGEVPVSI

SEQ ID NO: 854 - protein, 9 aa, Homo sapiens
LQAENVTGL

SEQ ID NO: 855 - protein, 9 aa, Homo sapiens
FQSAVKRTI

SEQ ID NO: 856 - protein, 9 aa, Homo sapiens
QLSLPVLQV

SEQ ID NO: 857 - protein, 9 aa, Homo sapiens
SLDNVLSTF

SEQ ID NO: 858 - protein, 9 aa, Homo sapiens
FLAHIQWMV

SEQ ID NO: 859 - protein, 9 aa, Homo sapiens
HANEYRLYL

SEQ ID NO: 860 - protein, 9 aa, Homo sapiens
QMAPISAMV

SEQ ID NO: 861 - protein, 9 aa, Homo sapiens
EQFKKGVQI

SEQ ID NO: 862 - protein, 9 aa, Homo sapiens
FVNEFYAYL

SEQ ID NO: 863 - protein, 9 aa, Homo sapiens
YRLFRKSNL

SEQ ID NO: 864 - protein, 9 aa, Homo sapiens
NVFAFPFTI

SEQ ID NO: 865 - protein, 9 aa, Homo sapiens
ILFTRFFYV

SEQ ID NO: 866 - protein, 9 aa, Homo sapiens
KLWAQCVQL

SEQ ID NO: 867 - protein, 9 aa, Homo sapiens
TLQPVSELL

SEQ ID NO: 868 - protein, 10 aa, Homo sapiens
ILDGISQYSL

SEQ ID NO: 869 - protein, 9 aa, Homo sapiens
KAYNVTQAF

SEQ ID NO: 870 - protein, 9 aa, Homo sapiens
KSHKPPISF

SEQ ID NO: 871 - protein, 9 aa, Homo sapiens
QSAPHGVVF

SEQ ID NO: 872 - protein, 9 aa, Homo sapiens
RTAPHGHVM

SEQ ID NO: 873 - protein, 9 aa, Homo sapiens
YVNTFSSTF

SEQ ID NO: 874 - protein, 9 aa, Homo sapiens
YLITPVHVM

SEQ ID NO: 875 - protein, 9 aa, Homo sapiens
YYTSNPTTF

SEQ ID NO: 876 - protein, 9 aa, Homo sapiens
VARDLSLQF

SEQ ID NO: 877 - protein, 9 aa, Homo sapiens
STVFPPTSF

SEQ ID NO: 878 - protein, 9 aa, Homo sapiens
YYHTTDPSF

SEQ ID NO: 879 - protein, 9 aa, Homo sapiens
LYDKLVSSF

SEQ ID NO: 880 - protein, 9 aa, Homo sapiens
VYDPLQPEL

SEQ ID NO: 881 - protein, 9 aa, Homo sapiens
VVYRGTTTY

SEQ ID NO: 882 - protein, 9 aa, Homo sapiens
MSMTYGQQF

SEQ ID NO: 883 - protein, 9 aa, Homo sapiens
TANPKTPKY

SEQ ID NO: 884 - protein, 9 aa, Homo sapiens
KAYKIEELF

SEQ ID NO: 885 - protein, 9 aa, Homo sapiens
KSYELQTPF

SEQ ID NO: 886 - protein, 9 aa, Homo sapiens
SSLPSYAAF

SEQ ID NO: 887 - protein, 9 aa, Homo sapiens
FAYANRNRF

SEQ ID NO: 888 - protein, 9 aa, Homo sapiens
YYKKDNSYF

SEQ ID NO: 889 - protein, 9 aa, Homo sapiens
YFIASFRLF

SEQ ID NO: 890 - protein, 9 aa, Homo sapiens
SFYEDFLEY

SEQ ID NO: 891 - protein, 9 aa, Homo sapiens
KVDGVVQQL

SEQ ID NO: 892 - protein, 9 aa, Homo sapiens
LAKDTTEAF

SEQ ID NO: 893 - protein, 9 aa, Homo sapiens
VATSRTLSY

SEQ ID NO: 894 - protein, 9 aa, Homo sapiens
KQFDTYNLW

SEQ ID NO: 895 - protein, 10 aa, Homo sapiens
VYYTSNPTTF

SEQ ID NO: 896 - protein, 9 aa, Homo sapiens
YFVVKRHTF

SEQ ID NO: 897 - protein, 9 aa, Homo sapiens
LMDGSIIQF

SEQ ID NO: 898 - protein, 9 aa, Homo sapiens
VYSTGSNVF

SEQ ID NO: 899 - protein, 9 aa, Homo sapiens
IYNDKVAGF

SEQ ID NO: 900 - protein, 9 aa, Homo sapiens
YLQPRTFLL

SEQ ID NO: 901 - protein, 9 aa, Homo sapiens
MFDAYVNTF

SEQ ID NO: 902 - protein, 9 aa, Homo sapiens
YLFDESGEF

SEQ ID NO: 903 - protein, 9 aa, Homo sapiens
QFAPSASAF

SEQ ID NO: 904 - protein, 9 aa, Homo sapiens
GYKSVNITF

SEQ ID NO: 905 - protein, 9 aa, Homo sapiens
FYLTNDVSF

SEQ ID NO: 906 - protein, 9 aa, Homo sapiens
VYFLQSINF

SEQ ID NO: 907 - protein, 9 aa, Homo sapiens
VASQSIIAY

SEQ ID NO: 908 - protein, 9 aa, Homo sapiens
AQLPAPRTL

SEQ ID NO: 909 - protein, 9 aa, Homo sapiens
VYMPASWVM

SEQ ID NO: 910 - protein, 9 aa, Homo sapiens
SAKSASVYY

SEQ ID NO: 911 - protein, 9 aa, Homo sapiens
TTLPVNVAF

SEQ ID NO: 912 - protein, 9 aa, Homo sapiens
VVIPDYNTY

SEQ ID NO: 913 - protein, 9 aa, Homo sapiens
SSVELKHFF

SEQ ID NO: 914 - protein, 9 aa, Homo sapiens
SQLGGLHLL

SEQ ID NO: 915 - protein, 9 aa, Homo sapiens
VAMPNLYKM

SEQ ID NO: 916 - protein, 9 aa, Homo sapiens
NYMPYFFTL

SEQ ID NO: 917 - protein, 9 aa, Homo sapiens
MFVKHKHAF

SEQ ID NO: 918 - protein, 9 aa, Homo sapiens
VYIGDPAQL

SEQ ID NO: 919 - protein, 9 aa, Homo sapiens
TFFKLVNKF

SEQ ID NO: 920 - protein, 9 aa, Homo sapiens
RYKLEGYAF

SEQ ID NO: 921 - protein, 9 aa, Homo sapiens
ASRELKVTF

SEQ ID NO: 922 - protein, 9 aa, Homo sapiens
KHWPQIAQF

SEQ ID NO: 923 - protein, 9 aa, Homo sapiens
FAVDAAKAY

SEQ ID NO: 924 - protein, 9 aa, Homo sapiens
TTNGDFLHF

SEQ ID NO: 925 - protein, 9 aa, Homo sapiens
LYQPPQTSI

SEQ ID NO: 926 - protein, 9 aa, Homo sapiens
KALNLGETF

SEQ ID NO: 927 - protein, 10 aa, Homo sapiens
AQFAPSASAF

SEQ ID NO: 928 - protein, 9 aa, Homo sapiens
IQLSSYSLF

SEQ ID NO: 929 - protein, 9 aa, Homo sapiens
EYADVFHLY

SEQ ID NO: 930 - protein, 9 aa, Homo sapiens
YTPSKLIEY

SEQ ID NO: 931 - protein, 9 aa, Homo sapiens
YYQLYSTQL

SEQ ID NO: 932 - protein, 9 aa, Homo sapiens
ATIPIQASL

SEQ ID NO: 933 - protein, 9 aa, Homo sapiens
SIIQFPNTY

SEQ ID NO: 934 - protein, 9 aa, Homo sapiens
MYASAVVLL

SEQ ID NO: 935 - protein, 9 aa, Homo sapiens
YYRSLPGVF

SEQ ID NO: 936 - protein, 9 aa, Homo sapiens
KLFDRYFKY

SEQ ID NO: 937 - protein, 9 aa, Homo sapiens
RFKESPFEL

SEQ ID NO: 938 - protein, 9 aa, Homo sapiens
LVSDIDITF

SEQ ID NO: 939 - protein, 9 aa, Homo sapiens
YQKVGMQKY

SEQ ID NO: 940 - protein, 9 aa, Homo sapiens
RTIKGTHHW

SEQ ID NO: 941 - protein, 9 aa, Homo sapiens
ITFDNLKTL

SEQ ID NO: 942 - protein, 9 aa, Homo sapiens
LMIERFVSL

SEQ ID NO: 943 - protein, 10 aa, Homo sapiens
KMFDAYVNTF

SEQ ID NO: 944 - protein, 9 aa, Homo sapiens
VMYMGTLSY

SEQ ID NO: 945 - protein, 9 aa, Homo sapiens
SYATHSDKF

SEQ ID NO: 946 - protein, 9 aa, Homo sapiens
KFYGGWHNM

SEQ ID NO: 947 - protein, 9 aa, Homo sapiens
SLYVNKHAF

SEQ ID NO: 948 - protein, 9 aa, Homo sapiens
FGADPIHSL

SEQ ID NO: 949 - protein, 9 aa, Homo sapiens
SFSASTSAF

SEQ ID NO: 950 - protein, 9 aa, Homo sapiens
LALSKGVHF

SEQ ID NO: 951 - protein, 9 aa, Homo sapiens
RFDNPVLPF

SEQ ID NO: 952 - protein, 9 aa, Homo sapiens
AMYTPHTVL

SEQ ID NO: 953 - protein, 9 aa, Homo sapiens
FLNRFTTTL

SEQ ID NO: 954 - protein, 9 aa, Homo sapiens
TYFTQSRNL

SEQ ID NO: 955 - protein, 8 aa, Homo sapiens
YAFEHIVY

SEQ ID NO: 956 - protein, 9 aa, Homo sapiens
LQIPFAMQM

SEQ ID NO: 957 - protein, 9 aa, Homo sapiens
AYILFTRFF

SEQ ID NO: 958 - protein, 10 aa, Homo sapiens
YYLFDESGEF

SEQ ID NO: 959 - protein, 10 aa, Homo sapiens
YSYATHSDKF

SEQ ID NO: 960 - protein, 11 aa, Homo sapiens
KFVRIQPGQTF

SEQ ID NO: 961 - protein, 9 aa, Homo sapiens
FTSDYYQLY

SEQ ID NO: 962 - protein, 9 aa, Homo sapiens
VYANGGKGF

SEQ ID NO: 963 - protein, 11 aa, Homo sapiens
SVYYTSNPTTF

SEQ ID NO: 964 - protein, 9 aa, Homo sapiens
YFIKGLNNL

SEQ ID NO: 965 - protein, 9 aa, Homo sapiens
TLKEILVTY

SEQ ID NO: 966 - protein, 9 aa, Homo sapiens
LYLQYIRKL

SEQ ID NO: 967 - protein, 9 aa, Homo sapiens
KHIDAYKTF

SEQ ID NO: 968 - protein, 9 aa, Homo sapiens
RSFIEDLLF

SEQ ID NO: 969 - protein, 10 aa, synthetic construct
ADLDDFSKQL

SEQ ID NO: 970 - protein, 9 aa, synthetic construct
AFPFTIYSL

SEQ ID NO: 971 - protein, 9 aa, synthetic construct
AHAEETRKL

SEQ ID NO: 972 - protein, 10 aa, synthetic construct
ALLADKFPVL

SEQ ID NO: 973 - protein, 9 aa, synthetic construct
ALVYFLQSI

SEQ ID NO: 974 - protein, 9 aa, synthetic construct
ALWEIQQVV

SEQ ID NO: 975 - protein, 10 aa, synthetic construct
ANPKTPKYKF

SEQ ID NO: 976 - protein, 9 aa, synthetic construct
APFLYLYAL

SEQ ID NO: 977 - protein, 8 aa, synthetic construct
APHGVVFL

SEQ ID NO: 978 - protein, 10 aa, synthetic construct
APHGVVFLHV

SEQ ID NO: 979 - protein, 11 aa, synthetic construct
AADLDDFSKQL

SEQ ID NO: 980 - protein, 10 aa, synthetic construct
ALLADKFPVL

SEQ ID NO: 981 - protein, 9 aa, synthetic construct
ALLAVFQSA

SEQ ID NO: 982 - protein, 9 aa, synthetic construct
ALQDAYYRA

SEQ ID NO: 983 - protein, 10 aa, synthetic construct
ALRKVPTDNY

SEQ ID NO: 984 - protein, 9 aa, synthetic construct
ALTCFSTQF

SEQ ID NO: 985 - protein, 9 aa, synthetic construct
ALWEIQQVV

SEQ ID NO: 986 - protein, 9 aa, synthetic construct
AMDEFIERY

SEQ ID NO: 987 - protein, 9 aa, synthetic construct
APISAMVRM

SEQ ID NO: 988 - protein, 8 aa, synthetic construct
APKEIIFL

SEQ ID NO: 989 - protein, 104 aa, synthetic construct
AFLLFLVLIFLAFLLFLVFYLCFLAFLFYLCFLAFLLIDFYLCFLAFIELSLIDFYLLIDFYLCFLLLFLVLIMLMIELSLIDFYSLIDFYLCFLYLCFLAFLL

SEQ ID NO: 990 - protein, 36 aa, synthetic construct
APHGVVFLAPHGVVFLHVGVVFLHVTYVVFLHVTYV

SEQ ID NO: 991 - protein, 46 aa, synthetic construct
ASQSIIAYTMRSVASQSIISIIAYTMSLSQSIIAYTMVASQSIIAY

SEQ ID NO: 992 - protein, 11 aa, synthetic construct
AYILFTRFFYV

SEQ ID NO: 993 - protein, 39 aa, synthetic construct
CELYHYQECVITLATCELYLITLATCELYTLATCELYHY

SEQ ID NO: 994 - protein, 29 aa, synthetic construct
CNDPFLGVYCNDPFLGVYYFCNDPFLGVY

SEQ ID NO: 995 - protein, 28 aa, synthetic construct
CTFEYVSQPFEYVSQPFLMFEYVSQPFL

SEQ ID NO: 996 - protein, 19 aa, synthetic construct
DLFMRIFTIMDLFMRIFTI

SEQ ID NO: 997 - protein, 32 aa, synthetic construct
ELIRQGTDYQELIRQGTDYQELIRQGTDYKHW

SEQ ID NO: 998 - protein, 28 aa, synthetic construct
FAYANRNRFLQFAYANRNRYANRNRFLY

SEQ ID NO: 999 - protein, 104 aa, synthetic construct; identical to SEQ ID NO: 989

SEQ ID NO: 1000 - protein, 66 aa, synthetic construct
AFPFTIYSLGYINVFAFPFINVFAFPFTIMGYINVFAFNVFAFPFTINVFAFPFTIYYINVFAFPF

SEQ ID NO: 1001 - protein, 9 aa, synthetic construct
AIMTRCLAV

SEQ ID NO: 1002 - protein, 18 aa, synthetic construct
ALNTLVKQLAQALNTLVK

SEQ ID NO: 1003 - protein, 37 aa, synthetic construct
APAHISTILIVNSVLLFLLLFLAFVVFLSVLLFLAFV

SEQ ID NO: 1004 - protein, 36 aa, synthetic construct; identical to SEQ ID NO: 990

SEQ ID NO: 1005 - protein, 19 aa, synthetic construct
APKEIIFLKEIIFLEGETL

SEQ ID NO: 1006 - protein, 46 aa, synthetic construct; identical to SEQ ID NO: 991

SEQ ID NO: 1007 - protein, 11 aa, synthetic construct
AYILFTRFFYV

SEQ ID NO: 1008 - protein, 39 aa, synthetic construct; identical to SEQ ID NO: 993

SEQ ID NO: 1009 - protein, 10 aa, synthetic construct
IELSLIDFYL

SEQ ID NO: 1010 - protein, 9 aa, synthetic construct
VVFLHVTYV

SEQ ID NO: 1011 - protein, 9 aa, synthetic construct
VASQSIIAY

SEQ ID NO: 1012 - protein, 11 aa, synthetic construct
AYILFTRFFYV

SEQ ID NO: 1013 - protein, 9 aa, synthetic construct
ITLATCELY

SEQ ID NO: 1014 - protein, 10 aa, synthetic construct
FCNDPFLGVY

SEQ ID NO: 1015 - protein, 9 aa, synthetic construct
FEYVSQPFL

SEQ ID NO: 1016 - protein, 9 aa, synthetic construct
DLFMRIFTI

SEQ ID NO: 1017 - protein, 9 aa, synthetic construct
ELIRQGTDY

SEQ ID NO: 1018 - protein, 9 aa, synthetic construct
FAYANRNRF

SEQ ID NO: 1019 - protein, 10 aa, synthetic construct
MIELSLIDFY

SEQ ID NO: 1020 - protein, 10 aa, synthetic construct
NVFAFPFTIY

SEQ ID NO: 1021 - protein, 9 aa, synthetic construct
AIMTRCLAV

SEQ ID NO: 1022 - protein, 9 aa, synthetic construct
ALNTLVKQL

SEQ ID NO: 1023 - protein, 9 aa, synthetic construct
SVLLFLAFV

SEQ ID NO: 1024 - protein, 9 aa, synthetic construct
VVFLHVTYV

SEQ ID NO: 1025 - protein, 11 aa, synthetic construct
KEIIFLEGETL

SEQ ID NO: 1026 - protein, 9 aa, synthetic construct
VASQSIIAY

SEQ ID NO: 1027 - protein, 11 aa, synthetic construct
AYILFTRFFYV

SEQ ID NO: 1028 - protein, 9 aa, synthetic construct
ITLATCELY

SEQ ID NO: 1029 - protein, 10 aa, synthetic construct
GRKKRRQRRR

SEQ ID NO: 1030 - protein, 8 aa, synthetic construct
RRRRRRRR

SEQ ID NO: 1031 - protein, 9 aa, synthetic construct
RRRRRRRRR

SEQ ID NO: 1032 - protein, 16 aa, synthetic construct
RQIKIWFQNRRMKWKK

SEQ ID NO: 1033 - protein, 18 aa, synthetic construct
KLALKLALKALKAALKLA

SEQ ID NO: 1034 - protein, 34 aa, synthetic construct
DAATATRGRSAASRPTERPRAPARSASRPRRPVD

SEQ ID NO: 1035 - protein, 27 aa, synthetic construct
GALFLGFLGAAGSTMGAWSQPKKKRKV

SEQ ID NO: 1036 - protein, 19 aa, synthetic construct
  MOD_RES 1: note = 1; note = Acetylation
  UNSURE 19: note = Cysteamine
GLWRALWRLLRSLWRLLWK

SEQ ID NO: 1037 - protein, 18 aa, synthetic construct
LLIILRRRIRKQAHAHSK

SEQ ID NO: 1038 - protein, 22 aa, synthetic construct
MVTVLFRRLRIRRASGPPRVRV

SEQ ID NO: 1039 - protein, 30 aa, synthetic construct
WEAKLAKALAKALAKHLAKALAKALKACEA

SEQ ID NO: 1040 - protein, 21 aa, synthetic construct
  MOD_RES 1: note = Acetylation
  UNSURE 21: note = Cysteamine
KETWWETWWTEWSQPKKKRKC

SEQ ID NO: 1041 - protein, 23 aa, synthetic construct
LIKLWSHLIHIWFQNRRLKWKKK

SEQ ID NO: 1042 - protein, 27 aa, synthetic construct
GWTLNSAGYLLGKINLKALAALAKKIL

SEQ ID NO: 1043 - protein, 16 aa, synthetic construct
RQIKIWFQNRRMKWKK

SEQ ID NO: 1044 - protein, 15 aa, synthetic construct
KFHTFPQTAIGVGAP

SEQ ID NO: 1045 - protein, 18 aa, synthetic construct
KLALKLALKALKAALKLA

SEQ ID NO: 1046 - protein, 9 aa, synthetic construct
CAGRRSAYC

SEQ ID NO: 1047 - protein, 9 aa, synthetic construct
CNRRTKAGC

SEQ ID NO: 1048 - protein, 9 aa, synthetic construct
CREAGRKAC

SEQ ID NO: 1049 - protein, 9 aa, synthetic construct
CLSDGKRKC

SEQ ID NO: 1050 - protein, 12 aa, synthetic construct
TSPLNIHNGQKL

SEQ ID NO: 1051 - protein, 12 aa, synthetic construct
APWHLSSQYSRT

SEQ ID NO: 1052 - protein, 12 aa, synthetic construct
SFHQFARATLAS

SEQ ID NO: 1053 - protein, 12 aa, synthetic construct
SNNNVRPIHIWP

SEQ ID NO: 1054 - protein, 9 aa, synthetic construct
NLVPMVATV

SEQ ID NO: 1055 - protein, 14 aa, synthetic construct
QYIKANSKFIGITE

SEQ ID NO: 1056 - protein, 34 aa, synthetic construct
KSSQYIKANSKFIGITERRRRRRRRLNVPMVATV

SEQ ID NO: 1057 - protein, 29 aa, synthetic construct
  UNSURE 1: note = Fluorescent label
KSSQYIKANSKFIGITEAAALNVPMVATV

SEQ ID NO: 1058 - protein, 21 aa, synthetic construct
  UNSURE 1: note = Fluorescent label
  UNSURE 21: note = Norbornene
KSSQYIKANSKFIGITERRRR

SEQ ID NO: 1059 - protein, 23 aa, synthetic construct
RGCSSSKYAPSYYHVMPKQNSTL

SEQ ID NO: 1060 - protein, 27 aa, synthetic construct
RLAIDKRFRVFTKPVDPDEVPDYVTVI

SEQ ID NO: 1061 - protein, 27 aa, synthetic construct
SVKKQFEELTLGEFLKLDRERAKNKIA

SEQ ID NO: 1062 - protein, 22 aa, synthetic construct
RQRLLEKIRVLEAEKEKNAYQL

SEQ ID NO: 1063 - protein, 29 aa, synthetic construct
RSEELLSQVQFLYTSLLKQQEEQTRVALL

SEQ ID NO: 1064 - protein, 21 aa, synthetic construct
PGQTLIYYVDEKAPEFSMQGL

SEQ ID NO: 1065 - protein, 24 aa, synthetic construct
NDLAYANDFASEYYLESEGQPLSA

SEQ ID NO: 1066 - protein, 28 aa, synthetic construct
DKRYRKIVDVNFQSLLDEDQAPPSSLLV

SEQ ID NO: 1067 - protein, 23 aa, synthetic construct
KEKGDLELEIIVLNDRVTDLVNQ

SEQ ID NO: 1068 - protein, 21 aa, synthetic construct
RSSTPPLHVHVDENTPVHVHI

SEQ ID NO: 1069 - protein, 21 aa, synthetic construct
SQGNELFRSGQFAEAAGKYSA

SEQ ID NO: 1070 - protein, 20 aa, synthetic construct
ENLDSVFAQDQEHQVELELL

SEQ ID NO: 1071 - protein, 24 aa, synthetic construct
VLNEKKQIYAIKYVNLEEADNQTL

SEQ ID NO: 1072 - protein, 22 aa, synthetic construct
AIRNQGYSTVTFDGTPSYGHTP

SEQ ID NO: 1073 - protein, 24 aa, synthetic construct
AALGTRPPINMEELDESYQKVIEL

SEQ ID NO: 1074 - protein, 29 aa, synthetic construct
ILNNFPHSIARQKRILVNLSMVENKLVEL

SEQ ID NO: 1075 - protein, 18 aa, synthetic construct
ALIVFWKYRRFQRNTGEM

SEQ ID NO: 1076 - protein, 22 aa, synthetic construct
TVSGNILTIRLTAADHRQLQLS

SEQ ID NO: 1077 - protein, 24 aa, synthetic construct
PRDDAQISEKLRKAFDDIAKYFSK

SEQ ID NO: 1078 - protein, 26 aa, synthetic construct
NQVERPQMTFGSLQRIFPKIMPKKPA

SEQ ID NO: 1079 - protein, 22 aa, synthetic construct
SDPKKKTTKVAKKGKAVRRGRR

SEQ ID NO: 1080 - protein, 24 aa, synthetic construct
TFPDLESEFQAALSRKVAELVHFL

SEQ ID NO: 1081 - protein, 26 aa, synthetic construct
KASDSLQLVFGIELMEVDPIGHVYIF

SEQ ID NO: 1082 - protein, 25 aa, synthetic construct
KVLEHVVRVNARVRIAYPSLREAAL

SEQ ID NO: 1083 - protein, 19 aa, synthetic construct
WGPRALVETSYVKVLHHLL

SEQ ID NO: 1084 - protein, 7 aa, synthetic construct
YLHPSYY

SEQ ID NO: 1085 - protein, 9 aa, synthetic construct
ALDDFSKQL

SEQ ID NO: 1086 - protein, 9 aa, synthetic construct
APKTPKYKF

SEQ ID NO: 1087 - protein, 9 aa, synthetic construct
APHXGVVFL

SEQ ID NO: 1088 - protein, 9 aa, synthetic construct
APHGVVLHV

SEQ ID NO: 1089 - protein, 9 aa, Synthetic construct
ALDDFSKQL

SEQ ID NO: 1090 - protein, 9 aa, synthetic construct
ALLDKFPVL

SEQ ID NO: 1091 - protein, 9 aa, synthetic construct
ALRKPTDNY

SEQ ID NO: 1092 - protein, 9 aa, synthetic construct
APKEIIFXL

SEQ ID NO: 1093 - protein, 9 aa, synthetic construct
ALADKFPVL

SEQ ID NO: 1094 - protein, 9 aa, Synthetic construct
AADDFSKQL

SEQ ID NO: 1095 - protein, 9 aa, synthetic construct
ALADKFPVL

SEQ ID NO: 1096 - protein, 9 aa, synthetic construct
IELSLIFYL

SEQ ID NO: 1097 - protein, 9 aa, synthetic construct
ALFTRFFYV

SEQ ID NO: 1098 - protein, 9 aa, synthetic construct
FCNDPFLVY

SEQ ID NO: 1099 - protein, 9 aa, synthetic construct
MELSLIDFY

SEQ ID NO: 1100 - protein, 9 aa, synthetic construct
NVFAFPFTY

SEQ ID NO: 1101 - protein, 9 aa, synthetic construct
KEFLEGETL

SEQ ID NO: 1102 - protein, 9 aa, synthetic construct
ALFTRFFYV

SEQ ID NO: 1103 - protein, 34 aa, synthetic construct; identical to SEQ ID NO: 1056
  UNSURE 1: note = Fluorescent label

SEQ ID NO: 1104 - protein, 33 aa, synthetic construct
  UNSURE 1: note = Fluorescent label
THVDIRTLEDLLMGTLRRRRRRRRRAHYNIVTF

SEQ ID NO: 1105 - protein, 25 aa, synthetic construct
  UNSURE 1: note = Fluorescent label
THVDIRTLEDLLMGTLRAHYNIVTF

SEQ ID NO: 1106 - protein, 34 aa, synthetic construct
  UNSURE 1: note = Fluorescent label
EEKEIIVIKDTEKKDQSRRRRRRRRSQFNVPMLY

SEQ ID NO: 1107 - protein, 26 aa, synthetic construct
  UNSURE 1: note = Fluorescent label
EEKEIIVIKDTEKKDQSSQFNVPMLY

SEQ ID NO: 1108 - protein, 34 aa, synthetic construct; identical to SEQ ID NO: 1056
  UNSURE 1: note = Fluorescent label

SEQ ID NO: 1109 - protein, 29 aa, synthetic construct
  UNSURE 1: note = Fluorescent label
KSSQYIKANSKFIGITEAAALNVPMVATV

SEQ ID NO: 1110 - protein, 21 aa, synthetic construct
  UNSURE 1: note = Fluorescent label
  UNSURE 21: note = Norbornene
KSSQYIKANSKFIGITERRRR

SEQ ID NO: 1111 - protein, 20 aa, synthetic construct
EKMKKDFRAMKDLAQQINLS

SEQ ID NO: 1112 - protein, 19 aa, synthetic construct
EKMKKDFRAMKDLAQQINL

SEQ ID NO: 1113 - protein, 19 aa, synthetic construct
KMKKDFRAMKDLAQQINLS

SEQ ID NO: 1114 - protein, 20 aa, synthetic construct
EKMKKDFRAMKDLAQQINLS

SEQ ID NO: 1115 - protein, 8 aa, synthetic construct
TLDSKTQS

SEQ ID NO: 1116 - protein, 8 aa, synthetic construct
LDSKTQSL

SEQ ID NO: 1117 - protein, 29 aa, synthetic construct
KSSQYIKANSKFIGITEAAALNVPMVATV

SEQ ID NO: 1118 - protein, 33 aa, synthetic construct
KSSQYIKANSKFIGITERRRRRRRRNVPMVATV

SEQ ID NO: 1119 - protein, 26 aa, synthetic construct
TGIPVVKPRQLIATDDAVPLGPGKGV

SEQ ID NO: 1120 - protein, 19 aa, synthetic construct
AFVAEDKFITLAAESVSLS

SEQ ID NO: 1121 - protein, 18 aa, synthetic construct
QCRDLLRATVFPETVPSL

SEQ ID NO: 1122 - protein, 27 aa, synthetic construct
RNQYKERLMELQEAVRWTEMIRASREN

SEQ ID NO: 1123 - protein, 26 aa, synthetic construct
VGDGVWVSIRLDSTLRLYHAHTYQHL

SEQ ID NO: 1124 - protein, 21 aa, synthetic construct
LENLDSVFAQDQEHQVELELL

SEQ ID NO: 1125 - protein, 19 aa, synthetic construct
LLLRTPYSSDNLYQMTSQL

SEQ ID NO: 1126 - protein, 29 aa, synthetic construct
AQYRIHTHGVFRGIQDVRRVPGVAPTLVR

SEQ ID NO: 1127 - protein, 25 aa, synthetic construct
DVDKLHFTPRIQRLNELEAQTRVKL

SEQ ID NO: 1128 - protein, 20 aa, synthetic construct
KLRYRYTLDDLYPMMNALKL

SEQ ID NO: 1129 - protein, 17 aa, synthetic construct
VMKTRLEADEVAAQLER

SEQ ID NO: 1130 - protein, 22 aa, synthetic construct
RSSTPPLHVHVDENTPVHVHIK

SEQ ID NO: 1131 - protein, 26 aa, synthetic construct
SLADVDPMQLDSSVRFDSVGGLSNHI

SEQ ID NO: 1132 - protein, 22 aa, synthetic construct
SERQLRLLFDQAYQMRPSIIFF

SEQ ID NO: 1133 - protein, 22 aa, synthetic construct
PLWHESDFAVDNVGNRTLRRRR